(19) United States
(12) Patent Application Publication
(10) Pub. No.: US 2005/0260713 A1
(43) Pub. Date: Nov. 24, 2005

(54) Polypeptides and nucleic acids encoding same

(76) Inventors: Esha A. Gangolli, Madison, CT (US); Meera Patturajan, Branford, CT (US); Corine A.M. Vernet, Branford, CT (US); Uriel M. Malyankar, Branford, CT (US); Ramesh Kekuda, Norwalk, CT (US); David J. Stone, Guilford, CT (US); David Anderson, Branford, CT (US); Richard A. Shimkets, Guilford, CT (US); Catherine E. Burgess, Wethersfield, CT (US); Bryan D. Zerhusen, Branford, CT (US); Xiaohong Liu, Branford, CT (US); Kimberly A. Spytek, New Haven, CT (US); Stacie J. Casman, North Haven, CT (US); Ference L. Boldog, North Haven, CT (US); Glennda Smithson, Guilford, CT (US); Li Li, Branford, CT (US); Weizhen Ji, Branford, CT (US); John R. MacDougall, Hamden, CT (US)

Correspondence Address: CURAGEN CORPORATION, 322 EAST MAIN STREET, BRANFORD, CT, 06405, US

(21) Appl. No.: 11/113,424

(22) Filed: Apr. 21, 2005

Related U.S. Application Data
(62) Division of application No. 10/029,020, filed on Dec. 19, 2001.
(60) Provisional application No. 60/256,704, filed on Dec. 19, 2000.
(60) Provisional application No. 60/311,590, filed on Aug. 10, 2001.
(60) Provisional application No. 60/257,314, filed on Dec. 20, 2000.
(60) Provisional application No. 60/311,613, filed on Aug. 10, 2001.
(60) Provisional application No. 60/315,617, filed on Aug. 29, 2001.
(60) Provisional application No. 60/307,506, filed on Jul. 24, 2001.
(60) Provisional application No. 60/322,358, filed on Sep. 14, 2001.
(60) Provisional application No. 60/294,075, filed on May 29, 2001.
(60) Provisional application No. 60/288,153, filed on May 2, 2001.

(51) Int. Cl. C07H 21/04; C12P 21/06; C07K 16/22; C07K 14/485

(52) U.S. Cl. 435/69.1; 435/320.1; 435/325; 530/399; 530/388.25; 536/23.5

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application is a Divisional of U.S. Ser. No. 10/029,020 filed Dec. 19, 2001, pending, which claims priority from U.S. provisional patent application Ser. Nos. 60/256,704 filed Dec. 19, 2000 (attorney docket CURA-525); 60/311,590 filed Aug. 10, 2001 (attorney docket CURA-525 IFC-01); 60/257,314 filed Dec. 20, 2000 (attorney docket CURA-526); 60/311,613, filed Aug. 10, 2001 (attorney docket CURA-526 IFC-01); 60/315,617 filed Aug. 29, 2001 (attorney docket CURA-526 IFC-02); 60/307,506 filed Jul. 24, 2001 (attorney docket CURA-526B1); 60/322,358 filed Sep. 14, 2001 (attorney docket CURA-526C1); 60/294,075 filed May 29, 2001 (attorney docket CURA-526E1); and 60/288,153 filed May 2, 2001 (attorney docket CURA-526F1), each of which is incorporated herein by reference.

### FIELD OF THE INVENTION

The invention generally relates to nucleic acids and polypeptides encoded thereby.

### BACKGROUND OF THE INVENTION

The epidermal growth factor (EGF) superfamily comprises a diverse group of proteins that function as secreted signaling molecules, growth factors, and components of the extracellular matrix, many with a role in vertebrate development. EGF-related proteins with C1s-like (CUB) domains have been reported. The CUB domain is found in 16 functionally diverse proteins such as the dorso-ventral patterning protein tolloid, bone morphogenetic protein-1, a family of spermadhesins, complement subcomponents C1s/C1r and the neuronal recognition molecule A5. Most of these proteins are known to be involved in developmental processes. The second domain is found mostly among developmentally-regulated proteins and spermadhesins.

The adipocyte complement related protein-3 (ACRP3), is a 30 kDa serum protein made and secreted exclusively from adipocyte cells, which is implicated in energy homeostasis and obesity. ACRP3 is structurally similar to complement factor C1q and to a hibernation-specific protein isolated from the plasma of Siberian chipmunks; it forms large homo-oligomers that undergo a series of post-translational modifications (see, Scherer P E, et al., J Biol Chem Nov. 10, 1995;270 (45):26746-9). ACRP30 is a close homologue of the complement protein C1q, which is involved in the recognition of microbial surfaces and antibody-antigen complexes in the classical pathway of complement. The crystal structure of a homotrimeric fragment of ACRP3 has been solved to 2.1 Å resolution. The structure reveals homology to the tumor necrosis factor (TNF) family. Identical folding topologies, key residue conservations, and similarity of trimer interfaces and intron positions establish an evolutionary link between the TNF and C1q families.

C1q is the first subcomponent of the C1 complex of the classical pathway of complement activation. Several functions have been assigned to C1q, which include antibody-dependent and independent immune functions, and are considered to be mediated by C1q receptors present on the effector cell surface. There remains some uncertainty about the identities of the receptors that mediate C1q functions. Some of the previously described C1q receptor molecules, such as gC1qR and cC1qR, now appear to have less of a role in C1q functions than in functions unrelated to C1q. The problem of identifying receptor proteins with complementary binding sites for C1q has been compounded by the highly charged nature of the different domains in C1q. Although newer candidate receptors like C1qR(p) and CR1 have emerged, full analysis of the C1q-C1q receptor interactions is still at an early stage. In view of the diverse functions that C1q is considered to perform, it has been speculated that several C1q-binding proteins may act in concert, as a C1q receptor complex, to bring about C1q mediated functions. Some major advances have been made in last few years. Experiments with gene targeted homozygous C1q-deficient mice have suggested a role for C1q in modulation of the humoral immune response, and also in protection against development of autoimmunity. The recently described crystal structure of ACRP-30, has revealed a new C1q/TNF superfamily of proteins. Although the members of this superfamily may have diverse functions, there may be a common theme in their phylogeny and modular organisation of their distinctive globular domains.

The first component of complement is a calcium-dependent complex of the 3 subcomponents C1q, C1r, and C1s. Subcomponent C1q binds to immunoglobulin complexes with resulting serial activation of C1r (enzyme), C1s (proenzyme) and the other 8 components of complement. C1q is composed of 3 different species of chains, called A, B, and C. Fragments of the A chain of C1q have been sequenced. The total A chain contains 190 amino acids. C1q shares with collagen the presence of hydroxyproline in its amino acid sequence.

Beta-adrenergic receptor kinase (beta-ARK1) phosphorylates the beta-2-adrenergic receptor and appears to mediate agonist-specific desensitization observed at high agonist concentrations. Beta-ARK1 is an ubiquitous cytosolic enzyme that specifically phosphorylates the activated form of the beta-adrenergic and related G-protein-coupled receptors. The beta-ARK1 gene spans approximately 23 kb and is composed of 21 exons. Beta-AR kinase (beta-ARK1) is known to be elevated in failing human heart tissue and its activity resulting in rapid desensitization via the abnormal coupling or uncoupling of beta-adrenergic receptor to G protein, receptor down-regulation, internalization and degradation, may account for some of the abnormalities of contractile function in the heart disease (see, Post, S. R., Hammond, H. K., Insel, P. A., 1999, Annu. Rev. Pharmacol. Vol. 39: 343-360) incorporated by reference.

The TEN-M4 protein belongs to the ODZ/TENM family of proteins. This family was first identified in Drosophila as being a pair-rule gene affecting segmentation of the early embryo. It was the first pair-rule gene identified that was not a transcription factor, but a type II transmembrane protein. Vertebrate homologs of the TENM family have been identified in mouse and zebrafish. In the mouse, TEN-M4 expression was found to be on the cell surface, in the brain, trachea as well as developing limb and bone. Analysis of the TEN-M1 protein reveals that it can bind to itself, making it likely that TEN-M4 may be a dimeric moiety as well. In cell culture experiments, fragments of the TEN-M proteins can bind the Drosophila PS2 integrins. In addition, members of the TEN-M family have been identified to be downstream of the endoplasmic reticulum stress response pathway, which alters the response of cells to their environment. This suggests that the ODZ/TENM family may be involved in cell adhesion, spreading and motility. Translocations leading to the fusion of this gene with the NRG1/HGL gene from chromosome 8 have been found to generate a paracrine growth factor for one mammary carcinoma cell line, termed gamma-heregulin.

Out At First is expressed in clusters of cells during germband extension, throughout the developing nervous system, and in the gonads of both sexes throughout the lifecycle. Mutation of the Drosophila gene is fatal and causes nervous system defects.

Butyrophilin plays several crucial roles in T-cell activation. The protein is known to be expressed in spleen and liver.

Sugar transport is a critical feature of many cell types in the body as energy storage and metabolism or defects thereof can cause a variety of human diseases. Glucose tranporter 4 (GLUT4) is critical to insulin-sensitive glucose uptake.

Mouse EphA6 (also known as m-ehk2) belongs to the superfamily of receptor tyrosine kinases, which constitute the largest family of oncogenes. This family includes prominent growth factor receptors such as those for epidermal growth factor, platelet-derived growth factor etc. Members of this superfamily influence cell shape, mobility, differentiation and proliferation. Within this superfamily, the Ephrin (Eph) receptors constitute the largest subfamily. Eph receptors and their ligands, ephrins, are known to be involved in several normal developmental processes, including formation of segmented structures, axon guidance, cell adhesion and development of vasculature. Ephrin receptors are classified into two main subtypes: EphA receptors bind to GPI-anchored ephrin-A ligands, while EphB receptors bind to ephrin-B proteins that have a transmembrane and cytoplasmic domain. The EphA6 receptor is highly expressed in the mouse brain and inner ear, including the cochlea. This receptor is also differentially expressed relative to the other ephrin receptors in certain regions of the primate neocortex during development. In addition, it is found in the developing retina and optic tectum in the chicken.

### SUMMARY OF THE INVENTION

The present invention is based in part on nucleic acids encoding proteins that are members of the following protein families: EGF related SCUBE1-like proteins, Adipocyte Complement Related proteins, complement C1q tumor necrosis factor-like proteins, β-Adrenergic Receptor Kinase-like proteins, TENM4-like proteins, Out At First-like proteins, EphA6-ehk2-like proteins, Glucose Transporter-like proteins, Type Ia Membrane Sushi-Containing Domain-like proteins, Type Ia Membrane Sushi-Containing Domain proteins, Butyrophilin-like proteins, and Butyrophilin Precursor B7-DC-like proteins. The novel polynucleotides and polypeptides are referred to herein as NOV1, NOV2a, NOV2b, NOV2c, NOV2d, NOV3, NOV4, NOV5a, NOV5b, NOV6a, NOV6b, NOV7, NOV8, NOV9, NOV10a, NOV10b and NOV11. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS:1, 3, 5, 7, 9, 11, 13, 15, 17, 19, 23, 25, 27, 29, 31 and 33. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 20, 22, 24, 26, 28, 30, 32 and 34. The nucleic acid can be, for example, a genomic DNA fragment or a cDNA molecule that includes the nucleic acid sequence of any of SEQ ID NOS:1, 3, 5, 7, 9, 11, 13, 15, 17, 19, 21, 23, 25, 27, 29, 31 and 33.

Also included in the invention is an oligonucleotide, e.g., an oligonucleotide which includes at least 6 contiguous nucleotides of a NOVX nucleic acid (e.g., SEQ ID NOS:1, 3, 5, 7, 9, 11, 13, 15, 17, 19, 21, 23, 25, 27, 29, 31 and 33) or a complement of said oligonucleotide. Also included in the invention are substantially purified NOVX polypeptides (SEQ ID NOS:2, 4, 6, 8, 10, 12, 14, 16, 18, 20, 22, 24, 26, 28, 30, 32 and 34). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., Von Hippel-Lindau (VHL) syndrome, cirrhosis, transplantation disorders, pancreatitis, obesity, diabetes, autoimmune disease, renal artery stenosis, interstitial nephritis, glomerulonephritis, polycystic kidney disease, systemic lupus erythematosus, renal tubular acidosis, IgA nephropathy, hypercalcemia, Lesch-Nyhan syndrome, developmental defects, cataract, spinal cord injury, Alzheimer's disease, muscular dystrophy, acoustic trauma, cancer, learning and memory defects, infertility, cardiomyopathies, atherosclerosis, hypertension, congenital heart defects, aortic stenosis, atrial septal defect, atrioventricular canal defect, ductus arteriosus, pulmonary stenosis, subaortic stenosis, ventricular septal defect, valve diseases, tuberous sclerosis, scleroderma, endometriosis, hemophilia, hypercoagulation, idiopathic thrombocytopenic purpura, immunodeficiencies, graft versus host disease, dementia, stroke, Parkinson's disease, Huntington's disease, cerebral palsy, epilepsy, multiple sclerosis, ataxia-telangiectasia, leukodystrophies, behavioral disorders, addiction, anxiety, pain, neurodegeneration, familial hypercholesterolemia, hyperlipoproteinemia II phenotype, tendinous xanthomas, corneal arcus, coronary artery disease, planar xanthomas, webbed digits, hypercholesterolemia, fertility, xanthomatosis, hepatitis C infection, regulation, synthesis, transport, recycling, or turnover of LDL receptors, cerebral arteriopathy with subcortical infarcts and leukoencephalopathy, epiphyseal dysplasia, multiple 1, ichthyosis, nonlamellar and nonerythrodermic, congenital, leukemia, T-cell acute lymphoblastoid, pseudoachondroplasia, SCID, autosomal recessive, T-negative/B-positive type, C3 deficiency, diabetes mellitus, insulin-resistant, with acanthosis nigricans, glutaricaciduria, type I, hypothyroidism, congenital, leprechaunism, liposarcoma, mucolipidosis IV, persistent mullerian duct syndrome, type I, Rabson-Mendenhall syndrome, thyroid carcinoma, with cell oxyphilia, erythrocytosis, malaria, to, bleeding disorder due to defective thromboxane A2 receptor, cerebellar ataxia, convulsions, familial febrile, cyclic hematopoiesis, fucosyltransferase-6 deficiency, GAMT deficiency, psoriasis, actinic keratosis, tuberous sclerosis, acne, hair growth, allopecia, pigmentation disorders, endocrine disorders, trauma, immunological disease, respiratory disease, gastro-intestinal diseases, reproductive health, neurological diseases, bone marrow transplantation, metabolic and endocrine diseases, allergy and inflammation, nephrological disorders, hematopoietic disorders, urinary system disorders, atopy; osteoporosis-pseudoglioma syndrome; Smith-Lemli-Opitz syndrome, type I; Smith-Lemli-Opitz syndrome, type II; xeroderma pigmentosum, Asthma, diabetes mellitus, susceptibility to IDDM; angioedema, paraganglioma, familial nonchromaffin, neuroprotection; Lambert-Eaton myasthenic syndrome, digestive system disorders, all or some of the protease/protease inhibitor deficiency disorders, acyl-CoA dehydrogenase, brachydactyly, carbamoylphosphate synthetase I deficiency, cardiomyopathy cataract Coppock-like, cataract crystalline aculeiform, cataract polymorphic congenital, cataract variable zonular pulverulent, cataracts punctate progressive juvenile-onse, choreoathetosis familial paroxysmal, craniofacial-deafness-hand syndrome, ichthyosis lamellar, type 2, myopathy, desmin-related cardioskeletal, resistance/susceptibility to TB, rhabdomyosarcoma alveolar, Waardenburg syndrome type I and type III, Alport syndrome autosomal recessive, Bjornstad syndrome, hematuria, hyperoxaluria primary, type 1, syndactyly type 1, hyperproglucagonemia, Bethlem myopathy, brachydactyly type E, brachydactyly-mental retardation syndrome, Finnish lethal neonatal metabolic syndrome, Simpson-Golabi-Behmel syndrome, Beckwith-Wiedemann syndrome, pathogen infections, heart disease, prostate cancer, angiogenesis and wound healing, modulation of apoptosis, neuropsychiatric disorders, age-related disorders, pathological disorders involving spleen, thymus, lung, and peritoneal macrophages and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

NOVX nucleic acids and polypeptides are further useful in the generation of antibodies that bind immuno-specifically to the novel NOVX substances for use in therapeutic or diagnostic methods. These NOVX antibodies may be generated according to methods known in the art, using prediction from hydrophobicity charts, as described in the “Anti-NOVX Antibodies” section below. The disclosed NOVX proteins have multiple hydrophilic regions, each of which can be used as an immunogen. These NOVX proteins can be used in assay systems for functional analysis of various human disorders, which will help in understanding of pathology of the disease and development of new drug targets for various disorders.

The NOVX nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides referred to herein as NOV1, NOV2a, NOV2b, NOV2c, NOV2d, NOV3, NOV4, NOV5a, NOV5b, NOV6a, NOV6b, NOV7, NOV8, NOV9, NOV10a, NOV10b and NOV11. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID NO | | |
|---|---|---|---|---|
| NOVX | Internal | (nucleic | SEQ ID NO | |
| ASSIGNMENT | Identification | acid) | (polypeptide) | Homology |
| 1 | CG55758-01 | 1 | 2 | SCUBE1-like |
| 2a | CG55724-01 | 3 | 4 | Adipocyte Complement |
| | | | | Related Protein |
| 2b | CG55724-03 | 5 | 6 | Cq1 TNF-like |
| 2c | CG55724-04 | 7 | 8 | Cq1 TNF-like |
| 2d | CG55724-06 | 9 | 10 | Cq1 TNF-like |
| 3 | CG50345-01 | 11 | 12 | β-Adrenergic Receptor |
| | | | | Kinase-like |
| 4 | CG50301-01 | 13 | 14 | TENM4-like |
| 5a | CG55764-01 | 15 | 16 | Out At First-like |
| 5b | CG55764-02 | 17 | 18 | Out At First-like |
| 6a | CG55704-01 | 19 | 20 | EphA6-ehk-like |
| 6b | CG55704-03 | 21 | 22 | EphA6-ehk-like |
| 7 | CG94323538 | 23 | 24 | Glucose Transporter-like |
| 8 | CG95545-01 | 25 | 26 | Type Ia Membrane Sushi- |
| | | | | containing domain |
| 9 | CG95545-02 | 27 | 28 | Type Ia Membrane Sushi- |
| | | | | containing domain |
| 10a | CG55746-01 | 29 | 30 | Butyrophilin-like |
| 10b | CG55746-05 | 31 | 32 | Butyrophilin Precursor B7- |
| | | | | DC |
| 11 | CG50329-01 | 33 | 34 | Butyrophilin-like |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

NOV1 is homologous to an EGF-Related SCUBE1-like family of proteins. Thus, the NOV1 nucleic acids, polypeptides, antibodies and related compounds according to the invention will be useful in therapeutic and diagnostic applications implicated in, for example; cancer, obesity, endometriosis, trauma, viral, bacterial, or parasitic infections, allergy, asthma, endocrine disfunctions, diabetes, growth and reproductive disorders, and other diseases, disorders and conditions of the like.

NOV2 is homologous to the adipocyte complement C1q Tumor Necrosis Factor-like family of proteins. Thus NOV2 nucleic acids, polypeptides, antibodies and related compounds according to the invention will be useful in therapeutic and diagnostic applications implicated in, for example; cancer, inflammation, neurological disorders, neuropsychiatric disorders, obesity, diabetes, viral/bacterial/parasitic infections, autoimmune diseases, renal artery stenosis, renal tubular acidosis, hypercalcemia, IgA nephropathy, Lesch-Nyhan syndrome, glomerulonephritis, interstitial nephritis, polycystic kidney disease, trauma, regeneration, Alzheimer's disease, allergies, addiction, anxiety, ataxia-telangiectasia, asthma, ARDS, atherosclerosis, behavioral disorders, aortic stenosis, atrial septal defect (ASD), atrioventricular (A-V) canal defect, ductus arteriosus, allergy, cerebral palsy, congenital adrenal hyperplasia, cirrhosis, cardiomyopathy, congenital heart defects, diabetes, diverticular disease, epilepsy, emphysema, endometriosis, endocrine dysfunctions, graft versus host disease, glomerulonephritis, graft versus host disease (GVHD), growth and reproductive disorders, hemophilia, hypercoagulation, hypercalceimia, Huntington's disease, hypertension, hypogonadism, idiopathic thrombocytopenic purpura, immunodeficiencies, interstitial nephritis, IgA nephropathy, lymphaedema, inflammatory bowel disease, leukodystrophies, multiple sclerosis, muscular dystrophy, myasthenia gravis, neurodegeneration, neuroprotection, obesity, Parkinson's disease, pain, polycystic kidney disease, pulmonary stenosis, pancreatitis, renal artery stenosis, renal tubular acidosis, stroke, systemic lupus erythematosus, scleroderma, subaortic stenosis, transplantation, tuberous sclerosis, Von Hippel-Lindau (VHL) syndrome, ventricular septal defect (VSD) and other diseases, disorders and conditions of the like.

NOV3 is homologous to a family of beta-adrenergic receptor kinase-like proteins. Thus, the NOV3 nucleic acids and polypeptides, antibodies and related compounds according to the invention will be useful in therapeutic and diagnostic applications implicated in, for example: cardiac disorders and disorders of myocontractility and the like.

NOV4 is homologous to the TEN-M4-like family of proteins. Thus, NOV4 nucleic acids, polypeptides, antibodies and related compounds according to the invention will be useful in therapeutic and diagnostic applications implicated in, for example: cancer, inflammation, neurological disorders, neuropsychiatric disorders, obesity, diabetes, viral/bacterial/parasitic infections, autoimmune diseases, renal artery stenosis, renal tubular acidosis, hypercalcemia, IgA nephropathy, Lesch-Nyhan syndrome, glomerulonephritis, interstitial nephritis, polycystic kidney disease, trauma, regeneration, Alzheimer's disease, allergies, addiction, anxiety, ataxia-telangiectasia, asthma, ARDS, atherosclerosis, behavioral disorders, aortic stenosis, atrial septal defect (ASD), atrioventricular (A-V) canal defect, ductus arteriosus, allergy, cerebral palsy, congenital adrenal hyperplasia, cirrhosis, cardiomyopathy, congenital heart defects, diabetes, diverticular disease, epilepsy, emphysema, endometriosis, endocrine dysfunctions, graft versus host disease, glomerulonephritis, graft versus host disease (GVHD), growth and reproductive disorders, hemophilia, hypercoagulation, hypercalceimia, Huntington's disease, hypertension, hypogonadism, idiopathic thrombocytopenic purpura, immunodeficiencies, interstitial nephritis, IgA nephropathy, lymphaedema, inflammatory bowel disease, leukodystrophies, multiple sclerosis, muscular dystrophy, myasthenia gravis, neurodegeneration, neuroprotection, obesity, Parkinson's disease, pain, polycystic kidney disease, pulmonary stenosis, pancreatitis, renal artery stenosis, renal tubular acidosis, stroke, systemic lupus erythematosus, scleroderma, subaortic stenosis, transplantation, tuberous sclerosis, Von Hippel-Lindau (VHL) syndrome, ventricular septal defect (VSD) and other diseases, disorders and conditions of the like.

NOV5 is homologous to the Out At First (OAF)-like family of proteins. Thus NOV5 nucleic acids, polypeptides, antibodies and related compounds according to the invention will be useful in therapeutic and diagnostic applications implicated in central nervous system diseases, disorders and conditions of the like.

NOV6 is homologous to the EphA6/ehk-2-like family of proteins. Thus NOV6 nucleic acids, polypeptides, antibodies and related compounds according to the invention will be useful in therapeutic and diagnostic applications implicated in, for example: cancer, inflammation, neurological disorders, neuropsychiatric disorders, obesity, diabetes, viral/bacterial/parasitic infections, autoimmune diseases, renal artery stenosis, renal tubular acidosis, hypercalcemia, IgA nephropathy, Lesch-Nyhan syndrome, glomerulonephritis, interstitial nephritis, polycystic kidney disease, trauma, regeneration, Alzheimer's disease, allergies, addiction, anxiety, ataxia-telangiectasia, asthma, ARDS, atherosclerosis, behavioral disorders, aortic stenosis, atrial septal defect (ASD), atrioventricular (A-V) canal defect, ductus arteriosus, allergy, cerebral palsy, congenital adrenal hyperplasia, cirrhosis, cardiomyopathy, congenital heart defects, diabetes, diverticular disease, epilepsy, emphysema, endometriosis, endocrine dysfunctions, graft versus host disease, glomerulonephritis, graft versus host disease (GVHD), growth and reproductive disorders, hemophilia, hypercoagulation, hypercalceimia, Huntington's disease, hypertension, hypogonadism, idiopathic thrombocytopenic purpura, immunodeficiencies, interstitial nephritis, IgA nephropathy, lymphaedema, inflammatory bowel disease, leukodystrophies, multiple sclerosis, muscular dystrophy, myasthenia gravis, neurodegeneration, neuroprotection, obesity, Parkinson's disease, pain, polycystic kidney disease, pulmonary stenosis, pancreatitis, renal artery stenosis, renal tubular acidosis, stroke, systemic lupus erythematosus, scleroderma, subaortic stenosis, transplantation, tuberous sclerosis, Von Hippel-Lindau (VHL) syndrome, ventricular septal defect (VSD) and other diseases, disorders and conditions of the like.

NOV7 is homologous to members of the glucose transporter-like family of proteins. Thus, the NOV7 nucleic acids, polypeptides, antibodies and related compounds according to the invention will be useful in therapeutic and diagnostic applications implicated in, for example; obesity, diabetes, cancer, inflammation, CNS diseases and other diseases, disorders and conditions of the like.

NOV8 is homologous to the Type Ia Membrane Sushi-Containing Domain-like family of proteins. Thus, NOV8 nucleic acids and polypeptides, antibodies and related compounds according to the invention will be useful in therapeutic and diagnostic applications implicated in, for example; cancer, inflammation, neurological disorders, neuropsychiatric disorders, obesity, diabetes, viral/bacterial/parasitic infections, autoimmune diseases, renal artery stenosis, renal tubular acidosis, hypercalcemia, IgA nephropathy, Lesch-Nyhan syndrome, glomerulonephritis, interstitial nephritis, polycystic kidney disease, trauma, regeneration, Alzheimer's disease, allergies, addiction, anxiety, ataxia-telangiectasia, asthma, ARDS, atherosclerosis, behavioral disorders, aortic stenosis, atrial septal defect (ASD), atrioventricular (A-V) canal defect, ductus arteriosus, allergy, cerebral palsy, congenital adrenal hyperplasia, cirrhosis, cardiomyopathy, congenital heart defects, diabetes, diverticular disease, epilepsy, emphysema, endometriosis, endocrine dysfunctions, graft versus host disease, glomerulonephritis, graft versus host disease (GVHD), growth and reproductive disorders, hemophilia, hypercoagulation, hypercalceimia, Huntington's disease, hypertension, hypogonadism, idiopathic thrombocytopenic purpura, immunodeficiencies, interstitial nephritis, IgA nephropathy, lymphaedema, inflammatory bowel disease, leukodystrophies, multiple sclerosis, muscular dystrophy, myasthenia gravis, neurodegeneration, neuroprotection, obesity, Parkinson's disease, pain, polycystic kidney disease, pulmonary stenosis, pancreatitis, renal artery stenosis, renal tubular acidosis, stroke, systemic lupus erythematosus, scleroderma, subaortic stenosis, transplantation, tuberous sclerosis, Von Hippel-Lindau (VHL) syndrome, ventricular septal defect (VSD) and other diseases, disorders and conditions of the like.

NOV9 is homologous to the Type Ia Membrane Sushi-Containing Domain-like family of proteins. Thus, NOV9 nucleic acids and polypeptides, antibodies and related compounds according to the invention will be useful in therapeutic and diagnostic applications implicated in, for example: cancer, inflammation, neurological disorders, neuropsychiatric disorders, obesity, diabetes, viral/bacterial/parasitic infections, autoimmune diseases, renal artery stenosis, renal tubular acidosis, hypercalcemia, IgA nephropathy, Lesch-Nyhan syndrome, glomerulonephritis, interstitial nephritis, polycystic kidney disease, trauma, regeneration, Alzheimer's disease, allergies, addiction, anxiety, ataxia-telangiectasia, asthma, ARDS, atherosclerosis, behavioral disorders, aortic stenosis, atrial septal defect (ASD), atrioventricular (A-V) canal defect, ductus arteriosus, allergy, cerebral palsy, congenital adrenal hyperplasia, cirrhosis, cardiomyopathy, congenital heart defects, diabetes, diverticular disease, epilepsy, emphysema, endometriosis, endocrine dysfunctions, graft versus host disease, glomerulonephritis, graft versus host disease (GVHD), growth and reproductive disorders, hemophilia, hypercoagulation, hypercalceimia, Huntington's disease, hypertension, hypogonadism, idiopathic thrombocytopenic purpura, immunodeficiencies, interstitial nephritis, IgA nephropathy, lymphaedema, inflammatory bowel disease, leukodystrophies, multiple sclerosis, muscular dystrophy, myasthenia gravis, neurodegeneration, neuroprotection, obesity, Parkinson's disease, pain, polycystic kidney disease, pulmonary stenosis, pancreatitis, renal artery stenosis, renal tubular acidosis, stroke, systemic lupus erythematosus, scleroderma, subaortic stenosis, transplantation, tuberous sclerosis, Von Hippel-Lindau (VHL) syndrome, ventricular septal defect (VSD) and other diseases, disorders and conditions of the like.

NOV10 is homologous to the butyrophilin-like family of proteins. Thus, NOV10 nucleic acids and polypeptides, antibodies and related compounds according to the invention will be useful in therapeutic and diagnostic applications implicated in, for example; cancer, inflammation, neurological disorders, neuropsychiatric disorders, obesity, diabetes, viral/bacterial/parasitic infections, autoimmune diseases, renal artery stenosis, renal tubular acidosis, hypercalcemia, IgA nephropathy, Lesch-Nyhan syndrome, glomerulonephritis, interstitial nephritis, polycystic kidney disease, trauma, regeneration, Alzheimer's disease, allergies, addiction, anxiety, ataxia-telangiectasia, asthma, ARDS, atherosclerosis, behavioral disorders, aortic stenosis, atrial septal defect (ASD), atrioventricular (A-V) canal defect, ductus arteriosus, allergy, cerebral palsy, congenital adrenal hyperplasia, cirrhosis, cardiomyopathy, congenital heart defects, diabetes, diverticular disease, epilepsy, emphysema, endometriosis, endocrine dysfunctions, graft versus host disease, glomerulonephritis, graft versus host disease (GVHD), growth and reproductive disorders, hemophilia, hypercoagulation, hypercalceimia, Huntington's disease, hypertension, hypogonadism, idiopathic thrombocytopenic purpura, immunodeficiencies, interstitial nephritis, lymphaedema, inflammatory bowel disease, leukodystrophies, multiple sclerosis, muscular dystrophy, myasthenia gravis, neurodegeneration, neuroprotection, obesity, Parkinson's disease, pain, polycystic kidney disease, pulmonary stenosis, pancreatitis, renal artery stenosis, renal tubular acidosis, stroke, systemic lupus erythematosus, scleroderma, subaortic stenosis, transplantation, tuberous sclerosis, Von Hippel-Lindau (VHL) syndrome, ventricular septal defect (VSD) and other diseases, disorders and conditions of the like.

NOV11 is homologous to the cysteine sulfinic acid decarboxylase-like family of proteins. Thus, NOV11 nucleic acids and polypeptides, antibodies and related compounds according to the invention will be useful in therapeutic and diagnostic applications implicated in, for example; cancer, inflammation, neurological disorders, neuropsychiatric disorders, obesity, diabetes, viral/bacterial/parasitic infections, autoimmune diseases, renal artery stenosis, renal tubular acidosis, hypercalcemia, IgA nephropathy, Lesch-Nyhan syndrome, glomerulonephritis, interstitial nephritis, polycystic kidney disease, trauma, regeneration, Alzheimer's disease, allergies, addiction, anxiety, ataxia-telangiectasia, asthma, ARDS, atherosclerosis, behavioral disorders, aortic stenosis, atrial septal defect (ASD), atrioventricular (A-V) canal defect, ductus arteriosus, allergy, cerebral palsy, congenital adrenal hyperplasia, cirrhosis, cardiomyopathy, congenital heart defects, diabetes, diverticular disease, epilepsy, emphysema, endometriosis, endocrine dysfunctions, graft versus host disease, glomerulonephritis, graft versus host disease (GVHD), growth and reproductive disorders, hemophilia, hypercoagulation, hypercalceimia, Huntington's disease, hypertension, hypogonadism, idiopathic thrombocytopenic purpura, immunodeficiencies, interstitial nephritis, IgA nephropathy, lymphaedema, inflammatory bowel disease, leukodystrophies, multiple sclerosis, muscular dystrophy, myasthenia gravis, neurodegeneration, neuroprotection, obesity, Parkinson's disease, pain, polycystic kidney disease, pulmonary stenosis, pancreatitis, renal artery stenosis, renal tubular acidosis, stroke, systemic lupus erythematosus, scleroderma, subaortic stenosis, transplantation, tuberous sclerosis, Von Hippel-Lindau (VHL) syndrome, ventricular septal defect (VSD) and other diseases, disorders and conditions of the like.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

A disclosed NOV1 nucleic acid of 3137 nucleotides (also referred to as CG55758-01) encoding a novel EGF-Related Protein (SCUBE1)-like protein is shown in Table 1A. An open reading frame was identified beginning with an ATG initiation codon at nucleotides 78-80 and ending with a TGA codon at nucleotides 2973-2975. A putative untranslated region upstream from the initiation codon and downstream from the termination codon is underlined in Table 1A. The start and stop codons are in bold letters.

TABLE 1A
NOV1 Polynucleotide SEQ ID NO:1

AGCGCCTGCGGGAGCGGCCGGTCGGTCGGGTCCCCGCGCCCCGCACGCCCGCACGCCCAGCGGGGCCCGC
ATTGAGCATGGGCGCGGCGGCCGTGCGCTGGCACTTGTGCGTGCTGCTGGCCCTGGGCACACGCGGGCGG
CTGGCCGGGGGCAGCGGGCTCCCAGGGTCAGTCGACGTGGATGAGTGCTCAGAGGGCACAGATGACTGCC
ACATCGATGCCATCTGTCAGAACACGCCCAAGTCCTACAAATGCCTCTGCAAGCCAGGCTACAAGGGGGA
AGGCAAGCAGTGTGAAGACATTGACGAGTGTGAGAATGACTACTACAATGGGGGCTGTGTCCACGAGTGC
ATCAACATCCCGGGGAACTACAGGTGTACCTGCTTTGATGGCTTCATGCTGGCACACGATGGACACAACT
GCCTGGATGTGGACGAGTGTCAGGACAATAATGGTGGCTGCCAGCAGATCTGCGTCAATGCCATGGGCAG
CTACGAGTGTCAGTGCCACAGTGGCTTCCTCCTTAGTGACAACCAGCATACCTGCATCCACCGCTCCAAT
GAGGGTATGAACTGCATGAACAAAGACCATGGCTGTGCCCACATCTGCCGGGAGACGCCCAAAGGTGGGG
TGGCCTGCGACTGCAGGCCCGGCTTTGACCTTGCCCAAAACCAGAAGGACTGCACACTAACCTGTAATTA
TGGAAACGGAGGCTGCCAGCACAGCTGTGAGGACACAGACACAGGCCCCACGTGTGGTTGCCACCAGAAG
TACGCCCTCCACTCAGACGGTCGCACGTGCATCGAGACGTGCGCAGTCAATAACGGAGGCTGCGACCGGA
CATGCAAGGACACAGCCACTGGCGTGCGATGCAGCTGCCCCGTTGGATTCACACTGCAGCCGGACGGGAA
GACATGCAAAGACATCAACGAGTGCCTGGTCAACAACGGAGGCTGCGACCACTTCTGCCGCAACACCGTG
GGCAGCTTCGAGTGCGGCTGCCGGAAGGGCTACAAGCTGCTCACCGACGAGCGCACCTGCCAGGACATCG
ACGAGTGCTCCTTCGAGCGGACCTGTGACCACATCTGCATCAACTCCCCGGGCAGCTTCCAGTGCCTGTG
TCACCGCGGCTACATCCTCTACGGGACAACCCACTGCGGAGATGTGGACGAGTGCAGCATGAGCAACGGG
AGCTGTGACCAGCGCTGCGTCAACACCAAGGGCAGCTACGAGTGCGTCTGTCCCCCGGGGAGGCGGCTCC
ACTGGAACGGGAAGGATTGCGTGGAGACAGGCAAGTGTCTTTCTCGCGCCAAGACCTCCCCCCGGGCCCA
GCTGTCCTGCAGCAAGGCAGGCGGTGTGGAGAGCTGCTTCCTTTCCTGCCCGGCTCACACACTCTTCGTG
CCACAAGACTCGGAAAATAGCTACGTCCTGAGCTGCGGAGTTCCAGGGCCGCAGGGCAAGGCGCTGCAGA
AACGCAACGGCACCAGCTCTGGCCTCGGGCCCAGCTGCTCAGATGCCCCCACCACCCCCATCAAACAGAA
GGCCCGCTTCAAGATCCGAGATGCCAAGTGCCACCTCCGGCCCCACAGCCAGGCACGAGCAAAGGAGACC
GCCAGGCAGCCGCTGCTGGACCACTGCCATGTGACTTTCGTGACCCTCAAGTGTGACTCCTCCAAGAAGA
GGCGCCGTGGCCGCAAGTCCCCATCCAAGGAGGTGTCCCACATCACAGCAGAGTTTGAGATCGAGACAAA
GATGGAAGAGGCCTCAGGTACATGCGAAGCGGACTGCTTGCGGAAGCGAGCAGAACAGAGCCTGCAGGCC
GCCATCAAGACCCTGCGCAAGTCCATCGGCCGGCAGCAGTTCTATGTCCAGGTCTCAGGCACTGAGTACG
AGGTAGCCCAGAGGCCAGCCAAGGCGCTGGAGGGGCAGGGGGCATGTGGCGCAGGCCAGGTGCTACAGGA
CAGCAAATGCGTTGCCTGTGGGCCTGGCACCCACTTCGGTGGTGAGCTCGGCCAGTGTGTGTCATGTATG
CCAGGAACATACCAGGACATGGAAGGCCAGCTCAGTTGCACACCGTGCCCCAGCAGCGACGGGCTTGGTC
TGCCTGGTGCCCGCAACGTGTCGGAATGTGGAGGCCAGTGTTCTCCAGGCTTCTTCTCGGCCGATGGCTT
CAAGCCCTGCCAGGCCTGCCCCGTGGGCACGTACCAGCCTGAGCCCGGGCGCACCGGCTGCTTCCCCTGT
GGAGGGGGTTTGCTCACCAAACACGAAGGCACCACCTCCTTCCAGGACTGCGAGGCTAAAGTGCACTGCT
CCCCCGGCCACCACTACAACACCACCACCCACCGCTGCATCCGCTGCCCCGTCGGCACCTACCAGCCCGA
GTTTGGCCAGAACCACTGCATCACCTGTCCGGGCAACACCAGCACAGACTTCGATGGCTCCACCAACGTC
ACACACTGCAAAAGTCAGCACTGCGGCGGCGAGCTTGGTGACTACACCGGCTACATCGAGTCCCCCAACT
ACCCTGGCGACTACCCAGCCAACGCTGAATGCGTCTGGCACATCGCGCCTCCCCCAAAGCGCAGGATCCT
CATCGTGGTCCCTGAGATCTTCCTGCCCATCGAGGATGAGTGCGGCGATGTTCTGGTCATGAGGAAGAGT
GCCTCTCCCACGTCCATCACCACCTATGAGACCTGCCAGACCTACGAGAGGCCCATCGCCTTCACCTCCC
GCTCCCGCAAGCTCTGGATCCAGTTCAAATCCAATGAAGGCAACAGCGGCAAAGGCTTCCAAGTGCCCTA
TGTCACCTACGATGGTAAGATCCACTGTCTTCACGGCCCACTGTGCACGGCTCAGGCGGGGCCCTGGAGA
CACAGAGATGAGTCGCACGTCCCCGCCCTCAGGGAGCTGCGACCTGGCAGGTACAGACCTGGAAGCAGAA
CGAACACTGTCAGGGGCCAGAGCCAGACAGGCTGAGGGTGGTACCGGGTGGTACAGGCAAGACAGCGGTT
AGTGGCCTCTGCAGGCTTCAGCTGAGGTGCTGCCCAAGCAGGGTTTTGAGGGCTAAATAGGGGGTTCTTA
GTGAAACCCCGAGGAGGACAATACAGGTGCAGGGAGCCCCAGGTTCAAAGGCACAGA

In a search of public sequence databases, the NOV1 nucleic acid sequence, located on chromosome 22q13, demonstrates 88% identity to Mus Musculus EGF-related protein SCUBE1 (Genbank AF276425). Public nucleotide databases include all GenBank databases and the GeneSeq patent database.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1 BLAST analysis, e.g., Mus Musculus EGF-related protein SCUBE1, matched the Query NOV1 sequence purely by chance is 1.1e -17. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. (Wootton and Federhen, Methods Enzymol 266:554-571, 1996).

The disclosed NOV1 polypeptide (SEQ ID NO:2) encoded by SEQ ID NO:1 has 965 amino acid residues and is presented in Table 1B using the one-letter amino acid codes. Signal P, Psort and/or Hydropathy results predict that NOV1 has a signal peptide and is likely to be localized outside the cell with a certainty of 0.3700. In other embodiments, NOV1 may also be localized to the lysosome (lumen) with a certainty of 0.1900, the nucleus with a certainty of 0.1800, or in the endoplasmic reticulum (membrane) with a certainty of 0.1000. The most likely cleavage site for a NOV1 signal peptide is between amino acids 23 and 24, at: RLA-GG.

TABLE 1B
NOV1 Polypeptide SEQ ID NO:2

MGAAAVRWHLCVLLALGTRGRLAGGSGLPGSVDVDECSEGTDDCHIDAICQNTPKSYKCLCKPGYKGEGK
QCEDIDECENDYYNGGCVHECINIPGNYRCTCFDGFMLAHDGHNCLDVDECQDNNGGCQQICVNAMGSYE
CQCHSGFLLSDNQHTCIHRSNEGMNCMNKDHGCAHICRETPKGGVACDCRPGFDLAQNQKDCTLTCNYGN
GGCQHSCEDTDTGPTCGCHQKYALHSDGRTCIETCAVNNGGCDRTCKDTATGVRCSCPVGFTLQPDGKTC
KDINECLVNNGGCDHFCRNTVGSFECGCRKGYKLLTDERTCQDIDECSFERTCDHICINSPGSFQCLCHR
GYILYGTTHCGDVDECSMSNGSCDQGCVNTKGSYECVCPPGRRLHWNGKDCVETGKCLSRAKTSPRAQLS
CSKAGGVESCFLSCPAHTLFVPQDSENSYVLSCGVPGPQGKALQKRNGTSSGLGPSCSDAPTTPIKQKAR
FKIRDAKCHLRPHSQARAKETARQPLLDHCHVTFVTLKCDSSKKRRRGRKSPSKEVSHITAEFEIETKME
EASGTCEADCLRKRAEQSLQAAIKTLRKSIGRQQFYVQVSGTEYEVAQRPAKALEGQGACGAGQVLQDSK
CVACGPGTHFGGELGQCVSCMPGTYQDMEGQLSCTPCPSSDGLGLFGARNVSECGGQCSPGFFSADGFKP
CQACPVGTYQPEPGRTGCFPCGGGLLTKHEGTTSFQDCEAKVHCSPGHHYNTTTHRCIRCPVGTYQPEFG
QNHCITCPGNTSTDFDGSTNVTHCKSQHCGGELGDYTGYIESPNYPGDYPANAECVWHIAPPPKRRILIV
VPEIFLPIEDECGDVLVMRKSASPTSITTYETCQTYERPIAFTSRSRKLWIQFKSNEGNSGKGFQVPYVT
YDGKIHCLHGPLCTAQAGPWRHRDESHVPALRELRPGRYRPGSRTNTVRGQSQTG

A search of sequence databases reveals that the NOV1 amino acid sequence has 145 of 489 amino acid residues (29%) identical to, and 216 of 489 amino acid residues (44%) similar to, the 2489 amino acid residue ptnr:SPTREMBL-ACC:Q16744 protein from Homo sapiens (Human) (COMPLEMENT RECEPTOR 1). Public amino acid databases include the GenBank databases, SwissProt, PDB and PIR.

NOV 1 is expressed in at least the pituitary gland, the ovaries, and the trachea. This information was derived by determining the tissue sources of the sequences that were included in the invention including but not limited to SeqCalling sources, public EST sources, literature sources, and/or RACE sources.

Homologies to the above NOV1 polypeptide will be shared by the other NOV1 protein insofar as they are homologous to each other as shown below. The disclosed NOV1 polypeptide has homology to the amino acid sequences shown in the BLASTP data listed in Table 1C.

TABLE 1C
BLAST results for NOV1

| Gene Index/ | | Length | Identity | Positives | |
|---|---|---|---|---|---|
| Identifier | Protein/Organism | (aa) | (%) | (%) | Expect |
| gi\|12738840\|ref\|NP_073560.1\| | signal peptide, | 961 | 88 | 92 | 0.0 |
| (NM_022723) | CUB domain, EGF- | | | | |
| | like 1 | | | | |
| | [Mus musculus] | | | | |
| gi\|0190748\|ref\|NP_066025.1\| | Type Ia Membrane | 999 | 61 | 72 | 0.0 |
| (NM_020974) | Sushi-Containing | | | | |
| | Domain protein | | | | |
| | [Homo sapiens] | | | | |
| gi\|9910154\|ref\|NP_064436.1\| | Type Ia Membrane | 997 | 59 | 72 | 0.0 |
| (NM_020052) | sushi-Containing | | | | |
| | Domain protein; | | | | |
| | ICRFP703B1614Q5.1 | | | | |
| | ICRF2703N2430Q5.1 | | | | |
| | [Mus musculus] | | | | |
| gi\|5050926\|emb\|CAB44772.1\| | dJ100N22.1 (novel | 161 | 99 | 99 | 0.0 |
| (Z99756) | EGF-like domain | | | | |
| | containing | | | | |
| | protein) | | | | |
| | [Homo sapiens] | | | | |
| gi\|13518037\|ref\|NP_002371.2\| | matrilin 2 | 956 | 37 | 51 | 0.0 |
| (NM_002380) | precursor | | | | |
| | [Homo sapiens] | | | | |

The homology between these and other sequences is shown graphically in the ClustalW analysis shown in Table 1D. In the ClustalW alignment of the NOV1 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

[image]

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results for NOV1 as disclosed in Table 1E, were collected from the Conserved Domain Database (CDD) with Reverse Position Specific BLAST analyses. This BLAST analysis software samples domains found in the Smart and Pfam collections. For Table 1E and all successive DOMAIN sequence alignments, fully conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, HY, FYW.

Table 1E lists the domain description from DOMAIN analysis results against NOV1. This indicates that the NOV1 sequence has properties similar to those of other proteins known to contain this domain.

TABLE 1E
Domain Analysis of NOV1
gnl|Smart|smart00042, CUB, Domain first found in Clr, Cls, uEGF, and
bone morphogenetic protein; This domain is found mostly among
developmentally-regulated proteins. Spermadhesins contain only this
domain.
CD-Length = 114 residues, 99.1% aligned
Score = 85.5 bits (210), Expect = 1e−17

| | | | |
|---|---|---|---|
| Query: | 799 | CGGELGDYTGYIESPNYPGDYPANAECVWHIAPPPKRRILIVVPEIFLPIEDECG-DVLV | 857 |
| | | CGG L +G I SPNYP YP N CVW I+ PP RI + + L D C D + | |
| Sbjct: | 1 | CGGTLTASSGTITSPNYPNSYPNNLNCVWTISAPPGYRIELKFTDFDLESSDNCTYDYVE | 60 |
| Query: | 858 | MRKSASPTSITTYETCQTYERPIAFTSRSRKLWIQFKSNEGNSGKGFQVPYVT | 910 |
| | | + S +S C + P +S S + + F S+ +GF Y | |
| Sbjct: | 61 | IYDGPSTSSPLLGRFCGSELPPPIISSSSNSMTVTFVSDSSVQKRGFSARYSA | 113 |

The epidermal growth factor (EGF) superfamily comprises a diverse group of proteins that function as secreted signaling molecules, growth factors, and components of the extracellular matrix, many with a role in vertebrate development. A novel mammalian gene encoding an EGF-related protein with a CUB (C1s-like) domain that defines a new mammalian gene family. The SCUBE1 (signal peptide-CUB domain-EGF-related 1) gene was isolated from a developing mouse urogenital ridge cDNA library and is expressed prominently in the developing gonad, nervous system, somites, surface ectoderm, and limb buds. Mouse SCUBE1 was mapped to chromosome 15 and shown that it is orthologous to a human gene in the syntenic region of chromosome 22q13.EGF-related proteins with C1s-like (CUB) domains have been reported. The CUB domain is found in 16 functionally diverse proteins such as the dorso-ventral patterning protein tolloid, bone morphogenetic protein-1, a family of spermadhesins, complement subcomponents C1s/C1r and the neuronal recognition molecule A5. Most of these proteins are known to be involved in developmental processes. The second domain is found mostly among developmentally-regulated proteins and spermadhesins.

The disclosed NOV1 nucleic acid of the invention encoding an EGF-Related Protein (SCUBE1)-like protein includes the nucleic acid or a fragment thereof whose sequence is provided in Table 1A. The invention also includes a mutant or variant nucleic acid any of whose bases may be changed from the corresponding base shown in Table 1A while still encoding a protein that maintains its EGF-Related Protein (SCUBE1)-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 30% percent of the bases may be so changed.

The disclosed NOV1 protein of the invention includes an EGF-Related Protein (SCUBE1)-like protein whose sequence is provided in Table 1B. The invention also includes a mutant or variant protein any of whose residues may be changed from the corresponding residue shown in Table 1B while still encoding a protein that maintains its EGF-Related Protein (SCUBE1)-like activities and physiological functions, or a functional fragment thereof. In the mutant or variant protein, up to about 12% percent of the residues may be so changed.

The invention further encompasses antibodies and antibody fragments, such as F_{ab} or (F_{ab})₂, that bind immunospecifically to any of the proteins of the invention.

The above defined information for this invention suggests that this EGF-Related Protein (SCUBE1)-like protein (NOV1 ) may function as a member of a EGF-Related Protein (SCUBE1)-like protein family. Therefore, the NOV1 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV1 nucleic acids and proteins of the invention are useful in potential therapeutic applications implicated in cancer including but not limited to various pathologies and disorders as indicated below. For example, a cDNA encoding EGF-Related Protein (SCUBE1)-like protein (NOV1) may be useful in gene therapy, and the EGF-Related Protein (SCUBE1)-like protein (NOV1) may be useful when administered to a subject in need thereof. By way of nonlimiting example, the compositions of the present invention will have efficacy for treatment of patients suffering from cancer, trauma, viral/bacterial/parasitic infections, endometriosis, fertility, asthma, allergy, endocrine dysfunctions, diabetes, obesity, growth and reproductive disorders and other diseases, disorders and conditions of the like. The NOV1 nucleic acid encoding the EGF-Related Protein (SCUBE1)-like protein of the invention, or fragments thereof, may further be useful in diagnostic applications, wherein the presence or amount of the nucleic acid or the protein are to be assessed.

NOV1 nucleic acids and polypeptides are further useful in the generation of antibodies that bind immuno-specifically to the novel NOV1 substances for use in therapeutic or diagnostic methods. These antibodies may be generated according to methods known in the art, using prediction from hydrophobicity charts, as described in the “Anti-NOVX Antibodies” section below. The disclosed NOV1 proteins have multiple hydrophilic regions, each of which can be used as an immunogen. In one embodiment, a contemplated NOV1 epitope is from about amino acids 400 to 450. In other embodiments, a NOV1 epitope is from about amino acids 500 to 600, from about 1000-1100, from about 1500-1600 and 2500-2800. These novel proteins can be used in assay systems for functional analysis of various human disorders, which will help in understanding of pathology of the disease and development of new drug targets for various disorders.

NOV2 includes four adipocyte complement-related C1q Tumor Necrosis Factor-like proteins and nucleic acids encoding the same. The disclosed sequences are identified herein as NOV2a, NOV2b, NOV2c, and NOV2d.

NOV2a

A disclosed NOV2a nucleic acid of 874 nucleotides identified as SEQ IDNO:3 (also referred to as CG55724-01) encoding an adipocyte complement-related C1q Tumor Necrosis Factor-like protein is shown in Table 2A. An open reading frame was identified beginning with an ATG initiation codon at nucleotides 11-13 and ending with a TGA codon at nucleotides 674-676. Putative upstream and downstream untranslated regions are underlined.

TABLE 2A
NOV2a Polynucleotide SEQ ID NO:3

| | |
|---|---|
| CTCATGCGGGATGCTTCCATATGGTCTTGTTTCAGGAGCTTTGCCCTGTTCTGTTGAATG | 60 |
| CTCTCTAGACCCAGAGGACGAAGCTCTAAGGAGGTCACAGATGAGGAAGGGTTCACTGAG | 120 |
| TGTAGTAGATGCTGTCAGTGGCCCACCCACACCTCCAGGCCTACCAGGACGAGGGCGGGC | 180 |
| GGGCCTGAGCGGGAAGAACGGTTTCCCTGGCGACGGATCCTCTGCTATGCGCTCGGCCTT | 240 |
| CTCGGCGGCACGCACCACCCCCCTGGAGGGCACGTCGGAGATGGCGGTGACCTTCGACAA | 300 |
| GGTGTACGTGAACATCGGGGGCGACTTCGACGCGGCGGCCGGCGTGTTCCGCTGCCGTCT | 360 |
| GCCCGGCGCCTACTTCTTCTCCTTCACGCTGGGCAAGCTGCCGCGTAAGACGCTGTCGGT | 420 |
| TAAGCTGATGAAGAACCGCGACGAGGTGCAGGCCATGATTTACGACGACGGCGCGTCGCG | 480 |
| GCGCCGCGAGATGCAGAGCCAGAGCGTGATGCTGGCCCTGCGGCGCGGCGACGCCGTCTG | 540 |
| GCTGCTCAGCCACGACCACGACGGCTACGGCGCCTACAGCAACCACGGCAAGTACATCAC | 600 |
| CTTCTCCGGCTTCCTGGTGTACCCCGACCTCGCCCCCGCCGCCCCGCCGGGCCTCGGGGC | 660 |
| CTCGGAGCTACTGTGAGCCCCGGGCCAGAGAAGAGCCCGGGAGGGCCAGGGGCGTGCATG | 720 |
| CCAGGCCGGGCCCGAGGCTCGAAAGTCCCGCGCGAGCGCCACGGCCTCCGGGCGCGCCTG | 780 |
| GACTCTGCCAATAAAGCGGAAAGCGGGCACGCGCAGCGCCCGGCAGCCCAGGACTAAGCC | 840 |
| GAATCTGCAAAATCCATCAACTGCCGGCGCTGAA | |

The disclosed NOV2a nucleic acid sequence, localized to chromosome 11, has 294 of 485 bases (60%) identical to a gb:GENBANK-ID:AF192499|acc:AF192499.1 mRNA from Mus musculus (Mus musculus putative secreted protein ZSIG37 (Zsig37) mRNA, complete cds).

A NOV2a polypeptide (SEQ ID NO:4) encoded by SEQ ID NO:3 has 221 amino acid residues and is presented using the one-letter code in Table 2B. Signal P, Psort and/or Hydropathy results predict that NOV2b does not have a signal peptide and the NOV2a polypeptide is likely to be localized to the cytoplasm with a certainty of 0.4500. In other embodiments, NOV2a may also be localized to peroxisomal microbodies with a certainty of 0.2688, lysosomes with a certainty of 0.1937, or the mitochondrial matrix space with a certainty of 0.1000.

TABLE 2B
NOV2a Polypeptide SEQ ID NO:4

| | |
|---|---|
| MLPYGLVSGALPCSVECSLDPEDEALRRSQMRKGSLSVVDAVSGPPTPPGLPGRGRAGLS | 60 |
| GKNGFPGDGSSAMRSAFSAARTTPLEGTSEMAVTFDKVYVNIGGDFDAAAGVFRCRLPGA | 120 |
| YFFSFTLGKLPRKTLSVKLMKNRDEVQAMIYDDGASRRREMQSQSVMLALRRGDAVWLLS | 180 |
| HDHDGYGAYSNHGKYITFSGFLVYPDLAPAAPPGLGASELL | 221 |

The disclosed NOV2a amino acid sequence has 55 of 158 amino acid residues (34%) identical to, and 84 of 158 amino acid residues (53%) identity to the 244 amino acid residue pntr:SWISSPROT ACC:Q15848 protein from Homo sapiens (Human) (30 kDa adipocyte complement related protein precursor, ACRP30). The NOV2a adipocyte complement-related protein precursor disclosed in this invention is expressed in at least the following tissues: testis, kidney, whole embryo. This information was derived by determining the tissue sources of the sequences that were included in the invention including but not limited to SeqCalling sources, public EST sources, literature sources, and/or RACE sources. In addition, the sequence is predicted to be expressed in the following tissues because of the expression pattern of (GENBANK-ID: gb:GENBANK-ID:AF192499|acc:AF192499.1) a closely related Mus musculus putative secreted protein ZSIG37 (Zsig37) mRNA, complete cds homolog in species Mus musculus: adipocytes.

NOV2b: A disclosed NOV2b nucleic acid of 1277 nucleotides (also referred to as CG55724-03) encoding a complement related C1q Tumor Necrosis Factor-like protein is shown in Table 2C as SEQ ID NO:5. An open reading frame was identified beginning with an ATG initiation codon at nucleotides 225-227 and ending with a TGA codon at nucleotides 1077-1079. Putative upstream and downstream untranslated regions are underlined.

TABLE 2C
NOV2b Polynucleotide SEQ ID NO:5

| | |
|---|---|
| GAATTCGGCACGAGGCGCCCGGCCCCTGGCCCCAGCACCCTGTCCGCTGCCGCCTCAGAG | 60 |
| CCGGGAAAAGCAGCCGGAGCCCCCGCCGCCCCTGCCGCAGCGCGGGCGGTCAGCGCGCAG | 120 |
| CCCGGCACCCGCAGCCTGCAGCCTGCAGCCCGCAGCCCGCAGCCCGGAGCCAGATCGCGG | 180 |
| GCTCAGACCGAACCCGACTCGACCGCCGCCCCCAGCCAGGCGCCATGCTGCCGCTTCTGC | 240 |
| TGGGCCTGCTGGGCCCAGCGGCCTGCTGGGCCCTGGGCCCGACCCCCGGCCCGGGATCCT | 300 |
| CTGAGCTGCGCTCGGCCTTCTCGGCGGCACGCACCACCCCCCTGGAGGGCACGTCGGAGA | 360 |
| TGGCGGTGACCTTCGACAAGGTGTACGTGAACATCGGGGGCGACTTCGATGTGGCCACCG | 420 |
| GCCAGTTTCGCTGCCGCGTGCCCGGCGCCTACTTCTTCTCCTTCACGGCTGGCAAGGCCC | 480 |
| CGCACAAGAGCCTGTCGGTGATGCTGGTGCGAAACCGCGACGAGGTGCAGGCGCTGGCCT | 540 |
| TCGACGAGCAGCGGCCGCCAGGCGCGCGGCGCGCAGCCAGCCAGAGCGCCATGCTGCAGC | 600 |
| TCGACTACGGCGACACAGTGTGGCTGCGGCTGCATGGCGCCCCGCAGTACGCGCTAGGCG | 660 |
| CGCCCGGCGCCACCTTCAGCGGCTACCTAGTCTACGCCGACGCCGAGTTCGTCAACATTG | 720 |
| GCGGCGACTTCGACGCGGCGGCCGGCGTGTTCCGCTGCCGTCTGCCCGGCGCCTACTTCT | 780 |
| TCTCCTTCACGCTGGGCAAGCTGCCGCGTAAGACGCTGTCGGTTAAGCTGATGAAGAACC | 840 |
| GCGACGAGGTGCAGGCCATGATTTACGACGACGGCGCGTCGCGGCGCCGCGAGATGCAGA | 900 |
| GCCAGAGCGTGATGCTGGCCCTGCGGCGCGGCGACGCCGTCTGGCTGCTCAGCCACGACC | 960 |
| ACGACGGCTACGGCGCCTACAGCAACCACGGCAAGTACATCACCTTCTCCGGCTTCCTGG | 1020 |
| TGTACCCCGACCTCGCCCCCGCCGCCCCGCCGGGCCTCGGGGCCTCGGAGCTACTGTGAG | 1080 |
| CCCCGGGCCAGAGAAGAGCCCGGGAGGGCCAGGGGCGTGCATGCCAGGCCGGGCCCGAGG | 1140 |
| CTCGAAAGTCCCGCGCGAGCGCCACGGCCTCCGGGCGCGCCTGGACTCTGCCAATAAAGC | 1200 |
| GGAAAGCGGGCACGCGCAGCGCCCGGCAGCCCAGGACTAAGCCGAATCTGCAAAATCCAT | 1260 |
| CAACTGCCGGCGCTGAA | 1277 |

The disclosed NOV2b nucleic acid sequence, localized to chromosome 11, has 767 of 814 bases (94%) identical to a gb:GENBANK-ID:AF329838|acc:AF329838.1 mRNA from Homo sapiens (Homo sapiens complement C1q Tumor Necrosis Factor-related protein CTRP4 mRNA, complete cds).

A NOV2b polypeptide (SEQ ID NO:6) encoded by SEQ ID NO:5 has 284 amino acid residues and is presented using the one-letter code in Table 2D. Signal P, Psort and/or Hydropathy results predict that NOV2b has a signal peptide and is likely to be localized outside the cell with a certainty of 0.4801. In other embodiments, NOV2b may also be localized to microsomal bodies with a certainty of 0.2178, the endoplasmic reticulum (membrane or lumen) with a certainty of 0.1000. The most likely cleavage site for a NOV2b signal peptide is between amino acids 16 and 17, at: CWA-LG.

TABLE 2D
NOV2b Polypeptide SEQ ID NO:6

| | |
|---|---|
| MLPLLLGLLGPAACWALGPTPGPGSSELRSAFSAARTTPLEGTSEMAVTFDKVYVNIGGD | 60 |
| FDVATGQFRCRVPGAYFFSFTAGKAPHKSLSVMLVRNRDEVQALAFDEQRRPGARRAASQ | 120 |
| SAMLQLDYGDTVWLRLHGAPQYALGAPGATFSGYLVYADAEFVNIGGDFDAAAGVFRCRL | 180 |
| PGAYFFSFTLGKLPRKTLSVKLMKNRDEVQAMIYDDGASRRREMQSQSVMLALRRGDAVW | 240 |
| LLSHDHDGYGAYSNHGKYITFSGFLVYPDLAPAAPPGLGASELL | |

The disclosed NOV2b amino acid sequence has 55 of 158 amino acid residues (34%) identical to, and 84 of 158 amino acid residues (53%) identity to the 244 amino acid residue pntr:SPTREMBL ACC:Q9BXJ3 protein from Homo sapiens (Human) (complement C1q Tumor Necrosis Factor-related protein). The NOV2b complement-C1q tumor necrosis factor-like gene disclosed in this invention is expressed in at least the following tissues: brain, germ cell, kidney, pooled, testis, whole embryo. Expression information was derived from the tissue sources of the sequences that were included in the derivation of the sequence of CuraGen Acc. No. CG55724-03, CG55724-04, or CG55724-06.

NOV2c: A disclosed NOV2c nucleic acid of 1322 nucleotides (also referred to as CG55724-04) encoding a complement related C1q Tumor Necrosis Factor-like protein is shown in Table 2E as SEQ ID NO:7. An open reading frame was identified beginning with an ATG initiation codon at nucleotides 225-227 and ending with a TGA codon at nucleotides 1122-1124. Putative upstream and downstream untranslated regions are underlined.

TABLE 2E
NOV2c Polynucleotide SEQ ID NO:7

| | |
|---|---|
| GAATTCGGCACGAGGCGCCCGGCCCCTGGCCCCAGCACCCTGTCCGCTGCCGCCTCAGAG | 60 |
| CCGGGAAAAGCAGCCGGAGCCCCCGCCGCCCCTGCCGCAGCGCGGGCGGTCAGCGCGCAG | 120 |
| CCCGGCACCCGCAGCCTGCAGCCTGCAGCCCGCAGCCCGCAGCCCGGAGCCAGATCGCGG | 180 |
| GCTCAGACCGAACCCGACTCGACCGCCGCCCCCAGCCAGGCGCCATGCTGCCGCTTCTGC | 240 |
| TGGGCCTGCTGGGCCCAGCGGCCTGCTGGGCCCTGGGCCCGACCCCCGGCCCGGGATCCT | 300 |
| CTGAGCTGCGCTCGGCCTTCTCGGCGGCACGCACCACCCCCCTGGAGGGCACGTCGGAGA | 360 |
| TGGCGGTGACCTTCGACAAGGTGTACGTGAACATCGGGGGCGACTTCGATGTGGCCACCG | 420 |
| GCCAGTTTCGCTGCCGCGTGCCCGGCGCCTACTTCTTCTCCTTCACGGCTGGCAAGGCCC | 480 |
| CGCACAAGAGCCTGTCGGTGATGCTGGTGCGAAACCGCGACGAGGTGCAGGCGCTGGCCT | 540 |
| TCGACGAGCAGCGGCGGCCAGGCGCGCGGCGCGCAGCCAGCCAGAGCGCCATGCTGCAGC | 600 |
| TCGACTACGGCGACACAGTGTGGCTGCGGCTGCATGGCGCCCCGCACTACGCGCTAGGCG | 660 |
| CGCCCGGCGCCACCTTCAGCGGCTACCTAGTCTACGCCGACGCCGACGCTGGCCCCGGGC | 720 |
| CGCGGCACCAACCACTCGCCTTCGACACCGAGTTCGTCAACATTGGCGGCGACTTCGACG | 780 |
| CGGCGGCCGACGTGTTCCGCTGCCGTCTGCCCGGCGCCTACTTCTTCTCCTTCACGCTGG | 840 |
| GCAAGCTGCCGCGTAAGACGCTGTCGGTTAAGCTGATGAAGAACCGCGACGAGGTGCAGG | 900 |
| CCATGATTTACGACGACGGCGCGTCGCGGCGCCGCGAGATGCAGAGCCAGAGCGTGATGC | 960 |
| TGGCCCTGCGGCGCGGCGACGCCGTCTGGCTGCTCAGCCACGACCACGACGGCTACGGCG | 1020 |
| CCTACAGCAACCACGGCAAGTACATCACCTTCTCCGGCTTCCTGGTGTACCCCGACCTCG | 1080 |
| CCCCCGCCGCCCCGCCGGGCCTCGGGGCCTCGGAGCTACTGTGAGCCCCGGGCCAGAGAA | 1140 |
| GAGCCCGGGAGGGCCAGGGGCGTGCATGCCAGGCCGGGCCCGAGGCTCGAAAGTCCCGCG | 1200 |
| CGAGCGCCACGGCCTCCGGGCGCGCCTGGACTCTGCCAATAAAGCGGAAAGCGGGCACGC | 1260 |
| GCAGCGCCCGGCAGCCCAGGACTAAGCCGAATCTGCAAAATCCATCAACTGCCGGCGCTG | 1320 |
| AA | |

The disclosed NOV2c nucleic acid sequence, localized to chromosome 11, has 949 of 1136 bases (83%) identical to a gb:GENBANK-ID:AF329838|acc:AF329838.1 mRNA from Homo sapiens (Homo sapiens complement C1q Tumor Necrosis Factor-related protein CTRP4 mRNA, complete cds).

A NOV2c polypeptide (SEQ ID NO:8) encoded by SEQ ID NO:7 has 299 amino acid residues and is presented using the one-letter code in Table 2F. Signal P, Psort and/or Hydropathy results predict that NOV2c has a signal peptide and is likely to be localized outside the cell with a certainty of 0.4801. In other embodiments, NOV2c may also be localized to microsomal bodies with a certainty of 0.2178, the endoplasmic reticulum (membrane or lumen) with a certainty of 0.1000. The most likely cleavage site for a NOV2c signal peptide is between amino acids 16 and 17, at: CWA-LG.

TABLE 2F
NOV2c Polypeptide SEQ ID NO:8

| | |
|---|---|
| MLPLLLGLLGPAACWALGPTPGPGSSELRSAFSAARTTPLEGTSEMAVTFDKVYVNIGGD | 60 |
| FDVATGQFRCRVPGAYFFSFTAGKAPHKSLSVMLVRNRDEVQALAFDEQRRPGARRAASQ | 120 |
| SAMLQLDYGDTVWLRLHGAPHYALGAPGATFSGYLVYADADAGPGPRHQPLAFDTEFVNI | 180 |
| GGDFDAAADVFRCRLPGAYFFSFTLGKLPRKTLSVKLMKNRDEVQAMIYDDGASRRREMQ | 240 |
| SQSVMLALRRGDAVWLLSHDHDGYGAYSNHGKYITFSGFLVYPDLAPAAPPGLGASELL | |

The disclosed NOV2c amino acid sequence has 164 of 170 amino acid residues (96%) identical to, and 164 of 170 amino acid residues (96%) identity to the 329 amino acid residue pntr:SPTREMBL ACC:Q9BXJ3 protein from Homo sapiens (Human) (complement C1q Tumor Necrosis Factor-related protein). The NOV2c complement-C1q tumor necrosis factor-like gene disclosed in this invention is expressed in at least the following tissues: brain, germ cell, kidney, pooled, testis, whole embryo. Expression information was derived from the tissue sources of the sequences that were included in the derivation of the sequence of CuraGen Acc. No. CG55724-03, CG55724-04, or CG55724-06.

NOV2d: A disclosed NOV2d nucleic acid of 409 nucleotides (also referred to as CG55724-06) encoding a complement related C1q Tumor Necrosis Factor-like protein is shown in Table 2G as SEQ ID NO:X. An open reading frame was identified beginning with an ATG initiation codon at nucleotides 4-6 and ending with a TGA codon at nucleotides 403-405. Putative upstream and downstream untranslated regions are underlined.

TABLE 2G
NOV2d Polynucleotide SEQ ID NO:9

| | |
|---|---|
| ATTATGCTGCCGCTTCTGCTGGGCCTGCTGGGCCCAGCGGCCTGCTGGGCCCTGGGCCCG | 60 |
| ACCCCCGGCCCGGGATCCTCTGAGCTGCGCTCGGCCTTCTCGGCGGCACGCACCACCCCC | 120 |
| CTGGAGGGCACGTCGGAGATGGCGGTGACCTTCGACAAGGTGTACGTGAACATCGGGGGC | 180 |
| GACTTCGATGTGGCCACCGGCCAGTTTCGCTGCCGCGAGATGCAGAGCCAGAGCGTGATG | 240 |
| CTGGCCCTGCGGCGCGGCGACGCCGTCTGGCTGCTCAGCCACGACCACGACGGCTACGGC | 300 |
| GCCTACAGCAACCACGGCAAGTACATCACCTTCTCCGGCTTCCTGGTGTACCCCGACCTC | 360 |
| GCCCCCGCCGCCCCGCCGGGCCTCGGGGCCTCGGAGCTACTGTGAGCCC | 409 |

The disclosed NOV2d nucleic acid sequence, localized to chromosome 11, has 239 of 260 bases (91%) identical to a gb:GENBANK-ID:AF329838|acc:AF329838.1 mRNA from Homo sapiens (Homo sapiens complement C1q Tumor Necrosis Factor-related protein CTRP4 mRNA, complete cds).

A NOV2d polypeptide (SEQ ID NO:10) encoded by SEQ ID NO:9 has 133 amino acid residues and is presented using the one-letter code in Table 2H. Signal P, Psort and/or Hydropathy results predict that NOV2d has a signal peptide and is likely to be localized outside the cell with a certainty of 0.4801. In other embodiments, NOV2d may also be localized to microsomal bodies with a certainty of 0.1972, the endoplasmic reticulum (membrane or lumen) with a certainty of 0.1000. The most likely cleavage site for a NOV2d signal peptide is between amino acids 16 and 17, at: CWA-LG.

TABLE 2H
NOV2d Polypeptide SEQ ID NO:10

| | |
|---|---|
| MLPLLLGLLGPAACWALGPTPGPGSSELRSAFSAARTTPLEGTSEMAVTFDKVYVNIGGD | 60 |
| FDVATGQFRCREMQSQSVMLALRRGDAVWLLSHDHDGYGAYSNHGKYITFSGFLVYPDLA | 120 |
| PAAPPGLGASELL | |

The disclosed NOV2d amino acid sequence has 164 of 170 amino acid residues (96%) positives to, and 164 of 170 amino acid residues (96%) positives to the 329 amino acid residue pntr:SPTREMBL ACC:Q9BXJ3 protein from Homo sapiens (Human) (complement C1q Tumor Necrosis Factor-related protein). The NOV2d complement-C1q tumor necrosis factor-like gene disclosed in this invention is expressed in at least the following tissues: brain, germ cell, kidney, pooled, testis, whole embryo. Expression information was derived from the tissue sources of the sequences that were included in the derivation of the sequence of CuraGen Acc. No. CG55724-03, CG55724-04, or CG55724-06.

The disclosed NOV2 nucleic acids of the present invention are expressed in at least bone marrow, brain, thalamus, testis, lung, kidney, and germ cells. This information was derived by determining the tissue sources of the sequences that were included in the invention. SeqCalling sources: Adrenal gland/Suprarenal gland, Amygdala, Bone, Bone Marrow, Brain, Colon, Coronary Artery, Dermis, Epidermis, Foreskin, Hair Follicles, Heart, Hippocampus, Hypothalamus, Kidney, Liver, Lung, Lymph node, Lymphoid tissue, Mammary gland/Breast, Esophagus, Ovary, Pancreas, Parathyroid Gland, Peripheral Blood, Pineal Gland, Pituitary Gland, Placenta, Prostate, Retina, Salivary Glands, Small Intestine, Spleen, Stomach, Testis, Thalamus, Thymus, Tonsils, Trachea, Umbilical Vein, and Uterus.

NOV2 also has homology to the amino acid sequences shown in the BLASTP data listed in Table 2I.

TABLE 2I
BLAST results for NOV2

| Gene Index/ | Protein/ | Length | Identity | Positives | |
|---|---|---|---|---|---|
| Identifier | Organism | (aa) | (%) | (%) | Expect |
| gi\|13994273\|ref\|NP_114115.1\| | complement- | 329 | 87 | 88 | 4e−67 |
| (NM_031909) | c1q tumor | | | | |
| | necrosis | | | | |
| | factor- | | | | |
| | related | | | | |
| | protein 4 | | | | |
| | [Homo | | | | |
| | sapiens] | | | | |
| gi\|12835488\|dbj\|BAB23268.1\| | putative | 205 | 80 | 81 | 5e−61 |
| (AK004340) | [Mus | | | | |
| | musculus] | | | | |
| gi\|13385666\|ref\|NP_080437.1\| | RIKEN cDNA | 205 | 79 | 80 | 4e−60 |
| (NM_026161) | 0710001E10 | | | | |
| | gene [Mus | | | | |
| | musculus] | | | | |
| gi\|13994278\|ref\|NP_114116.1\| | complement- | 278 | 35 | 43 | 5e−17 |
| (NM_031910) | c1q tumor | | | | |
| | necrosis | | | | |
| | factor- | | | | |
| | related | | | | |
| | protein 6 | | | | |
| | [Homo | | | | |
| | sapiens] | | | | |
| gi\|16550291\|dbj\|BAB70947.1\| | unnamed | 248 | 36 | 49 | 2e−16 |
| (AK055541) | protein | | | | |
| | product | | | | |
| | [Homo | | | | |
| | sapiens] | | | | |

The homology of these sequences is shown graphically in the ClustalW analysis shown in Table 2J.

[image]

[image]

[image]

[image]

Tables 2K list the domain description from DOMAIN analysis results against NOV2. This indicates that the NOV2 sequence has properties similar to those of other proteins known to contain this domain.

TABLE 2K

Domain Analysis of NOV2
gnl|Smart|smart00110, ClQ, Complement component Clq domain.;
Globular domain found in many collagens and eponymously in
complement Clq. When part of full length proteins these domains
form a ‘bouquet’ due to the multimerization of heterotrimers.
The Clq fold is similar to that of tumour necrosis factor.
CD-Length = 132 residues, 84.1% aligned
Score = 86.7 bits (213), Expect = 1e−18

| | | | |
|---|---|---|---|
| Query: | 91 | MAVTFDKVYVNIGGDFDAAAGVFRCRLPGAYFFSFTLGKLPRKTLSVKLMKNRDEVQAMI | 150 |
| | | VFDKV N G +D + G F C +PG Y+FS+ + + + + V LMKN +V | |
| Sbjct: | 20 | QPVRFDKVLYNQQGHYDPSTGKFTCPVPGVYYFSYHI-ESKGRNVKVSLMKNGIQVMRE- | 77 |
| Query: | 151 | YDDGASRRREMQSQSVMLALRRGDAVWLLSHDHDGYGAYSNHGKYITFSGFLVY | 204 |
| | | D+ ++ S +L LR+GD VW L D G Y+ TFSGFL++ | |
| Sbjct: | 78 | CDEYQKGLYQVASGGALLQLRQGDQVW-LELDDKKNGLYAGEEVDSTFSGFLLF | 130 |

C1q is the first subcomponent of the C1 complex of the classical pathway of complement activation. Several functions have been assigned to C1q, which include antibody-dependent and independent immune functions, and are considered to be mediated by C1q receptors present on the effector cell surface. There remains some uncertainty about the identities of the receptors that mediate C1q functions. Some of the previously described C1q receptor molecules, such as gC1qR and cC1qR, now appear to have less of a role in C1q functions than in functions unrelated to C1q. The problem of identifying receptor proteins with complementary binding sites for C1q has been compounded by the highly charged nature of the different domains in C1q. Although newer candidate receptors like C1qR(p) and CR1 have emerged, full analysis of the C1q-C1q receptor interactions is still at an early stage. In view of the diverse functions that C1q is considered to perform, it has been speculated that several C1q-binding proteins may act in concert, as a C1q receptor complex, to bring about C1q mediated functions. Some major advances have been made in last few years. Experiments with gene targeted homozygous C1q-deficient mice have suggested a role for C1q in modulation of the humoral immune response, and also in protection against development of autoimmunity. The recently described crystal structure of ACRP-30, has revealed a new C1q/TNF superfamily of proteins. Although the members of this superfamily may have diverse functions, there may be a common theme in their phylogeny and modular organisation of their distinctive globular domains.

The novel polypeptide described in this application is homologous to adipocyte complement related protein 3 (ACRP3). The ACRP3 protein is made exclusively in adipocytes and its mRNA is induced over 100-fold during adipocyte differentiation. ACRP3 is structurally similar to complement factor C1q and to a hibernation-specific protein isolated from the plasma of Siberian chipmunks; it forms large homo-oligomers that undergo a series of post-translational modifications. A similar protein has a cluster of aromatic residues near the C terminus having high local similarity with collagens X and VIII and complement factor C1q. C1q is a subunit of the C1 enzyme complex that activates the serum complement system. C1q comprises 6 A, 6 B and 6 C chains. These share the same topology, each possessing a small, globular N-terminal domain, a collagen-like Gly/Pro-rich central region, and a conserved C-terminal region, the C1q domain. The C1q protein is produced by collagen-producing cells and shows sequence and structural similarity to collagens VIII and X, (see, Scherer P E, et al., J Biol Chem Nov. 10, 1995;270(45):26746-9 and Maeda K, et al., Biochem Biophys Res Commun Apr. 16, 1996;221(2):286-9), incorporated herein by reference.

The present invention includes chimeric or fusion proteins of the complement-C1q tumor necrosis factor-like protein, in which the complement-C1q tumor necrosis factor-like protein of the present invention is joined to a second polypeptide or protein that is not substantially homologous to the present novel protein. The second polypeptide can be fused to either the amino-terminus or carboxyl-terminus of the present CG55724-01, CG55724-03, CG55724-04, or CG55724-06 polypeptide. In certain embodiments a third nonhomologous polypeptide or protein may also be fused to the novel complement-C1q tumor necrosis factor-like protein such that the second nonhomologous polypeptide or protein is joined at the amino terminus, and the third nonhomologous polypeptide or protein is joined at the carboxyl terminus, of the CG55724-01, CG55724-03, CG55724-04, or CG55724-06 polypeptide. Examples of nonhomologous sequences that may be incorporated as either a second or third polypeptide or protein include glutathione S-transferase, a heterologous signal sequence fused at the amino terminus of the complement-C1q tumor necrosis factor-like protein, an immunoglobulin sequence or domain, a serum protein or domain thereof (such as a serum albumin), an antigenic epitope, and a specificity motif such as (His)₆. The invention further includes nucleic acids encoding any of the chimeric or fusion proteins described above.

The disclosed NOV2 nucleic acids of the invention encoding a complement-related C1q Tumor Necrosis Related Protein-like protein includes the nucleic acids whose sequence is provided in Table 2A, 2C, 2E and 2G or a fragment thereof. The invention also includes a mutant or variant nucleic acid any of whose bases may be changed from the corresponding base shown in Table 2A, 2C, 2E and 2G while still encoding a protein that maintains its complement-related C1q Tumor Necrosis Related Protein-like protein activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 40% (NOV2a), 6% (NOV2b), 6% (NOV2c) and 9% (NOV2d) of the bases may be so changed.

The disclosed NOV2 protein of the invention includes the complement-related C1q Tumor Necrosis Related Protein-like protein whose sequence is provided in Table 2B, 2D, 2F and 2G. The invention also includes a mutant or variant protein any of whose residues may be changed from the corresponding residue shown in Table 2B, 2D, 2F and 2G while still encoding a protein that maintains its the complement-related C1q Tumor Necrosis Related Protein-like activities and physiological functions, or a functional fragment thereof. In the mutant or variant protein, up to about 66% (NOV2a), 2% (NOV2b, NOV2c), and 9% (NOV2d) of the residues may be so changed.

The NOV2 nucleic acids and proteins of the invention are useful in potential therapeutic applications implicated in cancers, adrenoleukodystrophy, Alzheimer's disease, autoimmune disease, allergies, addiction, anxiety, ataxia-telangiectasia, asthma, ARDS, atherosclerosis, behavioral disorders, aortic stenosis, atrial septal defect (ASD), atrioventricular (A-V) canal defect, ductus arteriosus, allergy, cerebral palsy, congenital adrenal hyperplasia, cirrhosis, cardiomyopathy, congenital heart defects, diabetes, diverticular disease, epilepsy, emphysema, endometriosis, endocrine dysfunctions, graft versus host disease, glomerulonephritis, graft versus host disease (GVHD), growth and reproductive disorders, hemophilia, hypercoagulation, hypercalceimia, Huntington's disease, hypertension, hypogonadism, fertility, idiopathic thrombocytopenic purpura, immunodeficiencies, interstitial nephritis, IgA nephropathy, lymphaedema, inflammatory bowel disease, Lesch-Nyhan syndrome, leukodystrophies, multiple sclerosis, muscular dystrophy, myasthenia gravis, neurodegeneration, neuroprotection,obesity, Parkinson's disease, pain, polycystic kidney disease, pulmonary stenosis, pancreatitis, renal artery stenosis, renal tubular acidosis, stroke, systemic lupus erythematosus, scleroderma, subaortic stenosis, transplantation, tuberous sclerosis, Von Hippel-Lindau (VHL) syndrome, ventricular septal defect (VSD), valve diseases, Von Hippel-Lindau (VHL) syndrome, ulcers, and other diseases, pathologies and disorders. The NOV2 nucleic acid encoding the complement-related C1q Tumor Necrosis Related Protein-like protein, and the protein of the invention, or fragments thereof, may further be useful in diagnostic applications, wherein the presence or amount of the nucleic acid or the protein are to be assessed.

NOV2 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. These antibodies may be generated according to methods known in the art, using prediction from hydrophobicity charts, as described in the “Anti-NOVX Antibodies” section below. The disclosed NOVa, NOV2b, NOV2c, and NOV2d proteins have multiple hydrophilic regions, each of which can be used as an immunogen.

In one embodiment, a contemplated NOV2a epitope is from about amino acids 25 to 100. In another embodiment, a contemplated NOV2a epitope is from about amino acids 110 to 275. In other specific embodiments, contemplated NOV1 epitopes are from about amino acids 280 to 325, 350 to 425, 450 to 625, 650 to 690, 700 to 825, and 850 to 965.

In one embodiment, a contemplated NOV2b epitope is from about amino acids 20 to 50. In another embodiment, a contemplated NOV2b epitope is from about amino acids 55 to 65. In other specific embodiments, contemplated NOV2b epitopes are from about amino acids 90 to 145, 195 to 235, and 240 to 260.

In one embodiment, a contemplated NOV2c epitope is from about amino acids 20 to 50. In another embodiment, a contemplated NOV2c epitope is from about amino acids 55 to 65. In other specific embodiments, contemplated NOV2c epitopes are from about amino acids 90 to 145, 195 to 235, and 240 to 260.

In one embodiment, a contemplated NOV2d epitope is from about amino acids 18 to 40. In another embodiment, a contemplated NOV2d epitope is from about amino acids 42 to 47. In other specific embodiments, contemplated NOV2d epitopes are from about amino acids 60 to 80, 85 to 105, and 106 to 110.

NOV3

A disclosed NOV3 nucleic acid of 3073 nucleotides is set forth as SEQ ID NO:11 also referred to as CG50345-01) encoding a beta adrenergic receptor kinase-like protein is shown in Table 3A. An open reading frame was identified beginning with an ATG initiation codon at nucleotides 108-110 and ending with a TGA codon at nucleotides 2112-2114.

TABLE 3A
NOV3 Polynucleotide SEQ ID NO:11

| | |
|---|---|
| GGGTACCGAGCTCGAATTCCGGCTCGGCCTCGGGCGCGGCCGAGCGCCGCGCGAGCAGGA | 60 |
| GCGGCGGCGGCGGCGGCGGCGGCGGGAGGAGGCAGCGCCGGCCCAAGATGGCGGACCTGG | 120 |
| AGGCGGTGCTGGCCGACGTGAGCTACCTGATGGCCATGGAGAAGAGCAAGGCCACGCCGG | 180 |
| CCGCGCGCGCCAGCAAGAAGATACTGCTGCCCGAGCCCAGCATCCGCAGTGTCATGCAGA | 240 |
| AGTACCTGGAGGACCGGGGCGAGGTGACCTTTGAGAAGATCTTTTCCCAGAAGCTGGGGT | 300 |
| ACCTGCTCTTCCGAGACTTCTGCCTGAACCACCTGGAGGAGGCCAGGCCCTTGGTGGAAT | 360 |
| TCTATGAGGAGATCAAGAAGTACGAGAAGCTGGAGACGGAGGAGGAGCGTGTGGCCCGCA | 420 |
| GCCGGGAGATCTTCGACTCATACATCATGAAGGAGCTGCTGGCCTGCTCGCATCCCTTCT | 480 |
| CGAAGAGTGCCACTGAGCATGTCCAAGGCCACCTGGGGAAGAAGCAGGTGCCTCCGGATC | 540 |
| TCTTCCAGCCATACATCGAAGAGATTTGTCAAAACCTCCGAGGGGACGTGTTCCAGAAAT | 600 |
| TCATTGAGAGCGATAAGTTCACACGGTTTTGCCAGTGGAAGAATGTGGAGCTCAACATCC | 660 |
| ACCTGACCATGAATGACTTCAGCGTGCATCGCATCATTGGGCGCGGGGGCTTTGGCGAGG | 720 |
| TCTATGGGTGCCGGAAGCGTGACACAGGCAAGATGTACGCCATGAAGTGCCTGGACAAAA | 780 |
| AGCGCATCAAGATGAAGCAGGGGGAGACCCTGGCCCTGAACGAGCGCATCATGCTCTCGC | 840 |
| TCGTCAGCACTGGGGACTGCCCATTCATTGTCTGCATGTCATACGCGTTCCACACGCCAG | 900 |
| ACAAGCTCAGCTTCATCCTGGACCTCATGAACGGTGGGGACCTGCACTACCACCTCTCCC | 960 |
| AGCACGGGGTCTTCTCAGAGGCTGACATGCGCTTCTATGCGGCCGAGATCATCCTGGGCC | 1020 |
| TGGAGCACATGCACAACCGCTTCGTGGTCTACCGGGACCTGAAGCCAGCCAACATCCTTC | 1080 |
| TGGACGAGCATGGCCACGTGCGGATCTCGGACCTGGGCCTGGCCTGTGACTTCTCCAAGA | 1140 |
| AGAAGCCCCATGCCAGCGTGGGCACCCACGGGTACATGGCTCCGGAGGTCCTGCAGAAGG | 1200 |
| GCGTGGCCTACGACAGCAGTGCCGACTGGTTCTCTCTGGGGTGCATGCTCTTCAAGTTGC | 1260 |
| TGCGGGGGCACAGCCCCTTCCGGCAGCACAAGACCAAAGACAAGCATGAGATCGACCGCA | 1320 |
| TGACGCTGACGATGGCCGTGGAGCTGCCCGACTCCTTCTCCCCTGAACTACACTCCCTGC | 1380 |
| TGGAGGGGTTGCTGCAGAGGGATGTCAACCGGAGATTGGGCTGCCTGGGCCGAGGGGCTC | 1440 |
| AGGAGGTGAAAGAGAGCCCCTTTTTCCGCTCCCTGGACTGGCAGATGGTCTTCTTGCAGA | 1500 |
| GGTACCCTCCCCCGCTGATCCCCCCACGAGGGGAGGTGAACGCGGCCGACGCCTTCGACA | 1560 |
| TTGGCTCCTTCGATGAGGAGGACACAAAAGGAATCAAGCAGGAGGTGGCAGAGACTGTCT | 1620 |
| TCGACACCATCAACGCTGAGACAGACCGGCTGGAGGCTCGCAAGAAAGCCAAGAACAAGC | 1680 |
| AGCTGGGCCATGAGGAAGACTACGCCCTGGGCAAGGACTGCATCATGCATGGCTACATGT | 1740 |
| CCAAGATGGGCAACCCCTTTCTGACCCAGTGGCAGCGGCGGTACTTCTACCTGTTCCCCA | 1800 |
| ACCGCCTCGAGTGGCGGGGCGAGGGCGAGGCCCCGCAGAGCCTGCTGACCATGGAGGAGA | 1860 |
| TCCAGTCGGTGGAGGAGACGCAGATCAAGGAGCGCAAGTGCCTGCTCCTCAAGATCCGCG | 1920 |
| GTGGGAAACAGTTCATTTTGCAGTGCGATAGCGACCCTGAGCTGGTGCAGTGGAAGAAGG | 1980 |
| AGCTGCGCGACGCCTACCGCGAGGCCCAGCAGCTGGTGCAGCGGGTGCCCAAGATGAAGA | 2040 |
| ACAAGCCGCGCTCGCCCGTGGTGGAGCTGAGCAAGGTGCCGCTGGTCCAGCGCGGCAGTG | 2100 |
| CCAACGGCCTCTGACCCGCCCACCCGCCTTTTATAAACCTCTAATTTATTTTGTCGAATT | 2160 |
| TTTATTATTTGTTTTCCCGCCAAGCGAAAAGGTTTTATTTTGTAATTATTGTGATTTCCC | 2220 |
| GTGGCCCCAGCCTGGCCCAGCTCCCCCGGGAGGCCCCGCTTGCCTCGGCTCCTGCTGCAC | 2280 |
| CAACCCAGCCGCTGCCCGGCGCCCTCTGTCCTGACTTCAGGGGCTGCCCGCTCCCAGTGT | 2340 |
| CTTCCTGTGGGGGAAGAGCACAGCCCTCCCGCCCCTTCCCCGAGGGATGATGCCACACCA | 2400 |
| AGCTGTGCCACCCTGGGCTCTGTGGGCTGCACTTGTGCCATGGGACTGTGGGTGGCCCAT | 2460 |
| CCCCCCTCACCAGGGGCAGGCACAGCACAGGGATCCGACTTGAATTTTCCCACTGCACCC | 2520 |
| CCTCCTGCTGCAGAGGGGCAGGCCCTGCACTGTCCTGCTCCACAGTGTTGGCGAGAGGAG | 2580 |
| GGGCCCGTTGTCTCCCTGGCCCTCAAGGCTCCCACAGTGACTCGGGCTCCTGTGCCCTTA | 2640 |
| TTCAGGAAAAGCCTCTGTGTCACTGGCTGCCTCCACTCCCACTTCCCTGACACTGCGGGG | 2700 |
| CTTGGCTGAGAGAGTGGCATTGGCAGCAGGTGCTGCTACCCTCCCTGCTGTCCCCTCTTG | 2760 |
| CCCCAACCCCCAGCACCCGGGCTCAGGGACCACAGCAAGGCACCTGCAGGTTGGGCCATA | 2820 |
| CTGGCCTCGCCTGGCCTGAGGTCTCGCTGATGCTGGGCTGGGTGCGACCCCATCTGCCCA | 2880 |
| GGACGGGGCCGGCCAGGTGGGCGGGCAGCACAGCAAGGAGGCTGGCTGGGGCCTATCAGT | 2940 |
| GTGCCCCCCATCCTGGCCCATCAGTGTACCCCCGCCCAGACTGGCCAGCCCCACAGCCCA | 3000 |
| CGTCCTGTCAGTGCCGCCGCCTCGCCCACCGCATGCCCCCTGTGCCAGTGCTCTGCCTGT | 3060 |
| GTGTGTGCACTCT | |

The disclosed NOV3 nucleic acid sequence maps to chromosome 11q13 and has 1638 of 1666 bases (98%) identical to a gb:GENBANK-ID:HSBARK|acc:X61157.1 mRNA from Homo sapiens (H. sapiens mRNA for beta-adrenergic receptor kinase).

A disclosed NOV3 protein (SEQ ID NO:12) encoded by SEQ ID NO:11 has 668 amino acid residues, and is presented using the one-letter code in Table 3B. Signal P, Psort and/or Hydropathy results predict that NOV3 does have a signal peptide, and is likely to be localized to the nucleus with a certainty of 0.8800. In other embodiments NOV3 is also likely to be localized to perioxisomal microbodies with a certainty of 0.1582, mitochondrial matrix space with a certainty of 0.1000, to the lysosomal lumen with a certainty of 0.1000.

TABLE 3B
NOV3 Polypeptide SEQ ID NO:12

| | |
|---|---|
| MADLEAVLADVSYLMAMEKSKATPAARASKKILLPEPSIRSVMQKYLEDRGEVTFEKIFS | 60 |
| QKLGYLLFRDFCLNHLEEARPLVEFYEEIKKYEKLETEEERVARSREIFDSYIMKELLAC | 120 |
| SHPFSKSATEHVQGHLGKKQVPPDLFQPYIEEICQNLRGDVFQKFIESDKFTRFCQWKNV | 180 |
| ELNIHLTMNDFSVHRIIGRGGFGEVYGCRKRDTGKMYAMKCLDKKRIKMKQGETLALNER | 240 |
| IMLSLVSTGDCPFIVCMSYAFHTPDKLSFILDLMNGGDLHYHLSQHGVFSEADMRFYAAE | 300 |
| IILGLEHMHNRFVVYRDLKPANILLDEHGHVRISDLGLACDFSKKKPHASVGTHGYMAPE | 360 |
| VLQKGVAYDSSADWFSLGCMLFKLLRGHSPFRQHKTKDKHEIDRMTLTMAVELPDSFSPE | 420 |
| LHSLLEGLLQRDVNRRLGCLGRGAQEVKESPFFRSLDWQMVFLQRYPPPLIPPRGEVNAA | 480 |
| DAFDIGSFDEEDTKGIKQEVAETVFDTINAETDRLEARKKAKNKQLGHEEDYALGKDCIM | 540 |
| HGYMSKMGNPFLTQWQRRYFYLFPNRLEWRGEGEAPQSLLTMEEIQSVEETQIKERKCLL | 600 |
| LKIRGGKQFILQCDSDPELVQWKKELRDAYREAQQLVQRVPKMKNKPRSPVVELSKVPLV | 660 |
| QRGSANGL | |

The disclosed NOV3 amino acid has 359 of 642 amino acid residues (55%) identical to, and 497 of 497 amino acid residues (100%) similar to 497 of the 689 amino acid residue ptnr:SWISSNEW ACC:P25098 protein from Homo sapiens (Human) beta-adrenergic receptor kinase 1 (beta-ARK1, G-Protein Coupled Receptor Kinase 2).

The NOV3 sequence is expressed in at least the following tissues: brain-the Adrenal Gland/Suprarenal gland, Amygdala, Aorta, Bone, Bone Marrow, Brain, Cerebellum, Cervix, Chorionic Villus,Cochlea, Colon, Dermis, Epidermis, Foreskin, Hair Follicles, Heart, Hippocampus, Hypothalamus, Kidney, Liver, Lung, Lymph node, Lymphoid tissue, Mammary gland/Breast, Muscle, Myometrium, Ovary, Pancreas, Parotid Salivary glands, Pituitary Gland, Placenta, Prostate, Proximal Convoluted Tubule, Small Intestine, Spinal Chord, Retina, Spleen, Stomach, Substantia Nigra, Testis, Thymus, Thyroid, Tonsils, Umbilical Vein, Urinary Bladder, Uterus.

NOV3 also has homology to the amino acid sequences shown in the BLASTP data listed in Table 3C.

TABLE 3C
BLAST results for NOV3

| Gene Index/ | Protein/ | Length | Identity | Positives | |
|---|---|---|---|---|---|
| Identifier | Organism | (aa) | (%) | (%) | Expect |
| gi\|6978467\|ref\|NP_037029.1\| | adrenergic | 688 | 78 | 88 | 0.0 |
| (NM_012897) | receptor | | | | |
| | kinase, | | | | |
| | beta 2 | | | | |
| | (G-protein- | | | | |
| | linked | | | | |
| | receptor | | | | |
| | kinase) | | | | |
| | [Rattus | | | | |
| | norvegicus] | | | | |
| gi\|4206092\|gb\|AAD11419.1\| | G protein | 689 | 90 | 93 | 0.0 |
| (AF087455) | receptor | | | | |
| | kinase 2 | | | | |
| | [Didelphis | | | | |
| | virginiana] | | | | |
| gi\|162684\|gb\|AAA30384.1\| | beta- | 689 | 94 | 94 | 0.0 |
| (M34019) | adrenergic | | | | |
| | receptor | | | | |
| | kinase [Bos taurus] | | | | |
| gi\|162735\|gb\|AAA30406.1\| | beta- | 688 | 82 | 89 | 0.0 |
| (M73216) | adrenergic | | | | |
| | receptor | | | | |
| | kinase 2 | | | | |
| | [Bos taurus] | | | | |
| gi\|5139484\|emb\|CAB45657.1\| | bK407F11.2 | 688 | 81 | 89 | 0.0 |
| (AL022329) | (adrenergic, | | | | |
| | beta, | | | | |
| | receptor | | | | |
| | kinase 2) | | | | |
| | [Homo sapiens] | | | | |

The homology of these sequences is shown graphically in the ClustalW analysis shown in Table 3D.

[image]

[image]

[image]

[image]

Table 3E lists the domain description from DOMAIN analysis results against NOV3. This indicates that the NOV3 sequence has properties similar to those of other proteins known to contain this domain.

TABLE 3E

Domain Analysis of NOV3
gnl|Smart|smart00220, S_TKc, Serine/Threonine protein kinases,
catalytic domain; Phosphotransferases. Serine or
threonine-specific kinase subfamily.
CD-Length = 256 residues, 100.0% aligned
Score = 237 bits (605), Expect = 1e−63

| | | | |
|---|---|---|---|
| Query: | 191 | FSVHRIIGRGGFGEVYGCRKRDTGKMYAMKCLDKKRIKMKQGETLALNERIMLSLVSTGD | 250 |
| | | + + ++G+G FG+VY R + TGK+ A+K + K+++K K+ E L E +L + D | |
| Sbjct: | 1 | YELLEVLGKGAFGKVYLARDKKTGKLVAIKVIKKEKLKKKKRER-ILREIKILKKL---D | 56 |
| Query: | 251 | CPFIVCMSYAFHTPDKLSFILDLMNGGDLHYHLSQHGVFSEADMRFYAAEIILGLEHMHN | 310 |
| | | P IV + F DKL +++ GGDL L + G SE + RFYA +I+ LE++H+ | |
| Sbjct: | 57 | HPNIVKLYDVFEDDDKLYLVMEYCEGGDLFDLLKKRGRLSEDEARFYARQILSALEYLHS | 116 |
| Query: | 311 | RFVVYRDLKPANILLDEHGHVRISDLGLACDFSKKKPHAS--VGTHGYMAPEVLQKGVAY | 368 |
| | | + +++RDLKP NILLD GHV+++D GLA + VGT YMAPEVL G Y | |
| Sbjct: | 117 | QGIIHRDLKPENILLDSDGHVKLADFGLAKQLDSGGTLLTTFVGTPEYMAPEVL-LGKGY | 175 |
| Query: | 369 | DSSADWFSLGCMLFKLLRGHSPFRQHKTKDK-HEIDRMTLTMAVELPDSFSPELHSLLEG | 427 |
| | | + D +SLG +L++LL G PF + SPE L++ | |
| Sbjct: | 176 | GKAVDIWSLGVILYELLTGKPPFPGDDQLLALFKKIGKPPPPFPPPEWKISPEAKDLIKK | 235 |
| Query: | 428 | LLQRDVNRRLGCLGRGAQEVKESPFF | 453 |
| | | LL +D +RL A+E E PFF | |
| Sbjct: | 236 | LLVKDPEKRL-----TAEEALEHPFF | 256 |

Beta-adrenergic receptor kinase (beta-ARK1) phosphorylates the beta-2-adrenergic receptor and appears to mediate agonist-specific desensitization observed at high agonist concentrations. Beta-ARK1 is an ubiquitous cytosolic enzyme that specifically phosphorylates the activated form of the beta-adrenergic and related G-protein-coupled receptors. The beta-ARK1 gene spans approximately 23 kb and is composed of 21 exons. Beta-AR kinase (beta-ARK1) is known to be elevated in failing human heart tissue and its activity resulting in rapid desensitization via the abnormal coupling or uncoupling of beta-adrenergic receptor to G protein, receptor down-regulation, internalization and degradation, may account for some of the abnormalities of contractile function in the heart disease (see, Post, S. R., Hammond, H. K., Insel, P. A., 1999, Annu. Rev. Pharmacol. Vol.39: 343-360) incorporated by reference.

Beta-adrenergic receptor kinase (beta-ARK1) phosphorylates the beta-2-adrenergic receptor and appears to mediate agonist-specific desensitization observed at high agonist concentrations. Beta-ARK1 is an ubiquitous cytosolic enzyme that specifically phosphorylates the activated form of the beta-adrenergic and related G-protein-coupled receptors. The beta-ARK1 gene spans approximately 23 kb and is composed of 21 exons. Beta-AR kinase (beta-ARK1) is known to be elevated in failing human heart tissue and its activity resulting in rapid desensitization via the abnormal coupling or uncoupling of beta-adrenergic receptor to G protein, receptor down-regulation, internalization and degradation, may account for some of the abnormalities of contractile function in the heart disease (see, Post, S. R., Hammond, H. K., Insel, P. A., 1999, Annu. Rev. Pharmacol. Vol. 39: 343-360, incorporated herein by reference)

The protein similarity information, expression pattern, and map location for the Beta-adrenergic receptor kinase-like protein and nucleic acid disclosed herein suggest that this Beta-adrenergic receptor kinase may have important structural and/or physiological functions characteristic of the Serine-threonine protein kinase family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: cardiac diseases, myocardial contractility in failing heart and other diseases, disorders and conditions of the like. The disclosed NOV3 nucleic acid of the invention encoding a beta adrenergic receptor kinase -like protein includes the nucleic acid whose sequence is provided in Table 3A or a fragment thereof. The invention also includes a mutant or variant nucleic acid any of whose bases may be changed from the corresponding base shown in Table 3A while still encoding a protein that maintains beta adrenergic receptor kinase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 2 percent of the bases may be so changed.

The disclosed NOV3 protein of the invention includes the beta adrenergic receptor kinase-like protein whose sequence is provided in Table 3B. The invention also includes a mutant or variant protein any of whose residues may be changed from the corresponding residue shown in Table 3B while still encoding a protein that maintains beta adrenergic receptor kinase-like activities and physiological functions, or a functional fragment thereof. In the mutant or variant protein, up to about 1 percent of the residues may be so changed.

The protein similarity information, expression pattern, and map location for the beta adrenergic receptor kinase-like protein and nucleic acid (NOV3) disclosed herein suggest that NOV3 may have important structural and/or physiological functions characteristic of the beta adrenergic receptor kinase-like family. Therefore, the NOV3 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV3 nucleic acids and proteins of the invention are useful in potential diagnostic and therapeutic applications implicated in various diseases and disorders described below. For example, the compositions of the present invention will have efficacy for treatment of patients suffering from cancer, inflammation, retinal disorders, neurological disorders, neuropsychiatric disorders, obesity, diabetes, bleeding disorders and/or other pathologies. The NOV3 nucleic acid, or fragments thereof, may further be useful in diagnostic applications, wherein the presence or amount of the nucleic acid or the protein are to be assessed.

NOV3 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV3 polypeptide has multiple hydrophilic regions, each of which can be used as an immunogen. In one embodiment, a contemplated NOV3 epitope is from about amino acids 20 to 70. In another embodiment, a contemplated NOV3 epitope is from about amino acids 95 to 115. In other specific embodiments, contemplated NOV3 epitopes are from about amino acids 120 to 190, 280 to 300, 305 to 375, 395 to 420, and 415 to 660.

NOV4

A disclosed NOV4 nucleic acid of 8354 nucleotides is set forth as SEQ ID NO:13 (designated CuraGen Acc. No. CG50301-01) encoding a TEN-M4-like protein is shown in Table 4A. An open reading frame was identified beginning with an ATG initiation codon at nucleotides 35-37 and ending with a TAG codon at nucleotides 8342-8344. Putative untranslated regions are indicated by underline.

TABLE 4A
NOV4 Polynucleotide SEQ ID NO:13

| | |
|---|---|
| GTTTGTGGATGTGGAGGAGCGCGGGCCGGAGGCCATGGACGTGAAGGAGAGGAAGCCTTA | 60 |
| CCGCTCGCTGACCCGGCGCCGCGACGCCGAGCGCCGCTACACCAGCTCGTCCGCGGACAG | 120 |
| CGAGGAGGGCAAAGCCCCGCAGAAATCGTACAGCTCCAGCGAGACCCTGAAGGCCTACGA | 180 |
| CCAGGACGCCCGCCTAGCCTATGGCAGCCGCGTCAAGGACATTGTGCCGCAGGAGGCCGA | 240 |
| GGAATTCTGCCGCACAGGTGCCAACTTCACCCTGCGGGAGCTGGGGCTGGAAGAAGTAAC | 300 |
| GCCCCCTCACGGGACCCTGTACCGGACAGACATTGGCCTGCCCCAATGCGGCTACTCCAT | 360 |
| GGGGGCTGGCTCTGATGCCGACATGGAGGCTGACACGGTGCTGTCCCCTGAGCACCCCGT | 420 |
| GCGTCTGTGGGGCCGGAGCACACGGTCAGGGCGCAGCTCCTGCCTGTCCAGCCGGGCCAA | 480 |
| TTCCAATCTCACACTCACCGACACCGAGCATGAAAACACTGAGACTGATCATCCGGGCGG | 540 |
| CCTGCAGAACCACGCGCGGCTCCGGACGCCGCCGCCGCCGCTCTCGCACGCCCACACCCC | 600 |
| CAACCAGCACCACGCGGCCTCCATTAACTCCCTGAACCGGGGCAACTTCACGCCGAGGAG | 660 |
| CAACCCCAGCCCGGCCCCCACGGACCACTCGCTCTCCGGAGAGCCCCCTGCCGGCGGCGC | 720 |
| CCAGGAGCCTGCCCACGCCCAGGAGAACTGGCTGCTCAACAGCAACATCCCCCTGGAGAC | 780 |
| CAGGAACCTAGGCAAGCAGCCATTCCTAGGGACATTGCAGGACAACCTCATTGAGATGGA | 840 |
| CATTCTCGGCGCCTCCCGCCATGATGGGGCTTACAGTGACGGGCACTTCCTCTTCAAGCC | 900 |
| TGGAGGCACCTCCCCGCTCTTCTGCACCACATCACCAGGGTACCCACTGACGTCCAGCAC | 960 |
| AGTGTACTCTCCTCCGCCCCGACCCCTGCCCCGCAGCACCTTCGCCCGGCCGGCCTTTAA | 1020 |
| CCTCAAGAAGCCCTCCAAGTACTGTAACTGGAAGTGCGCAGCCCTGAGCGCCATCGTCAT | 1080 |
| CTCAGCCACTCTGGTCATCCTGCTGGCATACTTTGTGGCCATGCACCTGTTTGGCCTAAA | 1140 |
| CTGGCACCTGCAGCCGATGGAGGGGCAGATGTATGAGATCACGGAGGACACAGCCAGCAG | 1200 |
| TTGGCCTGTGCCAACCGACGTCTCCCTATACCCCTCAGGGGGCACTGGCTTAGAGACCCC | 1260 |
| TGACAGGAAAGGCAAAGGAACCACAGAAGGAAAGCCCAGTAGTTTCTTTCCAGAGGACAG | 1320 |
| TTTCATAGATTCTGGAGAAATTGATGTGGGAAGGCGAGCCTCCCAGAAGATTCCTCCTGG | 1380 |
| CACTTTCTGGAGATCTCAAGTGTTCATAGACCATCCTGTGCATCTGAAATTCAATGTGTC | 1440 |
| TCTGGGAAAGGCAGCCCTGGTTGGCATTTATGGCAGAAAAGGCCTCCCTCCTTCACATAC | 1500 |
| ACAGTTTGACTTTGTGGAGCTGCTGGATGGCAGGAGGCTCCTAACCCAGGAGGCGCGGAG | 1560 |
| CCTAGAGGGGACCCCGCGCCAGTCTCGGGGAACTGTGCCCCCCTCCAGCCATGAGACAGG | 1620 |
| CTTCATCCAGTATTTGGATTCAGGAATCTGGCACTTGGCTTTTTACAATGACGGAAAGGA | 1680 |
| GTCAGAAGTGGTTTCCTTTCTCACCACTGCCATTGAGTCGGTGGATAACTGCCCCAGCAA | 1740 |
| CTGCTATGGCAATGGTGACTGCATCTCTGGGACCTGCCACTGCTTCCTGGGTTTCCTGGG | 1800 |
| CCCCGACTGTGGCAGAGCCTCCTGCCCCGTGCTCTGTAGCGGAAATGGCCAATACATGAA | 1860 |
| AGGCAGATGCTTGTGCCACAGTGGCTGGAAAGGCGCTGAGTGCGATGTGCCCACCAACCA | 1920 |
| GTGTATCGATGTGGCCTGCAGCAACCATGGCACCTGCATCACGGGCACCTGCATCTGCAA | 1980 |
| CCCTGGCTACAAGGGCGAGAGCTGTGAGGAAGTGGACTGCATGGACCCCACATGTTCAGG | 2040 |
| CCGGGGTGTCTGCGTGAGAGGCGAATGCCATTGCTTTGTGGGATGGGGAGGCACCAACTG | 2100 |
| CGAGACCCCCAGGGCCACATGCTTAGACCAGTGTTCAGGCCACGGAACCTTCCTCCCGGA | 2160 |
| CACCGGGCTTTGCAGCTGTGACCCAAGCTGGACTGGACACGACTGTTCTATCGAGATCTG | 2220 |
| TGCTGCCGACTGTGGTGGCCATGGCGTGTGCGTAGGGGGCACCTGCCGCTGCGAGGATGG | 2280 |
| CTGGATGGGGGCAGCCTGCGACCAGCGGGCCTGCCACCCGCGCTGTGCCGAGCATGGGAC | 2340 |
| CTGCCGCGACGGCAAGTGCGAGTGCAGCCCTGGCTGGAATGGCGAACACTGCACCATCGC | 2400 |
| TCACTATCTGGATAGGGTAGTTAAAGAGGGTTGCCCTGGGTTGTGCAATGGCAACGGCAG | 2460 |
| ATGTACCTTAGACCTGAATGGTTGGCACTGCGTCTGCCAGCTGGGCTGGAGAGGAGCTGG | 2520 |
| CTGTGACACTTCCATGGAGACTGCCTGCGGTGACAGCAAAGACAATGATGGAGATGGCCT | 2580 |
| GGTGGACTGCATGGACCCTGACTGCTGCCTCCAGCCCCTGTGCCATATCAACCCGCTGTG | 2640 |
| CCTTGGCTCCCCTAACCCTCTGGACATCATCCAGGAGACACAGGTCCCTGTGTCACAGCA | 2700 |
| GAACCTACACTCCTTCTATGACCGCATCAAGTTCCTCGTGGGCAGGGACAGCACGCACAT | 2760 |
| AATCCCCGGGGAGAACCCCTTTGATGGAGGGCATGCTTGTGTTATTCGTGGCCAAGTGAT | 2820 |
| GACATCAGATGGAACCCCCCTGGTTGGTGTGAACATCAGTTTTGTCAATAACCCTCTCTT | 2880 |
| TGGATATACAATCAGCAGGCAAGATGGCAGCTTTGACTTGGTGACAAATGGCGGCATCTC | 2940 |
| CATCATCCTGCGGTTCGAGCGGGCACCTTTCATCACACAGGAGCACACCCTGTGGCTGCC | 3000 |
| ATGGGATCGCTTCTTTGTCATGGAAACCATCATCATGAGACATGAGGAGAATGAGATTCC | 3060 |
| CAGCTGTGACCTGAGCAATTTTGCCCGCCCCAACCCAGTCGTCTCTCCATCCCCACTGAC | 3120 |
| GTCCTTCGCCAGCTCCTGTGCAGAGAAAGGCCCCATTGTGCCGGAAATTCAGGCTTTGCA | 3180 |
| GGAGGAAATCTCTATCTCTGGCTGCAAGATGAGGCTGAGCTACCTGAGCAGCCGGACCCC | 3240 |
| TGGCTACAAATCTGTCCTGAGGATCAGCCTCACCCACCCGACCATCCCCTTCAACCTCAT | 3300 |
| GAAGGTGCACCTCATGGTAGCGGTGGAGGGCCGCCTCTTCAGGAAGTGGTTCGCTGCAGC | 3360 |
| CCCAGACCTGTCCTATTATTTCATTTGGGACAAGACAGACGTCTACAACCAGAAGGTGTT | 3420 |
| TGGGCTTTCAGAAGCCTTTGTTTCCGTGGGTTATGAATATGAATCCTGCCCAGATCTAAT | 3480 |
| CCTGTGGGAAAAAAGAACAACAGTGCTGCAGGGCTATGAAATTGACGCGTCCAAGCTTGG | 3540 |
| AGGATGGAGCCTAGACAAACATCATGCCCTCAACATTCAAAGTGGTATCCTGCACAAAGG | 3600 |
| GAATGGGGAGAACCAGTTTGTGTCTCAGCAGCCTCCTGTCATTGGGAGCATCATGGGCAA | 3660 |
| TGGGCGCCGGAGAAGCATCTCCTGCCCCAGCTGCAACGGCCTTGCTGACGGCAACAAGCT | 3720 |
| CCTGGCCCCAGTGGCCCTCACCTGTGGCTCTGACGGGAGCCTCTATGTGGGTGATTTCAA | 3780 |
| CTACATTAGAAGGATCTTCCCCTCTGGAAATGTCACCAACATCCTAGAGCTGAGGAATAA | 3840 |
| AGATTTCAGACATAGTCACAGTCCAGCACACAAATACTACCTGGCCACAGACCCCATGAG | 3900 |
| TGGGGCCGTCTTCCTTTCTGACAGCAACAGCCGGCGGGTCTTTAAAATCAAGTCCACTGT | 3960 |
| GGTGGTGAAGGACCTTGTCAAGAACTCTGAGGTGGTTGCGGGGACAGGTGACCAGTGCCT | 4020 |
| CCCCTTTGATGACACTCGCTGCGGGGATGGTGGGAAGGCCACAGAAGCCACACTCACCAA | 4080 |
| TCCCAGGGGTATTACAGTGGACAAGTTTGGGCTGATCTACTTCGTGGATGGCACCATGAT | 4140 |
| CAGACGCATCGATCAGAATGGGATCATCTCCACCCTGCTCGGCTCTAATGATCTCACATC | 4200 |
| AGCCCGGCCACTCAGCTGTGATTCTGTCATGGATATTTCCCAGGTAAGACTGGAGTGGCC | 4260 |
| CACAGACTTAGCCATCAACCCAATGGACAACTCACTTTATGTCCTCGACAACAATGTGGT | 4320 |
| CCTGCAAATCTCTGAAAACCACCAGGTGCGCATTGTCGCCGGGAGGCCCATGCACTGCCA | 4380 |
| GGTCCCTGGCATTGACCACTTCCTGCTAAGCAAGGTGGCCATCCACGCAACCCTGGAGTC | 4440 |
| AGCCACCGCTTTGGCTGTTTCACACAATGGGGTCCTGTATATTGCTGAGACTGATGAGAA | 4500 |
| AAAGATCAACCGCATCAGGCAGGTCACCACTAGTGGAGAGATCTCACTCGTTGCTGGGGC | 4560 |
| CCCCAGTGGCTGTGACTGTAAAAATGATGCCAACTGTGATTGTTTTTCTGGAGACGATGG | 4620 |
| TTATGCCAAGGATGCAAAGTTAAATACCCCATCTTCCTTGGCTGTGTGTGCTGATGGGGA | 4680 |
| GCTCTACGTGGCCGACCTTGGGAACATCCGAATTCGGTTTATCCGGAAGAACAAGCCTTT | 4740 |
| CCTCAACACCCAGAACATGTATGAGCTGTCTTCACCAATTGACCAGGAGCTCTATCTGTT | 4800 |
| TGATACCACCGGCAAGCACCTGTACACCCAAAGCCTGCCCACAGGAGACTACCTGTACAA | 4860 |
| CTTCACCTACACTGGGGACGGCGACATCACACTCATCACAGACAACAATGGCAACATGGT | 4920 |
| AAATGTCCGCCGAGACTCTACTGGGATGCCCCTCTGGCTGGTGGTCCCAGATGGCCAGGT | 4980 |
| GTACTGGGTGACCATGGGCACCAACAGTGCACTCAAGAGTGTGACCACACAAGGACACGA | 5040 |
| GTTGGCCATGATGACATACCATGGCAATTCCGGCCTTCTGGCAACCAAAAGCAATGAAAA | 5100 |
| CGGATGGACAACATTTTATGAGTACGACAGCTTTGGCCGCCTGACAAATGTGACCTTCCC | 5160 |
| TACTGGCCAGGTGAGCAGTTTCCGAAGTGATACAGACAGTTCAGTGCATGTCCAGGTAGA | 5220 |
| GACCTCCAGCAAGGATGATGTCACCATAACCACCAACCTGTCTGCCTCAGGCGCCTTCTA | 5280 |
| CACACTGCTGCAAGACCAAGTCCGGAACAGCTACTACATCGGGGCCGATGGCTCCTTGCG | 5340 |
| GCTGCTGCTGGCCAACGGCATGGAGGTGGCGCTGCAGACTGAGCCCCACTTGCTGGCTGG | 5400 |
| CACCGTCAACCCCACCGTGGGCAAGAGGAATGTCACGCTGCCCATCGACAACGGCCTCAA | 5460 |
| CCTGGTGGAGTGGCGCCAGCGCAAAGAGCAGGCTCGGGGCCAGGTCACTGTCTTTGGGCG | 5520 |
| CCGGCTGCGGGTGCACAACCGAAATCTCCTATCTCTGGACTTTGATCGCGTAACACGCAC | 5580 |
| AGAGAAGATCTATGATGACCACCGCAAGTTCACCCTTCGGATTCTGTACGACCAGGCGGG | 5640 |
| GCGGCCCAGCCTCTGGTCACCCAGCAGCAGGCTGAATGGTGTCAACGTGACATACTCCCC | 5700 |
| TGGGGGTTACATTGCTGGCATCCAGAGGGGCATCATGTCTGAAAGAATGGAATACGACCA | 5760 |
| GGCGGGCCGCATCACATCCAGGATCTTCGCTGATGGGAAGACATGGAGCTACACATACTT | 5820 |
| AGAGAAGTCCATGGTGCTGCTACTACACAGCCAGAGGCAGTATATCTTTGAGTTCGACAA | 5880 |
| GAATGACCGCCTCTCTTCTGTGACGATGCCCAACGTGGCGCGGCAGACACTAGAGACCAT | 5940 |
| CCGCTCAGTGGGCTACTACAGAAACATCTATCAGCCCCCTGAGGGCAATGCCTCAGTCAT | 6000 |
| ACAGGACTTCACTGAGGATGGGCACCTCCTTCACACCTTCTACCTGGGCACTGGCCGCAG | 6060 |
| GGTGATATACAAGTATGGCAAACTGTCAAAGCTGGCAGAGACGCTCTATGACACCACCAA | 6120 |
| GGTCAGTTTCACCTATGACGAGACGGCAGGCATGCTGAAGACCATCAACCTACAGAATGA | 6180 |
| GGGCTTCACCTGCACCATCCGCTACCGTCAGATTGGGCCCCTGATTGACCGACAGATCTT | 6240 |
| CCGCTTCACTGAGGAAGGCATGGTCAACGCCCGTTTTGACTACAACTATGACAACAGCTT | 6300 |
| CCGGGTGACCAGCATGCAGGCTGTGATCAACGAGACCCCACTGCCCATTGATCTCTATCG | 6360 |
| CTATGATGATGTGTCAGGCAAGACAGAGCAGTTTGGGAAGTTTGGTGTCATTTACTATGA | 6420 |
| CATTAACCAGATCATCACCACAGCTGTCATGACCCACACCAAGCATTTTGATGCATATGG | 6480 |
| CAGGATGAAGGAAGTGCAGTATGAGATCTTCCGCTCGCTCATGTACTGGATGACCGTCCA | 6540 |
| GTATGATAACATGGGGCGAGTAGTGAAGAAGGAGCTGAAGGTAGGACCCTACGCCAATAC | 6600 |
| CACTCGCTACTCCTATGAGTATGATGCTGACGGCCAGCTGCAGACAGTCTCCATCAATGA | 6660 |
| CAAGCCACTCTGGCGCTACAGCTACGACCTCAATGGGAACCTGCACTTACTGAGCCCTGG | 6720 |
| GAACAGTGCACGGCTCACACCACTACGGTATGACATCCGCGACCGCATCACTCGGCTGGG | 6780 |
| TGACGTGCAATACAAGATGGATGAGGATGGCTTCCTGAGGCAGCGGGGCGGTGATATCTT | 6840 |
| TGAGTACAACTCAGCTGGCCTGCTCATCAAGGCCTACAACCGGGCTGGCAGCTGGAGTGT | 6900 |
| CAGGTACCGCTACGATGGCCTGGGGCGGCGCGTGTCCAGCAAGAGCAGCCACAGCCACCA | 6960 |
| CCTGCAGTTCTTCTATGCAGACCTGACCAACCCCACCAAGGTCACCCACCTGTACAACCA | 7020 |
| CTCCAGCTCTGAGATCACCTCCCTCTACTACGACTTGCAAGGACACCTCTTTGCCATGGA | 7080 |
| GCTGAGCAGTGGTGATGAGTTTTACATAGCTTGTGACAACATCGGGACCCCTCTTGCTGT | 7140 |
| CTTTAGTGGAACAGGTTTGATGATCAAGCAAATCCTGTACACAGCCTATGGGGAGATCTA | 7200 |
| CATGGATACCAACCCCAACTTTCAGATCATCATAGGCTACCATGGTGGCCTCTATGATCC | 7260 |
| ACTCACCAAGCTTGTCCACATGGGCCGGCGAGATTATGATGTGCTGGCCGGACGCTGGAC | 7320 |
| TAGCCCAGACCACGAGCTGTGGAAGCACCTTAGTAGCAGCAACGTCATGCCTTTTAATCT | 7380 |
| CTATATGTTCAAAAACAACAACCCCATCAGCAACTCCCAGGACATCAAGTGCTTCATGAC | 7440 |
| AGATGTTAACAGCTGGCTGCTCACCTTTGGATTCCAGCTACACAACGTGATCCCTGGTTA | 7500 |
| TCCCAAACCAGACATGGATGCCATGGAACCCTCCTACGAGCTCATCCACACACAGATGAA | 7560 |
| AACGCAGGAGTGGGACAACAGCAAGTCTATCCTCGGGGTACAGTGTGAAGTACAGAAGCA | 7620 |
| GCTCAAGGCCTTTGTCACCTTAGAACGGTTTGACCAGCTCTATGGCTCCACAATCACCAG | 7680 |
| CTGCCAGCAGGCTCCAAAGACCAAGAAGTTTGCATCCAGCGGCTCAGTCTTTGGCAAGGG | 7740 |
| GGTCAAGTTTGCCTTGAAGGATGCCCGAGTGACCACAGACATCATCAGTGTGGCCAATGA | 7800 |
| GGATGGGCGAAGGGTTGCTGCCATCTTGAACCATGCCCACTACCTAGAGAACCTGCACTT | 7860 |
| CACCATTGATGGGGTGGATACCCATTACTTTGTGAAACCAGGACCTTCAGAAGGTGACCT | 7920 |
| GGCCATCCTGGGCCTCAGTGGGGGGCGGCGAACCCTGGAGAATGGGGTCAACGTCACTGT | 7980 |
| GTCCCAGATCAACACAGTACTTAATGGCAGGACTAGACGCTACACAGACATCCAGCTCCA | 8040 |
| GTACGGGGCACTGTGCTTGAACACACGCTACGGGACAACGTTGGATGAGGAGAAGGCACG | 8100 |
| GGTCCTGGAGCTGGCCCGGCAGAGAGCCGTGCGCCAAGCGTGGGCCCGCGAGCAGCAGAG | 8160 |
| ACTGCGGGAAGGGGAGGAAGGCCTGCGGGCCTGGACAGAGGGGGAGAAGCAGCAGGTGCT | 8220 |
| GAGCACAGGGCGGGTGCAAGGCTACGACGGCTTTTTCGTGATCTCTGTCGAGCAGTACCC | 8280 |
| AGAACTGTCAGACAGCGCCAACAACATCCACTTCATGAGACAGAGCGAGATGGGCCGGAG | 8340 |
| GTGACAGAGAGGAC | |

A disclosed NOV4 nucleic acid maps to chromosome 11, and is found in at least brain, spinal chord, testis, heart, lung, parathyroid, stomach, breast, colon, epidermis, ovary and kidney. A NOV4 nucleic acid has 7504 of 8359 bases (89%) identical to a gb:GENBANK-ID:AB025413|acc: AB025413.1 mRNA from Mus musculus TEN-M4.

A NOV4 polypeptide (SEQ ID NO:14) encoded by SEQ ID NO:13 is 2769 amino acid residues and is presented using the one letter code in Table 4B. Signal P, Psort and/or Hydropathy results predict that NOV4 does not have a signal peptide and is likely to be localized mitochondrial inner membrane with a certainty of 0.8363. In other embodiments, NOV4 may also be localized to the plasma membrane with a certainty of 0.65 or to the nucleus with a certainty of 0.6000, or microbody with a certainty of 0.3936.

TABLE 4B
NOV4 Polypeptide SEQ ID NO:14

| | |
|---|---|
| MDVKERKPYRSLTRRRDAERRYTSSSADSEEGKAPQKSYSSSETLKAYDQDARLAYGSRV | 60 |
| KDIVPQEAEEFCRTGANFTLRELGLEEVTPPHGTLYRTDIGLPQCGYSMGAGSDADMEAD | 120 |
| TVLSPEHPVRLWGRSTRSGRSSCLSSRANSNLTLTDTEHENTETDHPGGLQNHARLRTPP | 180 |
| PPLSHAHTPNQHHAASINSLNRGNFTPRSNPSPAPTDHSLSGEPPAGGAQEPAHAQENWL | 240 |
| LNSNIPLETRNLGKQPFLGTLQDNLIEMDILGASRHDGAYSDGHFLFKPGGTSPLFCTTS | 300 |
| PGYPLTSSTVYSPPPRPLPRSTFARPAFNLKKPSKYCNWKCAALSAIVISATLVILLAYF | 360 |
| VAMHLFGLNWHLQPMEGQMYEITEDTASSWPVPTDVSLYPSGGTGLETPDRKGKGTTEGK | 420 |
| PSSFFPEDSFIDSGEIDVGRRASQKIPPGTFWRSQVFIDHPVHLKFNVSLGKAALVGIYG | 480 |
| RKGLPPSHTQFDFVELLDGRRLLTQEARSLEGTPRQSRGTVPPSSHETGFIQYLDSGIWH | 540 |
| LAFYNDGKESEVVSFLTTAIESVDNCPSNCYGNGDCISGTCHCFLGFLGPDCGRASCPVL | 600 |
| CSGNGQYMKGRCLCHSGWKGAECDVPTNQCIDVACSNHGTCITGTCICNPGYKGESCEEV | 660 |
| DCMDPTCSGRGVCVRGECHCFVGWGGTNCETPRATCLDQCSGHGTFLPDTGLCSCDPSWT | 720 |
| GHDCSIEICAADCGGHGVCVGGTCRCEDGWMGAACDQRACHPRCAEHGTCRDGKCECSPG | 780 |
| WNGEHCTIAHYLDRVVKEGCPGLCNGNGRCTLDLNGWHCVCQLGWRGAGCDTSMETACGD | 840 |
| SKDNDGDGLVDCMDPDCCLQPLCHINPLCLGSPNPLDIIQETQVPVSQQNLHSFYDRIKF | 900 |
| LVGRDSTHIIPGENPFDGGHACVIRGQVMTSDGTPLVGVNISFVNNPLFGYTISRQDGSF | 960 |
| DLVTNGGISIILRFERAPFITQEHTLWLPWDRFFVMETIIMRHEENEIPSCDLSNFARPN | 1020 |
| PVVSPSPLTSFASSCAEKGPIVPEIQALQEEISISGCKMRLSYLSSRTPGYKSVLRISLT | 1080 |
| HPTIPFNLMKVHLMVAVEGRLFRKWFAAAPDLSYYFIWDKTDVYNQKVFGLSEAFVSVGY | 1140 |
| EYESCPDLILWEKRTTVLQGYEIDASKLGGWSLDKHHALNIQSGILHKGNGENQFVSQQP | 1200 |
| PVIGSIMGNGRRRSISCPSCNGLADGNKLLAPVALTCGSDGSLYVGDFNYIRRIFPSGNV | 1260 |
| TNILELRNKDFRHSHSPAHKYYLATDPMSGAVFLSDSNSRRVFKIKSTVVVKDLVKNSEV | 1320 |
| VAGTGDQCLPFDDTRCGDGGKATEATLTNPRGITVDKFGLIYFVDGTMIRRIDQNGIIST | 1380 |
| LLGSNDLTSARPLSCDSVMDISQVRLEWPTDLAINPMDNSLYVLDNNVVLQISENHQVRI | 1440 |
| VAGRPMHCQVPGIDHFLLSKVAIHATLESATALAVSHNGVLYIAETDEKKINRIRQVTTS | 1500 |
| GEISLVAGAPSGCDCKNDANCDCFSGDDGYAKDAKLNTPSSLAVCADGELYVADLGNIRI | 1560 |
| RFIRKNKPFLNTQNMYELSSPIDQELYLFDTTGKHLYTQSLPTGDYLYNFTYTGDGDITL | 1620 |
| ITDNNGNMVNVRRDSTGMPLWLVVPDGQVYWVTMGTNSALKSVTTQGHELAMMTYHGNSG | 1680 |
| LLATKSNENGWTTFYEYDSFGRLTNVTFPTGQVSSFRSDTDSSVHVQVETSSKDDVTITT | 1740 |
| NLSASGAFYTLLQDQVRNSYYIGADGSLRLLLANGMEVALQTEPHLLAGTVNPTVGKRNV | 1800 |
| TLPIDNGLNLVEWRQRKEQARGQVTVFGRRLRVHNRNLLSLDFDRVTRTEKIYDDHRKFT | 1860 |
| LRILYDQAGRPSLWSPSSRLNGVNVTYSPGGYIAGIQRGIMSERMEYDQAGRITSRIFAD | 1920 |
| GKTWSYTYLEKSMVLLLHSQRQYIFEFDKNDRLSSVTMPNVARQTLETIRSVGYYRNIYQ | 1980 |
| PPEGNASVIQDFTEDGHLLHTFYLGTGRRVIYKYGKLSKLAETLYDTTKVSFTYDETAGM | 2040 |
| LKTINLQNEGFTCTIRYRQIGPLIDRQIFRFTEEGMVNARFDYNYDNSFRVTSMQAVINE | 2100 |
| TPLPIDLYRYDDVSGKTEQFGKFGVIYYDINQIITTAVMTHTKHFDAYGRMKEVQYEIFR | 2160 |
| SLMYWMTVQYDNMGRVVKKELKVGPYANTTRYSYEYDADGQLQTVSINDKPLWRYSYDLN | 2220 |
| GNLHLLSPGNSARLTPLRYDIRDRITRLGDVQYKMDEDGFLRQRGGDIFEYNSAGLLIKA | 2280 |
| YNRAGSWSVRYRYDGLGRRVSSKSSHSHHLQFFYADLTNPTKVTHLYNHSSSEITSLYYD | 2340 |
| LQGHLFAMELSSGDEFYIACDNIGTPLAVFSGTGLMIKQILYTAYGEIYMDTNPNFQIII | 2400 |
| GYHGGLYDPLTKLVHMGRRDYDVLAGRWTSPDHELWKHLSSSNVMPFNLYMFKNNNPISN | 2460 |
| SQDIKCFMTDVNSWLLTFGFQLHNVIPGYPKPDMDAMEPSYELIHTQMKTQEWDNSKSIL | 2520 |
| GVQCEVQKQLKAFVTLERFDQLYGSTITSCQQAPKTKKFASSGSVFGKGVKFALKDGRVT | 2580 |
| TDIISVANEDGRRVAAILNHAHYLENLHFTIDGVDTHYFVKPGPSEGDLAILGLSGGRRT | 2640 |
| LENGVNVTVSQINTVLNGRTRRYTDIQLQYGALCLNTRYGTTLDEEKARVLELARQRAVR | 2700 |
| QAWAREQQRLREGEEGLRAWTEGEKQQVLSTGRVQGYDGFFVISVEQYPELSDSANNIHF | 2760 |
| MRQSEMGRR | |

The full amino acid sequence of the protein of the invention was found to have 2688 of 2771 amino acid residues (97%) identical to, and 2728 of 2771 amino acid residues (98%) similar to, the 2771 amino acid residue ptnr:SPTREMBL-ACC:Q9WTS7 protein from Mus musculus TEN-M4.

NOV4 also has homology to the amino acid sequences shown in the BLASTP data listed in Table 4C.

TABLE 4C
BLAST results for NOV4

| Gene Index/ | | Length | Identity | Positives | |
|---|---|---|---|---|---|
| Identifier | Protein/Organism | (aa) | (%) | (%) | Expect |
| gi\|16551957\|dbj\|BAB71206.1\| | unnamed protein | 730 | 99 | 99 | 0.0 |
| (AK056531) | product [Homo | | | | |
| | sapiens] | | | | |
| gi\|7657417\|ref\|NP_035987.2\| | odd Oz/ten-m | 2715 | 66 | 79 | 0.0 |
| (NM_011857) | homolog 3 | | | | |
| | (Drosophila); | | | | |
| | odd Oz/ten-m | | | | |
| | homolog 1 | | | | |
| | (Drosophila) | | | | |
| | [Mus musculus] | | | | |
| gi\|13649010\|ref\|XP_010128.3\| | odz (odd Oz/ten- | 2725 | 62 | 76 | 0.0 |
| XM_010128 | m, Drosophila) | | | | |
| | homolog 1 [Homo | | | | |
| | sapiens] | | | | |
| gi\|1079143\|pir\|\|S47008 | tenascin-like | 2515 | 33 | 53 | 0.0 |
| | protein - fruit | | | | |
| | fly (Drosophila | | | | |
| | melanogaster) | | | | |
| gi\|8922444\|ref\|NP_060574.1\| | hypothetical | 1045 | 99 | 99 | 0.0 |
| (NM_018104) | protein | | | | |
| | FLJ10474; | | | | |
| | hypothetical | | | | |
| | protein FLJ10886 | | | | |
| | [Homo sapiens] | | | | |

The homology of these sequences is shown graphically in the ClustalW analysis shown in Table 4D.

Table 4D ClustalW Analysis of NOV4

Tables 4E lists the domain description from DOMAIN analysis results against NOV4. This indicates that the NOV4 sequence has properties similar to those of other proteins known to contain this domain.

[image]

[image]

[image]

[image]

[image]

[image]

[image]

[image]

[image]

[image]

[image]

TABLE 4E

Domain Analysis of NOV4
gnl|Pfam|pfam01500, Keratin_B2, Keratin, high sulfur B2 protein.
High sulfur proteins are cysteine-rich proteins synthesized
during the differentiation of hair matrix cells, and form hair
fibers in association with hair keratin intermediate filaments.
This family has been divided up into four regions, with the
second region containing 8 copies of a short repeat. This
family is also known as B2 or KAP1.
CD-Length = 144 residues, 87.5% aligned
Score = 38.9 bits (89), Expect = 0.004

| | | | |
|---|---|---|---|
| Query: | 630 | CIDVACSNHGTCITGTCICNPGYKGESCEEVDCMDPTCSGRGVCVRGECHCFVGWGGTNC | 689 |
| | | C CS GTC + C + SC + C P CS C R C + C | |
| Sbjct: | 5 | CGFPTCSTLGTCGSSCC------QPPSCCQPSCCQPVCSQTTCC-RPTCFQSSCCRPSCC | 57 |
| Query: | 690 | ETP---RATCLDQCSGHGTFLPDTGLCSCDPSWTGHDCSIEICAADCGGHGVCVGGTCRCE | 747 |
| | | +T + TC S G+ SC W DC +E | |
| Sbjct: | 58 | QTSCCQPTCCQSSSCQ----TGCGIGSCRTRWCRPDCRVE-------------------- | 93 |
| Query: | 748 | DGWMGAACDQRACHPRCAEHGTCRDGKCECS---PGWNGEHC | 786 |
| | | C C C C+ + S P + G+ C | |
| Sbjct: | 94 | -----GTCLPPCCVVSCTPPTCCQPVSAQASCCRPSYCGQSC | 130 |

The novel TEN-M-like protein encoded by the gene of invention has highest homology to the mouse TEN-M4 protein, which belongs to the ODZ/TENM family of proteins. This family was first identified in Drosophila as being a pair-rule gene affecting segmentation of the early embryo. It was the first pair-rule gene identified that was not a transcription factor, but a type II transmembrane protein. Vertebrate homologs of the TENM family have been identified in mouse and zebrafish. In the mouse, TEN-M4 expression was found to be on the cell surface, in the brain, trachea as well as developing limb and bone. Analysis of the TEN-M1 protein reveals that it can bind to itself, making it likely that TEN-M4 may be a dimeric moiety as well. In cell culture experiments, fragments of the TEN-M proteins can bind the Drosophila PS2 integrins. In addition, members of the TEN-M family have been identified to be downstream of the endoplasmic reticulum stress response pathway, which alters the response of cells to their environment. This suggests that the ODZ/TENM family may be involved in cell adhesion, spreading and motility. Translocations leading to the fusion of this gene with the NRG1/HGL gene from chromosome 8 have been found to generate a paracrine growth factor for one mammary carcinoma cell line, termed gamma-heregulin. Therefore this novel gene may have widespread implications in development, regeneration and carcinogenesis of various tissues.

Two new potential ligands of the Drosophila PS2 integrins have been characterized by functional interaction in cell culture. These potential ligands are a new Drosophila laminin alpha2 chain encoded by the wing blister locus and Ten-m, an extracellular protein known to be involved in embryonic pattern formation. As with previously identified PS2 ligands, both contain RGD sequences, and RGD-containing fragments of these two proteins (DLAM-RGD and TENM-RGD) can support PS2 integrin-mediated cell spreading. In all cases, this spreading is inhibited specifically by short RGD-containing peptides. As previously found for the PS2 ligand tiggrin (and the tiggrin fragment TIG-RGD), TENM-RGD induces maximal spreading of cells expressing integrin containing the alphaPS2C splice variant. This is in contrast to DLAM-RGD, which is the first Drosophila polypeptide shown to interact preferentially with cells expressing the alphaPS2 m8 splice variant. The betaPS integrin subunit also varies in the presumed ligand binding region as a result of alternative splicing. For TIG-RGD and TENM-RGD, the beta splice variant has little effect, but for DLAM-RGD, maximal cell spreading is supported only by the betaPS4A form of the protein. Thus, the diversity in PS2 integrins due to splicing variations, in combination with diversity of matrix ligands, can greatly enhance the functional complexity of PS2-ligand interactions in the developing animal. The data also suggest that the splice variants may alter regions of the subunits that are directly involved in ligand interactions, and this is discussed with respect to models of integrin structure.

A sequence of about thirty to forty amino-acid residues long found in the sequence of epidermal growth factor (EGF) has been shown to be present, in a more or less conserved form, in a large number of other, mostly animal proteins. The list of proteins currently known to contain one or more copies of an EGF-like pattern is large and varied. The functional significance of EGF domains in what appear to be unrelated proteins is not yet clear. However, a common feature is that these repeats are found in the extracellular domain of membrane-bound proteins or in proteins known to be secreted (exception: prostaglandin G/H synthase). The EGF domain includes six cysteine residues which have been shown (in EGF) to be involved in disulfide bonds. The main structure is a two-stranded beta-sheet followed by a loop to a C-terminal short two-stranded sheet. Subdomains between the conserved cysteines vary in length. The NHL (NCL-1, HT2A and LIN-41) repeat is found in a variety of enzymes of the copper type II, ascorbate-dependent monooxygenase family which catalyse the C-terminus alpha-amidation of biological peptides. The repeat also occurs in a human zinc finger protein that specifically interacts with the activation domain of lentiviral Tat proteins. The repeat domain that is often associated with RING finger and B-box motifs (see, Ben-Zur T, Dev Biol Jan. 1, 2000;217(1):107-20; Adelaide J, Int J Oncol April 2000;16(4):683-8; Wang X Z, Oncogene Oct. 7, 1999;18(41):5718-21; Schaefer G, Oncogene Sep. 18, 1997;15(12):1385-94 ; Wang X Z, EMBO J Jul. 1, 1998;17(13):3619-30; Baumgartner S, EMBO J Aug. 15, 1994;13(16):3728-40; Otaki J M, Dev Biol Aug. 1, 1999;212(1):165-81; Mieda M, Mech Dev September 1999;87(1-2):223-7; Oohashi T, J Cell Biol May 3, 1999;145(3):563-77; Graner M W, J Biol Chem Jul. 17, 1998;273(29):18235-41, incorporated herein by reference).

The protein similarity information, expression pattern, and map location for the TEN-M4-like protein and nucleic acid disclosed herein suggest that this TEN-M4-like protein may have important structural and/or physiological functions characteristic of this family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The NOV4 nucleic acids and proteins of the invention are useful in potential diagnostic and therapeutic applications implicated in various diseases and disorders described below and/or other pathologies. For example, the compositions of the present invention will have efficacy for treatment of patients suffering from: cardiac diseases, myocardial contractility in failing heart and other diseases, disorders and conditions of the like. The disclosed NOV4 nucleic acid of the invention encoding a TEN-M4-like protein includes the nucleic acid whose sequence is provided in Table 4A or a fragment thereof. The invention also includes a mutant or variant nucleic acid any of whose bases may be changed from the corresponding base shown in Table 4A while still encoding a protein that maintains TEN-M4-like protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 11 percent of the bases may be so changed.

The disclosed NOV4 protein of the invention includes the TEN-M4-like protein whose sequence is provided in Table 3B. The invention also includes a mutant or variant protein any of whose residues may be changed from the corresponding residue shown in Table 4B while still encoding a protein that maintains beta adrenergic receptor kinase-like activities and physiological functions, or a functional fragment thereof. In the mutant or variant protein, up to about 3 percent of the residues may be so changed.

The protein similarity information, expression pattern, and map location for TEN-M4-like protein and nucleic acid (NOV4) disclosed herein suggest that NOV4 may have important structural and/or physiological functions characteristic of the TEN-M4 protein family. Therefore, the NOV4 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV4 nucleic acids and proteins of the invention are useful in potential diagnostic and therapeutic applications implicated in various diseases and disorders described below. For example, the compositions of the present invention will have efficacy for treatment of patients suffering from: Von Hippel-Lindau (VHL) syndrome, Alzheimer's disease, stroke, tuberous sclerosis, hypocalcaemia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration, fertility disorders, hyperparathyroidism, hypoparathyroidism, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation disorders, diabetes, autoimmune disease, renal artery stenosis, interstitial nephritis, glomerulonephritis, polycystic kidney disease, systemic lupus erythematosus, renal tubular acidosis, IgA nephropathy, hypocalcaemia, asthma, emphysema, scleroderma, allergy, ARDS, Hirschsprung's disease, Crohn's disease, appendicitis, inflammatory bowel disease, gastric ulcers, psoriasis, actinic keratosis, acne, hair growth/loss, allopecia, pigmentation disorders, endocrine disorders and cancer and other diseases, disorders and conditions of the like. The NOV4 nucleic acid, or fragments thereof, may further be useful in diagnostic applications, wherein the presence or amount of the nucleic acid or the protein are to be assessed.

NOV4 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV4 polypeptide has multiple hydrophilic regions, each of which can be used as an immunogen. In one embodiment, a contemplated NOV4 epitope is from about amino acids 1 to 400. In another embodiment, a contemplated NOV4 epitope is from about amino acids 450 to 520. In other specific embodiments, contemplated NOV4 epitopes are from about amino acids 750 to 850, 1100 to 1200, 1250 to 1400, 1490 to 1750, 1760 to 2300, 2400 to 2600, and 2650 to 2725.

NOV5

NOV5 includes two Out At First-like proteins disclosed below. The disclosed sequences have been named NOV5a and NOV5b.

NOV5a: A disclosed NOV5a nucleic acid of 822 nucleotides identified as SEQ ID NO:15 (also referred to as CG55764-01) encoding an Out At First-like protein is shown in Table 5A. An open reading frame was identified beginning with an ATG initiation codon at nucleotides 1-3 and ending with a TGA codon at nucleotides 820-822.

TABLE 5A
NOV5a Polynucleotide SEQ ID NO:15

ATGCGCCTTCCCGGGGTACCCCTGGCGCGCCCTGCGCTGCTGCTGCTGCTGCCGCTGCTCGCGCCGCTGC
TGGGAACGGGTGCGCCGGCCGAGCTGCGGGTCCGCGTGCGGCTGCCGGACGGCCAGGTGACCGAGGAGAG
CCTGCAGGCGGACAGCGACGCGGACAGCATCAGCCTCGAGCTGCGCAAGCCCGACGGCACCCTCGTCTCC
TTCACCGCCGACTTCAAGAAGGATGTGAAGGTCTTCCGGGCCCTGATCCTGGGGGAGCTGGAGAAGGGGC
AGAGTCAGTTCCAGGCCCTCTGCTTTGTCACCCAGCTGCAGCACAATGAGATCATCCCCAGTGAGGCCAT
GGCCAAGCTCCGGCAGAAAAATCCCCGGGCAGTGCGGCAGGCGGAGGAGGTTCGGGGTCTGGAGCATCTG
CACATGGATGTCGCTGTCAACTTCAGCCAGGGGGCCCTGCTGAGCCCCCATCTCCACAACGTGTGTGCCG
AGGCCGTGGATGCCATCTACACCCGCCAGGAGGATGTCCGGTTCTGGCTGGAGCAAGGTGTGGACAGTTC
TGTGTTCGAGGCTCTGCCCAAGGCCTCAGAGCAGGCGGAGCTGCCTCGCTGCAGGCAGGTGGGGGACCGC
GGGAAGCCCTGCGTCTGCCACTATGGCCTGAGCCTGGCCTGGTACCCCTGCATGCTCAAGTACTGCCACA
GCCGCGACCGGCCCACGCCCTACAAGTGTGGCATCCGCAGCTGCCAGAAGAGCTACAGCTTTGACTTCTA
CGTGCCCCAGAGGCAGCTGTGTCTCTGGGATGAGGATCCCTACCCAGGCTAG

The NOV5a nucleic acid was identified on chromosome 11 and has 455 of 733 bases (62%) identical to a gb:GENBANK-ID:DROOAFPR|acc:L31349.1 mRNA from D. melanogaster (mRNA for out at first (oaf)).

A disclosed NOV5a polypeptide (SEQ ID NO:16) encoded by SEQ ID NO:15 is 273 amino acid residues and is presented using the one-letter code in Table 5B. Signal P, Psort and/or Hydropathy results predict that NOV5a has a signal peptide and is likely to be localized outside the cell with a certainty of 0.7523. In other embodiments, NOV5a may also be localized to the endoplasmic reticulum with a certainty of 0.1000 or microbody with a certainty of 0.1000. The most likely cleavage site is between positions 27 and 28: residues GTG-AP.

TABLE 5B
NOV5a Polypeptide SEQ ID NO:16

| | |
|---|---|
| MRLPGVPLARPALLLLLPLLAPLLGTGAPAELRVRVRLPDGQVTEESLQADSDADSISLE | 60 |
| LRKPDGTLVSFTADFKKDVKVFRALILGELEKGQSQFQALCFVTQLQHNEIIPSEAMAKL | 120 |
| RQKNPRAVRQAEEVRGLEHLHMDVAVNFSQGALLSPHLHNVCAEAVDAIYTRQEDVRFWL | 180 |
| EQGVDSSVFEALPKASEQAELPRCRQVGDRGKPCVCHYGLSLAWYPCMLKYCHSRDRPTP | 240 |
| YKCGIRSCQKSYSFDFYVPQRQLCLWDEDPYPG | |

The disclosed NOV5a amino acid sequence has 106 of 274 amino acid residues (38%) identical to, and 154 of 274 amino acid residues (56%) similar to, the 487 amino acid residue ptnr:SWISSNEW-ACC:Q9NLA6 protein from Drosophila melanogaster (fruit fly) (Out At First protein).

The Out At First Protein disclosed in this invention is expressed in at least the following tissues: Adipose, Adrenal Gland/Suprarenal gland, Amygdala, Aorta, Artery, Ascending Colon, Bone, Bone Marrow, Brain, Brown adipose, Cartilage, Cervix, Cochlea, Colon, Coronary Artery, Dermis, Duodenum, Epidermis, Hair Follicles, Heart, Hippocampus, Kidney, Kidney Cortex, Liver, Lung, Lymph node, Lymphoid tissue, Mammary gland/Breast, Myometrium, Esophagus, Ovary, Oviduct/Uterine Tube/Fallopian tube, Pancreas, Parotid Salivary glands, Peripheral Blood, Pituitary Gland, Prostate, Respiratory Bronchiole, Retina, Salivary Glands, Skin, Small Intestine, Spinal Chord, Spleen, Stomach, Synovium/Synovial membrane, Thalamus, Thymus, Thyroid, Trachea, Urinary Bladder, Uterus, Vein, Vulva, Whole Organism. This information was derived by determining the tissue sources of the sequences that were included in the invention including but not limited to SeqCalling sources, Public EST sources, Literature sources, and/or RACE sources.

NOV5b: A disclosed NOV5b nucleic acid of 1362 nucleotides identified as SEQ ID NO:17 (also referred to as CG55764-02) encoding a novel serine/threonine kinase-like protein is shown in Table 5C. An open reading flame was identified beginning with an ATG initiation codon at nucleotides 1-3 and ending with a TGA at nucleotides 820-822.

TABLE 5C
NOV5b Polynucleotide SEQ ID NO:17

ATGCGCCTTCCCGGGGTACCCCTGGCGCGCCCTGCGCTGCTGCTGCTGCTGCCGCTGCTCGCGCCGCTGC
TGGGAACGGGTGCGCCGGCCGAGCTGCGGGTCCGCGTGCGGCTGCCGGACGGCCAGGTGACCGAGGAGAG
CCTGCAGGCGGACAGCGACGCGGACAGCATCAGCCTCGAGCTGCGCAAGCCCGACGGCACCCTCGTCTCC
TTCACCGCCGACTTCAAGAAGGATGTGAAGGTCTTCCGGGCCCTGATCCTGGGGGAGCTCGAGAAGGGGC
AGAGTCAGTTCCAGGCCCTCTGCTTTGTCACCCAGCTGCAGCACAATGAGATCATCCCCAGTGAGGCCAT
GGCCAAGCTCCGGCAGAAAAATCCCCGGGCAGTGCGGCAGGCGGAGGAGGCTCGGGGTCTGGAGCATCTG
CACATGGATGTCGCTGTCAACTGCAGCCAGGGGGCCCTGCTGAGCCCCCATCTCCACAACGTGTGTGCCG
AGGCCGTGGATGCCATCTACACCCGCCAGGAGGATGTCCGGTTCTGGCTGGAGCAAGGTGTGGACAGTTC
TGTGTTCGAGGCTCTGCCCAAGGCCTCAGAGCAGGCGGAGCTGCCTCGCTGCAGGCAGGTGGGGGACCGC
GGGAAGCCCTGCGTCTGCCACTATGGCCTGAGCCTGGCCTGGTACCCCTGCATGCTCAAGTACTGCCACA
GCCGCGACCGGCCCACGCCCTACAAGTGTGGCATCCGCAGCTGCCAGAAGAGCTACAGCTTCGACTTCTA
CGTGCCCCAGAGGCAGCTGTGTCTCTGGGATGAGGATCCCTACCCAGGCTAGGGTGGGAGCAACCTGGCG
AGTGGCTGCTCTGGGCCCACTGCTCTTCACCAGCCACTAGAGGGGGTGGCAACCCCCACCTGAGGCCTTA
TTTCCCTCCCTCCCCACTCCCCTGGCCCTAGAGCCTGGGCCCCTCTGGCCCCATCTCACATGACTGTGAA
GGGGGTGTGGCATGGCAGGGGGTCTCATGAAGGCACCCCCATTCCCACCCTGTGCCTTCCTTGCGGGCAG
AGAGGGAGAGAAGGGCTCCCCAGATCTACACCCCTCCCTCCTGCATCTCCCCTGGAGTGTTCACTTGCAA
GCTGCCAAAACATGATGGCCTCTGGTTGTTCTGTTGAACTCCTTGAACGTTTAGACCCTAAAAGGAGTCT
ATACCTGGACACCCACCTCCCCAGACACAACTCCCTTCCCCATGCACACATCTGGAAGGAGCTGGCCCCT
CAGTCCCTTCCTACTCCCCAACAAGGGGCTCACTATCCCCAAAGAAGGAGCTGTTGGGGACCCACGACGC
AGCCCCTGTACTGGATTACAGCATATTCTCAT

The NOV5b nucleic acid was identified on chromosome 11 and has 456 of 733 bases (62%) identical to a gb:GENBANK-ID:DROOAFPR|acc:L31349.1 mRNA from D. melanogaster (mRNA for out at first (oaf)).

A disclosed NOV5b polypeptide (SEQ ID NO:18) encoded by SEQ ID NO:17 is 273 amino acid residues and is presented using the one-letter code in Table 5D. Signal P, Psort and/or Hydropathy results predict that NOV5b has a signal peptide and is likely to be localized outside the cell with a certainty of 0.7523. In other embodiments, NOV5b may also be localized to the endoplasmic reticulum with a certainty of0.1000 or microbody with a certainty of 0.1000. The most likely cleavage site is between positions 27 and 28: residues GTG-AP.

TABLE 5D
NOV5b Polypeptide SEQ ID NO:18

| | |
|---|---|
| MRLPGVPLARPALLLLLPLLAPLLGTGAPAELRVRVRLPDGQVTEESLQADSDADSISLE | 60 |
| LRKPDGTLVSFTADFKKDVKVFRALILGELEKGQSQFQALCFVTQLQHNEIIPSEAMAKL | 120 |
| RQKNPRAVRQAEEARGLEHLHMDVAVNCSQGALLSPHLHNVCAEAVDAIYTRQEDVRFWL | 180 |
| EQGVDSSVFEALPKASEQAELPRCRQVGDRGKPCVCHYGLSLAWYPCMLKYCHSRDRPTP | 240 |
| YKCGIRSCQKSYSFDFYVPQRQLCLWDEDPYPG | |

The disclosed NOV5b amino acid sequence has 106 of 274 amino acid residues (38%) identical to, and 154 of 274 amino acid residues (56%) similar to, the 487 amino acid residue ptnr:SWISSNEW-ACC:Q9NLA6 protein from Drosophila melanogaster (fruit fly) (Out At First protein).

The NOV5b Out At First Protein disclosed in this invention is expressed in at least the following tissues: Adipose, Adrenal Gland/Suprarenal gland, Amygdala, Aorta, Artery, Ascending Colon, Bone, Bone Marrow, Brain, Brown adipose, Cartilage, Cervix, Cochlea, Colon, Coronary Artery, Dermis, Duodenum, Epidermis, Hair Follicles, Heart, Hippocampus, Kidney, Kidney Cortex, Liver, Lung, Lymph node, Lymphoid tissue, Mammary gland/Breast, Myometrium, Esophagus, Ovary, Oviduct/Uterine Tube/Fallopian tube, Pancreas, Parotid Salivary glands, Peripheral Blood, Pituitary Gland, Prostate, Respiratory Bronchiole, Retina, Salivary Glands, Skin, Small Intestine, Spinal Chord, Spleen, Stomach, Synovium/Synovial membrane, Thalamus, Thymus, Thyroid, Trachea, Urinary Bladder, Uterus, Vein, Vulva, Whole Organism. This information was derived by determining the tissue sources of the sequences that were included in the invention including but not limited to SeqCalling sources, Public EST sources, Literature sources, and/or RACE sources.

NOV5b also has homology to the amino acid sequences shown in the BLASTP data listed in Table 5E.

TABLE 5E
BLAST results for NOV5

| | | Length | Identity | Positives | |
|---|---|---|---|---|---|
| Gene Index/Identifier | Protein/Organism | (aa) | (%) | (%) | Expect |
| gi\|17136996\|ref\|NP_477040.1\| | oaf-P1; | 332 | 38 | 55 | 2e−51 |
| (NM_057692) | transcript near | | | | |
| | decapentaplegic; | | | | |
| | transcript- | | | | |
| | near- | | | | |
| | decapentaplegic; | | | | |
| | near dpp | | | | |
| | complementation | | | | |
| | group 1 | | | | |
| | [Drosophila | | | | |
| | melanogaster] | | | | |
| gi\|7321824\|gb\|AAC37219.2\| | out at first | 487 | 38 | 55 | 5e−51 |
| (L31349) | [Drosophila | | | | |
| | melanogaster] | | | | |
| gi\|12643516\|sp\|Q9NLA6\|OAF_D | OUT AT FIRST | 487 | 38 | 55 | 5e−51 |
| ROME | PROTEIN | | | | |
| | [CONTAINS: OUT | | | | |
| | AT FIRST SHORT | | | | |
| | PROTEIN] | | | | |
| gi\|11386961\|sp\|O18638\|OAF_D | OUT AT FIRST | 305 | 40 | 58 | 1e−50 |
| ROVI | PROTEIN | | | | |

The homology of these sequences is shown graphically in the ClustalW analysis shown in Table 5F.

[image]

[image]

[image]

Tables 5G-I list the domain description from DOMAIN analysis results against NOV5a. This indicates that the NOV5a sequence has properties similar to those of other proteins known to contain this domain.

TABLE 5G
Domain Analysis of NOV5

gi|17136996|ref|NP_477040.1| oaf-P1; transcript near decapentaplegic;
transcript-near-decapentaplegic; near dpp
complementation group 1 [Drosophila melanogaster]
CD-Length = 332 Score = 202 bits (515), Expect = 2e−51

This sequence from human chromosome 11 encodes for a novel protein which shows some sequence similarity to the Drosophila melanogaster Out At First (OAF) protein. Out At First is expressed in clusters of cells during germband extension, throughout the developing nervous system, and in the gonads of both sexes throughout the lifecycle. Mutation of the Drosophila gene is fatal and causes nervous system defects.

The disclosed NOV5 nucleic acid of the invention encoding an Out At First-like protein includes the nucleic acid whose sequence is provided in Table 5A or a fragment thereof. The invention also includes a mutant or variant nucleic acid any of whose bases may be changed from the corresponding base shown in Table 5A while still encoding a protein that maintains its Out At First-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant NOV5a and NOV5b nucleic acids, and their complements, up to about 38 percent of the bases may be so changed.

The disclosed NOV5a protein of the invention includes the Out At First-like protein whose sequence is provided in Table 5B. The invention also includes a mutant or variant protein any of whose residues may be changed from the corresponding residue shown in Table 5B while still encoding a protein that maintains its Out At First-like activities and physiological functions, or a functional fragment thereof. In the mutant or variant protein, up to about 62 percent of the residues may be so changed.

The disclosed NOV5b protein of the invention includes the Out At First-like protein whose sequence is provided in Table 5D. The invention also includes a mutant or variant protein any of whose residues may be changed from the corresponding residue shown in Table 5D while still encoding a protein that maintains its Out At First-like activities and physiological functions, or a functional fragment thereof. In the mutant or variant protein, up to about 62 percent of the residues may be so changed.

The NOV5 nucleic acids and proteins of the invention are useful in potential therapeutic applications implicated in various diseases, disorders and conditions. The NOV5 nucleic acid, or fragments thereof, may further be useful in diagnostic applications, wherein the presence or amount of the nucleic acid or the protein are to be assessed.

NOV5 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below.

The disclosed NOV5a polypeptide has multiple hydrophilic regions, each of which can be used as an immunogen. In one embodiment, a contemplated NOV5a epitope is from about amino acids 40 to 75. In another embodiment, a contemplated NOV5a epitope is from about amino acids 80 to 87. In other specific embodiments, contemplated NOV5a epitopes are from about amino acids 95 to 105, 110 to 145, 155 to 180, and 225 to 260.

The disclosed NOV5b polypeptide has multiple hydrophilic regions, each of which can be used as an immunogen. In one embodiment, a contemplated NOV5b epitope is from about amino acids 40 to 75. In another embodiment, a contemplated NOV5b epitope is from about amino acids 80 to 90. In other specific embodiments, contemplated NOV5b epitopes are from about amino acids 95 to 105, 110 to 145, 160 to 220, and 225 to 260.

NOV6

NOV6 includes two EphA6/ehk-2-like proteins disclosed below. The disclosed sequences have been named NOV6a and NOV6b.

NOV6a: A disclosed NOV6a nucleic acid of 3641 nucleotides identified as SEQ ID NO:19 (also referred to as CG55704-01) encoding an EphA6/ehk-2-like protein is shown in Table 6A. An open reading flame was identified beginning with an ATG initiation codon at nucleotides 19-21and ending with a TGA codon at nucleotides 3124-3126. Putative untranslated regions are indicated by underline.

TABLE 6A
NOV6a Polynucleotide SEQ ID NO:19

| | |
|---|---|
| AGAGAACCAGCGAGAGCCATGGGGGGCTGCGAAGTCCGGGAATTTCTTTTGCAATTTGGT | 60 |
| TTCTTCTTGCCCCTGCTGACAGCTTGGACCGGCGACTGCAGTCACGTCTCCAACCAAGTT | 120 |
| GTGTTGCTTGATACATCTACAGTGATGGGAGAACTAGGATGGAAAACATATCCACTGAAT | 180 |
| GGGTGGGATGCCATTACTGAAATGGATGAACACAACAGGCCCATACATACATACCAGGTA | 240 |
| TGCAATGTCATGGAACCAAACCAGAACAACTGGCTTCGTACTAACTGGATCTCTCGTGAT | 300 |
| GCTGCTCAGAAAATCTATGTGGAAATGAAGTTCACATTGAGAGATTGTAACAGCATCCCA | 360 |
| TGGGTCTTGGGAACGTGTAAAGAAACATTTACTCTGTATTATATTGAATCTGACGAATCC | 420 |
| CACGGAACTAAATTCAAGCCAAGCCAATATATAAAGATTGACACAATTGCTGCGGATGAG | 480 |
| AGTTTTACTCAGATGGATTTGGGTGATCGCATCCTTAAACTCAACACTGAAATTCGTGAG | 540 |
| GTGGGGCCTATAGAAAGGAAAGGATTTTATCTGGCTTTTCAAGACATTGGGGCGTGCATT | 600 |
| GCCCTGGTTTCAGTCCGTGTTTTCTACAAGAAATGCCCCTTCACTGTTCGTAACTTGGCC | 660 |
| ATGTTTCCTGATACCATTCCAAGGGTTGATTCCTCCTCTTTGGTTGAAGTACGGGGTTCT | 720 |
| TGTGTGAAGAGTGCTCAAGAGCGTGACACTCCTAAACTGTATTGTGGAGCTGATGGAGAT | 780 |
| TGGCTGGTTCCTCTTCGAAGGTGCATCTGCAGTACAGGATATGAAGAAATTGAGGGTTCT | 840 |
| TGCCATGCTTGCAGACCAGGATTCTATAAAGCTTTTGCTGGGAACACAAAATGTTCTAAA | 900 |
| TGTCCTCCACACAGTTTAACATACATGGAAGCAACTTCTGTCTGTCAGTGTGAAAAGGGT | 960 |
| TATTTCCGAGCTGAAAAAGACCCACCTTCTATGGCATGTACCAGGCCACCTTCAGCTCCT | 1020 |
| AGGAATGTGGTTTTTAACATCAATGAAACAGCCCTTATTTTGGAATGGAGCCCACCAAGT | 1080 |
| GACACAGGAGGGAGAAAAGATCTCACATACAGTGTAATCTGTAAGAAATGTGGCTTAGAC | 1140 |
| ACCAGCCAGTGTGAGGACTGTGGTGGAGGACTCCGCTTCATCCCAAGACATACAGGCCTG | 1200 |
| ATCAACAATTCCGTGATAGTACTTGACTTTGTGTCTCACGTGAATTACACCTTTGAAATA | 1260 |
| GAAGCAATGAATGGAGTTTCTGAGTTGAGTTTTTCTCCCAAGCCATTCACAGCTATTACA | 1320 |
| GTGACCACGGATCAAGATGCACCTTCCCTGATAGGTGTGGTAAGGAAGGACTGGGCATCC | 1380 |
| CAAAATAGCATTGCCCTATCATGGCAAGCACCTGCTTTTTCCAATGGAGCCATTCTGGAC | 1440 |
| TACGAGATCAAGTACTATGAGAAAGAACATGAGCAGCTGACCTACTCTTCCACAAGGTCC | 1500 |
| AAAGCCCCCAGTGTCATCATCACAGGTCTTAAGCCAGCCACCAAATATGTATTTCACATC | 1560 |
| CGAGTGAGAACTGCGACAGGATACAGTGGCTACAGTCAGAAATTTGAATTTGAAACAGGA | 1620 |
| GATGAAACTTCTGACATGGCAGCAGAACAAGGACAGATTCTCGTGATAGCCACCGCCGCT | 1680 |
| GTTGGCGGATTCACTCTCCTCGTCATCCTCACTTTATTCTTCTTGATCACTGGGAGATGT | 1740 |
| CAGTGGTACATAAAAGCCAAGATGAAGTCAGAAGAGAAGAGAAGAAACCACTTACAGAAT | 1800 |
| GGGCATTTGCGCTTCCCGGGAATTAAAACTTACATTGATCCAGATACATATGAAGACCCA | 1860 |
| TCCCTAGCAGTCCATGAATTTGCAAAGGAGATTGATCCCTCAAGAATTCGTATTGAGAGA | 1920 |
| GTCATTGGGGCAGGTGAATTTGGAGAAGTCTGTAGTGGGCGTTTGAAGACACCAGGGAAA | 1980 |
| AGAGAGATCCCAGTTGCCATTAAAACTTTGAAAGGTGGCCACATGGATCGGCAAAGAAGA | 2040 |
| GATTTTCTAAGAGAAGCTAGTATCATGGGCCAGTTTGACCATCCAAACATCATTCGCCTA | 2100 |
| GAAGGGGTTGTCACCAAAAGATCCTTCCCGGCCATTGGGGTGGAGGCGTTTTGCCCCAGC | 2160 |
| TTCCTGAGGGCAGGGTTTTTAAATAGCATCCAGGCCCCGCATCCAGTGCCAGGGGGAGGA | 2220 |
| TCTTTGCCCCCCAGGATTCCTGCTGGCAGACCAGTAATGATTGTGGTGGAATATATGGAG | 2280 |
| AATGGATCCCTAGACTCCTTTTTGCGGAAGCATGATGGCCACTTCACAGTCATCCAGTTG | 2340 |
| GTCGGAATGCTCCGAGGCATTGCATCAGGCATGAAGTATCTTTCTGATATGGGTTATGTT | 2400 |
| CATCGAGACCTAGCGGCTCGGAATATACTGGTCAATAGCAACTTAGTATGCAAAGTTTCT | 2460 |
| GATTTTGGTCTCTCCAGAGTGCTGGAAGATGATCCAGAAGCTGCTTATACAACAACTGGT | 2520 |
| GGAAAAATCCCCATAAGGTGGACAGCCCCAGAAGCCATCGCCTACAGAAAATTCTCCTCA | 2580 |
| GCAAGCGATGCATGGAGCTATGGCATTGTCATGTGGGAGGTCATGTCCTATGGAGAGAGA | 2640 |
| CCTTATTGGGAAATGTCTAACCAAGATGTCATTCTGTCCATTGAAGAAGGGTACAGACTT | 2700 |
| CCAGCTCCCATGGGCTGTCCAGCATCTCTACACCAGCTGATGCTCCACTGCTGGCAGAAG | 2760 |
| GAGAGAAATCACAGACCAAAATTTACTGACATTGTCAGCTTCCTTGACAAACTGATCCGA | 2820 |
| AATCCCAGTGCCCTTCACACCCTGGTGGAGGACATCCTTGTAATGCCAGAGTCCCCTGGT | 2880 |
| GAAGTTCCGGAATATCCTTTGTTTGTCACAGTTGGTGACTGGCTAGATTCTATAAAGATG | 2940 |
| GGGCAATACAAGAATAACTTCGTGGCAGCAGGGTTTACAACATTTGACCTGATTTCAAGA | 3000 |
| ATGAGCATTGATGACATTAGAAGAATTGGAGTCATACTTATTGGACACCAGAGACGAATA | 3060 |
| GTCAGCAGCATACAGACTTTACGTTTACACATGATGCACATACAGGAGAAGGGATTTCAT | 3120 |
| GTATGAAAGTACCACAAGCACCTGTGTTTTGTGCCTCAGCATTTCTAAAATGAACGATAT | 3180 |
| CCTCTCTACTACTCTCTCTTCTGATTCTCCAAACATCACTTCACAAACTGCAGTCTTCTG | 3240 |
| TTCAGACTATAGGCACACACCTTATGTTTATGCTTCCAACCAGGATTTTAAAATCATGCT | 3300 |
| ACATAAATCCGTTCTGAATAACCTGCAACTAAAACCCTGGCCCACTGCAGATTATTGCTA | 3360 |
| CGCAATGCAACAGCTTTAAAACCTATCTAGGCCCATGAATGGAAAACAAATCCAAATCCC | 3420 |
| ATCCTTGAAAAGCAAAGGCTCTAAAGAAGCTCTTCAGAAGAGACGGTAAAGAATCAATTC | 3480 |
| TTTTACTTATCACCCAACCACATTTCTTAAAAATGTGTTTTGGTGTCTTTTCCTACCAAA | 3540 |
| TTTCTGCTCTACAAGGCAGTCAGTTAAATCTCTCATTTCATAATTTTCACTGTGATAGAT | 3600 |
| CCTTGCTCTCTCCTCTTTTAATAAATTTAATAAAACTTTAA | |

The disclosed NOV6a nucleic acid sequence, has 3028 of 3367 bases (89%) identical to a gb:GENBANK-ID:MMU58332|acc:U58332.1 mRNA from Mus musculus (Mus musculus receptor tyrosine kinase mRNA, complete cds). The EphA6/ehk-2 disclosed in this invention maps to chromosome 3

A disclosed NOV6a polypeptide (SEQ ID NO:20) encoded by SEQ ID NO:19 is 1035 amino acid residues and is presented using the one-letter amino acid code in Table 6B. Signal P, Psort and/or Hydropathy results predict that NOV6a appears to be a Type Ia membrane protein, contains a signal peptide, and is likely to be localized in the plasma membrane with a certainty of 0.4600. In other embodiments, NOV6a is also likely to be localized to the endoplasmic reticulum with a certainty of 0.1000, or outside the cell with a certainty of 0.1000. The most probable cleavage site is between positions 22 and 23: residues LTA-WT.

TABLE 6B
NOV6a Polypeptide SEQ ID NO:20

| | |
|---|---|
| MGGCEVREFLLQFGFFLPLLTAWTGDCSHVSNQVVLLDTSTVMGELGWKTYPLNGWDAIT | 60 |
| EMDEHNRPIHTYQVCNVMEPNQNNWLRTNWISRDAAQKIYVEMKFTLRDCNSIPWVLGTC | 120 |
| KETFTLYYIESDESHGTKFKPSQYIKIDTIAADESFTQMDLGDRILKLNTEIREVGPIER | 180 |
| KGFYLAFQDIGACIALVSVRVFYKKCPFTVRNLAMFPDTIPRVDSSSLVEVRGSCVKSAE | 240 |
| ERDTPKLYCGADGDWLVPLGRCICSTGYEEIEGSCHACRPGFYKAFAGNTKCSKCPPHSL | 300 |
| TYMEATSVCQCEKGYFRAEKDPPSMACTRPPSAPRNVVFNINETALILEWSPPSDTGGRK | 360 |
| DLTYSVICKKCGLDTSQCEDCGGGLRFIPRHTGLINNSVIVLDFVSHVNYTFEIEAMNGV | 420 |
| SELSFSPKPFTAITVTTDQDAPSLIGVVRKDWASQNSIALSWQAPAFSNGAILDYEIKYY | 480 |
| EKEHEQLTYSSTRSKAPSVIITGLKPATKYVFHIRVRTATGYSGYSQKFEFETGDETSDM | 540 |
| AAEQGQILVIATAAVGGFTLLVILTLFFLITGRCQWYIKAKMKSEEKRRNHLQNGHLRFP | 600 |
| GIKTYIDPDTYEDPSLAVHEFAKEIDPSRIRIERVIGAGEFGEVCSGRLKTPGKREIPVA | 660 |
| IKTLKGGHMDRQRRDFLREASIMGQFDHPNIIRLEGVVTKRSFPAIGVEAFCPSFLRAGF | 720 |
| LNSIQAPHPVPGGGSLPPRIPAGRPVMIVVEYMENGSLDSFLRKHDGHFTVIQLVGMLRG | 780 |
| IASGMKYLSDMGYVHRDLAARNILVNSNLVCKVSDFGLSRVLEDDPEAAYTTTGGKIPIR | 840 |
| WTAPEAIAYRKFSSASDAWSYGIVMWEVMSYGERPYWEMSNQDVILSIEEGYRLPAPMGC | 900 |
| PASLHQLMLHCWQKERNHRPKFTDIVSFLDKLIRNPSALHTLVEDILVMPESPGEVPEYP | 960 |
| LFVTVGDWLDSIKMGQYKNNFVAAGFTTFDLISRMSIDDIRRIGVILIGHQRRIVSSIQT | 1020 |
| LRLHMMHIQEKGFHV | |

The disclosed NOV6a amino acid sequence has 1008 of 1035 amino acid residues (97%) identical to, and 1021 of 1035 amino acid residues (98%) similar to, the 1035 amino acid residue ptnr:SWISSNEW-ACC:Q62413 protein from Mus musculus (Mouse) (EPHRIN TYPE-A RECEPTOR 6 PRECURSOR (EC 2.7.1.112) (TYROSINE-PROTEIN KINASE RECEPTOR EHK-2) (EPH HOMOLOGY KINASE-2)). NOV6a is expressed at least in lung, testis, and B-cells, brain, ear, ovary, thymus, and spleen.

NOV6b: A disclosed NOV6b nucleic acid of 3692 nucleotides identified as SEQ ID NO:21 (also referred to as CG55704-03) encoding an EphA6/ehk-2-like protein is shown in Table 6C. An open reading frame was identified beginning with an ATG initiation codon at nucleotides 19-21 and ending with a TGA codon at nucleotides 3124-3126. Putative untranslated regions are found upstream from the initiation codon and downstream from the termination codon, and are indicated by underline.

TABLE 6C
NOV6b Polynucleotide SEQ ID NO:21

| | |
|---|---|
| AGAGAACCAGCGAGAGCCATGGGGGGCTGCGAAGTCCGGGAATTTCTTTTGCAATTTGGT | 60 |
| TTCTTCTTGCCTCTGCTGACAGCGTGGCCAGGCGACTGCAGTCACGTCTCCAACAACCAA | 120 |
| GTTGTGTTGCTTGATACAACAACTGTACTGGGAGAGCTAGGATGGAAAACATATCCATTA | 180 |
| AATGGGTGGGATGCCATCACTGAAATGGATGAACATAATAGGCCCATTCACACATACCAG | 240 |
| GTATGTAATGTAATGGAACCAAACCAAAACAACTGGCTTCGTACAAACTGGATCTCCCGT | 300 |
| GATGCAGCTCAGAAAATTTATGTGGAAATGAAATTCACACTAAGGGATTGTAACAGCATC | 360 |
| CCATGGGTCTTGGGGACTTGCAAAGAAACATTTAATCTGTTTTATATGGAATCAGATGAG | 420 |
| TCCCACGGAATTAAATTCAAGCCAAACCAGTATACAAAGATCGACACAATTGCTGCTGAT | 480 |
| GAGAGTTTTACCCAGATGGATTTGGGTGATCGCATCCTCAAACTCAACACTGAAATTCGT | 540 |
| GAGGTGGGGCCTATAGAAAGGAAAGGATTTTATCTGGCTTTTCAAGACATTGGGGCGTGC | 600 |
| ATTGCCCTGGTTTCAGTCCGTGTTTTCTACAAGAAATGCCCCTTCACTGTTCGTAACTTG | 660 |
| GCCATGTTTCCTGATACCATTCCAAGGGTTGATTCCTCCTCTTTGGTTGAAGTACGGGGT | 720 |
| TCTTGTGTGAAGAGTGCTGAAGAGCGTGACACTCCTAAACTGTATTGTGGGGCTGATGGA | 780 |
| GATTGGCTGGTTCCTCTTGGAAGGTGCATCTGCAGTACAGGATATGAAGAAATTGAGGGT | 840 |
| TCTTGCCATGCTTGCAGACCAGGATTCTATAAAGCTTTTGCTGGGAACACAAAATGTTCT | 900 |
| AAATGTCCTCCACACAGTTTAACATACATGGAAGCAACTTCTGTCTGTCAGTGTGAAAAG | 960 |
| GGTTATTTCCGAGCTGAAAAAGACCCACCTTCTATGGCATGTACCAGGCCACCTTCAGCT | 1020 |
| CCTAGGAATGTGGTTTTTAACATCAATGAAACAGCCCTTATTTTGGAATGGAGCCCACCA | 1080 |
| AGTGACACAGGAGGGAGAAAAGATCTCACATACAGTGTAATCTGTAAGAAATGTGGCTTA | 1140 |
| GACACCAGCCAGTGTGAGGACTGTGGTGGAGGACTCCGCTTCATCCCAAGACATACAGGC | 1200 |
| CTGATCAACAATTCCGTGATAGTACTTGACTTTGTGTCTCACGTGAATTACACCTTTGAA | 1260 |
| ATAGAAGCAATGAATGGAGTTTCTGAGTTGAGTTTTTCTCCCAAGCCATTCACAGCTATT | 1320 |
| ACAGTGACCACGGATCAAGATGCACCTTCCCTGATAGGTGTGGTAAGGAAGGACTGGGCA | 1380 |
| TCCCAAAATAGCATTGCCCTATCATGGCAAGCACCTGCTTTTTCCAATGGAGCCATTCTG | 1440 |
| GACTACGAGATCAAGTACTATGAGAAAGTCTACCCACGGATAGCGCCGGCATTTTGGCAC | 1500 |
| TACCTGCGGGTAGAAGAACATGAGCAGCTGACCTACTCTTCCACAAGGTCCAAAGCCCCC | 1560 |
| AGTGTCATCATCACAGGTCTTAAGCCAGCCACCAAATATGTATTTCACATCCGAGTGAGA | 1620 |
| ACTGCGACAGGATACAGTGGCTACAGTCAGAAATTTGAATTTGAAACAGGAGATGAAACT | 1680 |
| TCTGACATGGCAGCAGAACAAGGACAGATTCTCGTGATAGCCACCGCCGCTGTTGGCGGA | 1740 |
| TTCACTCTCCTCGTCATCCTCACTTTATTCTTCTTGATCACTGGGAGATGTCAGTGGTAC | 1800 |
| ATAAAAGCCAAGATGAAGTCAGAAGAGAAGAGAAGAAACCACTTACAGAATGGGCATTTG | 1860 |
| CGCTTCCCGGGAATTAAAACTTACATTGATCCAGATACATATGAAGACCCATCCCTAGCA | 1920 |
| GTCCATGAATTTGCAAAGGAGATTGATCCCTCAAGAATTCGTATTGAGAGAGTCATTGGG | 1980 |
| GCAGGTGAATTTGGAGAAGTCTGTAGTGGGCGTTTGAAGACACCAGGGAAAAGAGAGATC | 2040 |
| CCAGTTGCCATTAAAACTTTGAAAGGTGGCCACATGGATCGGCAAAGAAGAGATTTTCTA | 2100 |
| AGAGAAGCTAGTATCATGGGCCAGTTTGACCATCCAAACATCATTCGCCTAGAAGGGGTT | 2160 |
| GTCACCAAAAGATCCTTCCCGGCCATTGGGGTGGAGGCGTTTTGCCCCAGCTTCCTGAGG | 2220 |
| GCAGGGTTTTTAAATAGCATCCAGGCCCCGCATCCAGTGCCAGGGGGAGGATCTTTGCCC | 2280 |
| CCCAGGATTCCTGCTGGCAGACCAGTAATGATTGTGGTGGAATATATGGAGAATGGATCC | 2340 |
| CTAGACTCCTTTTTGCGGAAGCATGATGGCCACTTCACAGTCATCCAGTTGGTCGGAATG | 2400 |
| CTCCGAGGCATTGCATCAGGCATGAAGTATCTTTCTGATATGGGTTATGTTCATCGAGAC | 2460 |
| CTAGCGGCTCGGAATATACTGGTCAATAGCAACTTAGTATGCAAAGTTTCTGATTTTGGT | 2520 |
| CTCTCCAGAGTGCTGGAAGATGATCCAGAAGCTGCTTATACAACAACTGGTGGAAAAATC | 2580 |
| CCCATAAGGTGGACAGCCCCAGAAGCCATCGCCTACAGAAAATTCTCCTCAGCAAGCGAT | 2640 |
| GCATGGAGCTATGGCATTGTCATGTGGGAGGTCATGTCCTATGGAGAGAGACCTTATTGG | 2700 |
| GAAATGTCTAACCAAGATGTCATTCTGTCCATTGAAGAAGGGTACAGACTTCCAGCTCCC | 2760 |
| ATGGGCTGTCCAGCATCTCTACACCAGCTGATGCTCCACTGCTGGCAGAAGGAGAGAAAT | 2820 |
| CACAGACCAAAATTTACTGACATTGTCAGCTTCCTTGACAAACTGATCCGAAATCCCAGT | 2880 |
| GCCCTTCACACCCTGGTGGAGGACATCCTTGTAATGCCAGAGTCCCCTGGTGAAGTTCCG | 2940 |
| GAATATCCTTTGTTTGTCACAGTTGGTGACTGGCTAGATTCTATAAAGATGGGGCAATAC | 3000 |
| AAGAATAACTTCGTGGCAGCAGGGTTTACAACATTTGACCTGATTTCAAGAATGAGCATT | 3060 |
| GATGACATTAGAAGAATTGGAGTCATACTTATTGGACACCAGAGACGAATAGTCAGCAGC | 3120 |
| ATACAGACTTTACGTTTACACATGATGCACATACAGGAGAAGGGATTTCATGTATGAAAG | 3180 |
| TACCACAAGCACCTGTGTTTTGTGCCTCAGCATTTCTAAAATGAACGATATCCTCTCTAC | 3240 |
| TACTCTCTCTTCTGATTCTCCAAACATCACTTCACAAACTGCAGTCTTCTGTTCAGACTA | 3300 |
| TAGGCACACACCTTATGTTTATGCTTCCAACCAGGATTTTAAAATCATGCTACATAAATC | 3360 |
| CGTTCTGAATAACCTGCAACTAAAACCCTGGCCCACTGCAGATTATTGCTACGCAATGCA | 3420 |
| ACAGCTTTAAAACCTATCTAGGCCCATGAATGGAAAACAAATCCAAATCCGATCCTTGAA | 3480 |
| AAGCAAAGGCTCTAAAGAAGCTCTTCAGAAGAGACGGTAAAGAATGAATTCTTTTACTTA | 3540 |
| TCACCCAACCACATTTCTTAAAAATGTGTTTTGGTGTCTTTTCCTACCAAATTTCTGCTC | 3600 |
| TACAAGGCAGTCAGTTAAATCTCTCATTTCATAATTTTCACTGTGATAGATCCTTGCTCT | 3660 |
| CTCCTCTTTTAATAAATTTAATAAAACTTTAA | |

The disclosed NOV6b nucleic acid sequence has 3028 of 3367 bases (89%) identical to a gb:GENBANK-ID:MMU58332|acc:U58332.1 mRNA from Mus musculus (Mus musculus receptor tyrosine kinase mRNA, complete cds)

A disclosed NOV6b polypeptide (SEQ ID NO:22) encoded by SEQ ID NO:21 is 1035 amino acid residues and is presented using the one-letter amino acid code in Table 6D. Signal P, Psort and/or Hydropathy results predict that NOV6a appears to be a Type Ia membrane protein, contains a signal peptide, and is likely to be localized in the plasma membrane with a certainty of 0.4600. In other embodiments, NOV6b is also likely to be localized to the endoplasmic reticulum with a certainty of 0.1000, or outside the cell with a certainty of 0.1000. The most probable cleavage site is between positions 22 and 23: residues LTA-WP.

TABLE 6D
NOV6b Polypeptide SEQ ID NO:22

| | |
|---|---|
| MGGCEVREFLLQFGFFLPLLTAWPGDCSHVSNNQVVLLDTTTVLGELGWKTYPLNGWDAI | 60 |
| TEMDEHNRPIHTYQVCNVMEPNQNNWLRTNWISRDAAQKIYVEMKFTLRDCNSIPWVLGT | 120 |
| CKETFNLFYMESDESHGIKFKPNQYTKIDTIAADESFTQMDLGDRILKLNTEIREVGPIE | 180 |
| RKGFYLAFQDIGACIALVSVRVFYKKCPFTVRNLAMFPDTIPRVDSSSLVEVRGSCVKSA | 240 |
| EERDTPKLYCGADGDWLVPLGRCICSTGYEEIEGSCHACRPGFYKAFAGNTKCSKCPPHS | 300 |
| LTYMEATSVCQCEKGYFRAEKDPPSMACTRPPSAPRNVVFNINETALILEWSPPSDTGGR | 360 |
| KDLTYSVICKKCGLDTSQCEDCGGGLRFIPRHTGLINNSVIVLDFVSHVNYTFEIEAMNG | 420 |
| VSELSFSPKPFTAITVTTDQDAPSLIGVVRKDWASQNSIALSWQAPAFSNGAILDYEIKY | 480 |
| YEKVYPRIAPAFWHYLRVEEHEQLTYSSTRSKAPSVIITGLKPATKYVFHIRVRTATGYS | 540 |
| GYSQKFEFETGDETSDMAAEQGQILVIATAAVGGFTLLVILTLFFLITGRCQWYIKAKMK | 600 |
| SEEKRRNHLQNGHLRFPGIKTYIDPDTYEDPSLAVHEFAKEIDPSRIRIERVIGAGEFGE | 660 |
| VCSGRLKTPGKREIPVAIKTLKGGHMDRQRRDFLREASIMGQFDHPNIIRLEGVVTKRSF | 720 |
| PAIGVEAFCPSFLRAGFLNSIQAPHPVPGGGSLPPRIPAGRPVMIVVEYMENGSLDSFLR | 780 |
| KHDGHFTVIQLVGMLRGIASGMKYLSDMGYVHRDLAARNILVNSNLVCKVSDFGLSRVLE | 840 |
| DDPEAAYTTTGGKIPIRWTAPEAIAYRKFSSASDAWSYGIVMWEVMSYGERPYWEMSNQD | 900 |
| VILSIEEGYRLPAPMGCPASLHQLMLHCWQKERNHRPKFTDIVSFLDKLIRNPSALHTLV | 960 |
| EDILVMPESPGEVPEYPLFVTVGDWLDSIKMGQYKNNFVAAGFTTFDLISRMSIDDIRRI | 1020 |
| GVILIGHQRRIVSSIQTLRLHMNHIQEKGFHV | |

The disclosed NOV6b amino acid sequence has 1008 of 1035 amino acid residues (97%) identical to, and 1021 of 1035 amino acid residues (98%) similar to, the 1035 amino acid residue ptnr:SWISSNEW-ACC:Q62413 protein from Mus musculus (Mouse) (EPHRIN TYPE-A RECEPTOR 6 PRECURSOR (EC 2.7.1.112) (TYROSINE-PROTEIN KINASE RECEPTOR EHK-2) (EPH HOMOLOGY KINASE-2)).NOV6b is expressed at least in lung, testis, and B-cells, brain, ear, ovary, thymus, and spleen.

TABLE 6E
BLAST results for NOV6

| Gene Index/ | Protein/ | Length | Identity | Positives | |
|---|---|---|---|---|---|
| Identifier | Organism | (aa) | (%) | (%) | Expect |
| gi\|4885211\|ref\|NP_005224.1\| | EphA3; Ephrin | 983 | 62 | 76 | 0.0 |
| (NM_005233) | receptor EphA3 | | | | |
| | (human embryo | | | | |
| | kinase 1); eph- | | | | |
| | like tyrosine | | | | |
| | kinase 1 (human | | | | |
| | embryo kinase | | | | |
| | 1); ephrin | | | | |
| | receptor EphA3 | | | | |
| | [Homo sapiens] | | | | |
| gi\|125340\|sp\|P09759\| | EPHRIN TYPE-B | 984 | 54 | 69 | 0.0 |
| EPB1_RAT | RECEPTOR 1 | | | | |
| | PRECURSOR | | | | |
| | (TYROSINE- | | | | |
| | PROTEIN KINASE | | | | |
| | RECEPTOR EPH-2) | | | | |
| | (ELK) | | | | |
| gi\|8134439\|sp\|Q91694\| | EPHRIN TYPE-A | 985 | 58 | 72 | 0.0 |
| EP4B_XENLA | RECEPTOR 4B | | | | |
| | PRECURSOR | | | | |
| | (TYROSINE- | | | | |
| | PROTEIN KINASE | | | | |
| | RECEPTOR PAG) | | | | |
| | (PAGLIACCIO) | | | | |
| gi\|1079403\|pir∥A56599 | embryo kinase 5 - | 995 | 53 | 69 | 0.0 |
| | chicken | | | | |
| gi\|1706629\|sp\|P54757\| | EPHRIN TYPE-A | 1005 | 59 | 73 | 0.0 |
| EPA5_RAT | RECEPTOR 5 | | | | |
| | PRECURSOR | | | | |
| | (TYROSINE- | | | | |
| | PROTEIN KINASE | | | | |
| | RECEPTOR EHK-1) | | | | |
| | (EPH HOMOLOGY | | | | |
| | KINASE-1) | | | | |

The homology of these sequences is shown graphically in the ClustalW analysis shown in Table 6F.

[image]

[image]

[image]

[image]

[image]

[image]

Tables 6G lists the domain description from DOMAIN analysis results against NOV6. This indicates that the NOV6 sequence has properties similar to those of other proteins known to contain this domain.

TABLE 6G

Domain Analysis of NOV6
gnl|Pfam|pfam01404, EPH_lbd, Ephrin receptor ligand binding
domain. The Eph receptors, which bind to ephrins pfam00812
are a large family of receptor tyrosine kinases. This family
represents the amino terminal domain which binds the ephrin
ligand.
CD-Length = 174 residues, 100.0% aligned
Score = 345 bits (886), Expect = 6e−96

| | | | |
|---|---|---|---|
| Query: | 33 | QVVLLDTSTVMGELGWKTYPLNGWDAITEMDEHNRPIHTYQVCNVMEPNQNNWLRTNWIS | 92 |
| | | +V LLDT+T GELGW TYP GW+ ++ +DE+NRPI TYQVCNVMEPNQNNWLRTNWI | |
| Sbjct: | 1 | EVTLLDTTTATGELGWLTYPPGGWEEVSGLDENNRPIRTYQVCNVMEPNQNNWLRTNWIP | 60 |
| | | ∘ ∘ ∘ ∘ ∘ ∘ | |
| Query: | 93 | RDAAQKIYVEMKFTLRDCNSIPWVLGTCKETFTLYYIESDESHGTKFKPSQYIKIDTIAA | 152 |
| | | R AQ++YVE+KFT+RDCNS+P VLGTCKETF LYY ESDE G ++ +QY K+DTIAA | |
| Sbjct: | 61 | RRGAQRVYVELKFTVRDCNSLPGVLGTCKETFNLYYYESDEDVGPAWRENQYTKVDTIAA | 120 |
| Query: | 153 | DESFTQMDLGDRILKLNTEIREVGPIERKGFYLAFQDIGACIALVSVRVFYKKC | 206 |
| | | DESFTQ+DLGDR++KLNTE+R VGP+ +KGFYLAFQD+GAC+ALVSVRVFYKKC | |
| Sbjct: | 121 | DESFTQVDLGDRVMKLNTEVRSVGPLSKKGFYLAFQDVGACMALVSVRVFYKKC | 174 |

The gene of invention is an ortholog of mouse EphA6 (also known as m-ehk2) which belongs to the superfamily of receptor tyrosine kinases, which constitute the largest family of oncogenes. This family includes prominent growth factor receptors such as those for epidermal growth factor, platelet-derived growth factor etc. Members of this superfamily influence cell shape, mobility, differentiation and proliferation.

Within this superfamily, the Ephrin (Eph) receptors constitute the largest subfamily. Eph receptors and their ligands, ephrins, are known to be involved in several normal developmental processes, including formation of segmented structures, axon guidance, cell adhesion and development of vasculature. Ephrin receptors are classified into two main subtypes: EphA receptors bind to GPI-anchored ephrin-A ligands, while EphB receptors bind to ephrin-B proteins that have a transmembrane and cytoplasmic domain. The EphA6 receptor is highly expressed in the mouse brain and inner ear, including the cochlea. This receptor is also differentially expressed relative to the other ephrin receptors in certain regions of the primate neocortex during development. In addition, it is found in the developing retina and optic tectum in the chicken. It may, therefore, be involved in the development of these structures. It shows the presence of conserved ephrin and protein kinase domains, similar to the protein of invention. The protein of invention, therefore, may be involved in the development and/or dysgenesis of a variety of tissues (see, Maisonpierre P C, et al., Oncogene December 1993;8(12):3277-88); Lee A M, et al., DNA Cell Biol October 1996;15(10):817-25; Dodelet V C, et al., Oncogene Nov. 20, 2000;19 (49):5614-9; Mellitzer G, et al., Curr Opin Neurobiol June 2000;10(3):400-8; Holder N. et al., Development May 1999;126(10):2033-44; Matsunaga T, et al., Eur J Neurosci May 2000;12(5):1599-616; Donoghue M J, et al., J Neurosci Jul. 15, 1999;19(14):5967-79; Connor R J et al., Dev Biol Jan. 1, 1998;193(1):21-35, incorporated by reference).

The ephrin domain (IPR001090) is a feature of ephrins and ephrin receptors. IPR000719 is a catalytic domain characteristic of eukaryotic protein kinases. In the N-terminal extremity of the catalytic domain there is a glycine-rich stretch of residues in the vicinity of a lysine residue, which has been shown to be involved in ATP binding. In the central part of the catalytic domain there is a conserved aspartic acid residue which is important for the catalytic activity of the enzyme. The fibronectin type III repeat region (IPR001777) is an approximately 100 amino acid domain, different tandem repeats of which contain binding sites for DNA, heparin and the cell surface. The superfamily of sequences believed to contain FnIII repeats represents 45 different families, the majority of which are involved in cell surface binding in some manner, or are receptor protein tyrosine kinases, or cytokine receptors. The sterile alpha motif (SAM) domain (IPR001660) is a putative protein interaction module present in a wide variety of proteins involved in many biological processes. SAM domains have been shown to homo-and hetero-oligomerize, mediating specific protein-protein interactions. This indicates that the sequence of the invention has properties similar to those of other proteins known to contain these domains and similar to the properties of these domains.

The disclosed NOV6a nucleic acid of the invention encoding an EphA6-like protein includes the nucleic acid whose sequence is provided in Table 6A or a fragment thereof. The invention also includes a mutant or variant nucleic acid any of whose bases may be changed from the corresponding base shown in Table 6A while still encoding a protein that maintains its EphA6-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant protein, up to about 3 percent of the residues may be so changed.

The disclosed NOV6b protein of the invention includes the EphA6-like protein whose sequence is provided in Table 6D. The invention also includes a mutant or variant protein any of whose residues may be changed from the corresponding residue shown in Table 6D while still encoding a protein that maintains its EphA6-like activities and physiological functions, or a functional fragment thereof. In the mutant or variant nucleic acids, and their complements, up to about 11 percent of the bases may be so changed. In the mutant or variant protein, up to about 3 percent of the residues may be so changed.

The above defined information for this invention suggests that EphA6-like proteins (NOV6) may function as a member of an Ephrin receptor family. Therefore, the NOV6 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of NOV6 are useful in, for example, treatment of patients suffering from: hemophilia, hypercoagulation, idiopathic thrombocytopenic purpura, immunodeficiencies, graft versus host disease, systemic lupus erythematosus, autoimmune disease, asthma, emphysema, scleroderma, allergy, ARDS, fertility, cancer, developmental disorders and other diseases, disorders and conditions of the like.

The novel NOV6 nucleic acid encoding NOV6 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

NOV6 nucleic acids and polypeptides are further useful in the generation of antibodies that bind immuno specifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV6a protein has multiple hydrophilic regions, each of which can be used as an immunogen. In one embodiment, contemplated NOV6a epitope is from about amino acids 50 to 125. In other embodiments, NOV6a epitope is from about amino acids 175 to 200, from about amino acids 210 to 400, or from about amino acids 420 to 675, from about 700 to 720, from about 760 to 780, from about 795 to 805, and from about 806 to 950. The disclosed NOV6b protein has multiple hydrophilic regions, each of which can be used as an immunogen. In one embodiment, contemplated NOV6b epitope is from about amino acids 50 to 125. In other embodiments, NOV6b epitope is from about amino acids 175 to 200, from about amino acids 210 to 400, or from about amino acids 420 to 675, from about 720 to 740, from about 770 to 790, from about 795 to 805, and from about 806 to 950. This novel protein also has value in development of powerful assay system for functional analysis of various human disorders, which will help in understanding of pathology of the disease and development of new drug targets for various disorders.

NOV7

A disclosed NOV7 nucleic acid of 1607 nucleotides identified as SEQ ID NO:23 (also referred to as CG94323538) encoding a glucose transporter-like protein is shown in Table 7A. An open reading frame was identified beginning with an ATG initiation codon at nucleotides 68-70 and ending with a TAG codon at nucleotides 1517-1519.

TABLE 7A
NOV7 Polynucleotide SEQ ID NO:23

| | |
|---|---|
| TGGGTTTAACTGTGTCTTATAGGTGTTAGCAGAAAAACCTCTCTGTACAATGACAAGTGG | 60 |
| CCACTGAGAACACTTTCTCATTTCTCATGAACTGCCCAATATTCTTAGCTGTGGATGGGG | 120 |
| CAATGTTTTCCAGGTCTTCAAGTCATTTTACAACGAAACCTACTTTGAGCGACACGCAAC | 180 |
| ATTCATGGACGGGAAGCTCATGCTGCTTCTATGGTCTTGCACCGTCTCCATGTTTCCTCT | 240 |
| GGGCGGCCTGTTGGGGTCATTGCTCGTGGGCCTGCTGGTTGATAGCTGCGGCAGAAAGGG | 300 |
| GACCCTGCTGATCAACAACATCTTTGCCATCATCCCCGCCATCCTGATGGGAGTCAGCAA | 360 |
| AGTGGCCAAGGCTTTTGAGCTGATCGTCTTTTCCCGAGTGGTGCTGGGAGTCTGTGCAGG | 420 |
| TATCTCCTACAGCGCCCTTCCCATGTACCTGGGAGAACTGGCCCCCAAGAACCTGAGAGG | 480 |
| CATGGTGGGAACAATGACCGAGGTTTTCGTCATCGTTGGAGTCTTCCTAGCACAGATCTT | 540 |
| CAGCCTCCAGGCCATCTTGGGCAACCCGGCAGGCTGGCCGGTGCTTCTGGCGCTCACAGG | 600 |
| GGTGCCCGCCCTGCTGCAGCTGCTGACCCTGCCCTTCTTCCCCGAAAGCCCCCGCTACTC | 660 |
| CCTGATTCAGAAAGGAGATGAAGCCACAGCGCGGCCTCTGAGGAGGCTGAGAGGCCACAC | 720 |
| GGACATGGAGGCCGAGCTGGAGGACATGCGTGCGGAGGCCCGGGCCGAGCGCGCCGAGGG | 780 |
| CCACCTGTCTGTGCTGCACCTCTGTGCCCTGCGGTCCCTGCGCTGGCAGCTCCTCTCCAT | 840 |
| CATCGTGCTCATGGCCGGCCAGCAGCTGTCGGGCATCAATGCGATCAACTACTATGCGGA | 900 |
| CACCATCTACACATCTGCGGGCGTGGAGGCCGCTCACTCCCAATATGTAACGGTGGGCTC | 960 |
| TGGCGTCGTCAACATAGTGATGACCATCACCTCGGTGGTCCTTGTGGAGCGGCTGGGACG | 1020 |
| GCGGCACCTCCTGCTGCCCGGCTACGGCATCTGCGGCTCTGCCTGCCTGGTGCTGACGGT | 1080 |
| CTCTCCCCCCCCACAGAACAGGGTCCCCGAGCTGTCCTACCTCGGCATCATCTGTGTCTT | 1140 |
| TGCCTACATCGCGGGACATTCCATTGGGCCCAGTCCTGTCCCCTCGGTGGTGAGGACCGA | 1200 |
| GATCTTCCTGCAGTCCTCCCGGCGGGCAGCTTTCATGGTGGACGGGGCAGTGCACTGGCT | 1260 |
| CACCAACTTCATCATAGGCTTCCTGTTCCCATCCATCCAGGAGGCCATCGGTGCCTACAG | 1320 |
| TTTCATCATCTTTGCCGGAATCTGCCTCCTCACTGCGATTTACATCTACGTGGTTATTCC | 1380 |
| GGAGACCAAGGGCAAAACATTTGTGGAGATAAACCGCATTTTTGCCAAGAGAAACAGGGT | 1440 |
| GAAGCTTCCAGAGGAGAAAGAAGAAACCATTGATGCTGGGCCTCCCACAGCCTCTCCTGC | 1500 |
| CAAGGAAACTTCCTTTTAGTGGCCCTGCATCAAGGACCGGAGCCCATATTCAAGGCTTCC | 1560 |
| TTCTATGACAATGGGCCTCCCGGCCCCAGGCTCTGGGGAGGATAATA | |

The disclosed NOV7 nucleic acid sequence, localized to chromosome 1, has 933 of 1328 bases (70%) identity to a gb:GENBANK-ID:HUMGLUT5|acc:M55531.1 mRNA from Homo sapiens (Human glucose transport-like 5 (GLUT5) mRNA, complete cds).

A disclosed NOV7 polypeptide (SEQ ID NO:24) encoded by SEQ ID NO:23 is 483 amino acid residues and is presented using the one-letter amino acid code in Table 7B. Signal P, Psort and/or Hydropathy results predict that NOV7 has a signal peptide and is likely to be localized at the plasma membrane with a certainty of 0.6000. In other embodiments, NOV7 is also likely to be localized to the golgi body with a certainty of 0.4000, to the endoplasmic reticulum (membrane) with a certainty of 0.3000, or the mitochondrial inner membrane with a certainty of 0.3000. The most likely cleavage site for a NOV7 peptide is between amino acids 18 and 19, at: GWG-NV.

TABLE 7B
NOV7 Polypeptide SEQ ID NO:24

| | |
|---|---|
| EHFLISHELPNILSCGWGNVFQVFKSFYNETYFERHATFMDGKLMLLLWSCTVSMFPLGG | 60 |
| LLGSLLVGLLVDSCGRKGTLLINNIFAIIPAILMGVSKVAKAFELIVFSRVVLGVCAGIS | 120 |
| YSALPMYLGELAPKNLRGMVGTMTEVFVIVGVFLAQIFSLQAILGNPAGWPVLLALTGVP | 180 |
| ALLQLLTLPFFPESPRYSLIQKGDEATARPLRRLRGHTDMEAELEDMRAEARAERAEGHL | 240 |
| SVLHLCALRSLRWQLLSIIVLMAGQQLSGINAINYYADTIYTSAGVEAAHSQYVTVGSGV | 300 |
| VNIVMTITSVVLVERLGRRHLLLAGYGICGSACLVLTVSPPPQNRVPELSYLGIICVFAY | 360 |
| IAGHSIGPSPVPSVVRTEIFLQSSRRAAFMVDGAVHWLTNFIIGFLFPSIQEAIGAYSFI | 420 |
| IFAGICLLTAIYIYVVIPETKGKTFVEINRIFAKRNRVKLPEEKEETIDAGPPTASPAKE | 480 |
| TSF | |

The disclosed NOV7 amino acid sequence has 272 of 455 amino acid residues (59%) identical to, and 348 of 455 amino acid residues (76%) similar to, the 501 amino acid residue ptnr:SWISSPROT-ACC:P22732 protein from Homo sapiens (Human) (GLUCOSE TRANSPORTER TYPE 5, SMALL INTESTINE (FRUCTOSE TRANSPORTER)). NOV7 also has homology to the amino acid sequence shown in the BLASTP data listed in Table 7C.

TABLE 7C
BLAST results for NOV7

| Gene Index/ | | Length | Identity | Positives | |
|---|---|---|---|---|---|
| Identifier | Protein/Organism | (aa) | (%) | (%) | Expect |
| gi\|17441225\|ref\|XP_060424.1\| | similar to | 524 | 98 | 98 | 0.0 |
| (XM_060424) | solute carrier | | | | |
| | family 2 | | | | |
| | (facilitated | | | | |
| | glucose | | | | |
| | transporter), | | | | |
| | member 5 (H. sapiens) | | | | |
| | [Homo | | | | |
| | sapiens] | | | | |
| gi\|1170105\|sp\|P43427\| | Solute carrier | 502 | 57 | 77 | e−146 |
| GTR5_RAT | family 2, | | | | |
| | facilitated | | | | |
| | glucose | | | | |
| | transporter, | | | | |
| | member 5 | | | | |
| | (Glucose | | | | |
| | transporter | | | | |
| | type 5, small | | | | |
| | intestine) | | | | |
| | (Fructose | | | | |
| | transporter) | | | | |
| gi\|13929044\|ref\|NP_113929.1\| | solute carrier | 502 | 57 | 78 | e−146 |
| (NM_031741) | family 2 | | | | |
| | (facilitated | | | | |
| | glucose | | | | |
| | transporter), | | | | |
| | member 5 | | | | |
| | [Rattus | | | | |
| | norvegicus] | | | | |
| gi\|204416\|gb\|AAA02627.1\| | fructose | 502 | 57 | 77 | e−146 |
| (L05195) | transporter | | | | |
| | [Rattus | | | | |
| | norvegicus] | | | | |
| gi\|9789967\|ref\|NP_062715.1\| | solute carrier | 501 | 56 | 75 | e−140 |
| (NM_019741) | family 2 | | | | |
| | (facilitated | | | | |
| | glucose | | | | |
| | transporter), | | | | |
| | member 5; | | | | |
| | fructose | | | | |
| | transporter | | | | |
| | [Mus musculus] | | | | |

The homology of these sequences is shown graphically in the ClustalW analysis shown in Table 7D.

[image]

[image]

[image]

Table 7E lists the domain description from DOMAIN analysis results against NOV7. This indicates that the NOV7 sequence has properties similar to those of other proteins known to contain this domain.

TABLE 7E

Domain Analysis of NOV7
gnl|Pfam|pfam00083, sugar_tr, Sugar (and other) transporter.
CD-Length = 447 residues, 96.6% aligned
Score = 246 bits (629), Expect = 2e−66

| | | | |
|---|---|---|---|
| Query: | 21 | FQVFKSFYNETYFERHATFM----DGKLMLLLWSCTVSMFPLGGLLGSLLVGLLVDSCGR | 76 |
| | | V F F + +L VS+F +G +GSL G L D GR | |
| Sbjct: | 16 | TGVIGGFATLIDFLFFFGGLTSSGSCAESTVLSGLVVSIFFVGRPIGSLFAGKLGDRFGR | 75 |
| Query: | 77 | KGTLLINNIFAIIPAILMGVSKVAKAFELIVFSRVVLGVCAGISYSALPMYLGELAPKNL | 136 |
| | | K +LLI + +I ++L G++ A F L++ RV++G+ G + +PMY+ E+APK L | |
| Sbjct: | 76 | KKSLLIGLVLFVIGSLLSGLAPGA--FYLLIVGRVLVGLGVGGASVLVPMYISEIAPKAL | 133 |
| Query: | 137 | RGMVGTMTEVFVIVGVFLAQIFSLQAILGNPAGWPVLLALTGVPALLQLLTLPFFPESPR | 196 |
| | | RG +G++ ++ + +G+ +A I L N GW + L L VPALL L+ L F PESPR | |
| Sbjct: | 134 | RGALGSLYQLGITIGILVAAIIGLGLNKTNNWGWRIPLGLQLVPALLLLIGLLFLPESPR | 193 |
| Query: | 197 | YSLIQKGDEATARPLRRLRGHTDMEAELEDMRAEARAERAEGHLSVLHLCALRSLRWQLL | 256 |
| | | + +++ E L +LRG D++ E+++ +AE A + + R +LL | |
| Sbjct: | 194 | WLVLKGKLEEARAVLAKLRGVEDVDQEIQEEKAELEAGVSSEKAGLELF--RGRTRQRLL | 251 |
| Query: | 257 | SIIVLMAGQQLSGINAINYYADTIYTSAGVEAAHSQYVTVGSGVVNIVMTITSVVLVERL | 316 |
| | | ++L QQL+GINAI YY+ TI+ S G+ + + VT+ GVVN V T ++ LV+R | |
| Sbjct: | 252 | MGVMLQIFQQLTGINAIFYYSPTIFKSVGMSDSVALLVTIIVGVVNFVATFVAIFLVDRF | 311 |
| Query: | 317 | GRRHLLLAGYGICGSACLVLTVSPPPQNRVPELSYLGIICVFAYIAGHSIGPSPVPSVVR | 376 |
| | | GRR LLL G L+L V+ P + I+ + +IA ++G P+P V+ | |
| Sbjct: | 312 | GRRPLLLLGAAGMAICFLILGVA-LLLLNKPGAGIVAIVFILLFIAFFALGWGPIPWVIL | 370 |
| Query: | 377 | TEIFLQSSRRAAFMVDGAVHWLTNFIIGFLFPSIQEAIG-AYSFIIFAGICLLTAIYIYV | 435 |
| | | +E+F R A + A +WL NFIIGFLFP I AIG Y F+ FAG+ +L +++Y | |
| Sbjct: | 371 | SELFPTGVRSKAMALATAANWLANFIIGFLFPYITGAIGGGYVFLFFAGLLVLFILFVYF | 430 |
| Query: | 436 | VIPETKGKTFVEINRIF | 452 |
| | | +PETKG+T EI+ +F | |
| Sbjct: | 431 | FVPETKGRTLEEIDELF | 447 |

Sugar transport is a critical feature of many cell types in the body as energy storage and metabolism or defects thereof can cause a variety of human diseases. For example, glucose tranporter 4 (GLUT4) is critical to insulin-sensitive glucose uptake. Novel sugar transporters can be important for obesity, diabetes, and cancer targets (see, Hundal H S, et al., Adv Exp Med Biol 1998;441:35-45).

Biochemical and immunocytochemical studies have revealed that, in addition to GLUT1 and GLUT4, human skeletal muscle also expresses the GLUT5 hexose transporter. The subcellular distribution of GLUT5 is distinct from that of GLUT4, being localised exclusively in the sarcolemmal membrane. The substrate selectivity of GLUT5 is also considered to be different to that of GLUT1 and GLUT4 in that it operates primarily as a fructose transporter. Consistent with this suggestion studies in isolated human sarcolemmal vesicles have shown that fructose transport obeys saturable kinetics with a Vmax of 477±37 pmol.mg protein-1 min-1 and a Km of 8.3±1.2 mM. Unlike glucose uptake, fructose transport in sarcolemmal vesicles was not inhibited by cytochalasin B suggesting that glucose and fructose are unlikely to share a common route of entry into human muscle. Muscle exercise, which stimulates glucose uptake through the increased translocation of GLUT4 to the plasma membrane, does not increase fructose transport or sarcolemmal GLUT5 content. In contrast, muscle inactivity, induced as a result of limb immobilization, caused a significant reduction in muscle GLUT4 expression with no detectable effects on GLUT5. The presence of a fructose transporter in human muscle is compatible with studies showing that this tissue can utilise fructose for both glycolysis and glycogenesis. However, the full extent to which provision of fructose via GLUT5 is important in meeting the energy requirements of human muscle during both physiological and pathophysiological circumstances remains an issue requiring further investigation.

The disclosed NOV7 nucleic acid of the invention encoding a glucose transporter-like protein includes the nucleic acid whose sequence is provided in Table 7A or a fragment thereof. The invention also includes a mutant or variant nucleic acid any of whose bases may be changed from the corresponding base shown in Table 7A while still encoding a protein that maintains its glucose transporter-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 30 percent of the bases may be so changed.

The disclosed NOV7 protein of the invention includes glucose transporter-like protein whose sequence is provided in Table 7B. The invention also includes a mutant or variant protein any of whose residues may be changed from the corresponding residue shown in Table 7B while still encoding a protein that maintains its glucose transporter-like activities and physiological functions, or a functional fragment thereof. In the mutant or variant protein, up to about 41 percent of the residues may be so changed.

The protein similarity information, expression pattern, and map location for the glucose transporter-like protein and nucleic acid (NOV7) disclosed herein suggest that NOV7 may have important structural and/or physiological functions characteristic of glucose transporter family. Therefore, the NOV7 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV7 nucleic acids and proteins of the invention are useful in potential diagnostic and therapeutic applications implicated in various diseases and disorders described below and/or other pathologies. For example, the compositions of the present invention will have efficacy for treatment of patients suffering from obesity, diabetes, cancer, inflammation, CNS diseases and other diseases, disorders and conditions of the like. The NOV7 nucleic acid, or fragments thereof, may further be useful in diagnostic applications, wherein the presence or amount of the nucleic acid or the protein are to be assessed.

NOV7 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV7 protein have multiple hydrophilic regions, each of which can be used as an immunogen. In one embodiment, contemplated NOV7 epitope is from about amino acids 20 to 40. In other embodiments, contemplated NOV7 epitopes are from amino acids 200 to 250, from amino acids 260 to 265, from amino acids 360 to 365, or from amino acids 440 to 460. This novel protein also has value in development of powerful assay system for functional analysis of various human disorders, which will help in understanding of pathology of the disease and development of new drug targets for various disorders.

NOV8

A disclosed NOV8 nucleic acid of 3270 nucleotides identified as SEQ ID NO:25 (also designated as Acc. No. CG95545-01) encoding a novel Type Ia Membrane Sushi-Containing Domain-like protein is shown in Table 8A. An open reading frame was identified beginning with an ATG initiation codon at nucleotides 309-311 and ending with a TGA codon at nucleotides 2550-2552.

TABLE 8A
NOV8 Polynucleotide SEQ ID NO:25

CGGGGCTCTGCGTCAGCTCTGTCATTATCCGATGAGTGTCTGTCCCCCTTTGCGAATGTGAGCGGCGAGA
GGGCAGCAAGTGCGGAGCCAGAGACGGACGCGGAACGGGCGTGTCCTAAGCCCAGGCCCCGACAGGAGGA
AGGACCCGCGCTCTGCGGCCTCCCGGGGACCCCGCAGCGCCCCCCGCTTCCCTCGGCGGCGCCGGAAGCC
GCCGGCTGGTCCCCTCCCCGCGGCGCCTGTAGCCTTATCTCTGCACCCTGAGGGCCCCGGGAGGAGGCGC
GGGCGCGCCGGGAGGGACCGGCGGCGGCATGGGCCGGGGGCCCTGGGATGCGGGCCCGTCTCGCCGCCTG
CTGCCGCTGTTGCTGCTGCTCGGCCTGGCCCGCGGCGCCGCGGGAGCGCCGGGCCCCGACGGTTTAGACG
TCTGTGCCACTTGCCATGAACATGCCACATGCCAGCAAAGAGAAGGGAAGAAGATCTGTATTTGCAACTA
TGGATTTGTAGGGAACGGGAGFGACTCAGTGTGTTGATAAAAATGAGTGCCAGTTTGGAGCCACTCTTGT
CTGTGGGAACCACACATCTTGCCACAACACCCCCGGGGGCTTCTATTGCATTTGCCTGGAAGGATATCGA
GCCACAAACAACAACAAGACATTCATTCCCAACGATGGCACCTTTTGTACAGACATAGATGAGTGTGAAG
TTTCTGGCCTGTGCAGGCATGGAGGGCGATGCGTGAACACTCATGGGAGCTTTGAATGCTACTGTATGGA
TGGATACTTGCCAAGGAATGGACCTGAACCTTTCCACCCGACCACCGATGCCACATCATGCACAGAAATA
GACTGTGGTACCCCTCCTGAGGTTCCAGATGGCTATATCATAGGAAATTATACGTCTAGTCTGGGCAGCC
AGGTTCGTTATGCTTGCAGAGAAGGATTCTTCAGTGTTCCAGAAGATACAGTTTCAAGCTGCACAGGCCT
GGGCACATGGGAGTCCCCAAAATTACATTGCCAAGAGATCAACTGTGGCAACCCTCCAGAAATGCGGCAC
GCCATCTTGGTAGGAAATCACAGCTCCAGGCTGGGCGGTGTGGCTCGCTATGTCTGTCAAGAGGGCTTTG
AGAGCCCTGGAGGAAAGATCACTTCTGTTTGCACAGAGAAAGGCACCTGGAGAGAAAGTACTTTAACATG
CACAGAAATTCTGACAAAGATTAATGATGTATCACTGTTTAATGATACCTGTGTGAGATGGCAAATAAAC
TCAAGAAGAATAAACCCCAAGATCTCATATGTGATATCCATAAAAGGACAACGGTTGGACCCTATGGAAT
CAGTTCGTGAGGAGACAGTCAACTTGACCACAGACAGCAGGACCCCAGAAGTGTGCCTAGCCCTGTACCC
AGGCACCAACTACACCGTGAACATCTCCACAGCACCTCCCAGGCGCTCGATGCCAGCCGTCATCGGTTTC
CAGACAGCTGAAGTTGATCTCTTAGAAGATGATGGAAGTTTCAATATTTCAATATTTAATGAAACTTGTT
TGAAATTGAACAGGCGTTCTAGGAAAGTTGGATCAGAACACATGTACCAATTTACCGTTCTGGGTCAGAG
GTGGTATCTGGCTAACTTTTCTCATGCAACATCGTTTAACTTCACAACGAGGGAACAAGTGCCTGTAGTG
TGTTTGGATCTGTACCCTACGACTGATTATACGGTGAATGTGACCCTGCTGAGATCTCCTAAGCGGCACT
CAGTGCAAATAACAATAGCAACTCCCCCAGCAGTAAAACAGACCATCAGTAACATTTCAGGATTTAATGA
AACCTGCTTGAGATGGAGAAGCATCAAGACAGCTGATATGGAGGAGATGTATTTATTCCACATTTGGGGC
CAGAGATGGTATCAGAAGGAATTTGCCCAGGAAATGACCTTTAATATCAGTAGCAGCAGCCGAGATCCCG
AGGTGTGCTTGGACCTACGTCCGGGTACCAACTACAATGTCAGTCTCCGGGCTCTGTCTTCGGAACTTCC
TGTGGTCATCTCCCTGACAACCCAGATAACAGAGCCTCCCCTCCCGGAAGTAGAATTTTTTACGGTGCAC
AGAGGACCTCTACCACGCCTCAGACTGAGGAAAGCCAAGGAGAAAAATGGACCAATCAGTTCATATCAGG
TGTTAGTGCTTCCCCTGGCCCTCCAAAGCACATTTTCTTGTGATTCTGAAGGCGCTTCCTCCTTCTTTAG
CAACGCCTCTGATGCTGATGGATACGTGGCTGCAGAACTACTGGCCAAAGATGTTCCAGATGATGCCATG
GAGATACCTATAGGAGACAGGCTGTACTATGGGGAATATTATAATGCACCCTTGAAAAGAGGGAGTGATT
ACTGCATTATATTACGAATCACAAGTGAATGGAATAAGGTGAGAAGACACTCCTGTGCAGTTTGGGCTCA
GGTGAAAGATTCGTCACTCATGCTGCTGCAGATGGCGGGTGTTGGACTGGGTTCCCTGGCTGTTGTGATC
ATTCTCACATTCCTCTCCTTCTCAGCGGTGTGATGGCAGATGGACACTGAGTGGGGAGGATGCACTGCTG
CTGGGCAGGTGTTCTGGCAGCTTCTCAGGTGCCCGCACAGAGGCTCCGTGTGACTTCCGTCCAGGGAGCA
TGTGGGCCTGCAACTTTCTCCATTCCCAGCTGGTCCCCATTCCTGGATTTAAGATGGTGGCTATCCCTGA
GGAGTCACCATAAGGAGAAAACTCAGGAATTCTGAGTCTTCCCTGCTACAGGACCAGTTCTGTGCAATGA
ACTTGAGACTCCTGATGTACACTGTGATATTGACCGAAGGCTACATACAGATCTGTGAATCTTGGCTGGG
ACTTCCTCTGAGTGATGCCTGAGGGTCAGCTCCTCTAGACATTGACTGCAAGAGAATCTCTGCAACCTCC
TATATAAAAGCATTTCTGTTAATTCATTCAGAATCCATTCTTTACAATATGCAGTGAGATGGGCTTAAGT
TTGGGCTAGAGTTTGACTTTATGAAGGAGGTCATTGAAAAAGAGAACAGTGACGTAGGCAAATGTTTCAA
GCACTTTAGAAACAGTACTTTTCCTATAATTAGTTGATATACTAATGAGAAAATATACTAGCCTGGCCAT
GCCAATAAGTTTCCTGCTGTGTCTGTTAGGCAGCATTGCTTTGATGCAATTTCTATTGTCCTATATATTC
AAAAGTAATGTCTACATTCCAGTAAAAATATCCCGTAATTAAGAAAAAAAA

In a search of sequence databases, it was found, for example, that the nucleic acid sequence of this invention has 2428 of 2431 bases (99%) identical to a gb:GENBANK-ID:HSM802135|acc:AL137432.1 mRNA from Homo sapiens (Homo sapiens mRNA; cDNA DKFZp761E1824 (from clone DKFZp761E1824); partial cds).

The disclosed NOV8 polypeptide (SEQ ID NO:26) encoded by SEQ ID NO:25 has 747 amino acid residues and is presented in Table 8B using the one-letter amino acid code. Signal P, Psort and/or Hydropathy results predict that NOV8 is a Type Ia membrane protein, has a signal peptide, and is likely to be localized at the plasma membrane with a certainty of 0.9190. In other embodiments, NOV8 may also be localized to the endoplasmic reticulum (membrane) with a certainty of 0.100, and lysosomes with a certainty of 0.2000. The most likely cleavage site for NOV8 is between positions 29 and 30, AAG-AP.

TABLE 8B
NOV8 Polypeptide SEQ ID NO:26

MGRGPWDAGPSRRLLPLLLLLGLARGAAGAPGPDGLDVCATCHEHATCQQREGKKICICNYGFVGNGRTQ
CVDKNECQFGATLVCGNHTSCHNTPGGFYCICLEGYRATNNNKTFIPNDGTFCTDIDECEVSGLCRHGGR
CVNTHGSFECYCMDGYLPRNGPEPFHPTTDATSCTEIDCGTPPEVPDGYIIGNYTSSLGSQVRYACREGF
FSVPEDTVSSCTGLGTWESPKLHCQEINCGNPPEMRHAILVGNHSSRLGGVARYVCQEGFESPGGKITSV
CTEKGTWRESTLTCTEILTKINDVSLFNDTCVRWQINSRRINPKISYVISIKGQRLDPMESVREETVNLT
TDSRTPEVCLALYPGTNYTVNISTAPPRRSMPAVIGFQTAEVDLLEDDGSFNISIFNETCLKLNRRSRKV
GSEHMYQFTVLGQRWYLANFSHATSFNFTTREQVPVVCLDLYPTTDYTVNVTLLRSPKRHSVQITIATPP
AVKQTISNISGFNETCLRWRSIKTADMEEMYLFHIWGQRWYQKEFAQEMTFNISSSSRDPEVCLDLRPGT
NYNVSLRALSSELPVVISLTTQITEPPLPEVEFFTVHRGPLPRLRLRKAKEKNGPISSYQVLVLPLALQS
TFSCDSEGASSFFSNASDADGYVAAELLAKDVPDDAMEIPIGDRLYYGEYYNAPLKRGSDYCIILRITSE
WNKVRRHSCAVWAQVKDSSLMLLQMAGVGLGSLAVVIILTFLSFSAV

A search of sequence databases reveals that the NOV8 amino acid sequence has 570 of 570 amino acid residues (100%) identical to, and 570 of 570 amino acid residues (100%) similar to, the 570 amino acid residue ptnr:SPTREMBL-ACC:Q9NTA7 protein from Homo sapiens (Human) (HYPOTHETICAL 63.7 KDA PROTEIN)(FIG. 3B).

NOV8 maps to chromosome 11p15.3, and is found in at least Adrenal Gland/Suprarenal gland, Amygdala, Aorta, Bone Marrow, Brain, Colon, Dermis, Duodenum, Heart, Hippocampus, Hypothalamus, Kidney, Liver, Lung, Lymph node, Lymphoid tissue, Pancreas, Pituitary Gland, Placenta, Retina, Small Intestine, Spinal Chord, Spleen, Substantia Nigra, Synovium/Synovial membrane, Testis, Thalamus, Urinary Bladder, Uterus. This information was derived by determining the tissue sources of the sequences that were included in the invention including but not limited to SeqCalling sources, Public EST sources, Literature sources, and/or RACE sources.

NOV8 also has homology to the amino acid sequence shown in the BLASTP data listed in Table 8C.

TABLE 8C
BLAST results for NOV8

| Gene Index/ | | Length | Identity | Positives | |
|---|---|---|---|---|---|
| Identifier | Protein/Organism | (aa) | (%) | (%) | Expect |
| gi\|11360234\|pir\|\|T46261 | hypothetical | 570 | 100 | 100 | 0.0 |
| | protein | | | | |
| | DKFZp761E1824.1 - | | | | |
| | human | | | | |
| | (fragment) | | | | |
| gi\|17402220\|emb\|CAD13445.1\| | bA4O1.1 (novel | 620 | 100 | 100 | 0.0 |
| (AL138756) | protein) [Homo | | | | |
| | sapiens] | | | | |
| gi\|16552183\|dbj\|BAB71259.1\| | unnamed protein | 570 | 98 | 98 | 0.0 |
| (AK056704) | product [Homo | | | | |
| | sapiens] | | | | |
| gi\|14740162\|ref\|XP_039183.1\| | hypothetical | 1037 | 100 | 100 | 0.0 |
| (XM_039183) | protein | | | | |
| | DKFZp761E1824 | | | | |
| | [Homo sapiens] | | | | |
| gi\|10438017\|dbj\|BAB15149.1\| | unnamed protein | 409 | 100 | 100 | 0.0 |
| (AK025486) | product [Homo | | | | |
| | sapiens] | | | | |

The homology of these sequences is shown graphically in the ClustalW analysis shown in Table 8D.

[image]

[image]

[image]

[image]

Table 8E lists the domain description from DOMAIN analysis results against NOV8. This indicates that the NOV8 sequence has properties similar to those of other proteins known to contain this domain.

TABLE 8E

Domain Analysis of NOV8
gnl|Smart|smart00179, EGF_CA, Calcium-
binding EGF-like domain
CD-Length = 41 residues, 80.5% aligned
Score = 52.8 bits (125), Expect = 7e−08

| | | | |
|---|---|---|---|
| Query: | 125 | DIDECEVSGLCRHGGRCVNTHGSFECY-CMDGY | 156 |
| | | DIDEC C++GG CVNT GS+ C C GY | |
| Sbjct: | 1 | DIDECASGNPCQNGGTCVNTVGSYRCEECPPGY | 33 |

The polynucleotide encoding a disclosed NOV8 Type Ia Membrane Sushi-Containing Domain-like protein is identified by the comparative sequencing of human chromosome 11p15 and mouse chromosome 7. This gene contains two very important domains associated with developmental proteins—the CUB domain and the domain first found in C1r, C1s, uEGF, and bone morphogenetic protein. The CUB domain is found in 16 functionally diverse proteins such as the dorso-ventral patterning protein tolloid, bone morphogenetic protein 1, a family of spermadhesins, complement subcomponents C1s/C1r and the neuronal recognition molecule A5. Most of these proteins are known to be involved in developmental processes. The second domain is found mostly among developmentally-regulated proteins and spermadhesins.

The disclosed NOV8 nucleic acid of the invention encoding a Type Ia Membrane Sushi-Containing Domain-like protein includes the nucleic acid whose sequence is provided in Table 8A, or a fragment thereof. The invention also includes a mutant or variant nucleic that enhances the chemical stability of the modified nucleic acid, such that they may be used, for example, as antisense binding nucleic acids in therapeutic applications in a subject. In the mutant or variant nucleic acids, and their complements, up to about 1% percent of the bases may be so changed.

The disclosed NOV8 protein of the invention includes Type Ia Membrane Sushi-Containing Domain-like protein whose sequence is provided in Table 8B. The invention also includes a mutant or variant protein any of whose residues may be changed from the corresponding residue shown in Table 8B while still encoding a protein that maintains its Type Ia Membrane Sushi-Containing Domain-like activities and physiological functions, or a functional fragment thereof. In the mutant or variant protein, up to about 0% percent of the residues may be so changed.

The invention further encompasses antibodies and antibody fragments, such as F_{ab} or (F_{ab})₂, that bind immuno specifically to any of the proteins of the invention.

The above defined information for this invention suggests that this Type Ia Membrane Sushi-Containing Domain-like protein (NOV8) may function as a member of a glucose transporter family. Therefore, the NOV8 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV8 nucleic acids and proteins of the invention are useful in potential therapeutic applications implicated in cancer including but not limited to Inflamation, Autoimmune disorders, Aging and Cancer. For example, a cDNA encoding the Type Ia Membrane Sushi-Containing Domain-like protein (NOV8) may be useful in gene therapy, and the Type Ia Membrane Sushi-Containing Domain-like protein (NOV8) may be useful when administered to a subject in need thereof. By way of nonlimiting example, the compositions of the present invention will have efficacy for treatment of patients suffering from cancer, trauma, regeneration (in vitro and in vivo), viral/bacterial/parasitic infections, Atherosclerosis, Aneurysm, Hypertension, Fibromuscular dysplasia, Stroke, Scleroderma, Obesity, Transplantation, Myocardial infarction, Embolism, Cardiovascular disorders, Bypass surgery, Adrenoleukodystrophy, Congenital Adrenal Hyperplasia, Diabetes, Von Hippel-Lindau (VHL) syndrome, Pancreatitis, Cirrhosis, Hemophilia, Hypercoagulation, Idiopathic thrombocytopenic purpura, Immunodeficiencies, Graft vesus host disease (GVHD), Lymphedema, Allergies, autoimmume disease, Alzheimer's disease, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Systemic lupus erythematosus, Asthma, Emphysema, Scleroderma, ARDS, Renal artery stenosis, Interstitial nephritis, Glomerulonephritis, Polycystic kidney disease, Systemic lupus erythematosus, Renal tubular acidosis, IgA nephropathy and other diseases, disorders and conditions of the like.

The NOV8 nucleic acid encoding Type Ia Membrane Sushi-Containing Domain-like protein, and the Type Ia Membrane Sushi-Containing Domain-like protein of the invention, or fragments thereof, may further be useful in diagnostic applications, wherein the presence or amount of the nucleic acid or the protein are to be assessed.

NOV8 nucleic acids and polypeptides are further useful in the generation of antibodies that bind immuno-specifically to the novel NOV8 substances for use in therapeutic or diagnostic methods. These antibodies may be generated according to methods known in the art, using prediction from hydrophobicity charts, as described in the “Anti-NOVX Antibodies” section below. The disclosed NOV8 protein has multiple hydrophilic regions, each of which can be used as an immunogen. In one embodiment, a contemplated NOV8 epitope is from about amino acids 40 to 300. In another embodiment, a NOV8 epitope is from about amino acids 305 to 360, from about 400 to 450, from about 500 to 560, from about 580 to 610, and from about 620 to 680. These novel proteins can be used in assay systems for functional analysis of various human disorders, which will help in understanding of pathology of the disease and development of new drug targets for various disorders.

NOV9

A disclosed NOV9 nucleic acid of 2507 nucleotides identified as SEQ ID NO:27 (designated CuraGen Acc. No. CG95545-02) encoding a novel Type Ia Membrane-Sushi Domain Containing Protein-like protein is shown in Table 9A. An open reading frame was identified beginning at nucleotides 309-311 and ending at nucleotides 2469-2471. Putative untranslated regions are indicated by underline.

TABLE 9A
NOV9 Polynucleotide SEQ ID NO:27

| | |
|---|---|
| CGGGGCTCTGCGTCAGCTGTGTCATTATCCGATGAGTGTCTGTCCCCCTTTGCGAATGTG | 60 |
| AGCGGCGAGAGGGCAGCAAGTGCGGAGCCAGAGACGGACGCGGAACGGGCGTGTCCTAAG | 120 |
| CCCAGGCCCCGACAGGAGGAAGGACCCGCGCTCTGCGGCCTCCCGGGGACCCCGCAGCGC | 180 |
| CCCCCGCTTCCCTCGGCGGCGCCGGAAGCCGCCGGCTGGTCCCCTCCCCGCGGCGCCTGT | 240 |
| AGCCTTATCTCTGCACCCTGAGGGCCCCGGGAGGAGGCGCGGGCGCGCCGGGAGGGACCG | 300 |
| GCGGCGGCATGGGCCGGGGGCCCTGGGATGCGGGCCCGTCTCGCCGCCTGCTGCCGCTGT | 360 |
| TGCTGCTGCTCGGCCTGGCCCGCGGCGCCGCGGGAGCGCCGGGCCCCGACGGTTTAGACG | 420 |
| TCTGTGCCACTTGCCATGAACATGCCACATGCCAGCAAAGAGAAGGGAAGAAGATCTGTA | 480 |
| TTTGCAACTATGGATTTGTAGGGAACGGGAGGACTCAGTGTGTTGATAAAAATGAGTGCC | 540 |
| AGTTTGGAGCCACTCTTGTCTGTGGGAACCACACATCTTGCCACAACACCCCCGGGGGCT | 600 |
| TCTATTGCATTTGCCTGGAAGGATATCGAGCCACAAACAACAACAAGACATTCATTCCCA | 660 |
| ACGATGGCACCTTTTGTACAGACATAGATGAGTGTGAAGTTTCTGGCCTGTGCAGGCATG | 720 |
| GAGGGCGATGCGTGAACACTCATGGGAGCTTTGAATGCTACTGTATGGATGGATACTTGC | 780 |
| CAAGGAATGGACCTGAACCTTTCCACCCGACCACCGATGCCACATCATGCACAGAAATAG | 840 |
| ACTGTGGTACCCCTCCTGAGGTTCCAGATGGCTATATCATAGGAAATTATACGTCTAGTC | 900 |
| TGGGCAGCCAGGTTCGTTATGCTTGCAGAGAAGGATTCTTCAGTGTTCCAGAAGATACAG | 960 |
| TTTCAAGCTGCACAGGCCTGGGCACATGGGAGTCCCCAAAATTACATTGCCAAGAGATCA | 1020 |
| ACTGTGGCAACCCTCCAGAAATGCGGCACGCCATCTTGGTAGGAAATCACAGCTCCAGGC | 1080 |
| TGGGCGGTGTGGCTCGCTATGTCTGTCAAGAGGGCTTTGAGAGCCCTGGAGGAAAGATCA | 1140 |
| CTTCTGTTTGCACAGAGAAAGGCACCTGGAGAGAAAGTACTTTAACATGCACAGAAATTC | 1200 |
| TGACAAAGATTAATGATGTATCACTGTTTAATGATACCTGTGTGAGATGGCAAATAAACT | 1260 |
| CAAGAAGAATAAACCCCAAGATCTCATATGTGATATCCATAAAAGGACAACGGTTGGACC | 1320 |
| CTATGGAATCAGTTCGTGAGGAGACAGTCAACTTGACCACAGACAGCAGGACCCCAGAAG | 1380 |
| TGTGCCTAGCCCTGTACCCAGGCACCAACTACACCGTGAACATCTCCACAGCACCTCCCA | 1440 |
| GGCGCTCGATGCCAGCCGTCATCGGTTTCCAGACAGCTGAAGTTGATCTCTTAGAAGATG | 1500 |
| ATGGAAGTTTCAATATTTCAATATTTAATGAAACTTGTTTGAAATTGAACAGGCGTTCTA | 1560 |
| GGAAAGTTGGATCAGAACACATGTACCAATTTACCGTTCTGGGTCAGAGGTGGTATCTGG | 1620 |
| CTAACTTTTCTCATGCAACATCGTTTAACTTCACAACGAGGGAACAAGTGCCTGTAGTGT | 1680 |
| GTTTGGATCTGTACCCTACGACTGATTATACGGTGAATGTGACCCTGCTGAGATCTCCTA | 1740 |
| AGCGGCACTCAGTGCAAATAACAATAGCAACTCCCCCAGCAGTAAAACAGACCATCAGTA | 1800 |
| ACATTTCAGGATTTAATGAAACCTGCTTGAGATGGAGAAGCATCAAGACAGCTGATATGG | 1860 |
| AGGAGATGTATTTATTCCACATTTGGGGCCAGAGATGGTATCAGAAGGAATTTGCCCAGG | 1920 |
| AAATGACCTTTAATATCAGTAGCAGCAGCCGAGATCCCGAGGTGTGCTTGGACCTACGTC | 1980 |
| CGGGTACCAACTACAATGTCAGTCTCCGGGCTCTGTCTTCGGAACTTCCTGTGGTCATCT | 2040 |
| CCCTGACAACCCAGATAACAGAGCCTCCCCTCCCGGAAGTAGAATTTTTTACGGTGCACA | 2100 |
| GAGGACCTCTACCACGCCTCAGACTGAGGAAAGCCAAGGAGAAAAATGGACCAATCAGCA | 2160 |
| ACGCCTCTGATGCTGATGGATACGTGGCTGCAGAACTACTGGCCAAAGATGTTCCAGATG | 2220 |
| ATGCCATGGAGATACCTATAGGAGACAGGCTGTACTATGGGGAATATTATAATGCACCCT | 2280 |
| TGAAAAGAGGGAGTGATTACTGCATTATATTACGAATCACAAGTGAATGGAATAAGGTGA | 2340 |
| GAAGACACTCCTGTGCAGTTTGGGCTCAGGTGAAAGATTCGTCACTCATGCTGCTGCAGA | 2400 |
| TGGCGGGTGTTGGACTGGGTTCCCTGGCTGTTGTGATCATTCTCACATTCCTCTCCTTCT | 2460 |
| CAGCGGTGTGATGGCAGATGGACACTGAGTGGGGAGGATGCACTGCT | |

The disclosed NOV9 nucleic acid sequence, localized to chromosome 9, has 1747 of 1747 bases (100%) identical to a gb:GENBANK-ID:AX050019|acc:AX050019.1 mRNA from Homo sapiens (Sequence 32 from Patent WO0071710) (FIG. 3A). The full amino acid sequence of the protein of the invention was found to have 440 of 441 amino acid residues (99%) identical to, and 441 of 441 amino acid residues (100%) similar to, the 570 amino acid residue ptnr:SPTREMBL-ACC:Q9NTA7 protein from Homo sapiens (Human) (HYPOTHETICAL 63.7 KDA PROTEIN).

The disclosed NOV9 polypeptide (SEQ ID NO:28) encoded by SEQ ID NO:27 has 720 amino acid residues and is presented in Table 9B using the one-letter amino acid code. Signal P, Psort and/or Hydropathy results predict that NOV9 is a Type Ia membrane protein, has a signal peptide, and is likely to be localized at the plasma membrane with a certainty of 0.9190. In other embodiments, NOV9 may also be localized to the endoplasmic reticulum (membrane) with a certainty of 0.100, and lysosomes with a certainty of 0.2000. The most likely cleavage site for NOV9 is between positions 29 and 30, AAG-AP.

TABLE 9B
NOV9 Polypeptide SEQ ID NO:28

| | |
|---|---|
| MGRGPWDAGPSRRLLPLLLLLGLARGAAGAPGPDGLDVCATCHEHATCQQREGKKICICN | 60 |
| YGFVGNGRTQCVDKNECQFGATLVCGNHTSCHNTPGGFYCICLEGYRATNNNKTFIPNDG | 120 |
| TFCTDIDECEVSGLCRHGGRCVNTHGSFECYCMDGYLPRNGPEPFHPTTDATSCTEIDCG | 180 |
| TPPEVPDGYIIGNYTSSLGSQVRYACREGFFSVPEDTVSSCTGLGTWESPKLHCQEINCG | 240 |
| NPPEMRHAILVGNHSSRLGGVARYVCQEGFESPGGKITSVCTEKGTWRESTLTCTEILTK | 300 |
| INDVSLFNDTCVRWQINSRRINPKISYVISIKGQRLDPMESVREETVNLTTDSRTPEVCL | 360 |
| ALYPGTNYTVNISTAPPRRSMPAVIGFQTAEVDLLEDDGSFNISIFNETCLKLNRRSRKV | 420 |
| GSEHMYQFTVLGQRWYLANFSHATSFNFTTREQVPVVCLDLYPTTDYTVNVTLLRSPKRH | 480 |
| SVQITIATPPAVKQTISNISGFNETCLRWRSIKTADMEEMYLFHIWGQRWYQKEFAQEMT | 540 |
| FNISSSSRDPEVCLDLRPGTNYNVSLRALSSELPVVISLTTQITEPPLPEVEFFTVHRGP | 600 |
| LPRLRLRKAKEKNGPISNASDADGYVAAELLAKDVPDDAMEIPIGDRLYYGEYYNAPLKR | 660 |
| GSDYCIILRITSEWNKVRRHSCAVWAQVKDSSLMLLQMAGVCLGSLAVVIILTFLSFSAV | 720 |

A search of sequence databases reveals that the NOV9 amino acid sequence has 440 of 441 amino acid residues (99%) identical to, and 441 of 441 amino acid residues (100%) similar to, the 570 amino acid residue ptnr:SPTREMBL-ACC:Q9NTA7 protein from Homo sapiens (Human) (HYPOTHETICAL 63.7 KDA PROTEIN). NOV9 is expressed in at least the pancreas, placenta, nervous system, tumor tissues, brain and the hypothalamus.

The disclosed NOV9 polypeptide has homology to the amino acid sequences shown in the BLASTP data listed in Table 9C.

TABLE 9C
BLAST results for NOV9

| Gene Index/ | | Length | Identity | Positives | |
|---|---|---|---|---|---|
| Identifier | Protein/Organism | (aa) | (%) | (%) | Expect |
| gi\|11360234\|pir∥T46261 | hypothetical | 570 | 100 | 100 | 0.0 |
| | protein | | | | |
| | DKFZp761E1824.1 - | | | | |
| | human | | | | |
| | (fragment) | | | | |
| gi\|17402220\|emb\|CAD13445.1\| | bA4O1.1 (novel | 620 | 100 | 100 | 0.0 |
| (AL138756) | protein) [Homo | | | | |
| | sapiens] | | | | |
| gi\|16552183\|dbj\|BAB71259.1\| | unnamed protein | 570 | 98 | 98 | 0.0 |
| (AK056704) | product [Homo | | | | |
| | sapiens] | | | | |
| gi\|14740162\|ref\|XP_039183.1\| | hypothetical | 1037 | 100 | 100 | 0.0 |
| (XM_039183) | protein | | | | |
| | DKFZp761E1824 | | | | |
| | [Homo sapiens] | | | | |
| gi\|10438017\|dbj\|BAB15149.1\| | unnamed protein | 409 | 100 | 100 | 0.0 |
| (AK025486) | product [Homo | | | | |
| | sapiens] | | | | |

The homology between these and other sequences is shown graphically in the ClustalW analysis shown in Table 9D. In the ClustalW alignment of the NOV9 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

Table 9E lists the domain description from DOMAIN analysis results against NOV9. This indicates that the NOV9 sequence has properties similar to those of other proteins known to contain this domain.

TABLE 9E

Domain Analysis of NOV9
gnl|Smart|smart00179, EGF_CA, Calcium-
binding EGF-like domain
CD-Length = 41 residues, 80.5% aligned
Score = 52.8 bits (125), Expect = 7e−08

| | | | |
|---|---|---|---|
| Query: | 125 | DIDECEVSGLCRHGGRCVNTHGSFECY-CMDGY | 156 |
| | | DIDEC C++GG CVNT GS+ C C GY | |
| Sbjct: | 1 | DIDECASGNPCQNGGTCVNTVGSYRCEECPPGY | 33 |

The disclosed NOV9 polynucleotide encodes a Type Ia Membrane Sushi-Containing Domain-like protein, identified by the comparative sequencing of human chromosome 1 1p 1 5 and mouse chromosome 7. This gene contains two very important domains associated with developmental proteins- the CUB domain and the domain first found in C1r, C1s, uEGF, and bone morphogenetic protein. The CUB domain is found in 16 functionally diverse proteins such as the dorso-ventral patterning protein tolloid, bone morphogenetic protein 1, a family of spermadhesins, complement subcomponents C1s/C1r and the neuronal recognition molecule A5. Most of these proteins are known to be involved in developmental processes. The second domain is found mostly among developmentally-regulated proteins and spermadhesins.

The disclosed NOV9 nucleic acid of the invention encoding a Type Ia membrane sushi-containing domain-like protein includes the nucleic acid whose sequence is provided in Table 9A, or a fragment thereof. The invention also includes a mutant or variant nucleic acid any of whose bases may be changed from the corresponding base shown in Table 9A while still encoding a protein that maintains its a Type Ia membrane sushi-containing domain-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

The disclosed NOV9 protein of the invention includes the Type Ia membrane sushi-containing domain-like protein whose sequence is provided in Table 9B. The invention also includes a mutant or variant protein any of whose residues may be changed from the corresponding residue shown in Table 2 while still encoding a protein that maintains its a Type Ia membrane sushi-containing domain-like activities and physiological functions, or a functional fragment thereof. In the mutant or variant protein, up to about 1 percent of the residues may be so changed.

The invention further encompasses antibodies and antibody fragments, such as F_{ab} or (F_{ab})₂, that bind immuno specifically to any of the proteins of the invention.

The above defined information for this invention suggests that this Type Ia membrane sushi-containing domain-like protein (NOV9) may function as a member of a family. Therefore, the NOV9 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV9 nucleic acids and proteins of the invention are useful in potential therapeutic applications implicated in liver toxicity and damage such as in cancer, cirrhosis, or troglitazone treatment for diabetes; brain and CNS disorders including cancer, Parkinson's, Alzheimer's, epilepsy, schizophrenia and other diseases, disorders and conditions of the like. For example, a cDNA encoding a Type Ia membrane sushi-containing domain-like protein (NOV9) may be useful in gene therapy, and the Type Ia membrane sushi-containing domain-like protein (NOV9) may be useful when administered to a subject in need thereof. By way of nonlimiting example, the compositions of the present invention will have efficacy for treatment of patients suffering from cancer, diabetes, obesity, fertility as well as other diseases, disorders and conditions. The NOV9 nucleic acid encoding a Type Ia membrane sushi-containing domain-like protein, and the a Type Ia membrane sushi-containing domain-like protein of the invention, or fragments thereof, may further be useful in diagnostic applications, wherein the presence or amount of the nucleic acid or the protein are to be assessed.

NOV9 nucleic acids and polypeptides are further useful in the generation of antibodies that bind immuno-specifically to the novel NOV9 substances for use in therapeutic or diagnostic methods. These antibodies may be generated according to methods known in the art, using prediction from hydrophobicity charts, as described in the “Anti-NOVX Antibodies” section below. The disclosed NOV9 protein has multiple hydrophilic regions, each of which can be used as an immunogen. In one embodiment, a contemplated NOV9 epitope is from about amino acids 40 to 300. In another embodiment, a NOV9 epitope is from about amino acids 305 to 360, from about 400 to 450, from about 500 to 560, from about 580 to 610, and from about 620 to 680. These novel proteins can be used in assay systems for functional analysis of various human disorders, which will help in understanding of pathology of the disease and development of new drug targets for various disorders.

NOV10

NOV10 includes two butyrophilin-like proteins disclosed below. The disclosed sequences have been named NOV10a and NOV10b.

NOV10a: A disclosed NOV10a nucleic acid of 861 nucleotides identified as SEQ ID NO:29 (designated CuraGen Acc. No. CG55746-01 ) encoding a novel butyrophilin-like protein is shown in Table 10A. An open reading frame was identified beginning with an ATG initiation codon at nucleotides 4648 and ending with a TGA codon at nucleotides 793-795. Putative untranslated regions, if any, are found upstream from the initiation codon and downstream from the termination codon.

TABLE 10A
NOV10A Polynucleotide SEQ ID NO:29

CAGGTTACACTTCGTAAGAACTGGAATGTAAAGTAAAGGCAGACAATGACAAAATATCTTGTTTTCTTTT
CAGCTTTATTCACAGTGACAGTCCCTAAGCACCTGTACATAATAAAGCACCCCAGCAATGTGACCCTGGA
ATGCAACTTTGACACTGGTAGTCATGTGAACCTTGGAGCAATAACAGTCAGTTTGCAAAAGGTGGAAAAT
GATACATCCCCACACCGTGAAAGAGCCACTTTGCTGGAGGAGCAGCTGCCCCTAGGGAAGGCCTCGTTCC
ACATACCTCAAGTCCAAGTGAGGGACGAAGGACAGTACCAATGCATAATCATCTATGGGGTCGCCTGGGA
CTACAAGTACCTGACTCTGAAAGTCAAAGGTGCTTCCTACAGGAAAATAAACACTCACATCCTAAAGGTT
CCAGAAACAGATGAGGTAGAGCTCACCTGCCAGGCTACAGGTTATCCTCTGGCAGAAGTATCCTGGCCAA
ACGTCAGCGTTCCTGCCAACACCAGCCACTCCAGGACCCCTGAAGGCCTCTACCAGGTCACCAGTGTTCT
GCGCCTAAAGCCACCCCCTGGCAGAAACTTCAGCTGTGTGTTCTGGAATACTCACGTGAGGGAACTTACT
TTGGCCAGCATTGACCTTCAAAGTAAGATGGAACCCAGGACCCATCCAACTTGGCTGCTTCACATTTTCA
TCCCCTTCTGCATCATTGCTTTCATTTTCATAGCCACAGTGATAGCCCTAAGAAAACAACTCTGTCAAAA
GCTGTATTCTTCAAAAGGTAAGTGAGTTTTATTCATGGTAACCCAATGCACTGGGTGTCTGCAGCATGAG
CCACTGCTTTGCACTGCAGGC

In a search of public sequence databases, the NOV10a nucleic acid sequence, which maps to chromosome 9, and has 467 of 473 bases (98%) identical to a gb:GENBANK-ID:AK001872|acc:AK001872.1 mRNA from Homo sapiens (Homo sapiens cDNA FLJ11010 fis, clonePLACE1003145).

The disclosed NOV10a polypeptide (SEQ ID NO:30) encoded by SEQ ID NO:29 has 249 amino acid residues and is presented in Table 10B using the one-letter amino acid code. Signal P, Psort and/or Hydropathy results predict that NOV10a is a Type Ia membrane protein, has a signal peptide, and is likely to be localized at the plasma membrane with a certainty of 0.4600. In other embodiments, NOV10a may also be localized to the endoplasmic reticulum (membrane) with a certainty of 0.3700, and lysosomes with a certainty of 0.3000. The most likely cleavage site for NOV8 is between positions 17 and 18, TVP-KH.

TABLE 10B
NOV10a Polypeptide SEQ ID NO:30

MTKYLVFFSALFTVTVPKHLYIIKHPSNVTLECNFDTGSHVNLGAITVSLQKVENDTSPHRERATLLEEQ
LPLGKASFHIPQVQVRDEGQYQCIIIYGVAWDYKYLTLKVKGASYRKINTHILKVPETDEVELTCQATGY
PLAEVSWPNVSVPANTSHSRTPEGLYQVTSVLRLKPPPGRNFSCVFWNTHVRELTLASIDLQSKMEPRTH
PTWLLHIFIPFCIIAFIFIATVIALRKQLCQKLYSSKGK

A search of sequence databases reveals that the NOV10a amino acid sequence has 159 of 231 amino acid residues (68%) identical to, and 182 of 231 amino acid residues (78%) similar to, the 247 amino acid residue ptnr: SPTREMBL-ACC:Q9WUL5 protein from Mus musculus (Mouse) (BUTYROPHILIN-LIKE PROTEIN). NOV10a is expressed in at least Bone Marrow, Lung, Testis, Thymus, Uterus, Whole Organism.

NOV10b: A disclosed NOV10b nucleic acid of 660 nucleotides identified as SEQ ID NO:31 (designated CuraGen Acc. No. CG55746-05) encoding a novel BUTYROPHILIN PRECURSOR B7-DC-like protein is shown in Table 10C. An open reading frame was identified beginning at nucleotides 34-36 and ending at nucleotides 583-585. Putative untranslated regions are indicated by underline.

TABLE 10C
NOV10b Polynucleotide SEQ ID NO:31

| | |
|---|---|
| AGCTGTGGCAAGTCCTCATATCAAATACAGAACATGATCTTCCTCCTGCTAATGTTGAGC | 60 |
| CTGGAATTGCAGCTTCACCAGATAGCAGCTTTATTCACAGTGACAGTCCCTAAGGAACTC | 120 |
| TACATAATAGAGCATGGCAGCAATGTGACCCTGGAATGCAACTTTGACACTGGAAGTCAT | 180 |
| GTGAACCTTGGAGCAATAACAACCAGTTTGCAAAAGGTGGAAAATGATACATCCCCACAC | 240 |
| CGTGAAAGAGCCACTTTGCTGGAGGAGCAGCTGCCCCTAGGGAAGGCCTCGTTCCACATA | 300 |
| CCTCAAGTCCAAGTGAGGGACGAAGGACACTACCAATGCATAATCATCTATGGGGTCGCC | 360 |
| TGGGACTACAAGTACCTGACTCTGAAAGTCAAAGGTCAGATGGAACCCAGGACCCATCCA | 420 |
| ACTTGGCTGCTTCACATTTTCATCCCCTCCTGCATCATTGCTTTCATTTTCATACCCACA | 480 |
| GTGATAGCCCTAAGAAAACAACTCTGTCAAAAGCTGTATTCTTCAAAAGACACAACAAAA | 540 |
| AGACCTGTCACCACAACAAAGAGGGAAGTGAACAGTGCTATCTGAACCTGTGGTCTTGGG | 600 |
| AGCCAGGGTGACCTGATATGACATTTAAAGAAGCTTCTGGACTCTGAACAAGAATTCGGT | 660 |

In a search of public sequence databases, the NOV10b nucleic acid sequence, which maps to chromosome 9, has 394 of 396 bases (99%) identical to a gb:GENBANK-ID:AF329193|acc:AF329193.1 mRNA from Homo sapiens (Homo sapiens butyrophilin precursor B7-DC mRNA, complete cds).

The disclosed NOV10b polypeptide (SEQ ID NO:32) encoded by SEQ ID NO:31 has 183 amino acid residues and is presented in Table 10D using the one-letter amino acid code. Signal P, Psort and/or Hydropathy results predict that NOV10b is a Type II membrane protein, has a signal peptide, and is likely to be localized to the mitochondrial inner membrane with a certainty of 0.8463. In other embodiments, NOV10b may also be localized to the plasma membrane with a certainty of 0.4400, mitochondrial intermembrane space with a certainty of 0.3008, and mitochondrial matrix space with a certainty of 0.2317. The most likely cleavage site for NOV10b is between positions 19 and 20, IAA-LF.

TABLE 10D
NOV10b Polypeptide SEQ ID NO:32

| | |
|---|---|
| MIFLLLMLSLELQLHQIAALFTVTVPKELYIIEHGSNVTLECNFDTGSHVNLGAITTSLQ | 60 |
| KVENDTSPHRERATLLEEQLPLGKASFHIPQVQVRDEGQYQCIIIYGVAWDYKYLTLKVK | 120 |
| GQMEPRTHPTWLLHIFIPSCIIAFIFIATVIALRKQLCQKLYSSKDTTKRPVTTTKREVN | 180 |
| SAI | |

A search of sequence databases reveals that the NOV10b amino acid sequence has 121 of 129 amino acid residues (93%) identical to, and 121 of 129 amino acid residues (93%) similar to, the 273 amino acid residue ptnr:SPTREMBL-ACC:Q9BQ51 protein from Homo sapiens (Human) (butyrophilin precursor B7-DC (PD-1-ligand 2 protein)). NOV10B, the butyrophilin precursor B7-DC-like gene disclosed in this invention is expressed in at least the following tissues: Bone Marrow, Lung, Testis, Thymus, Uterus, Whole Organism.

The disclosed NOV10b polypeptide has homology to the amino acid sequences shown in the BLASTP data listed in Table 10E.

TABLE 10E
BLAST results for NOV10A

| Gene Index/ | | Length | Identity | Positives | |
|---|---|---|---|---|---|
| Identifier | Protein/Organism | (aa) | (%) | (%) | Expect |
| gi\|13640665\|ref\|XP_016318.1\| | hypothetical | 273 | 97 | 98 | e−128 |
| (XM_016318) | protein | | | | |
| | XP_016318 [Homo | | | | |
| | sapiens] | | | | |
| gi\|13376850\|ref\|NP_079515.1\| | programmed death | 273 | 96 | 97 | e−127 |
| (NM_025239) | ligand 2 [Homo | | | | |
| | sapiens] | | | | |
| gi\|6912724\|ref\|NP_036597.1\| | butyrophilin- | 247 | 68 | 77 | 8e−85 |
| (NM_012465) | like protein | | | | |
| | [Mus musculus] | | | | |
| gi\|7661534\|ref\|NP_054862.1\| | B7-H1 protein | 290 | 38 | 52 | 5e−32 |
| (NM_014143) | [Homo sapiens] | | | | |
| gi\|11230798\|ref\|NP_068693.1\| | programmed cell | 290 | 39 | 52 | 1e−31 |
| (NM_021893) | death 1 ligand 1 | | | | |
| | [Mus musculus] | | | | |

The homology between these and other sequences is shown graphically in the ClustalW analysis shown in Table 10F. In the ClustalW alignment of the NOV10a and NOV10b proteins, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Tables 10G lists the domain description from DOMAIN analysis results against NOV10. This indicates that the NOV10 sequence has properties similar to those of other proteins known to contain this domain.

TABLE 10G

Domain Analysis of NOV10
gnl|Smart|smart00409, IG, Immunoglobulin
CD-Length = 86 residues, 89.5% aligned
Score = 37.0 bits (84), Expect = 0.001

| | | | |
|---|---|---|---|
| Query: | 27 | SNVTLECNFDTGSHVNLGAITVSLQKVENDTSPHRERATLLEEQLPLGKASFHIPQVQVR | 86 |
| | | +VTL C TV+ K R ++ G ++ I V | |
| Sbjct: | 10 | ESVTLSCEASGNPPP-----TVTWYKQGGKLLAESGRFSVSRSG---GNSTLTISNVTPE | 61 |
| Query: | 87 | DEGQYQCIIIYGVAWDYKYLTLKVK | 111 |
| | | D G Y C TL V | |
| Sbjct: | 62 | DSGTYTCAATNSSGSASSGTTLTVL | 86 |

The gene sequence of invention described herein encodes for a novel member of the B7-Immunoglobulin family of enzymes. Specifically, the sequence encodes a novel BUTYROPHILIN-like protein. BUTYROPHILIN molecules play crucial roles in T-cell activation making them plausible targets for cancer, AIDS, and inflammation therapies. The protein described here is known to be expressed in spleen, and liver which may indicate roles in lupus, endocrine disorders, inflammation, autoimmune disorders, and cancers including liver, bone, and leukemia.

The disclosed NOV10 nucleic acid of the invention encoding a butyrophilin-like protein includes the nucleic acid whose sequence is provided in Table 10 or a fragment thereof. The invention also includes a mutant or variant nucleic acid any of whose bases may be changed from the corresponding base shown in Table 10 while still encoding a protein that maintains its butyrophilin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 2 percent (NOV10a) or 1% (NOV10b) of the bases may be so changed.

The disclosed NOV10 protein of the invention includes the butyrophilin-like protein whose sequence is provided in Table 10A and 10C. The invention also includes a mutant or variant protein any of whose residues may be changed from the corresponding residue shown in Table 10B while still encoding a protein that maintains its butyrophilin-like activities and physiological functions, or a functional fragment thereof. In the mutant or variant protein, up to about 32 percent (NOV10a) or (NOV10b) of the residues may be so changed.

The invention further encompasses antibodies and antibody fragments, such as F_{ab} or (F_{ab})₂, that bind immunospecifically to any of the proteins of the invention.

The above defined information for this invention suggests that this butyrophilin-like protein (NOV10) may function as a member of a butyrophilin family. Therefore, the NOV10 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV10 nucleic acids and proteins of the invention are useful in potential therapeutic applications implicated in cancer including but not limited to various pathologies and disorders as indicated below. For example, a cDNA encoding the butyrophilin-like protein (NOV10) may be useful in gene therapy, and the butyrophilin-like protein (NOV10) may be useful when administered to a subject in need thereof. By way of nonlimiting example, the compositions of the present invention will have efficacy for treatment of patients suffering from : brain disorders including epilepsy, eating disorders, schizophrenia, ADD, and cancer; heart disease; inflammation and autoimmune disorders including Crohn's disease, IBD, lupus, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, and cancer; multiple sclerosis, pancreatic disorders including pancreatic insufficiency and cancer; and prostate disorders including prostate cancer and other diseases, disorders and conditions of the like.

The NOV10 nucleic acid encoding the butyrophilin-like protein of the invention, or fragments thereof, may further be useful in diagnostic applications, wherein the presence or amount of the nucleic acid or the protein are to be assessed.

NOV10 nucleic acids and polypeptides are further useful in the generation of antibodies that bind immuno-specifically to the novel NOV10 substances for use in therapeutic or diagnostic methods. These antibodies may be generated according to methods known in the art, using prediction from hydrophobicity charts, as described in the “Anti-NOVX Antibodies” section below. The disclosed NOV10a protein has multiple hydrophilic regions, each of which can be used as an immunogen. In one embodiment, a contemplated NOV10a epitope is from about amino acids 25 to 40. In another embodiment, a NOV10a epitope is from about amino acids 50 to 70, from about 60 to 100, from about 110 to 140, from about 145 to 185, and from about 190 to 210. The disclosed NOV10b protein has multiple hydrophilic regions, each of which can be used as an immunogen. In one embodiment, a contemplated NOV10b epitope is from about amino acids 50 to 80. In another embodiment, a NOV10b epitope is from about amino acids 80 to 110, from about 111 to 130, and from about 150 to 175.

These novel proteins can be used in assay systems for functional analysis of various human disorders, which will help in understanding of pathology of the disease and development of new drug targets for various disorders.

NOV11

A disclosed NOV11 nucleic acid of 1115 nucleotides identified as SEQ ID NO:33 (also designated as Acc. No. CG50329-01) encoding a novel BUTYROPHILIN-LIKE PROTEIN-like protein is shown in Table 11A. An open reading frame was identified beginning with an ATG initiation codon at nucleotides 46-48 and ending with a TAA codon at 931-933.

TABLE 11A
NOV11 Polynucleotide SEQ ID NO:33

AACCTGCTCTGAGGGGTGGGGAGAAAGACCCCATCACCTGCTAGGATGAGCAGAGCGTGGGGCGATGCAG
TCATTCCCTCACTGTCCGTGCTCCGCTCATTCATTCATCTCCTTGAACTCCTGACCTCAGGCAATGGGAA
AGCTGACTTTGATGTCACTGGGCCTCATGCCCCTATTCTGGCTATGGCTGGGGGACACGTGGAGTTACAG
TGCCAGCTGTTCCCCAATATCAGTGCCGAGGACATGGAGCTGAGGTGGTACAGGTGCCAGCCCTCCCTAG
CTGTGCACATGCATGAGAGAGGGATGGACATGGATGGAGAGCAAAAGTGGCAGTACAGAGGAAGGACCAC
CTTCATGAGTGACCACGTGGCCAGGGGCAAGGCCATGGTGAGGAGTCACAGGGTCACCACCTTTGACAAC
AGGACATACTGCTGCCGCTTCAAGGATGGTGTAAAGTTCGGCGAGGCCACTGTGCAGGTGCAGGTGGCAG
GTAAGTCAGGGCTGGGCAGAGAGCCCAGAATCCAGGTGACAGACCAGCAGGATGGAGTCAGGGCGGAGTG
CACATCAGCAGGCTGTTTCCCCAAGTCCTGGGTGGAACGGAGAGACTTCAGGGGCCAGGCTAGGCCTGCT
GTGACCAATCTATCAGCCTCAGCCACCACCAGGCTCTGGGCTGTGGCATCCAGCTTGACGCTCTGGGACA
GGGCTGTGGAGGGTCTCTCCTGCTCCATCTCCAGCCCCCTCCTCCCTGAAAGGTCAGTTTCAGGCATCCA
CTGGGGGTCATGGAATGTATCCCCCAAGGACAAGGGGGGCTTATTAGAGTCACACTCTGAGGTCCTGGGG
TTAGAACTTCAACAGATGACTGGGGGGCAGGGGATACAAAATGGAACCCATAACAATTCTCAAAATGCTT
TTTCCTCAAACCTGAAAGTGTAAAACCTGCTCTGAGGGGTGGGGAGAAAGACCCCATCACCTGCTAGGAT
GAGCAGAGCGTGGGGCGATGCAGTCATTCCCTCACTGAAGACATTTATGGGGCACCTCCCTATGCACCAG
ACAGGAAGGAAGGAATTACAGAAACAAAACCTCACAAATATATACAATTATTACGTGTTAATTAA

In a search of public sequence databases, the NOV11 nucleic acid sequence, located on chromosome I has 508 of 780 bases (65%) identical to a gb:GENBANK-ID:AF269232|acc:AF269232.1 mRNA from Mus musculus (Mus musculus butyrophilin-like protein BUTR-1 (Butr1) mRNA, complete cds).

The NOV11 protein (SEQ ID NO:34), encoded by SEQ ID NO:33, has 295 amino acids. Signal P, Psort and/or Hydropathy results predict that NOV11 is a Type Ia membrane protein, has a signal peptide, and is likely to be localized to the mitochondrial matrix space with a certainty of 0.6797. In other embodiments, NOV11 may also be localized to the microbodies with a certainty of 0.4023, mitochondrial inner membrane with a certainty of 0.3682, mitochondrial inner membrane space with a certainity of 0.3682. The most likely cleavage site for NOV11 is between positions 31 and 32, GNG-KA.

TABLE 11B
NOV11 Polypeptide SEQ ID NO:34

| | |
|---|---|
| MSRAWGDAVIPSLSVLRSFIHLLELLTSGNGKADFDVTGPHAPILAMAGGHVELQCQLFP | 60 |
| NISAEDMELRWYRCQPSLAVHMHERGMDMDGEQKWQYRGRTTFMSDHVARGKAMVRSHRV | 120 |
| TTFDNRTYCCRFKDGVKFGEATVQVQVAGKSGLGREPRIQVTDQQDGVRAECTSAGCFPK | 180 |
| SWVERRDFRGQARPAVTNLSASATTRLWAVASSLTLWDRAVEGLSCSISSPLLPERSVSG | 240 |
| IHWGSWNVSPKDKGGLLESHSEVLGLELQQMTGGQGIQNGTHNNSQNAFSSNLKV | 295 |

A search of sequence databases reveals that the NOV11 amino acid sequence has 140 of 274 amino acid residues (51%) identical to, and 185 of 274 amino acid residues (67%) similar to, the 275 amino acid residue ptnr:SPTREMBL-ACC:Q9JK39 protein from Mus musculus (Mouse) (BUTYROPHILIN-LIKE PROTEIN BUTR-1)

The disclosed NOV11 polypeptide has homology to the amino acid sequences shown in the BLASTP data listed in Table 11C.

TABLE 11C
BLAST results for NOV11

| Gene Index/ | | Length | Identity | Positives | |
|---|---|---|---|---|---|
| Identifier | Protein/Organism | (aa) | (%) | (%) | Expect |
| gi\|8101125\|gb\|AAF72554.1\| | butyrophilin- | 275 | 50 | 65 | 1e−60 |
| AF269232_{—} | like protein | | | | |
| 1 (AF269232) | BUTR-1 [Mus | | | | |
| | musculus] | | | | |
| gi\|16741730\|gb\|AAH16661.1\| | Similar to | 334 | 39 | 58 | 7e−36 |
| AAH16661 | butyrophilin, | | | | |
| (BC016661) | subfamily 2, | | | | |
| | member A1 [Homo | | | | |
| | sapiens] | | | | |
| gi\|5921461\|ref\|NP_{—} | butyrophilin, | 527 | 39 | 58 | 1e−35 |
| 008980.1\| | subfamily 2, | | | | |
| (NM_007049) | member A1 [Homo | | | | |
| | sapiens] | | | | |
| gi\|14751898\|ref\|XP_{—} | (XM_030089) | 529 | 39 | 58 | 2e−35 |
| 030089.1\| | hypothetical | | | | |
| (XM_030089) | protein | | | | |
| | XP_030089 [Homo | | | | |
| | sapiens] | | | | |
| gi\|17028375\|gb\|AAH17497.1\| | Similar to | 493 | 57 | 74 | e−168 |
| AAH17497 | butyrophilin, | | | | |
| (BC017497) | subfamily 2, | | | | |
| | member A2 [Homo | | | | |
| | sapiens] | | | | |

The homology between these and other sequences is shown graphically in the ClustalW analysis shown in Table 11D. In the ClustalW alignment of the NOV11 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Tables 11E lists the domain description from DOMAIN analysis results against NOV11. This indicates that the NOV11 sequence has properties similar to those of other proteins known to contain this domain.

TABLE 11E
Domain Analysis of NOV11
gnl|Smart|smart00406, IGv, Immunoglobulin V-Type
CD-Length = 80 residues, 96.2% aligned
Score = 34.7 bits (78), Expect = 0.008

| | | | |
|---|---|---|---|
| Query: | 52 | VELQCQLFPNISAEDMELRWYRCQPSLAVHMHERGMDMDGEQKWQYRGRTTFMSDHVARG | 111 |
| | | V L C+ + + W R P + + Y+GR T D+ ++ | |
| Sbjct: | 2 | VTLSCKASGF-TFSSYYVSWVRQPPGKGLEWLGYIGSDVSYSEASYKGRVTISKDN-SKN | 59 |
| Query: | 112 | KAMVRSHRVTTFDNRTYCC | 130 |
| | | + + D TY C | |
| Sbjct: | 60 | DVSLTISNLRVEDTGTYYC | 78 |

The gene sequence of invention described herein encodes for a novel member of the B7-Immunoglobulin family of enzymes. Specifically, the sequence encodes a novel BUTYROPHILIN-like protein. BUTYROPHILIN molecules play crucial roles in T-cell activation making them plausible targets for cancer, AIDS, and inflammation therapies. The protein described here is known to be expressed in spleen, and liver which may indicate roles in lupus, endocrine disorders, inflammation, autoimmune disorders, and cancers including liver, bone, and leukemia.

Despite the fact that many tumors express MHC class I molecules presenting “foreign” peptide antigens, a vigorous tumor-destructing immune response is seldom detected. A possible explanation is that tumors cannot provide adequate costimulatory signals as provided by professional antigen presenting cells. CD28, upon interacting with B7, triggers costimulatory signals critical for the T-cell response. Transfection of tumor cells with B7 augments the immunogenicity of the tumor so that an anti-tumor immune response can be amplified. When B7-CD28 costimulation is provided CTL specific for otherwise silent epitopes can be activated. Therefore, unresponsiveness of T cells to many tumor antigens should be considered as ignorance rather than tolerance. Immunological ignorance may thus contribute to the failure of the immune system to respond against the tumor antigens.

There is considerable evidence to support an important role for co-stimulatory molecules in regulating the proliferation and activation of T cells in the immune response. Of particular relevance is the interaction between CD28 on T cells and B7 expressed on the surface of antigen presenting cells (APCs). CTLA-4, another molecule present on activated T cells may downregulate T cell activity, but its role remains uncertain. CTLA4-Ig, a fusion protein consisting of the extracellular domain of CTLA4 and the Fc portion of human immunoglobulin G1 (IgG1), has been useful for studying the role of CD28/B7 interactions in immune responses. A number of studies have shown that CTLA4-Ig can switch off T cell activation. In an ovalbumin sensitive murine model of asthma, CTLA4-Ig treatment suppressed the response to inhaled allergen (increased airway hyperresponsiveness [AHR], IgE production, recruitment of eosinophils into the lungs, production of IL-4, IL-5, and IL-10 and increased IFNgamma production from CD3-TCR-activated T cells). Anti B7-2 treatment has similar effects suggesting that interaction of B7-2 with CD28 is important in the development of a Th-2 type inflammatory response in mice. Recent observations have been of relevance to human allergic disease. In vitro studies have shown that CTLA4-Ig or anti-B7-2 antibody can inhibit allergen-induced proliferation and cytokine production by peripheral blood mononuclear cells from atopic subjects. The role of co-stimulation has been studied in a human bronchial explant model of asthma. CTLA4-Ig fusion protein effectively blocked allergen-induced production of IL-5 and IL- 13 in bronchial explants from atopic asthmatics. These studies confirm the requirement for interaction between co-stimulatory molecules in cytokine production and allergic inflammation, and point to the CD28-B7 pathway as being important to the allergen-induced inflammation in asthma. Studies of organ transplantation in primates suggest that CTLA4-Ig is extremely effective in preventing organ rejection. While phase 1 clinical trials have shown CTLA-4-Ig treatment of patients with psoriasis vulgaris to be well tolerated and to result in clinical improvement, its role in asthma management merits further investigation.

The initiation and progression of autoimmune diseases, such as insulin-dependent diabetes mellitus (IDDM), are complex processes that depend on autoantigen exposure, genetic susceptibility, and secondary events that promote autoaggression. T-cell costimulation, largely mediated by CD28/B7 interactions, is a major regulatory pathway in the activation and differentiation of T-cells that cause IDDM in murine models. In this article, we summarize our results in two models of IDDM: the non obese diabetic (NOD) mouse and diabetes induced with multiple low doses of streptozotocin (MDSDM). In both of these models, blockade of CD28/B7 costimulation regulates the development of disease. The effects of blockade vary with the intensity of cognate signal delivered to the T-cells, the timing of the costimulatory signal, and perhaps even the CD28 ligand expressed on antigen-presenting cells (APCs). Our results suggest that targeting CD28/B7 signals is a feasible approach for treatment and prevention of recurrence of autoimmune diabetes. However, the dynamic nature of these interactions highlights the importance of a clear understanding of their role in regulation of the disease. PMID: 9048209, UI: 97200274

The disclosed NOV11 nucleic acid of the invention encoding a butyrophilin-like protein includes the nucleic acid whose sequence is provided in Table 11A or a fragment thereof. The invention also includes a mutant or variant nucleic acid any of whose bases may be changed from the corresponding base shown in Table 11A while still encoding a protein that maintains its butyrophilin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV11 protein of the invention includes butyrophilin-like protein whose sequence is provided in Table 11B. The invention also includes a mutant or variant protein any of whose residues may be changed from the corresponding residue shown in Table 11B while still encoding a protein that maintains its butyrophilin-like activities and physiological functions, or a functional fragment thereof. In the mutant or variant protein, up to about 7 percent of the residues may be so changed.

The invention further encompasses antibodies and antibody fragments, such as F_{ab} or (F_{ab})₂, that bind immunospecifically to any of the proteins of the invention.

The above defined information for this invention suggests that this butyrophilin-like protein (NOV11) may function as a member of a butyrophilin family. Therefore, the NOV11 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV11 nucleic acids and proteins of the invention are useful in potential therapeutic applications implicated in cancer including but not limited to various pathologies and disorders as indicated below. For example, a cDNA encoding butyrophilin-like protein (NOV11) may be useful in gene therapy, and the butyrophilin-like protein (NOV11) may be useful when administered to a subject in need thereof. By way of nonlimiting example, the compositions of the present invention will have efficacy for treatment of patients suffering from brain disorders including epilepsy, eating disorders, schizophrenia, ADD, and cancer; heart disease; inflammation and autoimmune disorders including Crohn's disease, IBD, lupus, allergies, rheumatoid and osteoarthritis, inflammatory skin disorders, blood disorders; psoriasis colon cancer, leukemia AIDS; thalamus disorders; metabolic disorders including diabetes and obesity; lung diseases such as asthma, emphysema, cystic fibrosis, and cancer; multiple sclerosis, pancreatic disorders including pancreatic insufficiency and cancer; and prostate disorders including prostate cancer as well as other diseases, disorders and conditions. The NOV11 nucleic acid encoding the butyrophilin-like protein of the invention, or fragments thereof, may further be useful in diagnostic applications, wherein the presence or amount of the nucleic acid or the protein are to be assessed.

NOV11 nucleic acids and polypeptides are further useful in the generation of antibodies that bind immuno-specifically to the novel NOV11 substances for use in therapeutic or diagnostic methods. These antibodies may be generated according to methods known in the art, using prediction from hydrophobicity charts, as described in the “Anti-NOVX Antibodies” section below. The disclosed NOV11 protein has multiple hydrophilic regions, each of which can be used as an immunogen. In one embodiment, a contemplated NOV 11 epitope is from about amino acids 25 to 50. In another embodiment, a NOV11 epitope is from about amino acids 60 to 140. In additional embodiments, a NOV11 epitope is from about amino acids 150 to 200, and from about amino acids 240 to 280. These novel proteins can be used in assay systems for functional analysis of various human disorders, which will help in understanding of pathology of the disease and development of new drug targets for various disorders.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defmed as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+l to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS:1, 3, 5, 7, 9, 11, 13, 15, 17, 19, 21, 23, 25, 27, 29, 31, and 33, or a complement of this aforementioned nucleotide sequence, can be isolated using standard molecular biology techniques and the sequence information provided herein. Using all or a portion of the nucleic acid sequence of SEQ ID NOS:1, 3, 5, 7, 9, 11, 13, 15, 17, 19, 21, 23, 25, 27, 29, 31, and 33, as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS:1, 3, 5, 7, 9, 11, 13, 15, 17, 19, 21, 23, 25, 27, 29, 31, and 33, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 17, 19, 21, 23, 25, 27, 29, 31, and 33, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS:1, 3, 5, 7, 9, 11, 13, 15, 17, 19, 21, 23, 25, 27, 29, 31, 3 and 33 is one that is sufficiently complementary to the nucleotide sequence shown SEQ ID NOS:1, 3, 5, 7, 9, 11, 13, 15, 17, 19, 21, 23, 25, 27, 29, 31, and 33 that it can hydrogen bond with little or no mismatches to the nucleotide sequence shown SEQ ID NOS:1, 3, 5, 7, 9, 11, 13, 15, 17, 19, 21, 23, 25, 27, 29, 31, and 33, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS:1, 3, 5, 7, 9, 11, 13, 15, 17, 19, 21, 23, 25, 27, 29, 31, and 33, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS:1, 3, 5, 7, 9, 11, 13, 15, 17, 19, 21, 23, 25, 27, 29, 31, and 33; or an anti-sense strand nucleotide sequence of SEQ ID NOS:1, 3, 5, 7, 9, 11, 13, 15, 17, 19, 21, 23,25, 27, 29, 31, and 33; or of a naturally occurring mutant of SEQ ID NOS:1, 3, 5, 7, 9, 11, 13, 15, 17, 19, 21, 23, 25, 27, 29, 31, and 33.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:1, 3, 5, 7, 9, 11, 13, 15, 17, 19, 21, 23, 25, 27, 29, 31, and 33, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS:1, 3, 5, 7, 9, 11, 13, 15, 17, 19, 21, 23, 25, 27, 29, 31, and 33 due to degeneracy of the genetic code and thus encode the same NOVX proteins as that encoded by the nucleotide sequences shown in SEQ ID NOS:1, 3, 5, 7, 9, 11, 13, 15, 17, 19, 21, 23, 25, 27, 29, 31, and 33. In another embodiment, an isolated nucleic acid molecule of the invention has a nucleotide sequence encoding a protein having an amino acid sequence shown in SEQ ID NOS:2, 4, 6, 8, 10, 12, 14, 16, 18, 20, 22, 24, 26, 28, 30, 32, and 33.

In addition to the human NOVX nucleotide sequences shown in SEQ ID NOS:1, 3, 5, 7, 9, 11, 13, 15, 17, 19, 21, 23, 25, 27, 29, 31, 33, and 33, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS:1, 3, 5, 7, 9, 11, 13, 15, 17, 19, 21, 23, 25, 27, 29, 31, and 33 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 17, 19, 21, 23, 25, 27, 29, 31, and 33. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defmed ionic strength and pH. The Tm is the temperature (under defmed ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons; N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS:1, 3, 5, 7, 9, 11, 13, 15, 17, 19, 21, 23, 25, 27, 29, 31, and 33, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17, 19, 21, 23, 25, 27, 29, 31, and 33, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS:1, 3, 5, 7, 9, 11, 13, 15, 17, 19, 21, 23, 25, 27, 29, 31, and 33, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS:1, 3, 5, 7, 9, 11, 13, 15, 17, 19, 21, 23, 25, 27, 29, 31, and 33, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 4, 6, 8, 10, 12, 14, 16, 18, 20, 22, 24, 26, 28, 30, and 34. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS:1, 3, 5, 7, 9, 11, 13, 15, 17, 19, 21, 23, 25, 27, 29, 31, and 33 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 4, 6, 8, 10, 12, 14, 16, 18, 20, 22, 24, 26, 28, 30, 32, and 34. Preferably, the protein encoded by the nucleic acid molecule is at least about 60% homologous to SEQ ID NOS:2, 4, 6, 8, 10, 12, 14, 16, 18, 20, 22, 24, 26, 28, 30, 32, and 34; more preferably at least about 70% homologous SEQ ID NOS:2, 4, 6, 8, 10, 12, 14, 16, 18, 20, 22, 24, 26, 28, 30, 32, and 34; still more preferably at least about 80% homologous to SEQ ID NOS:2, 4, 6, 8, 10, 12, 14, 16, 18, 20, 22, 24, 26, 28, 30, 32, and 34; even more preferably at least about 90% homologous to SEQ ID NOS:2, 4, 6, 8, 10, 12, 14, 16, 18, 20, 22, 24, 26, 28, 30, 32, and 34; and most preferably at least about 95% homologous to SEQ ID NOS:2, 4, 6, 8, 10, 12, 14, 16, 18, 20, 22, 24, 26, 28, 30, 32, and 34.

An isolated nucleic acid molecule encoding an NOVX protein homologous to the protein of SEQ ID NOS:2, 4, 6, 8, 10, 12, 14, 16, 18, 20, 22, 24, 26, 28, 30, 32, and 34, can be created by introducing one or more nucleotide substitutions, additions or deletions into the nucleotide sequence of SEQ ID NOS:1, 3, 5, 7, 9, 11, 13, 15, 17, 19, 21, 23, 25, 27, 29, 31, and 33, such that one or more amino acid substitutions, additions or deletions are introduced into the encoded protein.

Mutations can be introduced into SEQ ID NOS:1, 3, 5, 7, 9, 11, 13, 15, 17, 19, 21, 23, 25, 27, 29, 31, and 33 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defmed within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS:1, 3, 5,7, 9, 11, 13, 15, 17, 19, 21, 23, 25, 27, 29, 31, and 33, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof, (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17, 19, 21, 23, 25, 27, 29, 31, and 33, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 16, 18, 20, 22, 24, 26, 28, 30, 32, and 34, or antisense nucleic acids complementary to an NOVX nucleic acid sequence of SEQ ID NOS:1, 3, 5, 7, 9, 11, 13, 15, 17, 19, 21, 23, 25, 27, 29, 31, and 33, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS:1, 3, 5, 7, 9, 11, 13, 15, 17, 19, 21, 23, 25, 27, 29, 31, and 33). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. US.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2, 4, 6, 8, 10, 12, 14, 16, 18, 20, 22, 24, 26, 28, 30, 32, and 34. The invention also includes a mutant or variant protein any of whose residues may be changed from the corresponding residues shown in SEQ ID NOS:2, 4, 6, 8, 10, 12, 14, 16, 18, 20, 22, 24, 26, 28, 30, 32, and 34 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 4, 6, 8, 10, 12, 14, 16, 18, 20, 22, 24, 26, 28, 30, 32, and 34) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2, 4, 6, 8, 10, 12, 14, 16, 18, 20, 22, 24, 26, 28, 30, 32, and 34. In other embodiments, the NOVX protein is substantially homologous to SEQ ID NOS:2, 4, 6, 8, 10, 12, 14, 16, 18, 20, 22, 24, 26, 28, 30, 32, and 34, and retains the functional activity of the protein of SEQ ID NOS:2, 4, 6, 8, 10, 12, 14, 16, 18, 20, 22, 24, 26, 28, 30, 32, and 34, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2, 4, 6, 8, 10, 12, 14, 16, 18, 20, 22, 24, 26, 28, 30, 32, and 34, and retains the functional activity of the NOVX proteins of SEQ ID NOS:2, 4, 6, 8, 10, 12, 14, 16, 18, 20, 22, 24, 26, 28, 30, 32, and 34.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, 7, 9, 11, 13, 15, 17, 19, 21, 23, 25, 27, 29, 31, and 33.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2, 4, 6, 8, 10, 12, 14, 16, 18, 20, 22, 24, 26, 28, 30, 32, and 34, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F(_{ab′})₂ fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse^{TM} as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol comRho-Interacting Proteing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191 -1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec I (Baldari, et al., 1987. EMBO J. 6: 229-234), pMFa (Kuran and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:1, 3, 5, 7, 9, 11, 13, 15, 17, 19, 21, 23, 25, 27, 29, 31, 33, and 35 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS:1, 3, 5, 7, 9, 11, 13, 15, 17, 19, 21, 23, 25, 27, 29, 31, and 33), but more preferably, is a non-human homologue of a human NOVX gene. For example, a mouse homologue of human NOVX gene of SEQ ID NOS:1, 3, 5, 7, 9, 11, 13, 15, 17, 19, 21, 23, 25, 27, 29, 31, and 33 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage PI. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12:145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X- 114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GALA). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS:1, 3, 5, 7, 9, 11, 13, 15, 17, 19, 21, 23, 25, 27, 29, 31,and 33, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17, 19, 21, 23, 25, 27, 29, 31, and 33, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a fill-length NOVX nucleic acid, such as the nucleic acid of SEQ ID NOS:1, 3, 5, 7, 9, 11, 13, 15, 17, 19, 21, 23, 25, 27, 29, 31, and 33, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7:5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6:1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitroftirans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic puipura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

### Identification of NOVX Clones

The novel NOVX target sequences identified in the present invention were subjected to the exon linking process to confirm the sequence. PCR primers were designed by starting at the most upstream sequence available, for the forward primer, and at the most downstream sequence available for the reverse primer. Table 12 shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

TABLE 12A

PCR Primers for Exon Linking

| | |
|---|---|
| SEQ | SEQ |

| | | |
|---|---|---|
| NOVX | ID | ID |

| | | | | |
|---|---|---|---|---|
| Clone | Primer 1 (5′-3′) | NO | Primer 2 (5′-3′) | NO |

| | | | | |
|---|---|---|---|---|
| NOV2d | CCAGCCAGGCGCCATGCT | 84 | TCTCTGGCCCGGGGGCTCA | 85 |
| NOV3 | ACTGCGGGCGCCCTGAGC | 86 | ATCACCTGCTCCCGTATCCATGCCT | 87 |
| NOV5b | ATGCGCCTTCCCGGGGTA | 88 | CGCCACCTTGCTCCACCCTA | 89 |
| NOV9 | CGACGGTTTAGACGTCTGTGCCACT | 179 | AGCAGTGCATCCTCCCCACTCAGT | 180 |
| NOV10b | ATGAGTGATAAACCCAACTTGTCAG | 90 | GTGAGCCATCATGCCCAG | 91 |

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

### Example 2

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS neurodegeneration panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1× TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1× TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D: The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.In the results for Panels 1, 1.1, 1.2 and 1.3D, the following abbreviations are used: ca.=carcinoma; *=established from metastasis; met=metastasis; s cell var=small cell variant; non-s=non-sm=non-small; squam=squamous; pl. eff=pl effusion=pleural effusion, glio=glioma; astro=astrocytoma, and neuro=neuroblastoma.

General_screening_panel_v1.4: The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2: The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D: The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D: Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Penn.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1 % serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 100 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

Autoimmunity (AI)_comprehensive pane_v1.0: The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics. Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims. Surgical specimens of psoriatic tissues and adjacent matched tissues were provided as total RNA by Clinomics. Two male and two female patients were selected between the ages of 25 and 47. None of the patients were taking prescription drugs at the time samples were isolated. Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital. Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators. In the labels employed to identify tissues in the AI_comprehensive pane_v1.0 panel, the following abbreviations are used: AI=Autoimmunity; Syn=Synovial; Normal=No apparent disease; Rep22/Rep20=individual patients; RA=Rheumatoid arthritis; Backus=From Backus Hospital; OA=Osteoarthritis; (SS) (BA) (MF)=Individual patients; Adj=Adjacent tissue; Match control=adjacent tissues; -M=Male; -F=Female; COPD=Chronic obstructive pulmonary disease

Panels 5D and 5I: The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained. In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample.
Patient 2: Diabetic Hispanic, overweight, not on insulin
Patient 7-9: Nondiabetic Caucasian and obese (BMI>30)
Patient 10: Diabetic Hispanic, overweight, on insulin
Patient 11: Nondiabetic African American and overweight
Patient 12: Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:
Donor 2 and 3 U: Mesenchymal Stem cells, Undifferentiated Adipose
Donor 2 and 3 AM: Adipose, AdiposeMidway Differentiated
Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I. In the labels employed to identify tissues in the 5D and 5I panels, the following abbreviations are used: GO Adipose=Greater Omentum Adipose; SK=Skeletal Muscle; UT=Uterus; PL=Placenta; AD=Adipose Differentiated; AM=Adipose Midway Differentiated; U=Undifferentiated Stem Cells

Central Nervous System Panel CNSD.01: The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology. Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration. In the labels employed to identify tissues in the CNS panel, the following abbreviations are used: PSP=Progressive supranuclear palsy; Sub Nigra=Substantia nigra; Glob Palladus=Globus palladus; Temp Pole=Temporal pole; Cing Gyr=Cingulate gyrus;BA 4=Brodman Area 4

Panel CNS_Neurodegeneration_V1.0: The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology. Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases. In the labels employed to identify tissues in the CNS_Neurodegeneration_V1.0 panel, the following abbreviations are used: AD=Alzheimer's disease brain; patient was demented and showed AD-like pathology upon autopsy; Control=Control brains; patient not demented, showing no neuropathology; Control (Path)=Control brains; pateint not demented but showing sever AD-like pathology; SupTemporal Ctx=Superior Temporal Cortex; Inf Temporal Ctx=Inferior Temporal Cortex

A. CG55758-01: EGF-Related Protein (SCUBE1)-Like Protein

Expression of gene CG55758-01 was assessed using the primer-probe set Ag2442, described in Table 12AA. Results of the RTQ-PCR runs are shown in Tables 12AB, 12AC, AD, and AE.

TABLE 12AA

Probe Name Ag2442

Start

| | | | |
|---|---|---|---|
| Primers | Sequences | Length | Position |

| | | | |
|---|---|---|---|
| Forward | 5′-gtcagtcgacgtggatgagt-3′ (SEQ ID NO:110) | 20 | 167 |
| Probe | TET-5′-agatgactgccacatcgatgccatct-3′- | 26 | 200 |
| | TAMRA (SEQ ID NO:111) | | |
| Reverse | 5′-gtaggacttgggcgtgttct-3′ (SEQ ID NO:112) | 20 | 229 |

TABLE 12AB
Panel 1.3D

| | Rel. Exp. (%) | Rel. Exp. (%) | | Rel. Exp. (%) | Rel. Exp. (%) |
|---|---|---|---|---|---|
| | Ag2442, Run | Ag2442, Run | | Ag2442, Run | Ag2442, Run |
| Tissue Name | 159771448 | 165639093 | Tissue Name | 159771448 | 165639093 |
| Liver | 0.0 | 0.0 | Kidney (fetal) | 6.5 | 3.5 |
| adenocarcinoma | | | | | |
| Pancreas | 0.3 | 0.9 | Renal ca. 786-0 | 0.0 | 0.0 |
| Pancreatic ca. | 1.0 | 0.0 | Renal ca. A498 | 51.4 | 32.1 |
| CAPAN 2 | | | | | |
| Adrenal gland | 21.5 | 5.8 | Renal ca. RXF | 0.0 | 0.0 |
| | | | 393 | | |
| Thyroid | 5.5 | 1.5 | Renal ca. ACHN | 0.0 | 0.0 |
| Salivary gland | 3.8 | 2.3 | Renal ca. UO-31 | 0.0 | 0.0 |
| Pituitary gland | 6.7 | 3.4 | Renal ca. TK-10 | 0.2 | 0.0 |
| Brain (fetal) | 31.4 | 31.6 | Liver | 13.8 | 5.2 |
| Brain (whole) | 3.1 | 1.6 | Liver (fetal) | 0.0 | 0.0 |
| Brain (amygdala) | 5.6 | 4.6 | Liver ca. | 0.0 | 0.0 |
| | | | (hepatoblast) | | |
| | | | HepG2 | | |
| Brain | 0.0 | 1.4 | Lung | 22.7 | 12.4 |
| (cerebellum) | | | | | |
| Brain | 9.7 | 1.6 | Lung (fetal) | 5.1 | 2.9 |
| (hippocampus) | | | | | |
| Brain (substantia | 4.9 | 4.4 | Lung ca. (small | 0.0 | 0.0 |
| nigra) | | | cell) LX-1 | | |
| Brain (thalamus) | 0.4 | 1.2 | Lung ca. (small | 1.3 | 0.7 |
| | | | cell) NCI-H69 | | |
| Cerebral Cortex | 1.6 | 0.7 | Lung ca. | 24.7 | 11.5 |
| | | | (s.cell var.) | | |
| | | | SHP-77 | | |
| Spinal cord | 7.2 | 9.6 | Lung ca. (large | 6.2 | 4.2 |
| | | | cell)NCI-H460 | | |
| glio/astro U87-MG | 0.0 | 0.0 | Lung ca. (non- | 0.0 | 0.0 |
| | | | sm. cell) A549 | | |
| glio/astro U-118- | 0.0 | 0.0 | Lung ca. (non- | 0.0 | 0.7 |
| MG | | | s.cell) NCI-H23 | | |
| astrocytoma | 0.0 | 0.0 | Lung ca. (non- | 0.0 | 0.0 |
| SW1783 | | | s.cell) HOP-62 | | |
| neuro*; met SK-N- | 3.0 | 1.9 | Lung ca. (non- | 0.0 | 0.0 |
| AS | | | s.cl) NCI-H522 | | |
| astrocytoma SF- | 26.8 | 23.8 | Lung ca. | 0.0 | 0.0 |
| 539 | | | (squam.) SW 900 | | |
| astrocytoma SNB- | 100.0 | 100.0 | Lung ca. | 0.0 | 0.0 |
| 75 | | | (squam.) NCI- | | |
| | | | H596 | | |
| glioma SNB-19 | 0.0 | 0.0 | Mammary gland | 8.0 | 3.0 |
| glioma U251 | 0.2 | 0.0 | Breast ca.* | 0.3 | 0.6 |
| | | | (pl.ef) MCF-7 | | |
| glioma SF-295 | 11.9 | 4.6 | Breast ca.* | 0.0 | 0.4 |
| | | | (pl.ef) MDA-MB- | | |
| | | | 231 | | |
| Heart (fetal) | 0.0 | 0.0 | Breast ca.* | 0.0 | 0.0 |
| | | | (pl.ef) T47D | | |
| Heart | 0.8 | 0.9 | Breast ca. BT- | 0.0 | 0.7 |
| | | | 549 | | |
| Skeletal muscle | 1.9 | 0.0 | Breast ca. MDA-N | 0.0 | 0.0 |
| (fetal) | | | | | |
| Skeletal muscle | 0.3 | 0.0 | Ovary | 69.7 | 27.9 |
| Bone marrow | 5.3 | 2.3 | Ovarian ca. | 0.3 | 0.0 |
| | | | OVCAR-3 | | |
| Thymus | 0.0 | 0.0 | Ovarian ca. | 0.0 | 0.0 |
| | | | OVCAR-4 | | |
| Spleen | 42.3 | 15.1 | Ovarian ca. | 0.0 | 0.0 |
| | | | OVCAR-5 | | |
| Lymph node | 0.4 | 0.0 | Ovarian ca. | 0.0 | 0.0 |
| | | | OVCAR-8 | | |
| Colorectal | 5.4 | 5.8 | Ovarian ca. | 0.0 | 0.0 |
| | | | IGROV-1 | | |
| Stomach | 12.0 | 6.4 | Ovarian ca.* | 0.0 | 0.0 |
| | | | (ascites) SK- | | |
| | | | OV-3 | | |
| Small intestine | 22.8 | 19.9 | Uterus | 0.6 | 0.9 |
| Colon ca. SW480 | 0.0 | 0.0 | Plancenta | 3.1 | 2.5 |
| Colon ca.* | 0.0 | 0.0 | Prostate | 0.8 | 2.7 |
| SW620 (SW480 met) | | | | | |
| Colon ca. HT29 | 0.3 | 0.0 | Prostate ca.* | 0.0 | 0.0 |
| | | | (bone met)PC-3 | | |
| Colon ca. HCT-116 | 1.7 | 2.9 | Testis | 17.4 | 4.4 |
| Colon ca. CaCo-2 | 0.0 | 0.0 | Melanoma | 0.8 | 0.0 |
| | | | Hs688(A).T | | |
| Colon ca. | 0.4 | 0.0 | Melanoma* (met) | 0.0 | 0.0 |
| tissue(ODO3866) | | | Hs688(B).T | | |
| Colon ca. HCC- | 0.4 | 0.0 | Melanoma UACC- | 0.0 | 0.0 |
| 2998 | | | 62 | | |
| Gastric ca.* | 0.0 | 0.0 | Melanoma M14 | 0.0 | 0.0 |
| (liver met) NCI- | | | | | |
| N87 | | | | | |
| Bladder | 0.9 | 0.6 | Melanoma LOX | 0.0 | 0.0 |
| | | | IMVI | | |
| Trachea | 24.8 | 9.0 | Melanoma* (met) | 1.4 | 0.0 |
| | | | SK-MEL-5 | | |
| Kidney | 3.9 | 2.0 | Adipose | 0.8 | 2.7 |

TABLE 12AC
Panel 2D

| | Rel. Exp. (%) | | Rel. Exp. (%) |
|---|---|---|---|
| | Ag2442, Run | | Ag2442, Run |
| Tissue Name | 159629159 | Tissue Name | 159629159 |
| Normal Colon | 25.7 | Kidney Margin 8120608 | 15.9 |
| CC Well to Mod Diff | 1.3 | Kidney Cancer 8120613 | 0.0 |
| (ODO3866) | | | |
| CC Margin (ODO3866) | 16.5 | Kidney Margin 8120614 | 33.2 |
| CC Gr.2 rectosigmoid | 1.8 | Kidney Cancer 9010320 | 6.7 |
| (ODO3868) | | | |
| CC Margin (ODO3868) | 1.6 | Kidney Margin 9010321 | 18.6 |
| CC Mod Diff (ODO3920) | 8.8 | Normal Uterus | 0.6 |
| CC Margin (ODO3920) | 6.4 | Uterus Cancer 064011 | 4.6 |
| CC Gr.2 ascend colon | 23.3 | Normal Thyroid | 7.3 |
| (ODO3921) | | | |
| CC Margin (ODO3921) | 17.8 | Thyroid Cancer 064010 | 1.6 |
| CC from Partial | 3.9 | Thyroid Cancer | 0.0 |
| Hepatectomy (ODO4309) | | A302152 | |
| Mets | | | |
| Liver Margin (ODO4309) | 3.3 | Thyroid Margin | 2.0 |
| | | A302153 | |
| Colon mets to lung | 3.5 | Normal Breast | 8.3 |
| (OD04451-01) | | | |
| Lung Margin (OD04451-02) | 26.4 | Breast Cancer | 2.1 |
| | | (OD04566) | |
| Normal Prostate 6546-1 | 1.8 | Breast Cancer | 2.0 |
| | | (OD04590-01) | |
| Prostate Cancer | 3.4 | Breast Cancer Mets | 1.2 |
| (OD04410) | | (OD04590-03) | |
| Prostate Margin | 7.8 | Breast Cancer | 2.5 |
| (OD04410) | | Metastasis (OD04655- | |
| | | 05 | |
| Prostate Cancer | 3.2 | Breast Cancer 064006 | 2.8 |
| (OD04720-01) | | | |
| Prostate Margin | 6.7 | Breast Cancer 1024 | 3.7 |
| (OD04720-02) | | | |
| Normal Lung 061010 | 33.4 | Breast Cancer 9100266 | 42.3 |
| Lung Met to Muscle | 0.4 | Breast Margin 9100265 | 9.8 |
| (ODO4286) | | | |
| Muscle Margin (ODO4286) | 1.1 | Breast Cancer A209073 | 3.3 |
| Lung Malignant Cancer | 13.4 | Breast Margin | 4.2 |
| (OD03126) | | A2090734 | |
| Lung Margin (OD03126) | 69.7 | Normal Liver | 7.6 |
| Lung Cancer (OD04404) | 5.1 | Liver Cancer 064003 | 0.0 |
| Lung Margin (OD04404) | 39.8 | Liver Cancer 1025 | 0.0 |
| Lung Cancer (OD04565) | 0.4 | Liver Cancer 1026 | 34.4 |
| Lung Margin (OD04565) | 17.4 | Liver Cancer 6004-T | 13.0 |
| Lung Cancer (OD04237-01) | 4.6 | Liver Tissue 6004-N | 0.6 |
| Lung Margin (OD04237-02) | 37.6 | Liver Cancer 6005-T | 33.4 |
| Ocular Mel Met to Liver | 0.7 | Liver Tissue 6005-N | 7.2 |
| (ODO4310) | | | |
| Liver Margin (ODO4310) | 0.0 | Normal Bladder | 3.7 |
| Melanoma Mets to Lung | 1.0 | Bladder Cancer 1023 | 3.0 |
| (OD04321) | | | |
| Lung Margin (OD04321) | 89.5 | Bladder Cancer | 1.0 |
| | | A302173 | |
| Normal Kidney | 15.9 | Bladder Cancer | 0.9 |
| | | OD04718-01) | |
| Kidney Ca, Nuclear grade | 4.2 | Bladder Normal | 0.6 |
| 2 (OD04338) | | Adjacent (OD04718-03) | |
| Kidney Margin (OD04338) | 17.3 | Normal Ovary | 100.0 |
| Kidney Ca Nuclear grade | 4.3 | Ovarian Cancer 064008 | 6.3 |
| ½ (OD04339) | | | |
| Kidney Margin (OD04339) | 18.9 | Ovarian Cancer | 0.0 |
| | | (OD04768-07) | |
| Kidney Ca, Clear cell | 1.7 | Ovary Margin | 1.2 |
| type (OD04340) | | (OD04768-08) | |
| Kidney Margin (OD04340) | 17.2 | Normal Stomach | 33.7 |
| Kidney Ca Nuclear grade | 0.5 | Gastric Cancer | 5.9 |
| 3 (OD04348) | | 9060358 | |
| Kidney Margin (OD04348) | 14.8 | Stomach Margin | 13.9 |
| | | 9060359 | |
| Kidney Cancer (OD04622- | 3.1 | Gastric Cancer | 31.0 |
| 01) | | 9060395 | |
| Kidney Margin (OD04622- | 8.0 | Stomach Margin | 29.7 |
| 03) | | 9060394 | |
| Kidney Cancer (OD04450- | 0.5 | Gastric Cancer | 6.2 |
| 01) | | 9060397 | |
| Kidney Margin (OD04450- | 9.6 | Stomach Margin | 14.2 |
| 03) | | 9060396 | |
| Kidney Cancer 8120607 | 2.1 | Gastric Cancer 064005 | 12.3 |

TABLE 12AD
Panel 3D

| | Rel. Exp. (%) | | Rel. Exp. (%) |
|---|---|---|---|
| | Ag2442, Run | | Ag2442, Run |
| Tissue Name | 164632279 | Tissue Name | 164632279 |
| Daoy-Medulloblastoma | 2.0 | Ca Ski-Cervical epidermoid | 0.0 |
| | | carcinoma (metastasis) | |
| TE671-Medulloblastoma | 2.0 | ES-2-Ovarian Clear cell | 0.0 |
| | | carcinoma | |
| D283 Med- | 0.0 | Ramos-Stimulated with | 0.0 |
| Medulloblastoma | | PMA/ionomycin 6h | |
| PFSK-1-Primitive | 3.3 | Ramos-Stimulated with | 0.0 |
| Neuroectodermal | | PMA/ionomycin 14h | |
| XF-498-CNS | 0.0 | MEG-01-Chronic myelogenous | 4.6 |
| | | leukemia (megokaryoblast) | |
| SNB-78-Glioma | 0.0 | Raji-Burkitt's lymphoma | 0.0 |
| SF-268-Glioblastoma | 0.0 | Daudi-Burkitt's lymphoma | 0.4 |
| T98G-Glioblastoma | 29.9 | U266-B-cell plasmacytoma | 1.8 |
| SK-N-SH-Neuroblastoma | 17.6 | CA46-Burkitt's lymphoma | 0.0 |
| (metastasis) | | | |
| SF-295-Glioblastoma | 11.2 | RL-non-Hodgkin's B-cell | 0.0 |
| | | lymphoma | |
| Cerebellum | 2.0 | JM1-pre-B-cell lymphoma | 0.0 |
| Cerebellum | 1.0 | Jurkat-T cell leukemia | 0.4 |
| NCI-H292-Mucoepidermoid | 0.0 | TF-1-Erythroleukemia | 3.5 |
| lung carcinoma | | | |
| DMS-114-Small cell lung | 1.4 | HUT 78-T-cell lymphoma | 0.0 |
| cancer | | | |
| DMS-79-Small cell lung | 11.2 | U937-Histiocytic lymphoma | 0.0 |
| cancer | | | |
| NCI-H146-Small cell | 0.0 | KU-812-Myelogenous | 0.0 |
| lung cancer | | leukemia | |
| NCI-H526-Small cell | 7.2 | 769-P-Clear cell renal | 0.0 |
| lung cancer | | carcinoma | |
| NCI-N417-Small cell | 1.9 | Caki-2-Clear cell renal | 1.0 |
| lung cancer | | carcinoma | |
| NCI-H82-Small cell lung | 0.0 | SW 839-Clear cell renal | 0.0 |
| cancer | | carcinoma | |
| NCI-H157-Squamous cell | 0.0 | G401-Wilms' tumor | 100.0 |
| lung cancer (metastasis) | | | |
| NCI-H1155-Large cell | 0.0 | Hs766T-Pancreatic | 0.0 |
| lung cancer | | carcinoma (LN metastasis) | |
| NCI-H1299-Large cell | 0.0 | CAPAN-1-Pancreatic | 0.0 |
| lung cancer | | adenocarcinoma (liver | |
| | | metastasis) | |
| NCI-H727-Lung carcinoid | 22.8 | SU86.86-Pancreatic | 0.0 |
| | | carcinoma (liver | |
| | | metastasis) | |
| NCI-UMC-11-Lung | 1.8 | BxPC-3-Pancreatic | 0.0 |
| carcinoid | | adenocarcinoma | |
| LX-1-Small cell lung | 0.0 | HPAC-Pancreatic | 0.0 |
| cancer | | adenocarcinoma | |
| Colo-205-Colon cancer | 0.0 | MIA PaCa-2-Pancreatic | 0.0 |
| | | carcinoma | |
| KM12-Colon cancer | 0.0 | CFPAC-1-Pancreatic ductal | 0.0 |
| | | adenocarcinoma | |
| KM20L2-Colon cancer | 0.0 | PANC-1-Pancreatic | 0.0 |
| | | epithelioid ductal | |
| | | carcinoma | |
| NCI-H716-Colon cancer | 0.0 | T24-Bladder carcinma | 0.0 |
| | | (transitional cell) | |
| SW-48-Colon | 0.0 | 5637-Bladder carcinoma | 0.0 |
| (adenocarcinoma | | | |
| SW1116-Colon | 0.0 | HT-1197-Bladder carcinoma | 7.3 |
| adenocarcinoma | | | |
| LS 174T-Colon | 0.0 | UM-UC-3-Bladder carcinma | 0.0 |
| adenocarcinoma | | (transitional cell) | |
| SW-948-Colon | 0.0 | A204-Rhabdomyosarcoma | 0.0 |
| adenocarcinoma | | | |
| SW-480-Colon | 0.0 | HT-1080-Fibrosarcoma | 0.0 |
| adenocarcinoma | | | |
| NCI-SNU-5-Gastric | 0.0 | MG-63-Osteosarcoma | 4.4 |
| carcinoma | | | |
| KATO III-Gastric | 0.0 | SK-LMS-1-Leiomyosarcoma | 0.0 |
| carcinoma | | (vulva) | |
| NCI-SNU-16-Gastric | 0.0 | SJRH30-Rhabdomyosarcoma | 76.8 |
| carcinoma | | (met to bone marrow) | |
| NCI-SNU-1-Gastric | 0.0 | A431-EPidermoid carcinoma | 0.0 |
| carcinoma | | | |
| RF-1-Gastric | 0.0 | WM266-4-Melanoma | 0.0 |
| RF-48-Gastric | 0.0 | DU 145-Prostate carcinoma | 0.0 |
| adenocarcinoma | | (brain metastasis) | |
| MKN-45-Gastric | 0.0 | MDA-MB-468-Breast | 0.0 |
| carcinoma | | adenocarcinoma | |
| NCI-N87-Gastric | 0.0 | SCC-4-Squamous cell | 1.9 |
| carcinoma | | carcinoma of tongue | |
| OVCAR-5-Ovarian | 0.0 | SCC-9-Squamous cell | 0.0 |
| carcinoma | | carcinoma of tongue | |
| RL95-2-Uterine | 0.7 | SCC-15-Squamous cell | 0.0 |
| carcinoma | | carcinoma of tongue | |
| HelaS3-Cervical | 0.0 | CAL 27-Squamous cell | 0.0 |
| adenocarcinoma | | carcinoma of tongue | |

TABLE 12AE
Panel 4.1D

| | Rel. Exp. (%) | | Rel. Exp. (%) |
|---|---|---|---|
| | Ag2442, Run | | Ag2442, Run |
| Tissue Name | 170737037 | Tissue Name | 170737037 |
| Secondary Th1 act | 0.0 | HUVEC IL-1beta | 0.0 |
| Secondary Th2 act | 0.0 | HUVEC IFN gamma | 2.5 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 3.1 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 lymphocyte | 0.0 | Coronery artery SMC rest | 0.0 |
| act | | | |
| CD45RO CD4 lymphocyte | 2.1 | coronery artery SMC | 0.0 |
| act | | TNF alpha + IL-1beta | |
| CD8 lymphocyte act | 0.0 | Astrocytes rest | 0.0 |
| Secondary CD8 | 0.0 | Astrocytes TNF alpha + IL- | 5.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 7.7 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 9.0 |
| LAK cells IL-2 + IL-12 | 0.0 | NCI-H292 none | 0.0 |
| LAK cells IL-2 + IFN | 0.0 | NCI-H292 IL-4 | 0.0 |
| gamma | | | |
| LAK cells IL-2 + IL-18 | 1.7 | NCI-H292 IL-9 | 0.0 |
| LAK cells | 2.3 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL- | 0.0 |
| | | 1beta | |
| Two Way MLR 7 day | 0.0 | Lung fibroblast none | 0.0 |
| PBMC rest | 0.0 | Lung fibroblast TNF alpha + IL- | 0.0 |
| | | 1beta | |
| PBMC PWM | 0.0 | Lung fibroblast IL-4 | 8.5 |
| PBMC PHA-L | 0.0 | Lung fibroblast IL-9 | 5.6 |
| Ramos (B cell) none | 0.0 | Lung fibroblast IL-13 | 3.1 |
| Ramos (B cell) | 0.0 | Lung fibroblast IFN gamma | 0.0 |
| ionomycin | | | |
| B lymphocytes PWM | 0.0 | Dermal fibroblast CCD1070 | 0.0 |
| | | rest | |
| B lymphocytes CD40L | 0.0 | Dermal fibroblast CCD1070 | 0.0 |
| and IL-4 | | TNF alpha | |
| EOL-1 dbcAMP | 0.0 | Dermal fibroblast CCD1070 | 0.0 |
| | | IL-1beta | |
| EOL-1 dbcAMP | 2.4 | Dermal fibroblast IFN | 0.0 |
| PMA/ionomycin | | gamma | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti- | 0.0 | Neutrophils TNFa + LPS | 0.0 |
| CD40 | | | |
| Monocytes rest | 1.9 | Neutrophils rest | 0.0 |
| Monocytes LPS | 2.4 | Colon | 18.8 |
| Macrophages rest | 0.0 | Lung | 11.7 |
| Macrophages LPS | 0.0 | Thymus | 13.8 |
| HUVEC none | 0.0 | Kidney | 100.0 |
| HUVEC starved | 0.0 | | |

CNS_neurodegeneration_v1.0 Summary: Ag2442 Expression of CG55758-01 is low/undetectable in all samples in this panel (CT>35). (Data not shown.)

Panel 1.3D Summary: Ag2442 Two experiments with the same probe and primer set produce results that are in excellent agreement, with both experiments showing highest expression of the CG55758-01 gene in an astrocytoma cell line (CTs=29-30). It is also expressed at lower levels in two lung cancer cell lines and a renal cancer cell line. There is also low level expression in a number of normal tissues including testis, ovary, mammalian gland, lung, trachea, kidney, spleen and brain. The increased expression seen in the astrocytoma cell line suggests that this gene may play a role in the cancers used in the derivation of this cell line. Thus, therapeutic inhibition of the function of this gene product, through the use of antibodies or small molecule drugs, might be of utility in the treatment of this disease. Both runs show highest expression of this SCUBE1-like gene among metabolically relevant tissues in the small intestine. Lower levels of expression are also seen in the adrenal gland, liver, and stomach. This expression profile suggests that this gene and its product may be involved in the development of these organs and their interaction with the extracellular environment. Therefore, antibody or protein therapeutics targeted towards this gene product may be effective therapeutics against diseases and conditions involving these organs. This gene is a novel SCUBE1-like protein that is expresed in the developing brain. This gene or its protein product may therefore be of use in the treatment of developmental disorders such as autism, schizophrenia, attention deficit disorder, and Tourette syndrome.

Panel 2D Summary: Ag2442 The CG55758-01 gene is highly expressed in a normal ovary sample (CT=29.1). The level of expression in some lung, prostate, ovary and kidney normal samples appears to be increased when compared to the matched tumor tissue. The reverse appears to be true for liver, where expression is slightly higher in the tumor tissue than the matched normal tissues. Thus, based upon its profile, the expression of this gene could be of use as a marker for distinguishing some cancers from the normal adjacent tissue or as a marker for different grades/types of cancer. Therapeutic use of this gene, through the use of peptides, polypeptides or small molecule drugs, might be of utility in the treatment of lung, prostate, ovary and kidney cancer; while inhibition of its activity might be used for treatment of liver cancer.

Panel 3D Summary: Ag2442 The CG55758-01 gene is expressed in select cancer cell lines in this panel. The highest level of expression is in a cell line derived from Wilm's tumor, G401 (CT=30.3). A high level of expression is also seen in rhabdomyosarcoma and lung and brain cancer cell lines. Thus, therapeutic inhibition of the function of this gene, through the use of antibodies or small molecule drugs, might be of utility in the treatment of cancers from which these cell lines were derived.

Panel 4.1D Summary: Ag2442 The CG55758-01 transcript is expressed at low level in kidney and colon in this panel (CTs=32-35). The putative EGF-related protein encoded by this transcript may play an important role in the normal development and homeostasis of these tissues. Modulation of the expression or function of the protein encoded by this transcript could be important for maintaining or restoring normal function to these organs during inflammation.

Panel 4D Summary: Ag2442 Data from one experiment with this probe and primer set is not included because the amp plot suggests that there was a problem with one of the sample wells.

B. CG55724-01: Adipocyte Complement Related Protein

Expression of gene CG55724-01 was assessed using the primer-probe set Ag3094, described in Table 12BA. Results of the RTQ-PCR runs are shown in Table BB.

TABLE 12BA

Probe Name Ag3094

Start

| | | | |
|---|---|---|---|
| Primers | Sequences | Length | Position |

| | | | |
|---|---|---|---|
| Forward | 5′-gagctttgccctgttctgtt-3′ | 20 | 43 |
| | (SEQ ID NO:113) | | |
| Probe | TET-5′-tgctctctagacccagaggacgaagc- | 26 | 66 |
| | 3′-TAMRA (SEQ ID NO:114) | | |
| Reverse | 5′-acccttcctcatctgtgacc-3′ | 20 | 100 |
| | (SEQ ID NO:115) | | |

TABLE 12BB
Panel 1.3D

| | Rel. Exp. (%) Ag3094, | | Rel. Exp. (%) Ag3094, |
|---|---|---|---|
| Tissue Name | Run 167985247 | Tissue Name | Run 167985247 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 100.0 | Liver | 0.0 |
| Brain (whole) | 0.0 | Liver (fetal) | 0.0 |
| Brain (amygdala) | 0.0 | Liver ca. | 0.0 |
| | | (hepatoblast) HepG2 | |
| Brain (cerebellum) | 0.0 | Lung | 0.0 |
| Brain (hippocampus) | 0.0 | Lung (fetal) | 0.0 |
| Brain (substantia | 0.0 | Lung ca. (small | 0.0 |
| nigra) | | cell) LX-1 | |
| Brain (thalamus) | 0.0 | Lung ca. (small | 0.0 |
| | | cell) NCI-H69 | |
| Cerebral Cortex | 0.0 | Lung ca. (s.cell | 0.0 |
| | | var.) SHP-77 | |
| Spinal Cord | 0.0 | Lung ca. (large | 0.0 |
| | | cell) NCI-H460 | |
| glio/astro U87-MG | 0.0 | Lung ca. (non-sm. | 0.0 |
| | | cell) A549 | |
| glio/astro U-118-MG | 0.0 | Lung ca. (non- | 0.0 |
| | | s.cell) NCI-H23 | |
| astrocytoma SW1783 | 0.0 | Lung ca. (non- | 0.0 |
| | | s.cell) HOP-62 | |
| neuro*; met SK-N-AS | 0.0 | Lung ca. (non-s.cl) | 0.0 |
| | | NCI-H522 | |
| astrocytoma SF-539 | 0.0 | Lung ca. (squam.) SW | 0.0 |
| | | 900 | |
| astrocytoma SNB-75 | 0.0 | Lung ca. (squam.) | 0.0 |
| | | NCI-H596 | |
| glioma SNB-19 | 0.0 | Mammary gland | 0.0 |
| glioma U251 | 0.0 | Breast ca.* (pl.ef) | 0.0 |
| | | MCF-7 | |
| glioma SF-295 | 0.0 | Breast ca.* (pl.ef) | 0.0 |
| | | MDA-MB-231 | |
| Heart (fetal) | 0.0 | Breast ca.* (pl.ef) | 0.0 |
| | | T47D | |
| Heart | 0.0 | Breast ca. BT-549 | 0.0 |
| Skeletal muscle | 0.0 | Breast ca. MDA-N | 0.0 |
| (fetal) | | | |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca.* | 0.0 |
| | | (ascites) SK-OV-3 | |
| Small intestine | 0.0 | Uterus | 0.0 |
| Colon ca. SW480 | 0.0 | Plancenta | 0.0 |
| Colon ca.* SW620 (SW480 | 0.0 | Prostate | 0.0 |
| met) | | | |
| Colon ca. HT29 | 0.0 | Prostate ca.* (bone | 0.0 |
| | | met) PC-3 | |
| Colon ca. HCT-116 | 0.0 | Testis | 0.0 |
| Colon ca. CaCo-2 | 0.0 | Melanoma Hs688(A).T | 0.0 |
| Colon ca. | 0.0 | Melanoma* (met) | 0.0 |
| tissue (ODO3866) | | Hs688(B).T | |
| Colon ca. HCC-2998 | 0.0 | Melanoma UACC-62 | 0.0 |
| Gastric ca.* (liver | 0.0 | Melanoma M14 | 0.0 |
| met) NCI-N87 | | | |
| Bladder | 0.0 | Melanoma LOX IMVI | 0.0 |
| Trachea | 0.0 | Melanoma* (met) SK- | 0.0 |
| | | MEL-5 | |
| Kidney | 0.0 | Adipose | 0.0 |

CNS_neurodegeneration_v1.0 Summary: Ag3094 Expression of the CG55724-01 gene is low/undetectable in all samples on this panel (CTs>35). (Data not shown.)

Panel 1.3D Summary: Ag3094 The CG55724-01 gene is a novel adipocyte complement-related protein which is expresed in the developing brain. This gene or its protein product may therefore be of use in the treatment of developmental disorders such as autism, schizophrenia, attention deficit disorder, or Tourette syndrome.

Panel 2.2 Summary: Ag3094 Expression of the CG55724-01 gene is low/undetectable in all samples on this panel (CTs>35). (Data not shown.)

Panel 4D Summary: Ag3094 Expression of the CG55724-01 gene is low/undetectable in all samples on this panel (CTs>35). (Data not shown.)

C. CG50345-01: Beta-Adrenergic Receptor Kinase

Expression of gene CG50345-01 was assessed using the primer-probe set Ag2303, described in Table12CA. Results of the RTQ-PCR runs are shown in Tables CB, and CC.

TABLE 12CA

Probe Name Ag2303

Start

| | | | |
|---|---|---|---|
| Primers | Sequences | Length | Position |

| | | | |
|---|---|---|---|
| Forward | 5′-cattgagagcgataagttcaca-3′ | 22 | 602 |
| | SEQ ID NO:113 (SEQ ID NO:116) | | |
| Probe | TET-5′-agaatgtggagctcaacatccacctg- | 26 | 640 |
| | 3′-TAMRA (SEQ ID NO:117) | | |
| Reverse | 5′-gatgcacgctgaagtcattc-3′ | 20 | 671 |
| | (SEQ ID NO:118) | | |

TABLE 12CB
Panel 1.3D

| | Rel. Exp. (%) Ag2303, | | Rel. Exp. (%) Ag2303, |
|---|---|---|---|
| Tissue Name | Run 167985232 | Tissue Name | Run 167985232 |
| Liver adenocarcinoma | 19.1 | Kidney (fetal) | 25.5 |
| Pancreas | 5.1 | Renal ca. 786-0 | 7.4 |
| Pancreatic ca. CAPAN 2 | 20.0 | Renal ca. A498 | 6.8 |
| Adrenal gland | 2.7 | Renal ca. RXF 393 | 15.5 |
| Thyroid | 2.3 | Renal ca. ACHN | 3.9 |
| Salivary gland | 7.2 | Renal ca. UO-31 | 6.3 |
| Pituitary gland | 5.0 | Renal ca. TK-10 | 16.4 |
| Brain (fetal) | 31.9 | Liver | 6.1 |
| Brain (whole) | 58.2 | Liver (fetal) | 6.7 |
| Brain (amygdala) | 33.9 | Liver ca. | 11.7 |
| | | (hepatoblast) HepG2 | |
| Brain (cerebellum) | 55.5 | Lung | 14.7 |
| Brain (hippocampus) | 23.3 | Lung (fetal) | 11.0 |
| Brain (substantia | 15.3 | Lung ca. (small | 36.6 |
| nigra) | | cell) LX-1 | |
| Brain (thalamus) | 21.9 | Lung ca. (small | 15.0 |
| | | cell) NCI-H69 | |
| Cerebral Cortex | 80.1 | Lung ca. (s.cell | 60.7 |
| | | var.) SHP-77 | |
| Spinal cord | 8.4 | Lung ca. (large | 5.4 |
| | | cell)NCI-H460 | |
| glio/astro U87-MG | 12.0 | Lung ca. (non-sm. | 14.3 |
| | | cell) A549 | |
| glio/astro U-118-MG | 10.8 | Lung ca. (non- | 37.4 |
| | | s.cell) NCI-H23 | |
| astrocytoma SW1783 | 15.5 | Lung ca. (non- | 14.5 |
| | | s.cell) HOP-62 | |
| neuro*; met SK-N-AS | 7.0 | Lung ca. (non-s.cl) | 15.6 |
| | | NCI-H522 | |
| astrocytoma SF-539 | 9.9 | Lung ca. (squam.) SW | 16.2 |
| | | 900 | |
| astrocytoma SNB-75 | 15.9 | Lung ca. (squam.) | 33.2 |
| | | NCI-H596 | |
| glioma SNB-19 | 8.7 | Mammary gland | 17.6 |
| glioma U251 | 20.7 | Breast ca.* (pl.ef) | 17.1 |
| | | MCF-7 | |
| glioma SF-295 | 7.9 | Breast ca.* (pl.ef) | 6.7 |
| | | MDA-MB-231 | |
| Heart (fetal) | 46.0 | Breast ca.* (pl.ef) | 29.7 |
| | | T47D | |
| Heart | 9.8 | Breast ca. BT-549 | 4.0 |
| Skeletal muscle | 30.6 | Breast ca. MDA-N | 10.4 |
| (fetal) | | | |
| Skeletal muscle | 26.6 | Ovary | 7.9 |
| Bone marrow | 29.5 | Ovarian ca. OVCAR-3 | 13.3 |
| Thymus | 32.3 | Ovarian ca. OVCAR-4 | 14.3 |
| Spleen | 26.4 | Ovarian ca. OVCAR-5 | 62.4 |
| Lymph node | 26.2 | Ovarian ca. OVCAR-8 | 3.9 |
| Colorectal | 11.0 | Ovarian ca. IGROV-1 | 6.2 |
| Stomach | 7.9 | Ovarian ca.* | 47.0 |
| | | (ascites) SK-OV-3 | |
| Small intestine | 5.6 | Uterus | 5.0 |
| Colon ca. SW480 | 15.6 | Plancenta | 3.2 |
| Colon ca.* SW620(SW480 | 100.0 | Prostate | 8.0 |
| met) | | | |
| Colon ca. HT29 | 19.5 | Prostate ca.* (bone | 21.5 |
| | | met)PC-3 | |
| Colon ca. HCT-116 | 16.6 | Testis | 5.0 |
| Colon ca. CaCo-2 | 21.9 | Melanoma Hs688(A).T | 4.3 |
| Colon ca. | 13.1 | Melanoma* (met) | 3.6 |
| tissue(ODO3866) | | Hs688(B).T | |
| Colon ca. HCC-2998 | 33.9 | Melanoma UACC-62 | 7.0 |
| Gastric ca.* (liver | 18.8 | Melanoma M14 | 5.0 |
| met) NCI-N87 | | | |
| Bladder | 7.2 | Melanoma LOX IMVI | 13.3 |
| Trachea | 4.0 | Melanoma* (met) SK- | 7.8 |
| | | MEL-5 | |
| Kidney | 7.6 | Adipose | 13.8 |

TABLE 12CC
Panel 4D

| | Rel. Exp. (%) | | Rel. Exp. (%) |
|---|---|---|---|
| | Ag2303, Run | | Ag2303, Run |
| Tissue Name | 151630338 | Tissue Name | 151630338 |
| Secondary Th1 act | 69.7 | HUVEC IL-1beta | 2.8 |
| Secondary Th2 act | 51.4 | HUVEC IFN gamma | 15.7 |
| Secondary Tr1 act | 66.0 | HUVEC TNF alpha + IFN | 7.2 |
| | | gamma | |
| Secondary Th1 rest | 24.5 | HUVEC TNF alpha + IL4 | 7.2 |
| Secondary Th2 rest | 28.9 | HUVEC IL-11 | 5.9 |
| Secondary Tr1 rest | 29.1 | Lung Microvascular EC | 6.8 |
| | | none | |
| Primary Th1 act | 53.2 | Lung Microvascular EC | 5.4 |
| | | TNF alpha + IL-1beta | |
| Primary Th2 act | 44.4 | Microvascular Dermal EC | 10.1 |
| | | none | |
| Primary Tr1 act | 66.0 | Microsvasular Dermal EC | 6.7 |
| | | TNF alpha + IL-1beta | |
| Primary Th1 rest | 89.5 | Bronchial epithelium | 7.2 |
| | | TNF alpha + IL1beta | |
| Primary Th2 rest | 66.0 | Small airway epithelium | 4.1 |
| | | none | |
| Primary Tr1 rest | 46.7 | Small airway epithelium | 20.4 |
| | | TNF alpha + IL-1beta | |
| CD45RA CD4 lymphocyte | 36.3 | Coronery artery SMC rest | 7.7 |
| act | | | |
| CD45RO CD4 lymphocyte | 55.5 | Coronery artery SMC | 6.1 |
| act | | TNF alpha + IL-1beta | |
| CD8 lymphocyte act | 56.3 | Astrocytes rest | 4.4 |
| Secondary CD8 | 47.6 | Astrocytes TNF alpha + IL- | 3.0 |
| lymphocyte rest | | 1beta | |
| Secondary CD8 | 48.0 | KU-812 (Basophil) rest | 17.3 |
| lymphocyte act | | | |
| CD4 lymphocyte none | 15.2 | KU-812 (Basophil) | 31.2 |
| | | PMA/ionomycin | |
| 2ry Th1/Th2/Tr1_anti- | 41.2 | CCD1106 (Keratinocytes) | 11.8 |
| CD95 CH11 | | none | |
| LAK cells rest | 34.4 | CCD1106 (Keratinocytes) | 9.9 |
| | | TNF alpha + IL-1beta | |
| LAK cells IL-2 | 69.3 | Liver cirrhosis | 2.0 |
| LAK cells IL-2 + IL-12 | 55.9 | Lupus kidney | 2.1 |
| LAK cells IL-2 + IFN | 63.3 | NCI-H292 none | 21.0 |
| gamma | | | |
| LAK cells IL-2 + IL-18 | 57.0 | NCI-H292 IL-4 | 33.2 |
| LAK cells | 9.6 | NCI-H292 IL-9 | 33.2 |
| PMA/ionomycin | | | |
| NK Cells IL-2 rest | 47.6 | NCI-H292 IL-13 | 20.9 |
| Two Way MLR 3 day | 38.7 | NCI-H292 IFN gamma | 25.0 |
| Two Way MLR 5 day | 39.5 | HPAEC none | 8.2 |
| Two Way MLR 7 day | 42.0 | HPAEC TNF alpha + IL- | 8.6 |
| | | 1beta | |
| PBMC rest | 21.5 | Lung fibroblast none | 5.9 |
| PBMC PWM | 100.0 | Lung fibroblast TNF alpha + IL- | 6.4 |
| | | 1beta | |
| PBMC PHA-L | 73.7 | Lung fibroblast IL-4 | 12.2 |
| Ramos (B cell) none | 54.3 | Lung fibroblast IL-9 | 9.9 |
| Ramos (B cell) | 78.5 | Lung fibroblast IL-13 | 9.6 |
| ionomycin | | | |
| B lymphocytes PWM | 90.1 | Lung fibroblast IFN gamma | 11.6 |
| B lymphocytes CD40L | 53.6 | Dermal fibroblast CCD1070 | 12.5 |
| and IL-4 | | rest | |
| EOL-1 dbcAMP | 57.4 | Dermal fibroblast CCD1070 | 67.8 |
| | | TNF alpha | |
| EOL-1 dbcAMP | 18.8 | Dermal fibroblast CCD1070 | 9.7 |
| PMA/ionomycin | | IL-1beta | |
| Dendritic cells none | 22.1 | Dermal fibroblast IFN | 5.5 |
| | | gamma | |
| Dendritic cells LPS | 15.9 | Dermal fibroblast IL-4 | 7.4 |
| Dendritic cells anti- | 22.2 | IBD Colitis 2 | 2.0 |
| CD40 | | | |
| Monocytes rest | 45.4 | IBD Crohn's | 1.4 |
| Monocytes LPS | 17.3 | Colon | 20.4 |
| Macrophages rest | 36.1 | Lung | 14.0 |
| Macrophages LPS | 18.0 | Thymus | 10.6 |
| HUVEC none | 13.7 | Kidney | 31.6 |
| HUVEC starved | 19.8 | | |

Panel 1.3D Summary: The CG50345-01 gene is widely expressed across the panel, with highest expression in a colon cancer cell line SW620 (CT=26.4). Of note is the difference in expression between the related colon cancer cell lines SW620 and SW480. SW 480 represents the primary lesion from a patient with colon cancer, while SW620 represents a metastasis from the same patient. The difference in expression of this gene between the SW620 and SW480 cell lines indicates that it could be used to distinguish these cells, or others like them. Moreover, therapeutic modulation of the CG50345-01 gene, through the use of small molecule drugs, antibodies or protein therapeutics, may be of effective in the treatment of metastatic colon cancer. Among tissues with metabolic function, the CG50345-01 gene is moderately expressed in the pancreas, adrenal, thyroid, pituitary, adipose, adult and fetal heart, adult and fetal liver, and adult and fetal liver. This expression profile suggests that the CG50345-01 gene product may be an important small molecule target for the treatment of metabolic disease in any or all of these tissues, including obesity and diabetes. The CG50345-01 gene, which encodes a beta-adrenergic receptor kinase, also shows high expression in all regions of the brain examined, especially in the cerebral cortex (CT=26.7) The beta adrenergic receptors have been shown to play a role in memory formation and in clinical depression. Since many current anti-depressants produce undesired side effects as a result of non-specific binding (to other receptors), this gene is therefore an excellent small molecule target for the treatment of clinical depression without side effects. Furthermore, the role of beta adrenergic receptors in memory consolidation suggests that the CG50345-01 gene product would also be useful as a small molecule target for the treatment of Alzheimer's disease, vascular dementia, or any memory loss disorder. References: Feighner J P. Mechanism of action of antidepressant medications. J Clin Psychiatry 1999;60 Suppl 4:4-11; discussion 12-3. The psychopharmacology of depression is a field that has evolved rapidly in just under 5 decades. Early antidepressant medications—tricyclic antidepressants (TCAs) and monoamine oxidase inhibitors (MAOIs)—were discovered through astute clinical observations. These first-generation medications were effective because they enhanced serotonergic or noradrenergic mechanisms or both. Unfortunately, the TCAs also blocked histaminic, cholinergic, and alpha1-adrenergic receptor sites, and this action brought about unwanted side effects such as weight gain, dry mouth, constipation, drowsiness, and dizziness. MAOIs can interact with tyramine to cause potentially lethal hypertension and present potentially dangerous interactions with a number of medications and over-the-counter drugs. The newest generation of antidepressants, including the single-receptor selective serotonin reuptake inhibitors (SSRIs) and multiple-receptor antidepressants venlafaxine, mirtazapine, bupropion, trazodone, and nefazodone, target one or more specific brain receptor sites without, in most cases, activating unwanted sites such as histamine and acetylcholine. This paper discusses the new antidepressants, particularly with regard to mechanism of action, and looks at future developments in the treatment of depression. Ferry B, McGaugh J L. Role of amygdala norepinephrine in mediating stress hormone regulation of memory storage. Acta Pharmacol Sin 2000 June;21(6):481-93. There is extensive evidence indicating that the noradrenergic system of the amygdala, particularly the basolateral nucleus of the amygdala (BLA), is involved in memory consolidation. This article reviews the central hypothesis that stress hormones released during emotionally arousing experiences activate noradrenergic mechanisms in the BLA, resulting in enhanced memory for those events. Findings from experiments using rats have shown that the memory-modulatory effects of the adrenocortical stress hormones epinephrine and glucocorticoids involve activation of beta-adrenoceptors in the BLA. In addition, both behavioral and microdialysis studies have shown that the noradrenergic system of the BLA also mediates the influences of other neuromodulatory systems such as opioid peptidergic and GABAergic systems on memory storage. Other findings indicate that this stress hormone-induced activation of noradrenergic mechanisms in the BLA regulates memory storage in other brain regions.

Panel 2.2 Summary: Ag2303 Data from Panel 2.2 has not been included because a strange amp plot suggests that there were problems with this experiment.

Panel 4D Summary: The CG50345-01 gene, a beta-adrenergic receptor kinase homolog, is highly expressed (CTs=26-29) in a wide range of cells of significance in the immune response in health and disease. Highest expression of this gene is found in activated B and T cells. Therefore, inhibition of the function of the protein encoded by the CG50345-01 gene with a small molecule drug may block the functions of B cells or T cells and could be beneficial in the treatment of patients suffering from autoimmune and inflammatory diseases such as asthma, allergies, inflammatory bowel disease, lupus erythematosus, or rheumatoid arthritis.

D. CG50301-01: HumanTENM4

Expression of gene CG50301-01 was assessed using the primer-probe sets Ag2581 and Ag2910, described in Tables DA and DB. Results of the RTQ-PCR runs are shown in Tables 12DC, 12DD, 12DE, 12DF, and 12DG.

TABLE 12DA

Probe Name Ag2581

Start

| | | | |
|---|---|---|---|
| Primers | Sequences | Length | Position |

| | | | |
|---|---|---|---|
| Forward | 5′-tgaccacagacatcatcagtgt-3′ | 22 | 7770 |
| | (SEQ ID NO:119) | | |
| Probe | TET-5′-ccatcttgaaccatgcccactaccta- | 26 | 7821 |
| | 3′-TAMRA (SEQ ID NO:120) | | |
| Reverse | 5′-tcaatggtgaagtgcaggtt-3′ | 20 | 7850 |
| | (SEQ ID NO:121) | | |

TABLE 19DB

Probe Name Ag2910

Start

| | | | |
|---|---|---|---|
| Primers | Sequences | Length | Position |

| | | | |
|---|---|---|---|
| Forward | 5′-tgaccacagacatcatcagtgt-3′ | 22 | 7770 |
| | (SEQ ID NO:122) | | |
| Probe | 5′-ccatcttgaaccatgcccactaccta- | 26 | 7821 |
| | 3′-TAMRA (SEQ ID NO:123) | | |
| Reverse | 5′-tcaatggtgaagtgcaggtt-3′ | 20 | 7850 |
| | (SEQ ID NO:124) | | |

TABLE 12DC
CNS_neurodegeneration_v1.0

| | Rel. Exp. (%) | Rel. Exp. (%) | | Rel. Exp. (%) | Rel. Exp. (%) |
|---|---|---|---|---|---|
| | Ag2581, Run | Ag2910, Run | | Ag2581, Run | Ag2910, Run |
| Tissue Name | 208777162 | 209735201 | Tissue Name | 208777162 | 209735201 |
| AD 1 Hippo | 8.8 | 11.0 | Control | 1.5 | 2.1 |
| | | | (Path) 3 | | |
| | | | Temporal Ctx | | |
| AD 2 Hippo | 28.5 | 26.4 | Control | 27.7 | 25.2 |
| | | | (Path) 4 | | |
| | | | Temporal Ctx | | |
| AD 3 Hippo | 5.3 | 6.1 | AD 1 | 13.4 | 13.2 |
| | | | Occipital Ctx | | |
| AD 4 Hippo | 8.5 | 7.1 | AD 2 | 0.0 | 0.0 |
| | | | Occipital Ctx | | |
| | | | (Missing) | | |
| AD 5 Hippo | 94.0 | 100.0 | AD 3 | 1.7 | 3.7 |
| | | | Occipital Ctx | | |
| AD 6 Hippo | 67.8 | 66.9 | AD 4 | 31.0 | 14.3 |
| | | | Occipital Ctx | | |
| Control 2 | 42.6 | 45.1 | AD 5 | 57.0 | 55.9 |
| Hippo | | | Occipital Ctx | | |
| Control 4 | 9.7 | 11.0 | AD 6 | 16.2 | 15.8 |
| Hippo | | | Occipital Ctx | | |
| Control | 3.8 | 2.6 | Control 1 | 1.4 | 1.0 |
| (Path) 3 | | | Occipital Ctx | | |
| Hippo | | | | | |
| AD 1 Temporal | 9.3 | 11.8 | Control 2 | 72.7 | 69.7 |
| Ctx | | | Occipital Ctx | | |
| AD 2 Temporal | 26.8 | 27.0 | Control 3 | 16.0 | 13.2 |
| Ctx | | | Occipital Ctx | | |
| AD 3 Temporal | 5.0 | 4.0 | Control 4 | 5.4 | 6.8 |
| Ctx | | | Occipital Ctx | | |
| AD 4 Temporal | 22.8 | 24.1 | Control | 93.3 | 95.9 |
| Ctx | | | (Path) 1 | | |
| | | | Occipital Ctx | | |
| AD 5 Inf | 100.0 | 94.6 | Control | 8.6 | 9.4 |
| Temporal Ctx | | | (Path) 2 | | |
| | | | Occipital Ctx | | |
| AD 5 Sup | 34.2 | 36.9 | Control | 0.9 | 1.1 |
| Temporal Ctx | | | (Path) 3 | | |
| | | | Occipital Ctx | | |
| AD 6 Inf | 47.3 | 53.2 | Control | 17.1 | 15.2 |
| Temporal Ctx | | | (Path) 4 | | |
| | | | Occipital Ctx | | |
| AD 6 Sup | 47.6 | 40.9 | Control 1 | 2.1 | 5.1 |
| Temporal Ctx | | | Parietal Ctx | | |
| Control 1 | 2.4 | 1.9 | Control 2 | 35.6 | 44.4 |
| Temporal Ctx | | | Parietal Ctx | | |
| Control 2 | 44.8 | 44.8 | Control 3 | 17.8 | 14.6 |
| Temporal Ctx | | | Parietal Ctx | | |
| Control 3 | 10.4 | 11.1 | Control | 78.5 | 74.2 |
| Temporal Ctx | | | (Path) 1 | | |
| | | | Parietal Ctx | | |
| Control 3 | 8.2 | 7.5 | Control | 19.5 | 21.8 |
| Temporal Ctx | | | (Path) 2 | | |
| | | | Parietal Ctx | | |
| Control | 80.1 | 68.3 | Control | 1.1 | 2.0 |
| (Path) 1 | | | (Path) 3 | | |
| Temporal Ctx | | | Parietal Ctx | | |
| Control | 36.6 | 29.3 | Control | 43.2 | 37.9 |
| (Path) 2 | | | (Path) 4 | | |
| Temporal Ctx | | | Parietal Ctx | | |

TABLE 12DD
Panel 1.3D

| | Rel. Exp. (%) | Rel. Exp. (%) | | Rel. Exp. (%) | Rel. Exp. (%) |
|---|---|---|---|---|---|
| | Ag2581, Run | Ag2910, Run | | Ag2581, Run | Ag2910, Run |
| Tissue Name | 162292620 | 162556486 | Tissue Name | 162292620 | 162556486 |
| Liver | 0.0 | 0.0 | Kidney (fetal) | 5.8 | 4.7 |
| adenocarcinoma | | | | | |
| Pancreas | 0.2 | 0.0 | Renal ca. 786-0 | 1.7 | 0.1 |
| Pancreatic ca. | 0.5 | 0.0 | Renal ca. A498 | 0.8 | 0.9 |
| CAPAN 2 | | | | | |
| Adrenal gland | 0.3 | 0.4 | Renal ca. RXF | 8.8 | 4.7 |
| | | | 393 | | |
| Thyroid | 5.4 | 5.3 | Renal ca. ACHN | 4.0 | 5.0 |
| Salivary gland | 0.5 | 0.7 | Renal ca. UO-31 | 13.7 | 13.9 |
| Pituitary gland | 11.1 | 8.1 | Renal ca. TK-10 | 2.9 | 3.0 |
| Brain (fetal) | 6.6 | 11.7 | Liver | 0.0 | 0.0 |
| Brain (whole) | 10.9 | 7.2 | Liver (fetal) | 0.0 | 0.0 |
| Brain (amygdala) | 14.9 | 12.9 | Liver ca. | 0.4 | 0.0 |
| | | | (hepatoblast) | | |
| | | | HepG2 | | |
| Brain | 2.6 | 2.0 | Lung | 0.7 | 0.2 |
| (cerebellum) | | | | | |
| Brain | 13.5 | 12.3 | Lung (fetal) | 0.7 | 1.9 |
| (hippocampus) | | | | | |
| Brain (substantia | 1.5 | 0.7 | Lung ca. (small | 0.0 | 0.0 |
| nigra) | | | cell) LX-1 | | |
| Brain (thalamus) | 12.2 | 7.3 | Lung ca. (small | 13.8 | 9.9 |
| | | | cell) NCI-H69 | | |
| Cerebral Cortex | 100.0 | 68.8 | Lung ca. | 1.7 | 2.2 |
| | | | (s.cell var.) | | |
| | | | SHP-77 | | |
| Spinal cord | 13.0 | 10.2 | Lung ca. (large | 0.0 | 0.0 |
| | | | cell)NCI-H460 | | |
| glio/astro U87-MG | 14.5 | 15.5 | Lung ca. (non- | 0.0 | 0.0 |
| | | | sm. cell) A549 | | |
| glio/astro U-118- | 0.2 | 0.2 | Lung ca. (non- | 0.3 | 0.0 |
| MG | | | s.cell) NCI-H23 | | |
| astrocytoma | 2.4 | 2.8 | Lung ca. (non- | 0.1 | 0.6 |
| SW1783 | | | s.cell) HOP-62 | | |
| neuro*; met SK-N- | 4.0 | 3.8 | Lung ca. (non- | 0.0 | 0.0 |
| AS | | | s.cl) NCI-H522 | | |
| astrocytoma SF- | 0.2 | 0.0 | Lung ca. | 2.2 | 2.8 |
| 539 | | | (squam.) SW 900 | | |
| astrocytoma SNB | 0.8 | 2.5 | Lung ca. | 6.0 | 4.6 |
| 75 | | | (squam.) NCI- | | |
| | | | H596 | | |
| glioma SNB-19 | 15.0 | 12.2 | Mammary gland | 1.9 | 2.2 |
| glioma U251 | 5.7 | 5.9 | Breast ca.* | 0.3 | 1.4 |
| | | | (pl.ef) MCF-7 | | |
| glioma SF-295 | 1.3 | 1.5 | Breast ca.* | 0.0 | 0.0 |
| | | | (pl.ef) MDA-MB- | | |
| | | | 231 | | |
| Heart (fetal) | 1.3 | 1.2 | Breast ca.* | 0.0 | 0.0 |
| | | | (pl.ef) T47D | | |
| Heart | 0.5 | 0.5 | Breast ca. BT- | 0.2 | 0.0 |
| | | | 549 | | |
| Skeletal muscle | 42.9 | 36.1 | Breast ca. MDA-N | 0.0 | 0.0 |
| (fetal) | | | | | |
| Skeletal muscle | 0.8 | 0.6 | Ovary | 100.0 | 100.0 |
| Bone marrow | 0.2 | 0.7 | Ovarian ca. | 0.0 | 0.7 |
| | | | OVCAR-3 | | |
| Thymus | 8.7 | 3.7 | Ovarian ca. | 0.0 | 0.0 |
| | | | OVCAR-4 | | |
| Spleen | 0.0 | 0.2 | Ovarian ca. | 0.8 | 2.6 |
| | | | OVCAR-5 | | |
| Lymph node | 0.2 | 0.5 | Ovarian ca. | 1.7 | 0.5 |
| | | | OVCAR-8 | | |
| Colorectal | 3.0 | 2.0 | Ovarian ca. | 0.0 | 0.1 |
| | | | IGROV-1 | | |
| Stomach | 0.2 | 0.5 | Ovarian ca.* | 0.0 | 0.0 |
| | | | (ascites) SK- | | |
| | | | OV-3 | | |
| Small intestine | 0.0 | 0.1 | Uterus | 1.1 | 1.2 |
| Colon ca. SW480 | 0.0 | 0.0 | Plancenta | 0.2 | 0.0 |
| Colon ca.* | 0.0 | 0.2 | Prostate | 0.2 | 1.0 |
| SW620(SW480 met) | | | | | |
| Colon ca. HT29 | 0.0 | 0.0 | Prostate ca.* | 27.0 | 19.2 |
| | | | (bone met)PC-3 | | |
| Colon ca. HCT-116 | 0.0 | 0.0 | Testis | 1.9 | 2.5 |
| Colon ca. CaCo-2 | 1.3 | 0.3 | Melanoma | 1.6 | 2.2 |
| | | | Hs688(A).T | | |
| Colon ca. | 6.1 | 3.7 | Melanoma* (met) | 0.9 | 2.0 |
| tissue(ODO3866) | | | Hs688(B).T | | |
| Colon ca. HCC- | 0.0 | 0.0 | Melanoma UACC- | 0.7 | 0.3 |
| 2998 | | | 62 | | |
| Gastric ca.* | 3.3 | 3.7 | Melanoma M14 | 0.0 | 0.0 |
| (liver met) NCI- | | | | | |
| N87 | | | | | |
| Bladder | 1.9 | 2.1 | Melanoma LOX | 1.3 | 1.4 |
| | | | IMVI | | |
| Trachea | 5.1 | 6.1 | Melanoma* (met) | 0.0 | 0.0 |
| | | | SK-MEL-5 | | |
| Kidney | 3.7 | 3.0 | Adipose | 2.2 | 1.9 |

TABLE 12DE
Panel 2D

| | Rel. Exp. (%) | Rel. Exp. (%) | | Rel. Exp. (%) | Rel. Exp. (%) |
|---|---|---|---|---|---|
| | Ag2581, Run | Ag2910, Run | | Ag2581, Run | Ag2910, Run |
| Tissue Name | 161921268 | 162354453 | Tissue Name | 161921268 | 162354453 |
| Normal Colon | 13.2 | 7.1 | Kidney Margin | 3.8 | 2.6 |
| | | | 8120608 | | |
| CC Well to Mod | 6.5 | 11.0 | Kidney Cancer | 0.9 | 0.7 |
| Diff (ODO3866) | | | 8120613 | | |
| CC Margin | 2.7 | 2.0 | Kidney Margin | 7.5 | 4.4 |
| (ODO3866) | | | 8120614 | | |
| CC Gr.2 | 1.6 | 1.0 | Kidney Cancer | 18.4 | 22.4 |
| rectosigmoid | | | 9010320 | | |
| (ODO3868) | | | | | |
| CC Margin | 1.0 | 2.1 | Kidney Margin | 9.9 | 15.9 |
| (ODO3868) | | | 9010321 | | |
| CC Mod Diff | 0.5 | 1.5 | Normal Uterus | 2.4 | 4.9 |
| (ODO3920) | | | | | |
| CC Margin | 1.4 | 5.0 | Uterus Cancer | 6.8 | 8.7 |
| (ODO3920) | | | 064011 | | |
| CC Gr.2 ascend | 5.3 | 11.6 | Normal Thyroid | 19.1 | 29.5 |
| colon (ODO3921) | | | | | |
| CC Margin | 0.9 | 0.3 | Thyroid Cancer | 52.9 | 75.8 |
| (ODO3921) | | | 064010 | | |
| CC from Partial | 4.2 | 1.7 | Thyroid Cancer | 3.9 | 6.7 |
| Hepatectomy | | | A302152 | | |
| (ODO4309) Mets | | | | | |
| Liver Margin | 0.7 | 0.4 | Thyroid Margin | 31.9 | 35.4 |
| (ODO4309) | | | A302153 | | |
| Colon mets to | 2.9 | 3.1 | Normal Breast | 6.1 | 12.2 |
| lung (OD04451- | | | | | |
| 01) | | | | | |
| Lung Margin | 0.8 | 4.2 | Breast Cancer | 4.1 | 4.5 |
| (OD04451-02) | | | (OD04566) | | |
| Normal Prostate | 0.7 | 18.7 | Breast Cancer | 2.7 | 14.3 |
| 6546-1 | | | (OD04590-01) | | |
| Prostate Cancer | 6.8 | 8.8 | Breast Cancer | 21.0 | 21.0 |
| (OD04410) | | | Mets (OD04590- | | |
| | | | 03) | | |
| Prostate Margin | 3.4 | 5.7 | Breast Cancer | 3.4 | 5.3 |
| (OD04410) | | | Metastasis | | |
| | | | (OD04655-05) | | |
| Prostate Cancer | 10.3 | 12.7 | Breast Cancer | 9.7 | 26.2 |
| (OD04720-01) | | | 064006 | | |
| Prostate Margin | 7.4 | 16.2 | Breast Cancer | 11.3 | 15.3 |
| (OD04720-02) | | | 1024 | | |
| Normal Lung | 5.8 | 7.2 | Breast Cancer | 4.9 | 12.2 |
| 061010 | | | 9100266 | | |
| Lung Met to | 1.8 | 3.5 | Breast Margin | 10.5 | 16.8 |
| Muscle (ODO4286) | | | 9100265 | | |
| Muscle Margin | 6.8 | 5.8 | Breast Cancer | 17.0 | 32.3 |
| (ODO4286) | | | A209073 | | |
| Lung Malignant | 20.9 | 19.9 | Breast Margin | 6.9 | 8.2 |
| Cancer (OD03126) | | | A2090734 | | |
| Lung Margin | 4.7 | 4.9 | Normal Liver | 0.0 | 0.3 |
| (OD03126) | | | | | |
| Lung Cancer | 22.8 | 22.4 | Liver Cancer | 0.0 | 0.0 |
| (OD04404) | | | 064003 | | |
| Lung Margin | 5.0 | 4.1 | Liver Cancer | 0.3 | 0.7 |
| (OD04404) | | | 1025 | | |
| Lung Cancer | 13.2 | 14.6 | Liver Cancer | 0.7 | 0.9 |
| (OD04565) | | | 1026 | | |
| Lung Margin | 0.7 | 0.6 | Liver Cancer | 0.3 | 0.9 |
| (OD04565) | | | 6004-T | | |
| Lung Cancer | 37.6 | 57.8 | Liver Tissue | 0.0 | 0.8 |
| (OD04237-01) | | | 6004-N | | |
| Lung Margin | 2.4 | 1.3 | Liver Cancer | 0.5 | 2.1 |
| (OD04237-02) | | | 6005-T | | |
| Ocular Mel Met | 0.0 | 0.3 | Liver Tissue | 0.4 | 0.8 |
| to Liver | | | 6005-N | | |
| (ODO4310) | | | | | |
| Liver Margin | 0.0 | 0.0 | Normal Bladder | 6.8 | 8.1 |
| (ODO4310) | | | | | |
| Melanoma Mets to | 0.8 | 1.7 | Bladder Cancer | 6.7 | 8.0 |
| Lung (OD04321) | | | 1023 | | |
| Lung Margin | 1.9 | 4.7 | Bladder Cancer | 42.3 | 46.3 |
| (OD04321) | | | A302173 | | |
| Normal Kidney | 21.6 | 20.4 | Bladder Cancer | 2.8 | 4.2 |
| | | | (OD04718-01) | | |
| Kidney Ca, | 1.9 | 5.0 | Bladder Normal | 6.0 | 10.2 |
| Nuclear grade 2 | | | Adjacent | | |
| (OD04338) | | | (OD04718-03) | | |
| Kidney Margin | 15.0 | 18.2 | Normal Ovary | 63.7 | 75.3 |
| (OD04338) | | | | | |
| Kidney Ca | 1.5 | 3.1 | Ovarian Cancer | 100.0 | 100.0 |
| Nuclear grade | | | 064008 | | |
| ½ (OD04339) | | | | | |
| Kidney Margin | 13.7 | 20.9 | Ovarian Cancer | 1.1 | 0.6 |
| (OD04339) | | | (OD04768-07) | | |
| Kidney Ca, Clear | 4.0 | 6.5 | Ovary Margin | 3.4 | 8.5 |
| cell type | | | (OD04768-08) | | |
| (OD04340) | | | | | |
| Kidney Margin | 8.2 | 13.1 | Normal Stomach | 5.2 | 2.8 |
| (OD04340) | | | | | |
| Kidney Ca, | 1.3 | 2.0 | Gastric Cancer | 3.4 | 5.6 |
| Nuclear grade 3 | | | 9060358 | | |
| (OD04348) | | | | | |
| Kidney Margin | 7.3 | 14.3 | Stomach Margin | 2.0 | 2.2 |
| (OD04348) | | | 9060359 | | |
| Kidney Cancer | 15.4 | 20.0 | Gastric Cancer | 8.3 | 17.0 |
| (OD04622-01) | | | 9060395 | | |
| Kidney Margin | 1.9 | 4.0 | Stomach Margin | 6.2 | 5.2 |
| (OD04622-03) | | | 9060394 | | |
| Kidney Cancer | 0.0 | 2.6 | Gastric Cancer | 8.2 | 11.6 |
| (OD04450-01) | | | 9060397 | | |
| Kidney Margin | 10.5 | 9.5 | Stomach Margin | 0.9 | 0.3 |
| (OD04450-03) | | | 9060396 | | |
| Kidney Cancer | 9.2 | 15.4 | Gastric Cancer | 3.8 | 9.2 |
| 8120607 | | | 064005 | | |

TABLE 12DF
Panel 3D

| | Rel. Exp. (%) | | Rel. Exp. (%) |
|---|---|---|---|
| | Ag2581, Run | | Ag2581, Run |
| Tissue Name | 164827572 | Tissue Name | 164827572 |
| Daoy-Medulloblastoma | 2.3 | Ca Ski-Cervical epidermoid | 0.5 |
| | | carcinoma (metastasis) | |
| TE671-Medulloblastoma | 0.9 | ES-2-Ovarian clear cell | 1.2 |
| | | carcinoma | |
| D283 Med- | 0.4 | Ramos-Stimulated with | 0.0 |
| Medulloblastoma | | PMA/ionomycin 6h | |
| PFSK-1-Primitive | 11.3 | Ramos-Stimulated with | 0.0 |
| Neuroectodermal | | PMA/ionomycin 14h | |
| XF-498-CNS | 0.7 | MEG-01-Chronic myelogenous | 0.0 |
| | | leukemia (megokaryoblast) | |
| SNB-78-Glioma | 0.0 | Raji-Burkitt's lymphoma | 0.3 |
| SF-268-Glioblastoma | 5.1 | Daudi-Burkitt's lymphoma | 0.1 |
| T98G-Glioblastoma | 0.4 | U266-B-cell plasmacytoma | 0.1 |
| SK-N-SH-Neuroblastoma | 20.9 | CA46-Burkitt's lymphoma | 0.0 |
| (metastasis) | | | |
| SF-295-Glioblastoma | 0.0 | RL-non-Hodgkin's B-cell | 0.7 |
| | | lymphoma | |
| Cerebellum | 2.3 | JM1-pre-B-cell lymphoma | 0.0 |
| Cerebellum | 2.2 | Jurkat-T cell leukemia | 0.4 |
| NCI-H292-Mucoepidermoid | 1.3 | TF-1-Erythroleukemia | 0.4 |
| lung carcinoma | | | |
| DMS-114-Small cell lung | 0.0 | HUT 78-T-cell lymphoma | 0.3 |
| cancer | | | |
| DMS-79-Small cell lung | 4.3 | U937-Histiocytic lymphoma | 0.3 |
| cancer | | | |
| NCI-H146-Small cell | 6.6 | KU-812-Myelogenous | 0.0 |
| lung cancer | | leukemia | |
| NCI-H526-Small cell | 100.0 | 769-P-Clear cell renal | 1.0 |
| lung cancer | | carcinoma | |
| NCI-N417-Small cell | 1.8 | Caki-2-Clear cell renal | 0.5 |
| lung cancer | | carcinoma | |
| NCI-H82-Small cell lung | 0.3 | SW 839-Clear cell renal | 3.5 |
| cancer | | carcinoma | |
| NCI-H157-Squamous cell | 0.3 | G401-Wilms' tumor | 7.3 |
| lung cancer (metastasis) | | | |
| NCI-H1155-Large cell | 1.1 | Hs766T-Pancreatic | 4.3 |
| lung cancer | | carcinoma (LN metastasis) | |
| NCI-H1299-Large cell | 0.6 | CAPAN-1-Pancreatic | 0.0 |
| lung cancer | | adenocarcinoma (liver | |
| | | metastasis) | |
| NCI-H727-Lung carcinoid | 6.2 | SU86.86-Pancreatic | 0.8 |
| | | carcinoma (liver | |
| | | metastasis) | |
| NCI-UMC-11-Lung | 0.0 | BxPC-3-Pancreatic | 2.8 |
| carcinoid | | adenocarcinoma | |
| LX-1-Small cell lung | 0.0 | HPAC-Pancreatic | 0.0 |
| cancer | | adenocarcinoma | |
| Colo-205-Colon cancer | 0.0 | MIA PaCa-2-Pancreatic | 0.0 |
| | | carcinoma | |
| KM12-Colon cancer | 0.0 | CFPAC-1-Pancreatic ductal | 0.0 |
| | | adenocarcinoma | |
| KM20L2-Colon cancer | 0.0 | PANC-1-Pancreatic | 0.0 |
| | | epithelioid ductal | |
| | | carcinoma | |
| NCI-H716-Colon cancer | 0.9 | T24-Bladder carcinma | 3.1 |
| | | (transitional cell) | |
| SW-48-Colon | 0.0 | 5637-Bladder carcinoma | 1.0 |
| adenocarcinoma | | | |
| SW1116-Colon | 0.0 | HT-1197-Bladder carcinoma | 1.3 |
| adenocarcinoma | | | |
| LS 174T-Colon | 0.0 | UM-UC-3-Bladder carcinma | 1.3 |
| adenocarcinoma | | (transitional cell) | |
| SW-948-Colon | 0.0 | A204-Rhabdomyosarcoma | 0.3 |
| adenocarcinoma | | | |
| SW-480-Colon | 0.1 | HT-1080-Fibrosarcoma | 12.4 |
| adenocarcinoma | | | |
| NCI-SNU-5-Gastric | 0.0 | MG-63-Osteosarcoma | 0.2 |
| carcinoma | | | |
| KATO III-Gastric | 0.0 | SK-LMS-1-Leiomyosarcoma | 9.5 |
| carcinoma | | (vulva) | |
| NCI-SNU-16-Gastric | 0.2 | SJRH30-Rhabdomyosarcoma | 0.8 |
| carcinoma | | (met to bone marrow) | |
| NCI-SNU-1-Gastric | 0.0 | A431-Epidermoid carcinoma | 0.4 |
| carcinoma | | | |
| RF-1-Gastric | 0.0 | WM266-4-Melanoma | 1.8 |
| adenocarcinoma | | | |
| RF-48-Gastric | 0.0 | DU 145-Prostate carcinoma | 0.0 |
| adenocarcinoma | | (brain metastasis) | |
| MKN-45-Gastric | 0.5 | MDA-MB-468-Breast | 0.0 |
| carcinoma | | adenocarcinoma | |
| NCI-N87-Gastric | 0.6 | SCC-4-Squamous cell | 0.0 |
| carcinoma | | carcinoma of tongue | |
| OVCAR-5-Ovarian | 0.2 | SCC-9-Squamous cell | 0.0 |
| carcinoma | | carcinoma of tongue | |
| RL95-2-Uterine | 0.6 | SCC-15-Squamous cell | 0.5 |
| carcinoma | | carcinoma of tongue | |
| HelaS3-Cervical | 0.2 | CAL 27-Squamous cell | 0.0 |
| adenocarcinoma | | carcinoma of tongue | |

TABLE 12DG
Panel 4D

| | Rel. | Rel. | | Rel. | Rel. |
|---|---|---|---|---|---|
| | Exp. (%) | Exp. (%) | | Exp. (%) | Exp. (%) |
| | Ag2581, Run | Ag2910, Run | | Ag2581, Run | Ag2910, Run |
| Tissue Name | 164036199 | 159079044 | Tissue Name | 164036199 | 159079044 |
| Secondary Th1 act | 0.0 | 0.2 | HUVEC IL-1beta | 0.0 | 0.0 |
| Secondary Th2 act | 0.0 | 0.0 | HUVEC IFN gamma | 0.0 | 0.0 |
| Secondary Tr1 act | 0.0 | 0.6 | HUVEC TNF alpha + IFN | 0.0 | 0.0 |
| | | | gamma | | |
| Secondary Th1 rest | 0.0 | 0.0 | HUVEC TNF alpha + IL4 | 0.0 | 0.0 |
| Secondary Th2 rest | 0.0 | 0.0 | HUVEC IL-11 | 0.0 | 0.0 |
| Secondary Tr1 rest | 0.0 | 0.0 | Lung Microvascular | 0.0 | 0.0 |
| | | | EC none | | |
| Primary Th1 act | 0.0 | 0.0 | Lung Microvascular | 0.0 | 0.0 |
| | | | EC TNF alpha + IL- | | |
| | | | 1beta | | |
| Primary Th2 act | 0.0 | 0.0 | Microvascular | 0.0 | 0.5 |
| | | | Dermal EC none | | |
| Primary Tr1 act | 0.0 | 0.0 | Microsvasular | 0.0 | 0.6 |
| | | | Dermal EC TNF alpha + | | |
| | | | IL-1beta | | |
| Primary Th1 rest | 0.0 | 0.0 | Bronchial | 0.2 | 21.8 |
| | | | epithelium | | |
| | | | TNF alpha + IL1beta | | |
| Primary Th2 rest | 0.0 | 0.0 | Small airway | 0.6 | 4.4 |
| | | | epithelium none | | |
| Primary Tr1 rest | 0.0 | 0.0 | Small airway | 0.5 | 4.7 |
| | | | epithelium | | |
| | | | TNF alpha + IL- | | |
| | | | 1beta | | |
| CD45RA CD4 | 0.1 | 0.2 | Coronery artery | 0.0 | 2.4 |
| lymphocyte act | | | SMC rest | | |
| CD45RO CD4 | 0.0 | 0.3 | Coronery artery | 0.0 | 0.3 |
| lymphocyte act | | | SMC TNF alpha + IL- | | |
| | | | 1beta | | |
| CD8 lymphocyte act | 0.0 | 0.0 | Astrocytes rest | 2.9 | 19.3 |
| Secondary CD8 | 0.0 | 0.0 | Astrocytes | 1.9 | 17.0 |
| lymphocyte rest | | | TNF alpha + IL- | | |
| | | | 1beta | | |
| Secondary CD8 | 0.0 | 0.0 | KU-812 (Basophil) | 0.0 | 0.3 |
| lymphocyte act | | | rest | | |
| CD4 lmphocyte | 0.0 | 0.0 | KU-812 (BasoPhil) | 0.0 | 0.0 |
| none | | | PMA/ionomycin | | |
| 2ry | 0.0 | 0.0 | CCD1106 | 0.7 | 4.8 |
| Th1/Th2/Tr1_anti- | | | (Keratinocytes) | | |
| CD95 CH11 | | | none | | |
| LAK cells rest | 0.0 | 0.6 | CCD1106 | 0.0 | 1.7 |
| | | | (Keratinocytes) | | |
| | | | TNF alpha + IL- | | |
| | | | 1beta | | |
| LAK cells IL-2 | 0.0 | 0.0 | Liver cirrhosis | 0.1 | 1.7 |
| LAX cells IL-2 + IL- | 0.0 | 0.0 | Lupus kidney | 0.1 | 0.7 |
| 12 | | | | | |
| LAK cells IL-2 + IFN | 100.0 | 0.0 | NCI-H292 none | 0.1 | 0.5 |
| gamma | | | | | |
| LAK cells IL-2 + IL- | 0.0 | 0.2 | NCI-H292 IL-4 | 0.0 | 0.6 |
| 18 | | | | | |
| LAK cells | 0.0 | 0.0 | NCI-H292 IL-9 | 0.1 | 3.5 |
| PMA/ionomycin | | | | | |
| NK Cells IL-2 rest | 0.0 | 0.0 | NCI-H292 IL-13 | 0.1 | 0.1 |
| Two Way MLR 3 day | 0.0 | 0.0 | NCI-H292 IFN gamma | 0.2 | 0.0 |
| Two Way MLR 5 day | 0.0 | 0.0 | HPAEC none | 0.0 | 0.0 |
| Two Way MLR 7 day | 0.0 | 0.3 | HPAEC TNF alpha + IL- | 0.0 | 0.0 |
| | | | 1beta | | |
| PBMC rest | 0.0 | 0.0 | Lung fibroblast | 5.8 | 51.1 |
| | | | none | | |
| PBMC PWM | 0.0 | 0.6 | Lung fibroblast | 1.2 | 13.0 |
| | | | TNF alpha + IL-1beta | | |
| PBMC PHA-L | 0.0 | 0.0 | Lung fibroblast | 8.3 | 82.9 |
| | | | IL-4 | | |
| Ramos (B cell) | 0.0 | 0.0 | Lung fibroblast | 6.7 | 50.7 |
| none | | | IL-9 | | |
| Ramos (B cell) | 0.0 | 0.0 | Lung fibroblast | 6.3 | 67.4 |
| ionomycin | | | IL-13 | | |
| B lymphocytes PWM | 0.0 | 0.0 | Lung fibroblast | 8.4 | 100.0 |
| | | | IFN gamma | | |
| B lymphocytes | 0.1 | 0.0 | Dermal fibroblast | 0.5 | 8.4 |
| CD40L and IL-4 | | | CCD1070 rest | | |
| EOL-1 dbcAMP | 0.0 | 0.0 | Dermal fibroblast | 0.3 | 7.3 |
| | | | CCD1070 TNF alpha | | |
| EOL-1 dbcAMP | 0.0 | 0.0 | Dermal fibroblast | 0.3 | 2.0 |
| PMA/ionomycin | | | CCD1070 IL-1beta | | |
| Dendritic cells | 0.0 | 0.6 | Dermal fibroblast | 0.1 | 1.1 |
| none | | | IFN gamma | | |
| Dendritic cells | 0.0 | 0.0 | Dermal fibroblast | 0.3 | 11.7 |
| LPS | | | IL-4 | | |
| Dendritic cells | 0.0 | 0.0 | IBD Colitis 2 | 0.2 | 0.6 |
| anti-CD40 | | | | | |
| Monocytes rest | 0.1 | 2.7 | IBD Crohn's | 0.0 | 0.2 |
| Monocytes LPS | 0.0 | 0.2 | Colon | 0.1 | 3.1 |
| Macrophages rest | 0.0 | 0.0 | Lung | 0.8 | 20.4 |
| Macrophages LPS | 0.0 | 0.0 | Thymus | 1.7 | 20.4 |
| HUVEC none | 0.0 | 0.0 | Kidney | 1.2 | 16.7 |
| HUVEC starved | 0.0 | 0.0 | | | |

CNS_neurodegeneration_v1.0 Summary: Ag2910/Ag2581 No difference is detected in the expression of the CG50301-01 gene in the postmortem brains of Alzheimer's patients when compared normal controls. However, this panel demonstrates the expression of this gene in the CNS of an independent group of patients. See panel 1.3d for a discussion of utility of this gene in the central nervous system.

Panel 1.3D Summary: Ag2581/Ag2910 Two experiments with the same probe and primer set produce results with very good agreement. Highest expression of the CG5030 1-01 gene is seen in the ovary and the cerebral cortex (CTs=28). In contrast to the expression in normal ovary, ovarian cancer cell lines either do not express this gene or express it at very low levels. This expression profile suggests that expression of this gene could potentially be used as a marker for ovarian cancer. Conversely, this gene appears to be more highly expressed in prostate cancer cell lines than in the normal prostate, suggesting this gene may also be a diagnostic marker in prostate cancer as well. This gene is a homolog of the Drosophila TENM4 gene, and is expressed at moderate levels in all brain regions examined. TENM4 is believed to be important in neural development; therefore, this gene may be of use in the induction of compensatory synaptogenesis in the treatment of any diseases/conditions involving neuronal death (Alzheimer's, Parkinson's, Huntington's diseases, stroke, head or spinal cord trauma).

Among metabolic tissues, expression is highest in fetal skeletal muscle. Furthermore, this gene is more highly expressed in fetal skeletal muscle (CTs=29) than in adult skeletal muscle (CT=35). Thus, expression of this gene could be used to differentiate between adult and fetal skeletal muscle. In addition, the higher levels of expression in fetal skeletal muscle suggest that this gene product may play a role in the development of this organ. Therefore, the protein encoded by this gene may be effective in treating weak or dystrophic muscle in the adult. There is also low but significant expression in pituitary, thyroid and adipose. Thus, this gene may be involved in the development and signal transduction pathways of these tissues. Antibody and peptide therapeutics to this gene product may be used in the treatment of metabolic disorders involving these tissues, including obesity and diabetes.

Panel 2D Summary: Ag2581/Ag2910 Two experiments with the same probe and primer set show reasonable concordance, with both runs showing highest expression of the CG50301-01 gene in ovarian cancer. The level of expression of this gene appears to be increased in some lung and gastric cancer tissue samples when compared to the matched normal tissue. The reverse appears to be true for kidney, where expression is slightly higher in 6 of 9 normal tissues than in the matched cancer tissues. Thus, based upon its profile, the expression of this gene could be of use as a marker for distinguishing these cancers from the normal adjacent tissue or as a marker for different grades/types of cancer. Furthermore, therapeutic inhibition of the activity of the product of this gene, through the use of antibodies, peptides or polypeptides may be useful in the treatment of gastric and lung cancer.

Panel 3D Summary: Ag2581 The CG50301-01 gene is expressed at a low level by select cell lines used in this panel. The highest level of expression is seen in NCI-H526, a lung cancer cell line (CT=27.3). Other cell lines that express this gene include neuroblastoma, bladder carcinoma and renal cell cancer cell lines. Therefore, therapeutic inhibition of the activity of the product of this gene, through the use of antibodies, peptides or polypeptides may be useful in the therapy of cancers used in the derivation of these cell lines.

Panel 4D Summary: Ag2910 The CG50301-01 transcript is moderately expressed in lung fibroblasts and is slightly overexpressed in these cells after treatment with IFNg or IL-4 (CT 27.8).

This transcript encodes a human homolog of Ten-M4, a protein with EGF-repeats (reference) that may play a role in fibroblast growth. Modulation of the expression or activity of the protein encoded by this transcrpt through the application of antibodies or small molecules may be useful for treatment of symptoms associated with fibroplasia, chronic obstructive pulmonary disease, emphysema, asthma, psoriasis and ulcerative colitis. Please note that a second experiment with probe and primer set Ag2582 is not included. The amp plot indicates that there were experimental difficulties with this run. Reference: Mieda M, Kikuchi Y, Hirate Y, Aoki M, Okamoto H. Compartmentalized expression of zebrafish ten-m3 and ten-m4, homologues of the Drosophila ten(m)/odd Oz gene, in the central nervous system. Mech Dev 1999 September;87(1-2):223-7. Zebrafish ten-m3 and ten-m4 encode proteins highly similar to the product of Drosophila pair-rule gene ten(m)/odd Oz (odz). Their products contain eight epidermal growth factor (EGF)-like repeats that resemble mostly those of the extracellular matrix molecule tenascin. During segmentation period, ten-m3 is expressed in the somites, notochord, pharyngeal arches, and the brain, while expression of ten-m4 is mainly restricted to the brain. In the developing brain, ten-m3 and ten-m4 expression delineates several compartments.

Interestingly, ten-m3 and ten-m4 show expression patterns complementary to each other in the developing forebrain and midbrain along both rostrocaudal and dorsoventral axes, depending on developmental stages and locations

Panel CNS_{—}1 Summary: Ag2582/Ag2910 Two experiments with the same probe and primer set further confirm expression of the CG503 01-01 gene in the brain. Please see Panel 1.3D for discussion of potential utility in the central nervous system.

E. CG55764-01 and CG55764-02: Out-At-First-Like

Expression of gene CG55764-01 and variant CG55764-02 was assessed using the primer- probe set Ag3207, described in Table EA. Results of the RTQ-PCR runs are shown in Tables 12EB, 12EC, 12ED, 12EE and 12EF.

TABLE 12EA

Probe Name Ag3207

Start

| | | | |
|---|---|---|---|
| Primers | Sequences | Length | Position |

| | | | |
|---|---|---|---|
| Forward | 5′-gccgacttcaagaaggatgt-3′ | 20 | 217 |
| | (SEQ ID NO:125) | | |
| Probe | TET-5′-aaggtcttccgggccctgatcct-3′- | 23 | 238 |
| | TAMRA (SEQ ID NO:126) | | |
| Reverse | 5′-gaactgactctgccccttct-3′ | 20 | 272 |
| | (SEQ ID NO:127) | | |

TABLE 12EB
CNS_neurodegeneration_v1.0

| | Rel. Exp. (%) Ag3207, | | Rel. Exp. (%) Ag3207, |
|---|---|---|---|
| Tissue Name | Run 209861776 | Tissue Name | Run 209861776 |
| AD 1 Hippo | 23.3 | Control (Path) 3 | 22.5 |
| | | Temporal Ctx | |
| AD 2 Hippo | 82.9 | Control (Path) 4 | 84.7 |
| | | Temporal Ctx | |
| AD 3 Hippo | 21.9 | AD 1 Occipital Ctx | 24.0 |
| AD 4 Hippo | 27.7 | AD 2 Occipital Ctx | 0.0 |
| | | (Missing) | |
| AD 5 Hippo | 75.8 | AD 3 Occipital Ctx | 25.3 |
| AD 6 Hippo | 98.6 | AD 4 Occipital Ctx | 43.2 |
| Control 2 Hippo | 64.6 | AD 5 Occipital Ctx | 53.6 |
| Control 4 Hippo | 35.4 | AD 6 Occipital Ctx | 24.1 |
| Control (Path) 3 | 24.0 | Control 1 Occipital | 29.9 |
| Hippo | | Ctx | |
| AD 1 Temporal Ctx | 32.1 | Control 2 Occipital | 54.0 |
| | | Ctx | |
| AD 2 Temporal Ctx | 81.8 | Control 3 Occipital | 31.9 |
| | | Ctx | |
| AD 3 Temporal Ctx | 38.4 | Control 4 Occipital | 41.2 |
| | | Ctx | |
| AD 4 Temporal Ctx | 43.2 | Control (Path) 1 | 82.9 |
| | | Occipital Ctx | |
| AD 5 Inf Temporal | 100.0 | Control (Path) 2 | 20.4 |
| Ctx | | Occipital Ctx | |
| AD 5 Sup Temporal | 51.8 | Control (Path) 3 | 13.2 |
| Ctx | | Occipital Ctx | |
| AD 6 Inf Temporal | 82.9 | Control (Path) 4 | 29.7 |
| Ctx | | Occipital Ctx | |
| AD 6 Sup Temporal | 79.6 | Control 1 Parietal | 44.8 |
| Ctx | | Ctx | |
| Control 1 Temporal | 49.3 | Control 2 Parietal | 97.9 |
| Ctx | | Ctx | |
| Control 2 Temporal | 64.2 | Control 3 Parietal | 25.0 |
| Ctx | | Ctx | |
| Control 3 Temporal | 40.9 | Control (Path) 1 | 75.8 |
| Ctx | | Parietal Ctx | |
| Control 3 Temporal | 69.7 | Control (Path) 2 | 79.6 |
| Ctx | | Parietal Ctx | |
| Control (Path) 1 | 55.5 | Control (Path) 3 | 19.8 |
| Temporal Ctx | | Parietal Ctx | |
| Control (Path) 2 | 43.5 | Control (Path) 4 | 47.3 |
| Temporal Ctx | | Parietal Ctx | |

TABLE 12EC
Panel 1.3D

| | Rel. Exp.(%) Ag3207, | | Rel. Exp.(%) Ag3207, |
|---|---|---|---|
| Tissue Name | Run 167994683 | Tissue Name | Run 167994683 |
| Liver adenocarcinoma | 6.8 | Kidney (fetal) | 44.4 |
| Pancreas | 11.7 | Renal ca. 786-0 | 10.0 |
| Pancreatic ca. CAPAN 2 | 8.3 | Renal ca. A498 | 28.1 |
| Adrenal gland | 12.0 | Renal ca. RXF 393 | 20.2 |
| Thyroid | 4.2 | Renal ca. ACHN | 6.0 |
| Salivary gland | 14.0 | Renal ca. UO-31 | 6.3 |
| Pituitary gland | 2.2 | Renal ca. TK-10 | 3.0 |
| Brain (fetal) | 2.3 | Liver | 100.0 |
| Brain (whole) | 9.3 | Liver (fetal) | 31.4 |
| Brain (amygdala) | 8.7 | Liver ca. | 11.8 |
| | | (hepatoblast) HepG2 | |
| Brain (cerebellum) | 0.0 | Lung | 4.2 |
| Brain (hippocampus) | 9.6 | Lung (fetal) | 7.6 |
| Brain (substantianigra) | 3.1 | Lung ca. (small | 4.2 |
| | | cell) LX-1 | |
| Brain (thalamus) | 3.0 | Lung ca. (small | 0.2 |
| | | cell) NCI-H69 | |
| Cerebral Cortex | 26.8 | Lung ca. (s.cell | 0.0 |
| | | var.) SHP-77 | |
| Spinal Cord | 9.7 | Lung ca. (large | 0.3 |
| | | cell) NCI-H460 | |
| glio/astro U87-MG | 19.6 | Lung ca. (non-sm. | 5.0 |
| | | cell) A549 | |
| glio/astro U-118-MG | 8.9 | Lung ca. (non- | 2.1 |
| | | s.cell) NCI-H23 | |
| astrocytoma SW1783 | 16.8 | Lung ca. (non- | 5.3 |
| | | s.cell) HOP-62 | |
| neuro*; met SK-N-AS | 5.1 | Lung ca. (non-s.cl) | 2.7 |
| | | NCI-H522 | |
| astrocytoma SF-539 | 7.9 | Lung ca. (squam.) SW | 14.6 |
| | | 900 | |
| astrocytoma SNB-75 | 34.6 | Lung ca. (squam.) | 0.0 |
| | | NCI-H596 | |
| glioma SNB-19 | 6.6 | Mammary gland | 33.0 |
| glioma U251 | 13.6 | Breast ca.* (pl.ef) | 0.9 |
| | | MCF-7 | |
| glioma SF-295 | 31.6 | Breast ca.* (pl.ef) | 10.6 |
| | | MDA-MB-231 | |
| Heart (fetal) | 33.7 | Breast ca.* (pl.ef) | 3.2 |
| | | T47D | |
| Heart | 9.1 | Breast ca. BT-549 | 5.6 |
| Skeletal muscle | 44.1 | Breast ca. MDA-N | 31.2 |
| (fetal) | | | |
| Skeletal muscle | 5.6 | Ovary | 44.4 |
| Bone marrow | 0.4 | Ovarian ca. OVCAR-3 | 1.1 |
| Thymus | 3.0 | Ovarian ca. OVCA.R-4 | 4.0 |
| Spleen | 15.5 | Ovarian ca. OVCAR-5 | 45.7 |
| Lymph node | 2.9 | Ovarian ca. OVCAR-8 | 1.9 |
| Colorectal | 16.5 | Ovarian ca. IGROV-1 | 4.5 |
| Stomach | 5.2 | Ovarian ca.* | 17.4 |
| | | (ascites) SK-OV-3 | |
| Small intestine | 9.3 | Uterus | 10.0 |
| Colon ca. SW480 | 6.1 | Plancenta | 0.2 |
| Colon ca.* SW620(SW480 | 17.6 | Prostate | 2.1 |
| met) | | | |
| Colon ca. HT29 | 11.1 | Prostate ca.* (bone | 8.7 |
| | | met)PC-3 | |
| Colon ca. HCT-116 | 3.7 | Testis | 0.9 |
| Colon ca. CaCo-2 | 46.7 | Melanoma Hs688(A).T | 4.8 |
| Colon ca. | 17.7 | Melanoma* (met) | 10.9 |
| tissue (ODO3866) | | Hs688(B).T | |
| Colon ca. HCC-2998 | 4.4 | Melanoma UACC-62 | 44.8 |
| Gastric ca.* (liver | 15.9 | Melanoma M14 | 8.0 |
| met) NCI-N87 | | | |
| Bladder | 10.9 | Melanoma LOX IMVI | 16.8 |
| Trachea | 3.0 | Melanoma* (met) SK- | 9.6 |
| | | MEL-5 | |
| Kidney | 18.7 | Adipose | 29.1 |

TABLE 12ED
Panel 4D

| | Rel. Exp.(%) | | Rel. Exp.(%) |
|---|---|---|---|
| | Ag3207, Run | | Ag3207, Run |
| Tissue Name | 164531738 | Tissue Name | 164531738 |
| Secondary Th1 act | 2.7 | HUVEC IL-1beta | 8.4 |
| Secondary Th2 act | 3.9 | HUVEC IFN gamma | 37.9 |
| Secondary Tr1 act | 3.6 | HUVEC TNF alpha + IFN | 42.0 |
| | | gamma | |
| Secondary Th1 rest | 0.3 | HUVEC TNF alpha + IL4 | 12.8 |
| Secondary Th2 rest | 0.2 | HUVEC IL-11 | 19.1 |
| Secondary Tr1 rest | 1.0 | Lung Microvascular EC | 37.4 |
| | | none | |
| Primary Th1 act | 2.7 | Lung Microvascular EC | 31.4 |
| | | TNF alpha + IL-1beta | |
| Primary Th2 act | 0.8 | Microvascular Dermal EC | 49.3 |
| | | none | |
| Primary Tr1 act | 2.4 | Microsvasular Dermal EC | 49.3 |
| | | TNF alpha + IL-1beta | |
| Primary Th1 rest | 0.9 | Bronchial epithelium | 36.1 |
| | | TNF alpha + IL1beta | |
| Primary Th2 rest | 0.3 | Small airway epithelium | 13.8 |
| | | none | |
| Primary Tr1 rest | 0.0 | Small airway epithelium | 75.8 |
| | | TNF alpha + IL-1beta | |
| CD45RA CD4 lymphocyte | 14.8 | Coronery artery SMC rest | 54.7 |
| act | | | |
| CD45RO CD4 lymphocyte | 1.0 | Coronery artery SMC | 46.7 |
| act | | TNF alpha + IL-1beta | |
| CD8 lymphocyte act | 1.2 | Astrocytes rest | 8.8 |
| Secondary CD8 | 0.9 | Astrocytes TNF alpha + IL- | 11.4 |
| lymphocyte rest | | 1beta | |
| Secondary CD8 | 3.0 | KU-812 (Basophil) rest | 60.3 |
| lymphocyte act | | | |
| CD4 lymphocyte none | 1.0 | KU-812 (Basophil) | 30.8 |
| | | PMA/ionomycin | |
| 2ry Th1/Th2/Tr1_anti- | 0.3 | CCD1106 (Keratinocytes) | 10.3 |
| CD95 CH11 | | none | |
| LAK cells rest | 9.2 | CCD1106 (Keratinocytes) | 4.7 |
| | | TNF alpha + IL-1beta | |
| LAK cells IL-2 | 1.0 | Liver cirrhosis | 24.5 |
| LAK cells IL-2 + IL-12 | 2.1 | Lupus kidney | 8.8 |
| LAK cells IL-2 + IFN | 2.0 | NCI-H292 none | 18.4 |
| gamma | | | |
| LAK cells IL-2 + IL-18 | 1.0 | NCI-H292 IL-4 | 29.3 |
| LAK cells | 3.4 | NCI-H292 IL-9 | 26.2 |
| PMA/ionomycin | | | |
| NK Cells IL-2 rest | 1.1 | NCI-H292 IL-13 | 17.6 |
| Two Way MLR 3 day | 4.0 | NCI-H292 IFN gamma | 27.4 |
| Two Way MLR 5 day | 4.2 | HPAEC none | 11.3 |
| Two Way MLR 7 day | 0.5 | HPAEC TNF alpha + IL-1beta | 21.9 |
| PBMC rest | 4.7 | Lung fibroblast none | 24.8 |
| PBMC PWM | 6.3 | Lung fibroblast TNF alpha + IL- | 39.2 |
| | | 1beta | |
| PBMC PHA-L | 2.9 | Lung fibroblast IL-4 | 36.6 |
| Ramos (B cell) none | 0.0 | Lung fibroblast IL-9 | 31.9 |
| Ramos (B cell) | 0.0 | Lung fibroblast IL-13 | 29.9 |
| ionomycin | | | |
| B lymphocytes PWM | 4.8 | Lung fibroblast IFN gamma | 56.6 |
| B lymphocytes CD40L | 0.2 | Dermal fibroblast CCD1070 | 75.8 |
| and IL-4 | | rest | |
| EOL-1 dbcAMP | 7.4 | Dermal fibroblast CCD1070 | 50.3 |
| | | TNF alpha | |
| EOL-1 dbcAMP | 13.6 | Dermal fibroblast CCD1070 | 100.0 |
| PMA/ionomycin | | IL-1beta | |
| Dendritic cells none | 7.6 | Dermal fibroblast IFN | 35.8 |
| | | gamma | |
| Dendritic cells LPS | 0.9 | Dermal fibroblast IL-4 | 26.4 |
| Dendritic cells anti- | 3.6 | IBD Colitis 2 | 1.0 |
| CD40 | | | |
| Monocytes rest | 17.2 | IBD Crohn's | 4.5 |
| Monocytes LPS | 8.3 | Colon | 32.8 |
| Macrophages rest | 4.2 | Lung | 16.3 |
| Macrophages LPS | 4.5 | Thymus | 49.7 |
| HUVEC none | 25.3 | Kidney | 7.3 |
| HUVEC starved | 32.1 | | |

TABLE 12EE
Panel CNS_1

| | Rel. Exp.(%) Ag3207, Run | | Rel. Exp.(%) Ag3207, Run |
|---|---|---|---|
| Tissue Name | 190323248 | Tissue Name | 190323248 |
| BA4 Control | 46.0 | BA17 PSP | 13.2 |
| BA4 Control2 | 40.6 | BA17 PSP2 | 29.3 |
| BA4 Alzheimer's2 | 11.3 | Sub Nigra Control | 41.2 |
| BA4 Parkinson's | 49.0 | Sub Nigra Control2 | 4.6 |
| BA4 Parkinson's2 | 49.0 | Sub Nigra | 18.7 |
| | | Alzheimer's2 | |
| BA4 Huntington's | 30.6 | Sub Nigra | 19.2 |
| | | Parkinson's2 | |
| BA4 | 51.4 | Sub Nigra | 24.1 |
| Huntington's2 | | Huntington's | |
| BA4 PSP | 7.8 | Sub Nigra | 11.7 |
| | | Huntington's2 | |
| BA4 PSP2 | 24.7 | Sub Nigra PSP2 | 0.0 |
| BA4 Depression | 32.3 | Sub Nigra Depression | 4.2 |
| BA4 Depression2 | 39.5 | Sub Nigra | 19.5 |
| | | Depression2 | |
| BA7 Control | 58.6 | Glob Palladus | 37.6 |
| | | Control | |
| BA7 Control2 | 38.2 | Glob Palladus | 33.4 |
| | | Control2 | |
| BA7 Alzheimer's2 | 0.0 | Glob Palladus | 11.3 |
| | | Alzheimer's | |
| BA7 Parkinson's | 0.0 | Glob Palladus | 45.7 |
| | | Alzheimer's2 | |
| BA7 Parkinson's2 | 36.1 | Glob Palladus | 85.3 |
| | | Parkinson's | |
| BA7 Huntington's | 63.3 | Glob Palladus | 22.4 |
| | | Parkinson's2 | |
| BA7 | 50.3 | Glob Palladus PSP | 4.2 |
| Huntington's2 | | | |
| BA7 PSP | 28.3 | Glob Palladus PSP2 | 25.0 |
| BA7 PSP2 | 34.2 | Glob Palladus | 0.0 |
| | | Depression | |
| BA7 Depression | 5.2 | Temp Pole Control | 25.5 |
| BA9 Control | 34.4 | Temp Pole Control2 | 68.8 |
| BA9 Control2 | 56.6 | Temp Pole | 19.8 |
| | | Alzheimer's | |
| BA9 Alzheimer's | 19.1 | Temp Pole | 12.1 |
| | | Alzheimer's2 | |
| BA9 Alzheimer's2 | 47.6 | Temp Pole | 46.7 |
| | | Parkinson's | |
| BA9 Parkinson's | 23.7 | Temp Pole | 74.2 |
| | | Parkinson's2 | |
| BA9 Parkinson's2 | 33.7 | Temp Pole | 69.3 |
| | | Huntington's | |
| BA9 Huntington's | 100.0 | Temp Pole PSP | 0.0 |
| BA9 | 59.9 | Temp Pole PSP2 | 0.0 |
| Huntington's2 | | | |
| BA9 PSP | 20.0 | Temp Pole | 25.5 |
| | | Depression2 | |
| BA9 PSP2 | 17.3 | Cing Gyr Control | 46.3 |
| BA9 Depression | 16.5 | Cing Gyr Control2 | 41.5 |
| BA9 Depression2 | 20.7 | Cing Gyr Alzheimer's | 46.0 |
| BA17 Control | 44.1 | Cing Gyr | 26.8 |
| | | Alzheimer's2 | |
| BA17 Control2 | 54.0 | Cing Gyr Parkinson's | 45.7 |
| BA17 | 28.7 | Cing Gyr | 22.1 |
| Alzheimer's2 | | Parkinson's2 | |
| BA17 Parkinson's | 59.0 | Cing Gyr | 93.3 |
| | | Huntington's | |
| BA17 | 39.5 | Cing Gyr | 19.6 |
| Parkinson's2 | | Huntington's2 | |
| BA17 | 38.4 | Cing Gyr PSP | 0.0 |
| Huntington's | | | |
| BA17 | 24.0 | Cing Gyr PSP2 | 0.0 |
| Huntington's2 | | | |
| BA17 Depression | 42.0 | Cing Gyr Depression | 32.1 |
| BA17 Depression | 44.1 | Cing Gyr Depression2 | 32.3 |

TABLE 12EF
Panel CNS_1.1

| | Rel. Exp.(%) Ag3207, Run | | Rel. Exp.(%) Ag3207, Run |
|---|---|---|---|
| Tissue Name | 190072845 | Tissue Name | 190072845 |
| Cing Gyr Depression2 | 15.3 | BA17 PSP2 | 9.2 |
| Cing Gyr Depression | 25.2 | BA17 PSP | 17.2 |
| Cing Gyr PSP2 | 12.6 | BA17 | 25.2 |
| | | Huntington's2 | |
| Cing Gyr PSP | 18.3 | BA17 | 18.6 |
| | | Huntington's | |
| Cing Gyr | 23.8 | BA17 | 36.6 |
| Huntington's2 | | Parkinson's2 | |
| Cing Gyr | 61.1 | BA17 Parkinson's | 50.3 |
| Huntington's | | | |
| Cing Gyr | 9.4 | BA17 | 5.3 |
| Parkinson's2 | | Alzheimer's2 | |
| Cing Gyr Parkinson's | 49.0 | BA17 Control2 | 32.5 |
| Cing Gyr | 12.7 | BA17 Control | 48.3 |
| Alzheimer's2 | | | |
| Cing Gyr Alzheimer's | 25.9 | BA9 Depression2 | 27.2 |
| Cing Gyr Control2 | 39.5 | BA9 Depression | 10.4 |
| Cing Gyr Control | 32.3 | BA9 PSP2 | 7.6 |
| Temp Pole | 30.1 | BA9 PSP | 13.4 |
| Depression2 | | | |
| Temp Pole PSP2 | 13.8 | BA9 | 46.0 |
| | | Huntington's2 | |
| Temp Pole PSP | 2.6 | BA9 Huntington's | 58.6 |
| Temp Pole | 39.2 | BA9 Parkinson's2 | 42.0 |
| Huntington's | | | |
| Temp Pole | 41.2 | BA9 Parkinson's | 25.9 |
| Parkinson's2 | | | |
| Temp Pole | 47.0 | BA9 Alzheimer's2 | 17.3 |
| Parkinson's | | | |
| Temp Pole | 25.7 | BA9 Alzheimer's | 13.2 |
| Alzheimer's2 | | | |
| Temp Pole | 20.6 | BA9 Control2 | 57.8 |
| Alzheimer's | | | |
| Temp Pole Control2 | 55.5 | BA9 Control | 42.9 |
| Temp Pole Control | 23.0 | BA7 Depression | 13.4 |
| Glob Palladus | 22.4 | BA7 PSP2 | 26.1 |
| Depression | | | |
| Glob Palladus PSP2 | 8.3 | BA7 PSP | 25.5 |
| Glob Palladus PSP | 10.5 | BA7 | 31.2 |
| | | Huntington's2 | |
| Glob Palladus | 31.0 | BA7 Huntington's | 30.1 |
| Parkinson's2 | | | |
| Glob Palladus | 100.0 | BA7 Parkinson's2 | 15.0 |
| Parkinson's | | | |
| Glob Palladus | 43.2 | BA7 Parkinson's | 25.7 |
| Alzheimer's2 | | | |
| Glob Palladus | 24.0 | BA7 Alzheimer's2 | 9.2 |
| Alzheimer's | | | |
| Glob Palladus | 35.4 | BA7 Control2 | 28.1 |
| Control2 | | | |
| Glob Palladus | 48.6 | BA7 Control | 38.7 |
| Control | | | |
| Sub Nigra | 9.9 | BA4 Depression2 | 26.4 |
| Depression2 | | | |
| Sub Nigra Depression | 6.0 | BA4 Depression | 18.7 |
| Sub Nigra PSP2 | 5.5 | BA4 PSP2 | 22.7 |
| Sub Nigra | 24.0 | BA4 PSP | 14.9 |
| Huntington's2 | | | |
| Sub Nigra | 10.8 | BA4 | 33.0 |
| Huntington's | | Huntington's2 | |
| Sub Nigra | 22.5 | BA4 Huntington's | 15.9 |
| Parkinson's2 | | | |
| Sub Nigra | 13.9 | BA4 Parkinson's2 | 32.8 |
| Alzheimer's2 | | | |
| Sub Nigra Control2 | 13.1 | BA4 Parkinson's | 29.9 |
| Sub Nigra Control | 17.9 | BA4 Alzheimer's2 | 5.1 |
| BA17 Depression2 | 46.3 | BA4 Control2 | 35.6 |
| BA17 Depression | 30.1 | BA4 Control | 52.1 |

CNS_neurodegeneration_v1.0 Summary: Ag3207 No difference is detected in the expression of the CG55764-01 gene in the postmortem brains of Alzheimer's patients when compared normal controls. However, this panel demonstrates the expression of this gene in the CNS of an independent group of patients. See panel 1.3d for a discussion of utility of this gene in the central nervous system.

Panel 1.3D Summary: Ag3207 Highest expression of the CG55764-01 gene is seen in the liver (CT=28.5). Other metabolic tissues that express this gene at more moderate levels include fetal skeletal muscle, fetal kidney, fetal liver and adipose. Low but significant levels of expression are also seen in the heart, kidney, fetal heart, pancreas, adrenal, salivary gland, small intestine, skeletal muscle, pituitary and stomach. The widespread expression of this gene among tissues with metabolic function suggests that antibody or peptide therapeutics to this gene product may be useful in metabolic disorders involving these tissues, including obesity and diabetes. In addition, this gene may be used to differentiate between the fetal (CT=29.7) and adult(CT=32.7) sources of skeletal muscle. Furthermore, the higher levels of expression in fetal skeletal muscle, when compared to expression in the adult suggest that the protein encoded by this gene may be involved in the development of this organ. Thus, therapeutic modulation of the activity or function of this gene product may restore muscle mass or function to weak or dystrophic muscle. This gene is a homolog of the Drosophila Out-At-First protein and is expressed at moderate levels in all brain regions examined, except for the cerebellum where it is not expressed. This protein is believed to be involved in neural development, and may therefore be of use in the treatment of developmental disorders such as autism, schizophrenia, attention deficit disorder, or Tourette syndrome. Overall, this gene is expressed at moderate levels in almost all cell types on this panel. The ubiquitous expression of this gene suggests that is required for growth and proliferation of cells.

Panel 4D Summary: Ag 3207 The CG55764-01 transcript is found at moderate levels in dermal fibroblasts, small aiway epithelium and lung fibroblasts. The expression of this transcript appears to be up-regulated in these cell types by the inflammatory cytokines TNF-a, IL-1b and IFN-g. This gene is also expressed in KU-812, a basophil cell line. Basophils play an important role in atopic and inflammatory diseases such as asthma, Crohn's disease, and ulcerative colitis. Therefore, the modulation of the expression or activity of the protein encoded by this transcript through the application of antibody or peptide therapeutics may be useful for the treatment of lung inflammatory diseases such as asthma, and chronic obstructive pulmonary diseases, for inflammatory skin diseases such as psoriasis, atopic dermatitis and ulcerative dermatitis, inflammatory bowel diseases and osteoarthritis.

Panel CNS_{—}1 Summary: Ag3207 This experiment further confirms expression of the CG55764-01 gene in the brain. Please see Panel 1.3D for discussion of potential utility of this gene in the central nervous system.

Panel CNS_{—}1.1 Summary: Ag3207 This experiment further confirms expression of the CG55764-01 gene in the brain. Please see Panel 1.3D for discussion of potential utility of this gene in the central nervous system.

F. CG55704-01: Ephrin Type-A Receptor 6 Precursor

Expression of gene CG55704-01 was assessed using the primer-probe sets Ag4155, Ag568, Ag1486, Ag2879 and Ag1302, described in Tables 12FA, 12FB, 12FC, 12FD and 12FE. Results of the RTQ-PCR runs are shown in Tables 12FF, 12FG, 12FH, 12FI, 12FJ, and 12FK.

TABLE 12FA

Probe Name Ag4155

Start

| | | | |
|---|---|---|---|
| Primers | Sequences | Length | Position |

| | | | |
|---|---|---|---|
| Forward | 5′-acccaccttctatggcatgta-3′ | 21 | 980 |
| | (SEQ ID NO:128) | | |
| Probe | TET-5′-aggccaccttcagctcctaggaatgt-3′- | 26 | 1003 |
| | TAMRA (SEQ ID NO:129) | | |
| Reverse | 5′-gggctgtttcattgatgttaaa-3′ | 22 | 1033 |
| | (SEQ ID NO:130) | | |

TABLE 12FB

Probe Name Ag568

Start

| | | | |
|---|---|---|---|
| Primers | Sequences | Length | Position |

| | | | |
|---|---|---|---|
| Forward | 5′-agccccagaagccatcg-3′ | 17 | 2544 |
| | (SEQ ID NO:131) | | |
| Probe | TET-5′-ttctcctcagcaagcgatgcatgga-3′- | 25 | 2572 |
| | TAMRA SEQ ID NO:132 | | |
| Reverse | 5′-ctcccacatgacaatgccatag-3′ | 22 | 2598 |
| | (SEQ ID NO:133) | | |

TABLE 12FC

Probe Name Ag1486

Start

| | | | |
|---|---|---|---|
| Primers | Sequences | Length | Position |

| | | | |
|---|---|---|---|
| Forward | 5′-tcccgggaattaaaacttacat-3′ | 22 | 1814 |
| | (SEQ ID NO:134) | | |
| Probe | TET-5′-cccatccctagcagtccatgaatttg-3′- | 26 | 1857 |
| | TAMRA (SEQ ID NO:135) | | |
| Reverse | 5′-tcttgagggatcaatctccttt-3′ | 22 | 1884 |
| | (SEQ ID NO:136) | | |

TABLE 12FD

Probe Name Ag2879

Start

| | | | |
|---|---|---|---|
| Primers | Sequences | Length | Position |

| | | | |
|---|---|---|---|
| Forward | 5′-gcagattattgctacgcaatg-3′ | 21 | 3347 |
| | (SEQ ID NO:137) | | |
| Probe | TET-5′-aaacctatctaggcccatgaatggaa-3′- | 26 | 3379 |
| | TAMRA (SEQ ID NO:138) | | |
| Reverse | 5′-aggatcggatttggatttgtt-3′ | 21 | 3405 |
| | (SEQ ID NO:139) | | |

TABLE 12FE

Probe Name Ag1302

Start

| | | | |
|---|---|---|---|
| Primers | Sequences | Length | Position |

| | | | |
|---|---|---|---|
| Forward | 5′-ggcagaaggagagaaatcaca-3′ | 21 | 2753 |
| | (SEQ ID NO:140) | | |
| Probe | TET-5′-actgacattgtcagcttccttgacaa-3′- | 26 | 2785 |
| | TAMRA (SEQ ID NO:141) | | |
| Reverse | 5′-cactgggatttcggatcagt-3′ | 20 | 2811 |
| | (SEQ ID NO:142) | | |

TABLE 12FF
CNS_neurodegeneration_v1.0

| | Rel. Exp.(%) Ag4155, | | Rel. Exp.(%) Ag4155, |
|---|---|---|---|
| Tissue Name | Run 215328490 | Tissue Name | Run 215328490 |
| AD 1 Hippo | 21.3 | Control (Path) 3 | 8.4 |
| | | Temporal Ctx | |
| AD 2 Hippo | 61.1 | Control (Path) 4 | 47.6 |
| | | Temporal Ctx | |
| AD 3 Hippo | 16.8 | AD 1 Occipital Ctx | 17.4 |
| AD 4 Hippo | 22.4 | AD 2 Occipital Ctx | 0.0 |
| | | (Missing) | |
| AD 5 hippo | 79.0 | AD 3 Occipital Ctx | 4.2 |
| AD 6 Hippo | 69.3 | AD 4 Occipital Ctx | 39.2 |
| Control 2 Hippo | 76.3 | AD 5 Occipital Ctx | 25.3 |
| Control 4 Hippo | 7.2 | AD 6 Occipital Ctx | 63.3 |
| Control (Path) 3 | 10.0 | Control 1 Occipital | 4.0 |
| Hippo | | Ctx | |
| AD 1 Temporal Ctx | 16.6 | Control 2 Occipital | 61.6 |
| | | Ctx | |
| AD 2 Temporal Ctx | 52.9 | Control 3 Occipital | 18.4 |
| | | Ctx | |
| AD 3 Temporal Ctx | 6.8 | Control 4 Occipital | 7.9 |
| | | Ctx | |
| AD 4 Temporal Ctx | 46.7 | Control (Path) 1 | 81.2 |
| | | Occipital Ctx | |
| AD 5 Inf Temporal | 100.0 | Control (Path) 2 | 16.3 |
| Ctx | | Occipital Ctx | |
| AD 5 SupTemporal Ctx | 74.7 | Control (Path) 3 | 2.8 |
| | | Occipital Ctx | |
| AD 6 Inf Temporal | 31.2 | Control (Path) 4 | 18.9 |
| Ctx | | Occipital Ctx | |
| AD 6 Sup Temporal | 54.3 | Control 1 Parietal | 7.5 |
| Ctx | | Ctx | |
| Control 1 Temporal | 8.4 | Control 2 Parietal | 36.9 |
| Ctx | | Ctx | |
| Control 2 Temporal | 49.7 | Control 3 Parietal | 20.6 |
| Ctx | | Ctx | |
| Control 3 Temporal | 21.6 | Control (Path) 1 | 97.9 |
| Ctx | | Parietal Ctx | |
| Control 4 Temporal | 15.3 | Control (Path) 2 | 43.5 |
| Ctx | | Parietal Ctx | |
| Control (Path) 1 | 89.5 | Control (Path) 3 | 6.3 |
| Temporal Ctx | | Parietal Ctx | |
| Control (Path) 2 | 55.5 | Control (Path) 4 | 57.0 |
| Temporal Ctx | | Parietal Ctx | |

TABLE 12FG
General_screening_panel_v1.4

| | Rel. Exp.(%) Ag4155, | | Rel. Exp.(%) Ag4155, |
|---|---|---|---|
| Tissue Name | Run 222001153 | Tissue Name | Run 222001153 |
| Adipose | 0.8 | Renal ca. TK-10 | 7.0 |
| Melanoma* | 0.0 | Bladder | 1.1 |
| Hs688(A).T | | | |
| Melanoma* | 0.0 | Gastric ca. (liver | 0.2 |
| Hs688(B).T | | met.) NCI-N87 | |
| Melanoma* M14 | 0.0 | Gastric ca. KATO III | 0.0 |
| Melanoma* LOXIMVI | 0.3 | Colon ca. SW-948 | 0.0 |
| Melanoma* SK-MEL-5 | 0.0 | Colon ca. SW480 | 0.0 |
| Squamous cell | 0.0 | Colon ca.* (SW480 met) | 0.0 |
| carcinoma SCC-4 | | SW620 | |
| Testis Pool | 2.8 | Colon ca. HT29 | 0.0 |
| Prostate ca.* (bone | 6.9 | Colon ca. HCT-116 | 0.0 |
| met) PC-3 | | | |
| Prostate Pool | 7.2 | Colon ca. CaCo-2 | 6.7 |
| Placenta | 0.0 | Colon cancer tissue | 0.4 |
| Uterus Pool | 2.2 | Colon ca. SW1116 | 0.0 |
| Ovarian ca. OVCAR-3 | 2.2 | Colon ca. Colo-205 | 0.1 |
| Ovarian ca. SK-OV-3 | 3.5 | Colon ca. SW-48 | 0.0 |
| Ovarian ca. OVCAR-4 | 0.6 | Colon Pool | 10.8 |
| Ovarian ca. OVCAR-5 | 13.4 | Small Intestine Pool | 7.9 |
| Ovarian ca. IGROV-1 | 2.0 | Stomach Pool | 8.7 |
| Ovarian ca. OVCAR-8 | 1.2 | Bone Marrow Pool | 3.8 |
| Ovary | 3.8 | Fetal Heart | 0.8 |
| Breast ca. MCF-7 | 4.4 | Heart Pool | 3.1 |
| Breast ca. MDA-MB- | 0.0 | Lymph Node Pool | 7.2 |
| 231 | | | |
| Breast ca. BT 549 | 0.9 | Fetal Skeletal Muscle | 0.3 |
| Breast ca. T47D | 12.2 | Skeletal Muscle Pool | 0.1 |
| Breast ca. MDA-N | 0.0 | Spleen Pool | 0.0 |
| Breast Pool | 7.2 | Thymus Pool | 7.6 |
| Trachea | 0.6 | CNS cancer | 0.0 |
| | | (glio/astro) U87-MG | |
| Lung | 8.2 | CNS cancer | 0.6 |
| | | (glio/astro) U-118-MG | |
| Fetal Lung | 0.6 | CNS cancer (neuro; met) | 4.0 |
| | | SK-N-AS | |
| Lung ca. NCI-N417 | 2.2 | CNS cancer (astro) SF- | 0.0 |
| | | 539 | |
| Lung ca. LX-1 | 0.0 | CNS cancer (astro) | 0.0 |
| | | SNB-75 | |
| Lung ca. NCI-H146 | 2.4 | CNS cancer (glio) SNB- | 1.2 |
| | | 19 | |
| Lung ca. SHP-77 | 33.9 | CNS cancer (glio) SF- | 0.7 |
| | | 295 | |
| Lung ca. A549 | 0.0 | Brain (Amygdala) Pool | 22.1 |
| Lung ca. NCI-H526 | 0.5 | Brain (cerebellum) | 12.2 |
| Lung ca. NCI-H23 | 23.2 | Brain (fetal) | 100.0 |
| Lung ca. NCI-H460 | 0.0 | Brain (Hippocampus) | 37.9 |
| | | Pool | |
| Lung ca. HOP-62 | 0.5 | Cerebral Cortex Pool | 31.0 |
| Lung ca. NCI-H522 | 0.1 | Brain (Substantia | 21.2 |
| | | nigra) Pool | |
| Liver | 0.0 | Brain (Thalamus) Pool | 40.6 |
| Fetal Liver | 0.3 | Brain (whole) | 28.5 |
| Liver ca. HepG2 | 0.0 | Spinal Cord Pool | 4.5 |
| Kidney Pool | 15.1 | Adrenal Gland | 0.1 |
| Fetal Kidney | 2.5 | Pituitary gland Pool | 0.6 |
| Renal ca. 786-0 | 13.8 | Salivary Gland | 0.1 |
| Renal ca. A498 | 1.2 | Thyroid (female) | 1.5 |
| Renal ca. ACHN | 2.4 | Pancreatic ca. CAPAN2 | 0.0 |
| Renal ca. UO-31 | 0.4 | Pancreas Pool | 7.3 |

TABLE 12FH
Panel 1.1

| | Rel. Exp.(%) Ag568, | | Rel. Exp.(%) Ag568, |
|---|---|---|---|
| Tissue Name | Run 109491840 | Tissue Name | Run 109491840 |
| Adrenal gland | 0.1 | Renal ca. UO-31 | 0.0 |
| Bladder | 0.2 | Renal ca. RXF 393 | 0.0 |
| Brain (amygdala) | 17.9 | Liver | 0.0 |
| Brain (cerebellum) | 49.0 | Liver (fetal) | 0.0 |
| Brain (hippocampus) | 48.0 | Liver ca. | 0.0 |
| | | (hepatoblast) HepG2 | |
| Brain (substantia | 17.6 | Lung | 0.0 |
| nigra) | | | |
| Brain (thalamus) | 21.9 | Lung (fetal) | 0.0 |
| Cerebral Cortex | 24.3 | Lung ca. (non-s.cell) | 0.0 |
| | | HOP-62 | |
| Brain (fetal) | 54.7 | Lung ca. (large | 0.0 |
| | | cell)NCI-H460 | |
| Brain (whole) | 67.4 | Lung ca. (non-s.cell) | 4.8 |
| | | NCI-H23 | |
| glio/astro U-118-MG | 0.0 | Lung ca. (non-s.cl) | 0.0 |
| | | NCI-H522 | |
| astrocytoma SF-539 | 0.0 | Lung ca. (non-sm. | 0.0 |
| | | cell) A549 | |
| astrocytoma SNB-75 | 0.0 | Lung ca. (s.cell | 12.8 |
| | | var.) SHP-77 | |
| astrocytoma SW1783 | 0.0 | Lung ca. (small cell) | 0.0 |
| | | LX-1 | |
| glioma U251 | 0.0 | Lung ca. (small cell) | 5.8 |
| | | NCI-H69 | |
| glioma SF-295 | 0.0 | Lung ca. (squam.) SW | 0.5 |
| | | 900 | |
| glioma SNB-19 | 0.0 | Lung ca. (squam.) | 1.2 |
| | | NCI-H596 | |
| glio/astro U87-MG | 0.0 | Lymph node | 0.0 |
| neuro*; met SK-N-AS | 5.5 | Spleen | 0.0 |
| Mammary gland | 0.0 | Thymus | 0.0 |
| Breast ca. BT-549 | 0.0 | Ovary | 1.7 |
| Breast ca. MDA-N | 0.1 | Ovarian ca. IGROV-1 | 0.4 |
| Breast ca.* (pl.ef) | 1.1 | Ovarian ca. OVCAR-3 | 0.1 |
| T47D | | | |
| Breast ca.* (pl.ef) | 1.9 | Ovarian ca. OVCAR-4 | 0.0 |
| MCF-7 | | | |
| Breast ca.* (pl.ef) | 0.0 | Ovarian ca. OVCAR-5 | 8.8 |
| MDA-MB-231 | | | |
| Small intestine | 5.4 | Ovarian ca. OVCAR-8 | 0.5 |
| Colorectal | 0.6 | Ovarian ca.* | 0.4 |
| | | (ascites) SK-OV-3 | |
| Colon ca. HT29 | 0.2 | Pancreas | 2.8 |
| Colon ca. CaCo-2 | 0.0 | Pancreatic ca. CAPAN 2 | 0.0 |
| Colon ca. HCT-15 | 0.0 | Pituitary gland | 0.1 |
| Colon ca. HCT-116 | 0.0 | Placenta | 0.0 |
| Colon ca. HCC-2998 | 0.0 | Prostate | 3.6 |
| Colon ca. SW480 | 0.0 | Prostate ca.* (bone | 0.4 |
| | | met) PC-3 | |
| Colon ca.* SW620 | 0.0 | Salivary gland | 0.1 |
| (SW480 met) | | | |
| Stomach | 1.9 | Trachea | 0.1 |
| Gastric ca. (liver | 0.0 | Spinal cord | 1.5 |
| met) NCI-N87 | | | |
| Heart | 0.7 | Testis | 100.0 |
| Skeletal muscle | 0.0 | Thyroid | 3.0 |
| (Fetal) | | | |
| Skeletal muscle | 0.0 | Uterus | 0.3 |
| Endothelial cells | 0.0 | Melanoma M14 | 0.0 |
| Heart (Fetal) | 0.0 | Melanoma LOX IMVI | 0.0 |
| Kidney | 0.1 | Melanoma UACC-62 | 0.0 |
| Kidney (fetal) | 0.2 | Melanoma SK-MEL-28 | 0.0 |
| Renal ca. 786-0 | 1.4 | Melanoma* (met) SK- | 0.0 |
| | | MEL-5 | |
| Renal ca. A498 | 0.1 | Melanoma Hs688(A).T | 0.0 |
| Renal ca. ACHN | 0.0 | Melanoma* (met) | 0.0 |
| | | Hs688(B).T | |
| Renal ca. TK-10 | 2.6 | | |

TABLE 12FI
Panel 2.2

| | Rel. Exp.(%) | | Rel. Exp.(%) |
|---|---|---|---|
| | Ag1486, Run | | Ag1486, Run |
| Tissue Name | 173949464 | Tissue Name | 173949464 |
| Normal Colon | 3.3 | Kidney Margin | 7.6 |
| | | (OD04348) | |
| Colon cancer (OD06064) | 3.1 | Kidney malignant | 0.0 |
| | | cancer (OD06204B) | |
| Colon Margin (OD06064) | 1.0 | Kidney normal adjacent | 0.0 |
| | | tissue (OD06204E) | |
| Colon cancer (OD06159) | 0.0 | Kidney Cancer | 0.0 |
| | | (OD04450-01) | |
| Colon Margin (OD06159) | 7.9 | Kidney Margin | 0.0 |
| | | (OD04450-03) | |
| Colon cancer (OD06297- | 0.0 | Kidney Cancer 8120613 | 3.3 |
| 04) | | | |
| Colon Margin (OD06297- | 100.0 | Kidney Margin 8120614 | 0.0 |
| 015) | | | |
| CC Gr.2 ascend colon | 0.0 | Kidney Cancer 9010320 | 3.1 |
| (OD03921) | | | |
| CC Margin (OD03921) | 0.0 | Kidney Margin 9010321 | 0.0 |
| Colon cancer metastasis | 0.0 | Kidney Cancer 8120607 | 2.3 |
| (OD06104) | | | |
| Lung Margin (OD06104) | 1.6 | Kidney Margin 8120608 | 0.0 |
| Colon mets to lung | 0.0 | Normal Uterus | 40.1 |
| (OD04451-01) | | | |
| Lung Margin (OD04451- | 5.1 | Uterine Cancer 064011 | 11.7 |
| 02) | | | |
| Normal Prostate | 0.0 | Normal Thyroid | 0.0 |
| Prostate Cancer | 0.0 | Thyroid Cancer 064010 | 0.0 |
| (OD04410) | | | |
| Prostate Margin | 18.6 | Thyroid Cancer A302152 | 0.0 |
| (OD04410) | | | |
| Normal Ovary | 5.3 | Thyroid Margin A302153 | 3.0 |
| Ovarian cancer | 0.0 | Normal Breast | 10.0 |
| (OD06283-03) | | | |
| Ovarian Margin | 0.0 | Breast Cancer | 0.0 |
| (OD06283-07) | | (OD04566) | |
| Ovarian Cancer 064008 | 5.2 | Breast Cancer 1024 | 0.0 |
| Ovarian cancer | 1.6 | Breast Cancer | 3.0 |
| (OD06145) | | (OD04590-01) | |
| Ovarian Margin | 17.1 | Breast Cancer Mets | 0.0 |
| (OD06145) | | (OD04590-03) | |
| Ovarian cancer | 4.6 | Breast Cancer | 0.0 |
| (OD06455-03) | | Metastasis (OD04655- | |
| | | 05) | |
| Ovarian Margin | 3.8 | Breast Cancer 064006 | 0.0 |
| (OD06455-07) | | | |
| Normal Lung | 2.5 | Breast Cancer 9100266 | 0.0 |
| Invasive poor diff. | 0.0 | Breast Margin 9100265 | 0.0 |
| lung adeno (ODO4945-01 | | | |
| Lung Margin (ODO4945- | 0.0 | Breast Cancer A209073 | 0.0 |
| 03) | | | |
| Lung Malignant Cancer | 0.0 | Breast Margin A2090734 | 0.0 |
| (OD03126) | | | |
| Lung Margin (OD03126) | 0.0 | Breast cancer | 0.0 |
| | | (OD06083) | |
| Lung Cancer (OD05014A) | 0.0 | Breast cancer node | 0.0 |
| | | metastasis (OD06083) | |
| Lung Margin (OD05014B) | 0.0 | Normal Liver | 0.0 |
| Lung cancer (OD06081) | 0.0 | Liver Cancer 1026 | 0.0 |
| Lung Margin (OD06081) | 0.0 | Liver Cancer 1025 | 0.0 |
| Lung Cancer (OD0237- | 0.0 | Liver Cancer 6004-T | 0.0 |
| 01) | | | |
| Lung Margin (OD04237- | 3.7 | Liver Tissue 6004-N | 0.0 |
| 02) | | | |
| Ocular Melanoma | 0.0 | Liver Cancer 6005-T | 0.0 |
| Metastasis | | | |
| Ocular Melanoma Margin | 0.0 | Liver Tissue 6005-N | 0.0 |
| (Liver) | | | |
| Melanoma Metastasis | 0.0 | Liver Cancer 064003 | 0.0 |
| Melanoma Margin (Lung) | 0.0 | Normal Bladder | 4.3 |
| Normal Kidney | 0.0 | Bladder Cancer 1023 | 0.0 |
| Kidney Ca, Nuclear | 0.0 | Bladder Cancer A302173 | 0.0 |
| grade 2 (OD04338) | | | |
| Kidney Margin (OD04338) | 0.0 | Normal Stomach | 55.9 |
| Kidney Ca Nuclear grade | 0.0 | Gastric Cancer 9060397 | 0.0 |
| ½ (OD04339) | | | |
| Kidney Margin (OD04339) | 0.0 | Stomach Margin 9060396 | 13.3 |
| Kidney Ca, Clear cell | 0.0 | Gastric Cancer 9060395 | 4.8 |
| type (OD04340) | | | |
| Kidney Margin (OD04340) | 3.8 | Stomach Margin 9060394 | 6.9 |
| Kidney Ca, Nuclear | 6.2 | Gastric Cancer 064005 | 0.0 |
| grade 3 (OD04348) | | | |

TABLE 12FJ
Panel 4.1D

| | Rel. | Rel. | | Rel. | Rel. |
|---|---|---|---|---|---|
| | Exp. (%) | Exp. (%) | | Exp. (%) | Exp. (%) |
| | Ag4155, Run | Ag4155, Run | | Ag4155, Run | Ag4155, Run |
| Tissue Name | 173124973 | 174261191 | Tissue Name | 173124973 | 174261191 |
| Secondary Th1 act | 0.0 | 0.0 | HUVEC IL-1beta | 0.0 | 7.4 |
| Secondary Th2 act | 0.0 | 0.0 | HUVEC IFN gamma | 0.8 | 5.6 |
| Secondary Tr1 act | 0.0 | 0.0 | HUVEC TNF alpha + IFN | 0.6 | 0.0 |
| | | | gamma | | |
| Secondary Th1 rest | 0.0 | 0.0 | HUVEC TNF alpha + IL4 | 0.3 | 20.0 |
| Secondary Th2 rest | 0.0 | 0.0 | HUVEC IL-11 | 0.3 | 4.2 |
| Secondary Tr1 rest | 0.0 | 0.0 | Lung Microvascular | 0.9 | 14.6 |
| | | | Ec none | | |
| Primary Th1 act | 0.0 | 0.0 | Lung Microvascular | 2.2 | 63.7 |
| | | | EC TNF alpha + IL- | | |
| | | | 1beta | | |
| Primary Th2 act | 0.0 | 0.0 | Microvascular | 0.0 | 0.0 |
| | | | Dermal EC none | | |
| Primary Tr1 act | 0.0 | 0.0 | Microsvasular | 0.0 | 9.8 |
| | | | Dermal EC TNF alpha + | | |
| | | | IL-1beta | | |
| Primary Th1 rest | 0.0 | 0.0 | Bronchial | 0.0 | 0.0 |
| | | | epithelium | | |
| | | | TNF alpha + IL1beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium | | |
| | | | TNF alpha + IL- | | |
| | | | 1beta | | |
| CD45RA CD4 | 0.0 | 0.0 | Coronery artery | 0.0 | 0.0 |
| lymphocyte act | | | SMC rest | | |
| CD45RO CD4 | 0.0 | 0.0 | Coronery artery | 0.0 | 11.7 |
| lymphocyte act | | | SMC TNF alpha + IL- | | |
| | | | 1beta | | |
| CD8 lymphocyte act | 0.0 | 0.0 | Astrocytes rest | 0.0 | 0.0 |
| Secondary CD8 | 0.0 | 0.0 | Astrocytes | 0.0 | 0.0 |
| lymphocyte rest | 0.0 | 0.0 | TNF alpha + IL- | 0.0 | 0.0 |
| | | | 1beta | | |
| Secondary CD8 | 0.0 | 0.0 | KU-812 (Basophil) | 100.0 | 0.0 |
| lymphocyte act | | | rest | | |
| CD4 lymphocyte | 0.0 | 0.0 | KU-812 (Basophil) | 0.0 | 0.0 |
| none | | | PMA/ionomycin | | |
| 2ry | 0.0 | 0.0 | CCD1106 | 0.0 | 3.8 |
| Th1/Th2/Tr1_anti- | | | (Keratinocytes) | | |
| CD95 CH11 | | | none | | |
| LAK cells rest | 0.0 | 0.0 | CCD1106 | 0.0 | 0.0 |
| | | | (Keratinocytes) | | |
| | | | TNF alpha + IL- | | |
| | | | 1beta | | |
| LAK cells IL-2 | 0.0 | 0.0 | Liver cirrhosis | 3.7 | 100.0 |
| LAK cells IL-2 + IL- | 0.0 | 6.0 | NCI-H292 none | 0.0 | 0.0 |
| 12 | | | | | |
| LAK cells IL-2 + IFN | 0.0 | 0.0 | NCI-H292 IL-4 | 0.0 | 0.0 |
| gamma | | | | | |
| LAK cells IL-2 + IL- | 0.0 | 0.0 | NCI-H292 IL-9 | 0.0 | 0.0 |
| 18 | | | | | |
| LAK cells | 0.0 | 0.0 | NCI-H292 IL-13 | 0.3 | 12.6 |
| PMA/ionomycin | | | | | |
| NK Cells IL-2 rest | 0.0 | 0.0 | NCI-H292 IFN gamma | 0.0 | 0.0 |
| Two Way MLR 3 day | 0.0 | 0.0 | HPAEC none | 0.0 | 0.0 |
| Two Way MLR 5 day | 0.0 | 0.0 | HPAEC TNF alpha + IL- | 0.0 | 0.0 |
| | | | 1beta | | |
| Two Way MLR 7 day | 0.0 | 0.0 | Lung fibroblast | 0.3 | 0.0 |
| | | | none | | |
| PBMC rest | 0.0 | 0.0 | Lung fibroblast | 0.0 | 0.0 |
| | | | TNF alpha + IL-1beta | | |
| PBMC PWM | 0.0 | 0.0 | Lung fibroblast | 0.0 | 2.8 |
| | | | IL-4 | | |
| PBMC PHA-L | 0.0 | 0.0 | Lung fibroblast | 0.0 | 0.0 |
| | | | IL-9 | | |
| Ramos (B cell) | 0.0 | 0.0 | Lung fibroblast | 0.0 | 0.0 |
| none | | | IL-13 | | |
| Ramos (B cell) | 0.0 | 0.0 | Lung fibroblast | 0.0 | 0.0 |
| ionomycin | | | IFN gamma | | |
| B lymphocytes PWM | 0.0 | 0.0 | Dermal fibroblast | 0.0 | 0.0 |
| | | | CCD1070 rest | | |
| B lymphocytes | 0.0 | 0.0 | Dermal fibroblast | 0.0 | 0.0 |
| CD40L and IL-4 | | | CCD1070 TNF alpha | | |
| EOL-1 dbcAMP | 0.0 | 0.0 | Dermal fibroblast | 0.0 | 0.0 |
| | | | CCD1070 IL-1beta | | |
| EOL-1 dbcAMP | 0.0 | 0.0 | Dermal fibroblast | 0.0 | 0.0 |
| PMA/ionomycin | | | IFN gamma | | |
| Dendritic cells | 0.0 | 0.0 | Dermal fibroblast | 0.0 | 12.7 |
| none | | | IL-4 | | |
| Dendritic cells | 0.0 | 0.0 | Dermal Fibroblasts | 0.0 | 0.0 |
| LPS | | | rest | | |
| Dendritic cells | 0.0 | 00 | Neutrophils | 0.0 | 0.0 |
| anti-CD40 | | | TNFa + LPS | | |
| Monocytes rest | 0.0 | 0.0 | Neutrophils rest | 0.0 | 4.1 |
| Monocytes LPS | 0.0 | 0.0 | Colon | 2.2 | 35.4 |
| Macrophages rest | 0.0 | 0.0 | Lung | 1.7 | 9.8 |
| Macrophages LPS | 0.0 | 0.0 | Thymus | 0.9 | 28.3 |
| HUVEC none | 0.0 | 0.0 | Kidney | 0.7 | 15.1 |
| HUVEC starved | 0.0 | 3.5 | | | |

TABLE 12FK
Panel 4D

| | Rel. | Rel. | | Rel. | Rel. |
|---|---|---|---|---|---|
| | Exp. (%) | Exp. (%) | | Exp. (%) | Exp. (%) |
| | Ag1302, Run | Ag1486, Run | | Ag1302, Run | Ag1486, Run |
| Tissue Name | 138881940 | 162599619 | Tissue Name | 138881940 | 162599619 |
| Secondary Th1 act | 0.0 | 0.0 | HUVEC IL-1beta | 0.0 | 0.0 |
| Secondary Th2 act | 0.0 | 0.0 | HUVEC IFN gamma | 6.2 | 0.0 |
| Secondary Tr1 act | 0.0 | 0.0 | HUVEC TNF alpha + IFN | 0.0 | 11.4 |
| | | | gamma | | |
| Secondary Th1 rest | 0.0 | 0.0 | HUVEC TNF alpha + IL4 | 6.6 | 0.0 |
| Secondary Th2 rest | 0.0 | 0.0 | HUVEC IL-11 | 6.9 | 0.0 |
| Secondary Tr1 rest | 0.0 | 0.0 | Lung Microvascular | 6.0 | 15.0 |
| | | | EC none | | |
| Primary Th1 act | 0.0 | 0.0 | Lung Microvascular | 0.0 | 0.0 |
| | | | EC TNF alpha + IL- | | |
| | | | 1beta | | |
| Primary Th2 act | 0.0 | 0.0 | Microvascular | 7.0 | 0.0 |
| | | | Dermal EC none | | |
| Primary Tr1 act | 0.0 | 0.0 | Microsvascular | 0.0 | 0.0 |
| | | | Dermal EC TNF alpha + | | |
| | | | IL-1beta | | |
| Primary Th1 rest | 0.0 | 0.0 | Bronchial | 0.0 | 0.0 |
| | | | epithelium | | |
| | | | TNF alpha + IL1beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 6.7 | 0.0 |
| | | | epithelium | | |
| | | | TNF alpha + IL- | | |
| | | | 1beta | | |
| CD45RA CD4 | 0.0 | 0.0 | Coronery artery | 0.0 | 0.0 |
| lymphocyte act | | | SMC rest | | |
| CD45RO CD4 | 0.0 | 0.0 | Coronery artery | 0.0 | 0.0 |
| lymphocyte act | | | SMC TNF alpha + IL- | | |
| | | | 1beta | | |
| CD8 lymphocyte act | 0.0 | 0.0 | Astrocytes rest | 0.0 | 0.0 |
| Secondary CD8 | 0.0 | 0.0 | Astrocytes | 0.0 | 0.0 |
| lymphocyte rest | | | TNF alpha + IL- | | |
| | | | 1beta | | |
| Secondary CD8 | 0.0 | 0.0 | KU-812 (Basophil) | 0.0 | 0.0 |
| lymphocyte act | | | rest | | |
| CD4 lymphocyte | 0.0 | 0.0 | KU-812 (Basophil) | 6.3 | 0.0 |
| none | | | PMA/ionomycin | | |
| 2ry | 9.0 | 0.0 | CCD1106 | 6.9 | 0.0 |
| Th1/Th2/Tr1_anti- | | | (Keratinocytes) | | |
| CD95 CH11 | | | none | | |
| LAK cells rest | 0.0 | 0.0 | CCD1106 | 6.9 | 0.0 |
| | | | (Keratinocytes) | | |
| | | | TNF alpha + IL- | | |
| | | | 1beta | | |
| LAK cells IL-2 | 0.0 | 0.0 | Liver cirrhosis | 34.9 | 27.9 |
| LAK cells IL-2 + IL- | 0.0 | 0.0 | Lupus kidney | 0.0 | 8.2 |
| 12 | | | | | |
| LAK cells IL-2 + IFN | 9.2 | 0.0 | NCI-H292 none | 0.0 | 0.0 |
| gamma | | | | | |
| LAK cells IL-2 + | 0.0 | 0.0 | NCI-H292 IL-4 | 0.0 | 0.0 |
| IL-18 | | | | | |
| LAK cells | 0.0 | 0.0 | NCI-H292 IL-9 | 15.0 | 0.0 |
| PMA/ionomycin | | | | | |
| NK Cells IL-2 rest | 0.0 | 0.0 | NCI-H292 IL-13 | 0.0 | 0.0 |
| Two Way MLR 3 day | 0.0 | 0.0 | NCI-H292 IFN gamma | 0.0 | 0.0 |
| Two Way MLR 5 day | 0.0 | 0.0 | HPAEC none | 0.0 | 0.0 |
| Two Way MLR 7 day | 0.0 | 0.0 | HPAEC TNF alpha + IL- | 0.0 | 0.0 |
| | | | 1beta | | |
| PBMC rest | 0.0 | 0.0 | Lung fibroblast | 0.0 | 0.0 |
| | | | none | | |
| PBMC PWM | 0.0 | 0.0 | Lung fibroblast | 0.0 | 0.0 |
| | | | TNF alpha + IL-1 | | |
| | | | beta | | |
| PBMC PHA-L | 15.3 | 0.0 | Lung fibroblast | 0.0 | 0.0 |
| | | | IL-4 | | |
| Ramos (B cell) | 0.0 | 0.0 | Lung fibroblast | 0.0 | 0.0 |
| none | | | IL-9 | | |
| Ramos (B cell) | 0.0 | 0.0 | Lung fibroblast | 0.0 | 0.0 |
| ionomycin | | | IL-13 | | |
| B lymphocytes PWM | 0.0 | 0.0 | Lung fibroblast | 0.0 | 0.0 |
| | | | IFN gamma | | |
| B lymphocytes | 0.0 | 0.0 | Dermal fibroblast | 0.0 | 0.0 |
| CD40L and IL-4 | | | CCD1070 rest | | |
| EOL-1 dbcAMP | 0.0 | 0.0 | Dermal fibroblast | 0.0 | 0.0 |
| | | | CCD1070 TNF alpha | | |
| EOL-1 dbcAMP | 0.0 | 0.0 | Dermal fibroblast | 0.0 | 0.0 |
| PMA/ionomycin | | | CCD1070 IL-1beta | | |
| Dendritic cells | 0.0 | 0.0 | Dermal fibroblast | 0.0 | 0.0 |
| none | | | IFN gamma | | |
| Dendritic cells | 0.0 | 0.0 | Dermal fibroblast | 0.0 | 0.0 |
| LPS | | | IL-4 | | |
| Dendritic cells | 0.0 | 0.0 | IBD Colitis 2 | 100.0 | 58.2 |
| anti-CD40 | | | | | |
| Monocytes rest | 0.0 | 0.0 | IBD Crohn's | 30.1 | 13.3 |
| Monocytes LPS | 0.0 | 0.0 | Colon | 81.8 | 97.3 |
| Macrophages rest | 0.0 | 0.0 | Lung | 0.0 | 15.7 |
| Macrophages LPS | 0.0 | 0.0 | Thymus | 45.7 | 100.0 |
| HUVEC none | 0.0 | 0.0 | Kidney | 16.0 | 12.2 |
| HUVEC starved | 0.0 | 0.0 | | | |

CNS_neurodegeneration_v1.0 Summary: Ag4155 The CG55704-01 gene encodes a putative ephrin receptor, and shows a significant downregulation in the AD temporal cortex compared to nondemented controls when CT values are analyzed by ANCOVA. The temporal cortex (Brodmann area 21) shows severe neurodegeneration in Alzheimer's disease, though not as early as the hippocampus or entorhinal cortex. It is therefore likely that this gene is downregulated during the process of neurodegeneration, rather than the downregulation being a result of neuron loss. The ephrin receptors have been implicated in axonal and synapse guidance. Furthermore, individuals with Alzheimer's disease (especially late-onset AD with apoE4 genotype) show impaired compensatory synaptogenesis and dendritic arborization. Therefore, this gene is an excellent small molecule target for the treatment of Alzheimer's disease. Please note that a second experiment with the probe and primer set Ag2879 is not included because the amp plot suggests that there were experimental difficulties with this run. References: Lai K O, Ip F C, Cheung J, Fu A K, Ip N Y. Expression of Eph receptors in skeletal muscle and their localization at the neuromuscular junction. Mol Cell Neurosci 2001 June; 17(6):1034-47. The participation of ephrins and Eph receptors in guiding motor axons during muscle innervation has been well documented, but little is known about their expression and functional significance in muscle at later developmental stages. Our present study investigates the expression and localization of Eph receptors and ephrins in skeletal muscle. Prominent expression of EphA4, EphA7, and ephrin-A ligands was detected in muscle during embryonic development. More importantly, both EphA4 and EphA7, as well as ephrin-A2, were localized at the neuromuscular junction (NMJ) of adult muscle. Despite their relative abundance, they were not localized at the synapses during embryonic stages. The concentration of EphA4, EphA7, and ephrin-A2 at the NMJ was observed at postnatal stages and the synaptic localization became prominent at later developmental stages. In addition, expression of Eph receptors was increased by neuregulin and after nerve injury. Furthermore, we demonstrated that overexpression of EphA4 led to tyrosine phosphorylation of the actin-binding protein cortactin and that EphA4 was coimmunoprecipitated with cortactin in muscle. Taken together, our findings indicate that EphA4 is associated with the actin cytoskeleton. Since actin cytoskeleton is critical to the formation and stability of NMJ, the present findings raise the intriguing possibility that Eph receptors may have a novel role in NMJ formation and/or maintenance. Arendt T, Schindler C, Bruckner M K, Eschrich K, Bigl V, Zedlick D, Marcova L. Plastic neuronal remodeling is impaired in patients with Alzheimer's disease carrying apolipoprotein epsilon 4 allele. J Neurosci 1997 Jan. 15; 17(2):516-29. A relationship between the apolipoprotein E (apoE) genotype and the risk to develop Alzheimer's disease has been established recently. Apolipoprotein synthesis is implicated in developmental processes and in neuronal repair of the adult nervous system. In the present study, we investigated the influence of the apolipoprotein polymorphism on the severity of neuronal degeneration and the extent of plastic dendritic remodeling in Alzheimer's disease. Changes in length and arborization of dendrites of Golgi-impregnated neurons in the basal nucleus of Meynert, locus coeruleus, raphe magnus nucleus, medial amygdaloid nucleus, pedunculopontine tegmental nucleus, and substantia nigra were analyzed after three-dimensional reconstruction. Patients with either one or two apoE epsilon 4 alleles not only showed a more severe degeneration in all areas investigated than in patients lacking the apoE 4 allele but also revealed significantly less plastic dendritic changes. apoE epsilon 4 allele copy number, furthermore, had a significant effect on the pattern of dendritic arborization. Moreover, the relationship between the intensity of dendritic growth and both the extent of neuronal degeneration and the stage of the disease seen in patients lacking the apoE epsilon 4 allele was very weak in the presence of one epsilon 4 allele and completely lost in patients homozygous for the epsilon 4 allele. The results provide direct evidence that neuronal reorganization is affected severely in patients with Alzheimer's disease carrying the apoE epsilon 4 allele. This impairment of neuronal repair might lead to a more rapid functional decompensation, thereby contributing to an earlier onset and more rapid progression of the disease. Feldheim D A, Vanderhaeghen P, Hansen M J, Frisen J, Lu Q, Barbacid M, Flanagan J G. Topographic guidance labels in a sensory projection to the forebrain. Neuron 1998 December;21(6):1303-13. Visual connections to the mammalian forebrain are known to be patterned by neural activity, but it remains unknown whether the map topography of such higher sensory projections depends on axon guidance labels. Here, we show complementary expression and binding for the receptor EphA5 in mouse retina and its ligands ephrin-A2 and ephrin-A5 in multiple retinal targets, including the major forebrain target, the dorsal lateral geniculate nucleus (dLGN). These ligands can act in vitro as topographically specific repellents for mammalian retinal axons and are necessary for normal dLGN mapping in vivo. The results suggest a general and economic modular mechanism for brain mapping whereby a projecting field is mapped onto multiple targets by repeated use of the same labels. They also indicate the nature of a coordinate system for the mapping of sensory connections to the forebrain.

General_screening_panel_v1.4 Summary: Ag4155 The CG55704-01 gene shows a tissue expression profile that is highly brain-preferential, with highest expression in the fetal brain (CT=27.3). Please see panel CNS_Neurodegeneration for a discussion of utility of this gene in the central nervous system. Among metabolically relevant tissues, expression of this gene is highest in stomach, small intestine and pancreas, with lower levels in thyroid and very low levels in pituitary, fetal heart and adipose. Therefore, small molecule, peptide or antibody therapeutics designed using this gene product may be effective in modulating the development or activity of cellular processes in tissues that express this gene. Alternatively, these therapeutics may be used to alter the activity of these organs by modifying their innervation. In addition, this gene is expressed at higher levels in the adult lung (CT=30.9) when compared to expression in the fetal lung (CT=34.8). Thus, expression of this gene could be used to differentiate between adult and fetal sources of lung tissue. This gene is expressed at a low level in most of the cancer cell lines and normal tissues on this panel. Interestingly, pancreatic and brain cancer cell lines do not express this gene. Hence, the absence of expression of this gene could potentially be used as a diagnostic marker for pancreatic and brain cancer.

Panel 1.1 Summary: Ag568 Highest expression of the CG55704-01 gene is seen in the testis (CT=23.1). In addition, this gene is expressed at much higher levels in the testis than in any other samples on this panel. Thus, expression of this gene could be used as a marker of testis tissue. In addition, therapeutic modulation of the expression or function of this gene product may be beneficial in the treatment of male infertility. Expression of this gene among metabolically relevant tissues is highest in the small intestine, stomach and pancreas, with correlates well with expression in panel 1.4. Lower levels of expression are seen in heart, pituitary and adrenal. Therefore, small molecule, peptide or antibody therapeutics designed using this gene product may be effective in modulating the development or activity of cellular processes in tissues that express this gene. Alternatively, these therapeutics may be used to alter the activity of these organs by modifying their innervation. This panel also confirms a tissue expression profile that is highly brain-preferential for this gene. Please see panel CNS_Neurodegeneration for a discusion of utility of this gene in the central nervous system. Overall, this gene is expressed at a low level in most of the cancer cell lines and normal tissues on this panel. Interestingly, pancreatic and brain cancer cell lines do not express this gene. Hence the lack of expression of this gene can be used as a diagnostic marker for pancreatic and brain cancer.

Panel 1.3DSummary: Ag2879 Expression of the CG55704-01 gene is low/undetectable in all samples on this panel (CTs>35). (Data not shown.) A second experiment with probe and primer set Ag1486 is not included because the amp plot suggests that there were experimental difficulties with this run.

Panel 2.2 Summary: Ag1486 This gene is expressed at low but significant levels in this panel with highest expression seen in a normal colon tissue sample (CT=32.85). Single representatives of normal prostate, stomach, uterus and ovary samples also show higher expression compared to the adjacent cancer tissue. Hence, expression of this gene might be used as a marker to identify normal tissue from cancerous tissue in these organs.

Panel 2D Summary: Ag2879 Expression of the CG55704-01 gene is low/undetectable in all samples on this panel (CTs>35). (Data not shown.)

Panel 4.1D Summary: Ag4155 In two experiments with the same probe and primer set, the CG55704-01 transcript is expressed at low but significant levels in lung microvasculature treated with TNF-a and LA-4 and in colon. This transcript encodes an ephrin type receptor homolog, that belongs to a family of proteins which may play a role in integrin activity. Some members of this family have been described in vascular development. The regulation of the expression or activity of this protein product through the application of antibodies or small molecules may be important in controlling vascular morphogenesis, angiogenesis, leukocyte extravasation, and chemotaxis. Therefore, this gene product may be beneficial in the treatment of cancer. In addition, the protein encoded by this gene may also be useful in preventing the migration and accumulation to the lung to treat inflammatory lung diseases such asthma, emphysema or bronchitis. The presence of this transcript in the colon suggests that the protein encoded by this gene may also play a role in the development of the colon.Therapeutics that aim to regulate the function of this protein may function to regulate cellular processes within these tissues. Please note that a third run, Run 173333201, with the same probe and primer is not included, because the amp plot suggests that there were experimental difficulties with this run. References: Gu C, Park S. The EphA8 receptor regulates integrin activity through p110gamma phosphatidylinositol-3 kinase in a tyrosine kinase activity-independent manner. Mol Cell Biol 2001 July;21(14):4579-97. Recent genetic studies suggest that ephrins may function in a kinase-independent Eph receptor pathway. Here we report that expression of EphA8 in either NIH 3T3 or HEK293 cells enhanced cell adhesion to fibronectin via alpha(5)beta(1)- or beta(3) integrins. Interestingly, a kinase-inactive EphA8 mutant also markedly promoted cell attachment to fibronectin in these cell lines. Using a panel of EphA8 point mutants, we have demonstrated that EphA8 kinase activity does not correlate with its ability to promote cell attachment to fibronectin. Analysis using EphA8 extracellular and intracellular domain mutants has revealed that enhanced cell adhesion is dependent on ephrin A binding to the extracellular domain and the juxtamembrane segment of the cytoplasmic domain of the receptor. EphA8-promoted adhesion was efficiently inhibited by wortmannin, a phosphatidylinositol 3-kinase (PI 3-kinase) inhibitor. Additionally, we found that EphA8 had associated PI 3-kinase activity and that the p110gamma isoform of PI 3-kinase is associated with EphA8. In vitro binding experiments revealed that the EphA8 juxtamembrane segment was sufficient for the formation of a stable complex with p110gamma. Similar results were obtained in assay using cells stripped of endogenous ephrin A ligands by treatment with preclustered ephrin A5-Fc proteins. In addition, a membrane-targeted lipid kinase-inactive p110gamma mutant was demonstrated to stably associate with EphA8 and suppress EphA8-promoted cell adhesion to fibronectin. Taken together, these results suggest the presence of a novel mechanism by which the EphA8 receptor localizes p110gamma PI 3-kinase to the plasma membrane in a tyrosine kinase-independent fashion, thereby allowing access to lipid substrates to enable the signals required for integrin-mediated cell adhesion. Adams R H, Klein R. Eph receptors and ephrin ligands. essential mediators of vascular development. Trends Cardiovasc Med 2000 July; 10(5):183-8. The molecular and cellular mechanisms governing vascular development are still poorly understood. Prominent among the intercellular signals that control the initial establishment of the vascular network (termed vasculogenesis) and the subsequent remodeling process (called angiogenesis) are soluble ligands that signal through receptor tyrosine kinases (RTKs). Recent reports have added cell-bound ephrin ligands and their cognate Eph RTKs to the list of key players in vascular development.: J Biol Chem Apr. 27, 2001; 276(17):13771-7 Related Articles, Books, LinkOut. Adams R H, Wilkinson G A, Weiss C, Diella F, Gale N W, Deutsch U, Risau W, Klein R. Roles of ephrinB ligands and EphB receptors in cardiovascular development: demarcation of arterial/venous domains, Genes Dev Feb. 1, 1999; 13(3):295-306. Eph receptor tyrosine kinases and their cell-surface-bound ligands, the ephrins, regulate axon guidance and bundling in the developing brain, control cell migration and adhesion, and help patterning the embryo. Here we report that two ephrinB ligands and three EphB receptors are expressed in and regulate the formation of the vascular network. Mice lacking ephrinB2 and a proportion of double mutants deficient in EphB2 and EphB3 receptor signaling die in utero before embryonic day 11.5 (E11.5) because of defects in the remodeling of the embryonic vascular system. Our phenotypic analysis suggests complex interactions and multiple functions of Eph receptors and ephrins in the embryonic vasculature. Interaction between ephrinB2 on arteries and its EphB receptors on veins suggests a role in defining boundaries between arterial and venous domains. Expression of ephrinB1 by arterial and venous endothelial cells and EphB3 by veins and some arteries indicates that endothelial cell-to-cell interactions between ephrins and Eph receptors are not restricted to the border between arteries and veins. Furthermore, expression of ephrinB2 and EphB2 in mesenchyme adjacent to vessels and vascular defects in ephB2/ephB3 double mutants indicate a requirement for ephrin-Eph signaling between endothelial cells and surrounding mesenchymal cells. Finally, ephrinB ligands induce capillary sprouting in vitro with a similar efficiency as angiopoietin-1 (Ang1) and vascular endothelial growth factor (VEGF), demonstrating a stimulatory role of ephrins in the remodeling of the developing vascular system

Panel 4D Summary: Ag 1302/Ag1486 Two experiments with two different probe and primer sets show low but significant expression of the CG55704-01 gene in the colon and thymus. This expression is in agreement with the results from Panel 4.1D. The presence of this transcript in the thymus, and the colon suggests that the protein encoded by this gene may play a role in the development of these tissues. Thus, therapeutics that aim to regulate the function of the protein product may act to regulate the cellular processes within these tissues. Please note that a third experiment with the probe and primer set Ag2879 showed low/undetectable expression in all the samples on this panel (CTs>35).

G. CG55704-03: Ephrin Type-A Receptor 6 Precursor

Expression of gene CG55704-03 was assessed using the primer-probe sets Ag4155, Ag781, Ag568, Ag1486, Ag2879 and Ag1302, described in Tables 12GA, 12GB, 12GC, 12GD 12GE and 12GF. Results of the RTQ-PCR runs are shown in Tables 12GG, 12GH, 12GI, 12GJ, 12GK, 12GL, 12GM, and 12GN.

TABLE 12GA

Probe Name Ag4155

Start

| | | | |
|---|---|---|---|
| Primers | Sequences | Length | Position |

| | | | |
|---|---|---|---|
| Forward | 5′-acccaccttctatggcatgta-3′ | 21 | 983 |
| | (SEQ ID NO:143) | | |
| Probe | TET-5′-aggccaccttcagctcctaggaatgt-3′- | 26 | 1006 |
| | TAMRA (SEQ ID NO:144) | | |
| Reverse | 5′-gggctgtttcattgatgttaaa-3′ | 22 | 1036 |
| | (SEQ ID NO:145) | | |

TABLE 12GB

Probe Name Ag781

Start

| | | | |
|---|---|---|---|
| Primers | Sequences | Length | Position |

| | | | |
|---|---|---|---|
| Forward | 5′-aagagtaggtcagctgctcatg-3′ | 22 | 1519 |
| | (SEQ ID NO:146) | | |
| Probe | TET-5′-tcttctacccgcaggtagtgccaaaa-3′- | 26 | 1492 |
| | TAMRA (SEQ ID NO:147) | | |
| Reverse | 5′-agaaagtctacccacggatagc-3′ | 22 | 1463 |
| | (SEQ ID NO:148) | | |

TABLE 12GC

Probe Name Ag568

Start

| | | | |
|---|---|---|---|
| Primers | Sequences | Length | Position |

| | | | |
|---|---|---|---|
| Forward | 5′-agccccagaagccatcg-3′ | 17 | 2595 |
| | (SEQ ID NO:149) | | |
| Probe | TET-5′-ttctcctcagcaagcgatgcatgga-3′- | 25 | 2623 |
| | TAMRA (SEQ ID NO:150) | | |
| Reverse | 5′-ctcccacatgacaatgccatag-3′ | 22 | 2649 |
| | (SEQ ID NO:151) | | |

TABLE 12GD

Probe Name Ag1486

Start

| | | | |
|---|---|---|---|
| Primers | Sequences | Length | Position |

| | | | |
|---|---|---|---|
| Forward | 5′-tcccgggaattaaaacttacat-3′ | 22 | 1865 |
| | (SEQ ID NO:152) | | |
| Probe | TET-5′-cccatccctagcagtccatgaatttg-3′- | 26 | 1908 |
| | TAMRA (SEQ ID NO:153) | | |
| Reverse | 5′-tcttgagggatcaatctccttt-3′ | 22 | 1935 |
| | (SEQ ID NO:154) | | |

TABLE 12GE

Probe Name Ag2879

Start

| | | | |
|---|---|---|---|
| Primers | Sequences | Length | Position |

| | | | |
|---|---|---|---|
| Forward | 5′-gcagattattgctacgcaatg-3′ | 21 | 3398 |
| | (SEQ ID NO:155) | | |
| Probe | TET-5′-aaacctatctaggcccatgaatggaa-3′- | 26 | 3430 |
| | TAMRA (SEQ ID NO:156) | | |
| Reverse | 5′-aggatcggatttggatttgtt-3′ | 21 | 3456 |
| | (SEQ ID NO:157) | | |

TABLE 12GF

Probe Name Ag1302

Start

| | | | |
|---|---|---|---|
| Primers | Sequences | Length | Position |

| | | | |
|---|---|---|---|
| Forward | 5′-ggcagaaggagagaaatcaca-3′ | 21 | 2804 |
| | (SEQ ID NO:158) | | |
| Probe | TET-5′-actgacattgtcagcttccttgacaa-3′- | 26 | 2836 |
| | TAMRA (SEQ ID NO:159) | | |
| Reverse | 5′-cactgggatttcggatcagt-3′ | 20 | 2862 |
| | (SEQ ID NO:160) | | |

TABLE 12GG
CNS_neurodegeneration_v1.0

| | Rel. | | | | Rel. | | |
|---|---|---|---|---|---|---|---|
| | Exp. (%) | Rel. | Rel. | | Exp. (%) | Rel. | Rel. |
| | Ag4155, | EXP. (%) | Exp. (%) | | Ag4155, | Exp. (%) | Exp. (%) |
| | Run | Ag781, Run | Ag781, Run | Tissue | Run | Ag781, Run | Ag781, Run |
| Tissue Name | 215328490 | 225000477 | 237982181 | Name | 215328490 | 225000477 | 237982181 |
| AD 1 Hippo | 21.3 | 17.8 | 25.7 | Control | 8.4 | 11.0 | 7.6 |
| | | | | (Path) 3 | | | |
| | | | | Temporal | | | |
| | | | | Ctx | | | |
| AD 2 Hippo | 61.1 | 52.5 | 39.2 | Control | 47.6 | 49.0 | 33.2 |
| | | | | (Path) 4 | | | |
| | | | | Temporal | | | |
| | | | | Ctx | | | |
| AD 3 Hippo | 16.8 | 23.5 | 13.1 | AD 1 | 17.4 | 8.0 | 4.9 |
| | | | | Occipital | | | |
| | | | | Ctx | | | |
| AD 4 Hippo | 22.4 | 18.4 | 22.2 | AD 2 | 0.0 | 0.0 | 0.0 |
| | | | | Occipital | | | |
| | | | | Ctx | | | |
| | | | | (Missing) | | | |
| AD 5 hippo | 79.0 | 47.0 | 46.0 | AD 3 | 4.2 | 2.1 | 1.5 |
| | | | | Occipital | | | |
| | | | | Ctx | | | |
| AD 6 Hippo | 69.3 | 52.9 | 55.5 | AD 4 | 39.2 | 19.6 | 19.9 |
| | | | | Occipital | | | |
| | | | | Ctx | | | |
| Control 2 | 76.3 | 100.0 | 100.0 | AD 5 | 25.3 | 73.2 | 12.8 |
| Hippo | | | | Occipital | | | |
| | | | | Ctx | | | |
| Control 4 | 7.2 | 7.4 | 5.2 | AD 6 | 63.3 | 13.5 | 59.0 |
| Hippo | | | | Occipital | | | |
| | | | | Ctx | | | |
| Control | 10.0 | 17.1 | 11.7 | Control 1 | 4.0 | 3.4 | 2.8 |
| (Path) 3 | | | | Occipital | | | |
| Hippo | | | | Ctx | | | |
| AD 1 | 16.6 | 7.3 | 5.5 | Control 2 | 61.6 | 46.0 | 34.4 |
| Temporal | | | | Occipital | | | |
| Ctx | | | | Ctx | | | |
| AD 2 | 52.9 | 42.0 | 27.5 | Control 3 | 18.4 | 4.6 | 8.6 |
| Temporal | | | | OcciPital | | | |
| Ctx | | | | Ctx | | | |
| AD 3 | 6.8 | 3.6 | 7.2 | Control 4 | 7.9 | 8.0 | 4.5 |
| Temporal | | | | Occipital | | | |
| Ctx | | | | Ctx | | | |
| AD 4 | 46.7 | 33.9 | 26.6 | Control | 81.2 | 65.1 | 55.9 |
| Temporal | | | | (Path) 1 | | | |
| Ctx | | | | Occipital | | | |
| | | | | Ctx | | | |
| AD 5 Inf | 100.0 | 52.9 | 52.5 | Control | 16.3 | 8.7 | 6.3 |
| Temporal | | | | (Path) 2 | | | |
| Ctx | | | | Occipital | | | |
| | | | | Ctx | | | |
| AD 5 | 74.7 | 45.4 | 37.1 | Control | 2.8 | 2.1 | 2.6 |
| SupTemporal | | | | (Path) 3 | | | |
| Ctx | | | | Occipital | | | |
| | | | | Ctx | | | |
| AD 6 Inf | 31.2 | 14.3 | 15.6 | Control | 18.9 | 7.3 | 4.9 |
| Temporal | | | | (Path) 4 | | | |
| Ctx | | | | Occipital | | | |
| | | | | Ctx | | | |
| AD 6 Sup | 54.3 | 26.4 | 18.4 | Control 1 | 7.5 | 9.2 | 7.7 |
| Temporal | | | | Parietal | | | |
| Ctx | | | | Ctx | | | |
| Control 1 | 8.4 | 12.4 | 9.1 | Control 2 | 36.9 | 18.6 | 20.9 |
| Temporal | | | | Parietal | | | |
| Ctx | | | | Ctx | | | |
| Control 2 | 49.7 | 58.2 | 49.0 | Control 3 | 20.6 | 17.9 | 13.4 |
| Temporal | | | | Parietal | | | |
| Ctx | | | | Ctx | | | |
| Control 3 | 21.6 | 18.8 | 15.2 | Control | 97.9 | 96.6 | 72.2 |
| Temporal | | | | (Path) 1 | | | |
| Ctx | | | | Parietal | | | |
| | | | | Ctx | | | |
| Control 4 | 15.3 | 10.5 | 9.6 | Control | 43.5 | 25.0 | 15.9 |
| Temporal | | | | (Path) 2 | | | |
| Ctx | | | | Parietal | | | |
| | | | | Ctx | | | |
| Control | 89.5 | 78.5 | 66.4 | Control | 6.3 | 2.0 | 4.1 |
| (Path) 1 | | | | (Path) 3 | | | |
| Temporal | | | | Parietal | | | |
| Ctx | | | | Ctx | | | |
| Control | 55.5 | 41.5 | 33.7 | Control | 57.0 | 46.0 | 53.2 |
| (Path) 2 | | | | (Path) 4 | | | |
| Temporal | | | | Parietal | | | |
| Ctx | | | | Ctx | | | |

TABLE 12GH
General_screening_panel_v1.4

| | Rel. Exp.(%) Ag4155, | | Rel. Exp.(%) Ag4155, |
|---|---|---|---|
| Tissue Name | Run 222001153 | Tissue Name | Run 222001153 |
| Adipose | 0.8 | Renal ca. TK-10 | 7.0 |
| Melanoma* | 0.0 | Bladder | 1.1 |
| Hs688(A).T | | | |
| Melanoma* | 0.0 | Gastric ca. (liver | 0.2 |
| Hs688(B).T | | met.) NCI-N87 | |
| Melanoma* M14 | 0.0 | Gastric ca. KATO III | 0.0 |
| Melanoma* LOXIMVI | 0.3 | Colon ca. SW-948 | 0.0 |
| Melanoma* SK-MEL-5 | 0.0 | Colon ca. SW480 | 0.0 |
| Squamous cell | 0.0 | Colon ca.* (SW480 met) | 0.0 |
| carcinoma SCC-4 | | SW620 | |
| Testis Pool | 2.8 | Colon ca. HT29 | 0.0 |
| Prostate ca.* (bone | 6.9 | Colon ca. HCT-116 | 0.0 |
| met) PC-3 | | | |
| Prostate Pool | 7.2 | Colon ca. CaCo-2 | 6.7 |
| Placenta | 0.0 | Colon cancer tissue | 0.4 |
| Uterus Pool | 2.2 | Colon ca. SW1116 | 0.0 |
| Ovarian ca. OVCAR-3 | 2.2 | Colon ca. Colo-205 | 0.1 |
| Ovarian ca. SK-OV-3 | 3.5 | Colon ca. SW-48 | 0.0 |
| Ovarian ca. OVCAR-4 | 0.6 | Colon Pool | 10.8 |
| Ovarian ca. OVCAR-5 | 13.4 | Small Intestine Pool | 7.9 |
| Ovarian ca. IGROV-1 | 2.0 | Stomach Pool | 8.7 |
| Ovarian ca. OVCAR-8 | 1.2 | Bone Marrow Pool | 3.8 |
| Ovary | 3.8 | Fetal Heart | 0.8 |
| Breast ca. MCF-7 | 4.4 | Heart Pool | 3.1 |
| Breast ca. MDA-MB- | 0.0 | Lymph Node Pool | 7.2 |
| 231 | | | |
| Breast ca. BT 549 | 0.9 | Fetal Skeletal Muscle | 0.3 |
| Breast ca. T47D | 12.2 | Skeletal Muscle Pool | 0.1 |
| Breast ca. MDA-N | 0.0 | Spleen Pool | 0.0 |
| Breast Pool | 7.2 | Thymus Pool | 7.6 |
| Trachea | 0.6 | CNS cancer | 0.0 |
| | | (glio/astro) U87-MG | |
| Lung | 8.2 | CNS cancer | 0.6 |
| | | (glio/astro) U-118-MG | |
| Fetal Lung | 0.6 | CNS cancer (neuro;met) | 4.0 |
| | | SK-N-AS | |
| Lung ca. NCI-N417 | 2.2 | CNS cancer (astro) SF- | 0.0 |
| | | 539 | |
| Lung ca. LX-1 | 0.0 | CNS cancer (astro) | 0.0 |
| | | SNB-75 | |
| Lung ca. NCI-H146 | 2.4 | CNS cancer (glio) SNB- | 1.2 |
| | | 19 | |
| Lung ca. SHP-77 | 33.9 | CNS cancer (glio) SF- | 0.7 |
| | | 295 | |
| Lung ca. A549 | 0.0 | Brain (Amygdala) Pool | 22.1 |
| Lung ca. NCI-H526 | 0.5 | Brain (cerebellum) | 12.2 |
| Lung ca. NCI-H23 | 23.2 | Brain (fetal) | 100.0 |
| Lung ca. NCI-H460 | 0.0 | Brain (Hippocampus) | 37.9 |
| | | Pool | |
| Lung ca. HOP-62 | 0.5 | Cerebral Cortex Pool | 31.0 |
| Lung ca. NCI-H522 | 0.1 | Brain (Substantia | 21.2 |
| | | nigra) Pool | |
| Liver | 0.0 | Brain (Thalamus) Pool | 40.6 |
| Fetal Liver | 0.3 | Brain (whole) | 28.5 |
| Liver ca. HepG2 | 0.0 | Spinal Cord Pool | 4.5 |
| Kidney Pool | 15.1 | Adrenal Gland | 0.1 |
| Fetal Kidney | 2.5 | Pituitary gland Pool | 0.6 |
| Renal ca. 786-0 | 13.8 | Salivary Gland | 0.1 |
| Renal ca. A498 | 1.2 | Thyroid (female) | 1.5 |
| Renal ca. ACHN | 2.4 | Pancreatic ca. CAPAN2 | 0.0 |
| Renal ca. UO-31 | 0.4 | Pancreas Pool | 7.3 |

TABLE 12GI
Panel 1.1

| | Rel. Exp.(%) Ag568, | | Rel. Exp.(%) Ag568, |
|---|---|---|---|
| Tissue Name | Run 109491840 | Tissue Name | Run 109491840 |
| Adrenal gland | 0.1 | Renal ca. UO-31 | 0.0 |
| Bladder | 0.2 | Renal ca. RXF 393 | 0.0 |
| Brain (amygdala) | 17.9 | Liver | 0.0 |
| Brain (cerebellum) | 49.0 | Liver (fetal) | 0.0 |
| Brain (hippocampus) | 48.0 | Liver ca. | 0.0 |
| | | (hepatoblast) HepG2 | |
| Brain (substantia | 17.6 | Lung | 0.0 |
| nigra) | | | |
| Brain (thalamus) | 21.9 | Lung (fetal) | 0.0 |
| Cerebral Cortex | 24.3 | Lung ca. (non-s.cell) | 0.0 |
| | | HOP-62 | |
| Brain (fetal) | 54.7 | Lung ca. (large | 0.0 |
| | | cell)NCI-H460 | |
| Brain (whole) | 67.4 | Lung ca. (non-s.cell) | 4.8 |
| | | NCI-H23 | |
| glio/astro U-118-MG | 0.0 | Lung ca. (non-s.cl) | 0.0 |
| | | NCI-H522 | |
| astrocytoma SF-539 | 0.0 | Lung ca. (non-sm. | 0.0 |
| | | cell) A549 | |
| astrocytoma SNB-75 | 0.0 | Lung ca. (s.cell | 12.8 |
| | | var.) SHP-77 | |
| astrocytoma SW1783 | 0.0 | Lung ca. (small cell) | 0.0 |
| | | LX-1 | |
| glioma U251 | 0.0 | Lung ca. (small cell) | 5.8 |
| | | NCI-H69 | |
| glioma SF-295 | 0.0 | Lung ca. (squam.) SW | 0.5 |
| | | 900 | |
| glioma SNB-19 | 0.0 | Lung ca. (squam.) | 1.2 |
| | | NCI-H596 | |
| glio/astro U87-MG | 0.0 | Lymph node | 0.0 |
| neuro*; met SK-N-AS | 5.5 | Spleen | 0.0 |
| Mammary gland | 0.0 | Thymus | 0.0 |
| Breast ca. BT-549 | 0.0 | Ovary | 1.7 |
| Breast ca. MDA-N | 0.1 | Ovarian ca. IGROV-1 | 0.4 |
| Breast ca.* (pl.ef) | 1.1 | Ovarian ca. OVCAR-3 | 0.1 |
| T47D | | | |
| Breast ca.* (pl.ef) | 1.9 | Ovarian ca. OVCAR-4 | 0.0 |
| MCF-7 | | | |
| Breast ca.* (pl.ef) | 0.0 | Ovarian ca. OVCAR-5 | 8.8 |
| MDA-MB-231 | | | |
| Small intestine | 5.4 | Ovarian ca. OVCAR-8 | 0.5 |
| Colorectal | 0.6 | Ovarian ca.* | 0.4 |
| | | (ascites) SK-OV-3 | |
| Colon ca. HT29 | 0.2 | Pancreas | 2.8 |
| Colon ca. CaCo-2 | 0.0 | Pancreatic ca. CAPAN 2 | 0.0 |
| Colon ca. HCT-15 | 0.0 | Pituitary gland | 0.1 |
| Colon ca. HCT-116 | 0.0 | Placenta | 0.0 |
| Colon ca. HCC-2998 | 0.0 | Prostate | 3.6 |
| Colon ca. SW480 | 0.0 | Prostate ca.* (bone | 0.4 |
| | | met) PC-3 | |
| Colon ca.* SW620 | 0.0 | Salivary gland | 0.1 |
| (SW480 met) | | | |
| Stomach | 1.9 | Trachea | 0.1 |
| Gastric ca. (liver | 0.0 | Spinal cord | 1.5 |
| met) NCI-N87 | | | |
| Heart | 0.7 | Testis | 100.0 |
| Skeletal muscle | 0.0 | Thyroid | 3.0 |
| (Fetal) | | | |
| Skeletal muscle | 0.0 | Uterus | 0.3 |
| Endothelial cells | 0.0 | Melanoma M14 | 0.0 |
| Heart (Fetal) | 0.0 | Melanoma LOX IMVI | 0.0 |
| Kidney | 0.1 | Melanoma UACC-62 | 0.0 |
| Kidney (fetal) | 0.2 | Melanoma SK-MEL-28 | 0.0 |
| Renal ca. 786-0 | 1.4 | Melanoma* (met) SK- | 0.0 |
| | | MEL-5 | |
| Renal ca. A498 | 0.1 | Melanoma Hs688(A).T | 0.0 |
| Renal ca. ACHN | 0.0 | Melanoma* (met) | 0.0 |
| | | Hs688(B).T | |
| Renal ca. TK-10 | 2.6 | | |

TABLE 12GJ
Panel 1.2

| | Rel. Exp. (%) Ag781, | | Rel. Exp. (%) Ag781, |
|---|---|---|---|
| Tissue Name | Run 116762951 | Tissue Name | Run 116762951 |
| Endothelial cells | 1.4 | Renal ca. 786-0 | 2.3 |
| Heart (Fetal) | 0.4 | Renal ca. A498 | 1.8 |
| Pancreas | 3.3 | Renal ca. RXF 393 | 0.0 |
| Pancreatic ca. CAPAN 2 | 0.0 | Renal ca. ACHN | 1.7 |
| Adrenal Gland | 0.4 | Renal ca. UO-31 | 0.0 |
| Thyroid | 7.5 | Renal ca. TK-10 | 4.0 |
| Salivary gland | 0.7 | Liver | 0.2 |
| Pituitary gland | 1.3 | Liver (fetal) | 0.0 |
| Brain (fetal) | 18.2 | Liver ca. | 0.0 |
| | | (hepatoblast) HepG2 | |
| Brain (whole) | 41.5 | Lung | 0.0 |
| Brain (amygdala) | 23.8 | Lung (fetal) | 0.0 |
| Brain (cerebellum) | 17.8 | Lung ca. (small cell) | 0.0 |
| | | LX-1 | |
| Brain (hippocampus) | 34.9 | Lung ca. (small cell) | 0.2 |
| | | NCI-H69 | |
| Brain (thalamus) | 15.0 | Lung ca. (s.cell | 6.1 |
| | | var.) SHP-77 | |
| Cerebral Cortex | 100.0 | Lung ca. (large | 0.0 |
| | | cell) NCI-H460 | |
| Spinal cord | 2.3 | Lung ca. (non-sm. | 0.0 |
| | | cell) A549 | |
| glio/astro U87-MG | 0.0 | Lung ca. (non-s.cell) | 5.4 |
| | | NCI-H23 | |
| glio/astro U-118-MG | 0.0 | Lung ca. (non-s.cell) | 0.9 |
| | | HOP-62 | |
| astrocytoma SW1783 | 0.0 | Lung ca. (non-s.cl) | 0.2 |
| | | NCI-H522 | |
| neuro*; met SK-N-AS | 4.2 | Lung ca. (squam.) SW | 1.2 |
| | | 900 | |
| astrocytoma SF-539 | 0.0 | Lung ca. (squam.) | 0.0 |
| | | NCI-H596 | |
| astrocytoma SNB-75 | 0.0 | Mammary gland | 0.9 |
| glioma SNB-19 | 0.1 | Breast ca.* (pl.ef) | 2.6 |
| | | MCF-7 | |
| glioma U251 | 1.5 | Breast ca.* (pl.ef) | 0.0 |
| | | MDA-MB-231 | |
| glioma SF-295 | 0.1 | Breast ca.* (pl. ef) | 0.5 |
| | | T47D | |
| Heart | 1.3 | Breast ca. BT-549 | 0.4 |
| Skeletal Muscle | 0.3 | Breast ca. MDA-N | 0.0 |
| Bone marrow | 0.0 | Ovary | 8.3 |
| Thymus | 0.5 | Ovarian ca. OVCAR-3 | 3.1 |
| Spleen | 0.0 | Ovarian ca. OVCAR-4 | 0.5 |
| Lymph node | 0.0 | Ovarian ca. OVCAR-5 | 9.0 |
| Colorectal Tissue | 7.9 | Ovarian ca. OVCAR-8 | 0.9 |
| Stomach | 1.9 | Ovarian ca. IGROV-1 | 3.5 |
| Small intestine | 3.3 | Ovarian ca. (ascites) | 2.5 |
| | | SK-OV-3 | |
| Colon ca. SW480 | 0.0 | Uterus | 2.5 |
| Colon ca.* SW620 | 0.0 | Placenta | 0.0 |
| (SW480 met) | | | |
| Colon ca. HT29 | 0.0 | Prostate | 5.7 |
| Colon ca. HCT-116 | 0.0 | Prostate ca.* (bone | 0.6 |
| | | met) PC-3 | |
| Colon ca. CaCo-2 | 1.8 | Testis | 54.0 |
| Colon ca. Tissue | 0.0 | Melanoma Hs688(A).T | 0.0 |
| (ODO3866) | | | |
| Colon ca. HCC-2998 | 0.0 | Melanoma* (met) | 0.2 |
| | | Hs688(B).T | |
| Gastric ca.* (liver | 0.4 | Melanoma UACC-62 | 0.0 |
| met) NCI-N87 | | | |
| Bladder | 2.7 | Melanoma M14 | 0.0 |
| Trachea | 0.8 | Melanoma LOX IMVI | 0.0 |
| Kidney | 0.5 | Melanoma* (met) SK- | 0.0 |
| | | MEL-5 | |
| Kidney (fetal) | 1.2 | | |

TABLE 12GK
Panel 2.2

| | Rel. Exp. (%) | | Rel. Exp. (%) |
|---|---|---|---|
| | Ag1486, Run | | Ag1486, Run |
| Tissue Name | 173949464 | Tissue Name | 1173949464 |
| Normal Colon | 3.3 | Kidney Margin | 7.6 |
| | | (OD04348) | |
| Colon cancer (OD06064) | 3.1 | Kidney malignant | 0.0 |
| | | cancer (OD06204B) | |
| Colon Margin (OD06064) | 1.0 | Kidney normal adjacent | 0.0 |
| | | tissue (OD06204E) | |
| Colon cancer (OD06159) | 0.0 | Kidney Cancer | 0.0 |
| | | (OD04450-01) | |
| Colon Margin (OD06159) | 7.9 | Kidney Margin | 0.0 |
| | | (OD04450-03) | |
| Colon cancer (OD06297- | 0.0 | Kidney Cancer 8120613 | 3.3 |
| 04) | | | |
| Colon Margin (OD06297- | 100.0 | Kidney Margin 8120614 | 0.0 |
| 015) | | | |
| CC Gr.2 ascend colon | 0.0 | Kidney Cancer 9010320 | 3.1 |
| (ODO3921) | | | |
| CC Margin (ODO3921) | 0.0 | Kidney Margin 9010321 | 0.0 |
| Colon cancer metastasis | 0.0 | Kidney Cancer 8120607 | 2.3 |
| (OD06104) | | | |
| Lung Margin (OD06104) | 1.6 | Kidney Margin 8120608 | 0.0 |
| Colon mets to lung | 0.0 | Normal Uterus | 40.1 |
| (OD04451-01) | | | |
| Lung Margin (OD04451- | 5.1 | Uterine Cancer 064011 | 11.7 |
| 02) | | | |
| Normal Prostate | 0.0 | Normal Thyroid | 0.0 |
| Prostate Cancer | 0.0 | Thyroid Cancer 064010 | 0.0 |
| (OD04410) | | | |
| Prostate Margin | 18.6 | Thyroid Cancer A302152 | 0.0 |
| (OD04410) | | | |
| Normal Ovary | 5.3 | Thyroid Margin A302153 | 3.0 |
| Ovarian cancer | 0.0 | Normal Breast | 10.0 |
| (OD06283-03) | | | |
| Ovarian Margin | 0.0 | Breast Cancer | 0.0 |
| (OD06283-07) | | (OD04566) | |
| Ovarian Cancer 064008 | 5.2 | Breast Cancer 1024 | 0.0 |
| Ovarian cancer | 1.6 | Breast Cancer | 3.0 |
| (OD06145) | | (OD04590-01) | |
| Ovarian Margin | 17.1 | Breast Cancer Mets | 0.0 |
| (OD06145) | | (OD04590-03) | |
| Ovarian cancer | 4.6 | Breast Cancer | 0.0 |
| (OD06455-03) | | Metastasis (OD04655- | |
| | | 05) | |
| Ovarian Margin | 3.8 | Breast Cancer 064006 | 0.0 |
| (OD06455-07) | | | |
| Normal Lung | 2.5 | Breast Cancer 9100266 | 0.0 |
| Invasive poor diff. | 0.0 | Breast Margin 9100265 | 0.0 |
| lung adeno (ODO4945-01 | | | |
| Lung Margin (ODO4945- | 0.0 | Breast Cancer A209073 | 0.0 |
| 03) | | | |
| Lung Malignant Cancer | 0.0 | Breast Margin A2090734 | 0.0 |
| (OD03126) | | | |
| Lung Margin (OD03126) | 0.0 | Breast cancer | 0.0 |
| | | (OD06083) | |
| Lung Cancer (OD05014A) | 0.0 | Breast cancer node | 0.0 |
| | | metastasis (OD06083) | |
| Lung Margin (OD05014B) | 0.0 | Normal Liver | 0.0 |
| Lung cancer (OD06081) | 0.0 | Liver Cancer 1026 | 0.0 |
| Lung Margin (OD06081) | 0.0 | Liver Cancer 1025 | 0.0 |
| Lung Cancer (OD04237- | 0.0 | Liver Cancer 6004-T | 0.0 |
| 01) | | | |
| Lung Margin (OD04237- | 3.7 | Liver Tissue 6004-N | 0.0 |
| 02) | | | |
| Ocular Melanoma | 0.0 | Liver Cancer 6005-T | 0.0 |
| Metastasis | | | |
| Ocular Melanoma Margin | 0.0 | Liver Tissue 6005-N | 0.0 |
| (Liver) | | | |
| Melanoma Metastasis | 0.0 | Liver Cancer 064003 | 0.0 |
| Melanoma Margin (Lung) | 0.0 | Normal Bladder | 4.3 |
| Normal Kidney | 0.0 | Bladder Cancer 1023 | 0.0 |
| Kidney Ca, Nuclear | 0.0 | Bladder Cancer A302173 | 0.0 |
| grade 2 (OD04338) | | | |
| Kidney Margin (OD04338) | 0.0 | Normal Stomach | 55.9 |
| Kidney Ca Nuclear grade | 0.0 | Gastric Cancer 9060397 | 0.0 |
| ½ (OD04339) | | | |
| Kidney Margin (OD04339) | 0.0 | Stomach Margin 9060396 | 13.3 |
| Kidney Ca, Clear cell | 0.0 | Gastric Cancer 9060395 | 4.8 |
| type (OD04340) | | | |
| Kidney Margin (OD04340) | 3.8 | Stomach Margin 9060394 | 6.9 |
| Kidney Ca, Nuclear | 6.2 | Gastric Cancer 064005 | 0.0 |
| grade 3 (OD04348) | | | |

TABLE 12GL
Panel 4.1D

| | Rel. | Rel. | | Rel. | Rel. |
|---|---|---|---|---|---|
| | Exp.(%) | Exp.(%) | | Exp.(%) | Exp.(%) |
| | Ag4155, Run | Ag4155, Run | | Ag4155, Run | Ag4155, Run |
| Tissue Name | 173124973 | 174261191 | Tissue Name | 173124973 | 174261191 |
| Secondary Th1 act | 0.0 | 0.0 | HUVEC IL-1beta | 0.0 | 7.4 |
| Secondary Th2 act | 0.0 | 0.0 | HUVEC IFN gamma | 0.8 | 5.6 |
| Secondary Tr1 act | 0.0 | 0.0 | HUVEC TNF alpha + IFN | 0.6 | 0.0 |
| | | | gamma | | |
| Secondary Th1 rest | 0.0 | 0.0 | HUVEC TNF alpha + IL4 | 0.3 | 20.0 |
| Secondary Th2 rest | 0.0 | 0.0 | HUVEC IL-11 | 0.3 | 4.2 |
| Secondary Tr1 rest | 0.0 | 0.0 | Lung Microvascular | 0.9 | 14.6 |
| | | | EC none | | |
| Primary Th1 act | 0.0 | 0.0 | Lung Microvascular | 2.2 | 63.7 |
| | | | EC TNF alpha + IL- | | |
| | | | 1beta | | |
| Primary Th2 act | 0.0 | 0.0 | Microvascular | 0.0 | 0.0 |
| | | | Dermal EC none | | |
| Primary Tr1 act | 0.0 | 0.0 | Microvascular | 0.0 | 9.8 |
| | | | Dermal EC TNF alPha + | | |
| | | | IL-1beta | | |
| Primary Th1 rest | 0.0 | 0.0 | Bronchial | 0.0 | 0.0 |
| | | | epithelium | | |
| | | | TNF alPha + IL1beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 0.0 | 0.0 |
| | | | epithelium | | |
| | | | TNF alpha + IL-1beta | | |
| CD45RA CD4 | 0.0 | 0.0 | Coronery artery | 0.0 | 0.0 |
| lymphocyte act | | | SMC rest | | |
| CD45RO CD4 | 0.0 | 0.0 | Coronery artery | 0.0 | 11.7 |
| lymphocyte act | | | SMC TNF alpha + IL- | | |
| | | | 1beta | | |
| CD8 lymphocyte act | 0.0 | 0.0 | Astrocytes rest | 0.0 | 0.0 |
| Secondary CD8 | 0.0 | 0.0 | Astrocytes | 0.0 | 0.0 |
| lymphocyte rest | | | TNF alpha + IL-1beta | | |
| Secondary CD8 | 0.0 | 0.0 | KU-812 (Basophil) | 100.0 | 0.0 |
| lymphocyte act | | | rest | | |
| CD4 lymphocyte | 0.0 | 0.0 | KU-812 (Basophil) | 0.0 | 0.0 |
| none | | | PMA/ionomycin | | |
| 2ry | 0.0 | 0.0 | CCD01106 | 0.0 | 3.8 |
| Th1/Th2/Tr1_anti- | | | (Keratinocytes) | | |
| CD95 CH11 | | | none | | |
| LAK cells rest | 0.0 | 0.0 | CCD1106 | 0.0 | 0.0 |
| | | | (Keratinocytes) | | |
| | | | TNF alpha + IL- | | |
| | | | 1beta | | |
| LAK cells IL-2 | 0.0 | 0.0 | Liver cirrhosis | 3.7 | 100.0 |
| LAK cells IL-2 + IL- | 0.0 | 6.0 | NCI-H292 none | 0.0 | 0.0 |
| 12 | | | | | |
| LAK cells IL-2 + IFN | 0.0 | 0.0 | NCI-H292 IL-4 | 0.0 | 0.0 |
| gamma | | | | | |
| LAK cells IL-2 + IL- | 0.0 | 0.0 | NCI-H292 IL-9 | 0.0 | 0.0 |
| 18 | | | | | |
| LAK cells | 0.0 | 0.0 | NCI-H292 IL-13 | 0.3 | 12.6 |
| PMA/ionomycin | | | | | |
| NK Cells IL-2 rest | 0.0 | 0.0 | NCI-H292 IFN gamma | 0.0 | 0.0 |
| Two Way MLR 3 day | 0.0 | 0.0 | HPAEC none | 0.0 | 0.0 |
| Two Way MLR 5 day | 0.0 | 0.0 | HPAEC TNF alpha + IL- | 0.0 | 0.0 |
| | | | 1beta | | |
| Two Way MLR 7 day | 0.0 | 0.0 | Lung fibroblast | 0.3 | 0.0 |
| | | | none | | |
| PBMC rest | 0.0 | 0.0 | Lung fibroblast | 0.0 | 0.0 |
| | | | TNF alpha + IL-1 | | |
| | | | beta | | |
| PBMC PWM | 0.0 | 0.0 | Lung fibroblast | 0.0 | 2.8 |
| | | | IL-4 | | |
| PBMC PHA-L | 0.0 | 0.0 | Lung fibroblast | 0.0 | 0.0 |
| | | | IL-9 | | |
| Ramos (B cell) | 0.0 | 0.0 | Lung fibroblast | 0.0 | 0.0 |
| none | | | IL-13 | | |
| Ramos (B cell) | 0.0 | 0.0 | Lung fibroblast | 0.0 | 0.0 |
| ionomycin | | | IFN gamma | | |
| B lymphocytes PWM | 0.0 | 0.0 | Dermal fibroblast | 0.0 | 0.0 |
| | | | CCD1070 rest | | |
| B lymphocytes | 0.0 | 0.0 | Dermal fibroblast | 0.0 | 0.0 |
| CD40L and IL-4 | | | CCD1070 TNF alpha | | |
| EOL-1 dbcAMP | 0.0 | 0.0 | Dermal fibroblast | 0.0 | 0.0 |
| | | | CCD1070 IL-1beta | | |
| EOL-1 dbcAMP | 0.0 | 0.0 | Dermal fibroblast | 0.0 | 0.0 |
| PMA/ionomycin | | | IFN gamma | | |
| Dendritic cells | 0.0 | 0.0 | Dermal fibroblast | 0.0 | 12.7 |
| none | | | IL-4 | | |
| Dendritic cells | 0.0 | 0.0 | Dermal Fibroblasts | 0.0 | 0.0 |
| LPS | | | rest | | |
| Dendritic cells | 0.0 | 0.0 | Neutrophils | 0.0 | 0.0 |
| anti-CD40 | | | TNFa + LPS | | |
| Monocytes rest | 0.0 | 0.0 | Neutrophils rest | 0.0 | 4.1 |
| Monocytes LPS | 0.0 | 0.0 | Colon | 2.2 | 35.4 |
| Macrophages rest | 0.0 | 0.0 | Lung | 1.7 | 9.8 |
| Macrophages LPS | 0.0 | 0.0 | Thymus | 0.9 | 28.3 |
| HUVEC none | 0.0 | 0.0 | Kidney | 0.7 | 15.1 |
| HUVEC starved | 0.0 | 3.5 | | | |

TABLE 12GM
Panel 4D

| | Rel. | Rel. | | Rel. | Rel. |
|---|---|---|---|---|---|
| | Exp. (%) | Exp. (%) | | Exp. (%) | Exp. (%) |
| | Ag1302, Run | Ag1486, Run | | Ag1302, Run | Ag1486, Run |
| Tissue Name | 138881940 | 162599619 | Tissue Name | 138881940 | 162599619 |
| Secondary Th1 act | 0.0 | 0.0 | HUVEC IL-1beta | 0.0 | 0.0 |
| Secondary Th2 act | 0.0 | 0.0 | HUVEC IFN gamma | 6.2 | 0.0 |
| Secondary Tr1 act | 0.0 | 0.0 | HUVEC TNF alpha + IFN | 0.0 | 11.4 |
| | | | gamma | | |
| Secondary Th1 rest | 0.0 | 0.0 | HUVEC TNF alpha + IL4 | 6.6 | 0.0 |
| Secondary Th2 rest | 0.0 | 0.0 | HUVEC IL-11 | 6.9 | 0.0 |
| Secondary Tr1 rest | 0.0 | 0.0 | Lung Microvascular | 6.0 | 15.0 |
| | | | EC none | | |
| Primary Th1 act | 0.0 | 0.0 | Lung Microvascular | 0.0 | 0.0 |
| | | | EC TNF alpha + IL- | | |
| | | | 1beta | | |
| Primary Th2 act | 0.0 | 0.0 | Microvascular | 7.0 | 0.0 |
| | | | Dermal EC none | | |
| Primary Tr1 act | 0.0 | 0.0 | Microsvasular Dermal | 0.0 | 0.0 |
| | | | EC TNF alpha + IL- | | |
| | | | 1beta | | |
| Primary Th1 rest | 0.0 | 0.0 | Bronchial | 0.0 | 0.0 |
| | | | epithelium | | |
| | | | TNF alpha + IL1beta | | |
| Primary Th2 rest | 0.0 | 0.0 | Small airway | 0.0 | 0.0 |
| | | | epithelium none | | |
| Primary Tr1 rest | 0.0 | 0.0 | Small airway | 6.7 | 0.0 |
| | | | epithelium | | |
| | | | TNF alpha + IL- | | |
| | | | 1beta | | |
| CD45RA CD4 | 0.0 | 0.0 | Coronery artery | 0.0 | 0.0 |
| lymphocyte act | | | SMC rest | | |
| CD45RO CD4 | 0.0 | 0.0 | Coronery artery | 0.0 | 0.0 |
| lymphocyte act | | | SMC TNF alpha + IL- | | |
| | | | 1beta | | |
| CD8 lymphocyte act | 0.0 | 0.0 | Astrocytes rest | 0.0 | 0.0 |
| Secondary CD8 | 0.0 | 0.0 | Astrocytes | 0.0 | 0.0 |
| lymphocyte rest | | | TNF alpha + IL- | | |
| | | | 1beta | | |
| Secondary CD8 | 0.0 | 0.0 | KU-812 (Basophil) | 0.0 | 0.0 |
| lymphocyte act | | | rest | | |
| CD4 lymphocyte | 0.0 | 0.0 | KU-812 (Basophil) | 6.3 | 0.0 |
| none | | | PMA/ionomycin | | |
| 2ry | 9.0 | 0.0 | CCD1106 | 0.0 | 0.0 |
| Th1/Th2/Tr1_anti- | | | (Keratinocytes) | | |
| CD95 CH11 | | | none | | |
| LAK cells rest | 0.0 | 0.0 | CCD1106 | 6.9 | 0.0 |
| | | | (Keratinocytes) | | |
| | | | TNF alpha + IL- | | |
| | | | 1beta | | |
| LAK cells IL-2 | 0.0 | 0.0 | Liver cirrhosis | 34.9 | 27.9 |
| LAK cells IL-2 + IL- | 0.0 | 0.0 | Lupus kidney | 0.0 | 8.2 |
| 12 | | | | | |
| LAK cells IL-2 + IFN | 9.2 | 0.0 | NCI-H292 none | 0.0 | 0.0 |
| gamma | | | | | |
| LAK cells IL-2 + IL- | 0.0 | 0.0 | NCI-H292 IL-4 | 0.0 | 0.0 |
| 18 | | | | | |
| LAK cells | 0.0 | 0.0 | NCI-H292 IL-9 | 15.0 | 0.0 |
| PMA/ionomycin | | | | | |
| NK Cells IL-2 rest | 0.0 | 0.0 | NCI-H292 IL-13 | 0.0 | 0.0 |
| Two Way MLR 3 day | 0.0 | 0.0 | NCI-H292 IFN gamma | 0.0 | 0.0 |
| Two Way MLR 5 day | 0.0 | 0.0 | HPAEC none | 0.0 | 0.0 |
| Two Way MLR 7 day | 0.0 | 0.0 | HPAEC TNF alpha + IL- | 0.0 | 0.0 |
| | | | 1 beta | | |
| PBMC rest | 0.0 | 0.0 | Lung fibroblast | 0.0 | 0.0 |
| | | | none | | |
| PBMC PWM | 0.0 | 0.0 | Lung fibroblast | 0.0 | 0.0 |
| | | | TNF alpha + IL-1 | | |
| | | | beta | | |
| PBMC PHA-L | 15.3 | 0.0 | Lung fibroblast | 0.0 | 0.0 |
| | | | IL-4 | | |
| Ramos (B cell) | 0.0 | 0.0 | Lung fibroblast | 0.0 | 0.0 |
| none | | | IL-9 | | |
| Ramos (B cell) | 0.0 | 0.0 | Lung fibroblast | 0.0 | 0.0 |
| ionomycin | | | IL-13 | | |
| B lymphocytes PWM | 0.0 | 0.0 | Lung fibroblast | 0.0 | 0.0 |
| | | | IFN gamma | | |
| B lymphocytes | 0.0 | 0.0 | Dermal fibroblast | 0.0 | 0.0 |
| CD40L and IL-4 | | | CCD1070 rest | | |
| EOL-1 dbcAMP | 0.0 | 0.0 | Dermal fibroblast | 0.0 | 0.0 |
| | | | CCD1070 TNF alpha | | |
| EOL-1 dbcAMP | 0.0 | 0.0 | Dermal fibroblast | 0.0 | 0.0 |
| PMA/ionomycin | | | CCD1070 IL-1 beta | | |
| Dendritic cells | 0.0 | 0.0 | Dermal fibroblast | 0.0 | 0.0 |
| none | | | IFN gamma | | |
| Dendritic cells | 0.0 | 0.0 | Dermal fibroblast | 0.0 | 0.0 |
| LPS | | | IL-4 | | |
| Dendritic cells | 0.0 | 0.0 | IBD Colitis 2 | 100.0 | 58.2 |
| anti-CD40 | | | | | |
| Monocytes rest | 0.0 | 0.0 | IBD Crohn's | 30.1 | 13.3 |
| Monocytes LPS | 0.0 | 0.0 | Colon | 81.8 | 97.3 |
| Macrophages rest | 0.0 | 0.0 | Lung | 0.0 | 15.7 |
| Macrophages LPS | 0.0 | 0.0 | Thymus | 45.7 | 100.0 |
| HUVEC none | 0.0 | 0.0 | Kidney | 16.0 | 12.2 |
| HUVEC starved | 0.0 | 0.0 | | | |

TABLE 12GN
Panel CNS_1

| | Rel. Exp. (%) Ag781, Run | | Rel. Exp. (%) Ag781, Run |
|---|---|---|---|
| Tissue Name | 171694577 | Tissue Name | 171694577 |
| BA4 Control | 36.1 | BA17 PSP | 6.2 |
| BA4 Control2 | 59.9 | BA17 PSP2 | 11.7 |
| BA4 Alzheimer's2 | 0.0 | Sub Nigra Control | 21.5 |
| BA4 Parkinson's | 52.5 | Sub Nigra Control2 | 40.3 |
| BA4 Parkinson's2 | 74.2 | Sub Nigra | 25.5 |
| | | Alzheimer's2 | |
| BA4 Huntington's | 51.8 | Sub Nigra | 26.6 |
| | | Parkinson's2 | |
| BA4 | 7.3 | Sub Nigra | 55.1 |
| Huntington's2 | | Huntington's | |
| BA4 PSP | 3.3 | Sub Nigra | 14.1 |
| | | Huntington's2 | |
| BA4 PSP2 | 21.2 | Sub Nigra PSP2 | 1.2 |
| BA4 Depression | 14.1 | Sub Nigra Depression | 8.4 |
| BA4 Depression2 | 4.1 | Sub Nigra Depression2 | 5.9 |
| BA7 Control | 21.3 | Glob Palladus Control | 10.5 |
| BA7 Control2 | 41.2 | Glob Palladus | 22.1 |
| | | Control2 | |
| BA7 Alzheimer's2 | 11.0 | Glob Palladus | 11.7 |
| | | Alzheimer's | |
| BA7 Parkinson's | 11.3 | Glob Palladus | 0.0 |
| | | Alzheimer's2 | |
| BA7 Parkinson's2 | 43.5 | Glob Palladus | 39.8 |
| | | Parkinson's | |
| BA7 Huntington's | 18.6 | Glob Palladus | 9.5 |
| | | Parkinson's2 | |
| BA7 | 10.0 | Glob Palladus PSP | 7.6 |
| Huntington's2 | | | |
| BA7 PSP | 20.3 | Glob Palladus PSP2 | 10.4 |
| BA7 PSP2 | 13.4 | Glob Palladus | 11.1 |
| | | Depression | |
| BA7 Depression | 10.8 | Temp Pole Control | 30.6 |
| BA9 Control | 33.4 | Temp Pole Control2 | 100.0 |
| BA9 Control2 | 78.5 | Temp Pole Alzheimer's | 1.3 |
| BA9 Alzheimer's | 9.4 | Temp Pole | 6.7 |
| | | Alzheimer's2 | |
| BA9 Alzheimer's2 | 15.0 | Temp Pole Parkinson's | 26.8 |
| BA9 Parkinson's | 22.2 | Temp Pole | 40.6 |
| | | Parkinson's2 | |
| BA9 Parkinson's2 | 25.5 | Temp Pole | 67.8 |
| | | Huntington's | |
| BA9 Huntington's | 34.2 | Temp Pole PSP | 9.2 |
| BA9 | 7.5 | Temp Pole PSP2 | 11.7 |
| Huntington's2 | | | |
| BA9 PSP | 14.7 | Temp Pole Depression2 | 6.3 |
| BA9 PSP2 | 0.0 | Cing Gyr Control | 27.0 |
| BA9 Depression | 8.2 | Cing Gyr Control2 | 23.8 |
| BA9 Depression2 | 8.8 | Cing Gyr Alzheimer's | 10.4 |
| BA17 Control | 10.4 | Cing Gyr Alzheimer's2 | 9.7 |
| BA17 Control2 | 43.8 | Cing Gyr Parkinson's | 21.2 |
| BA17 | 3.1 | Cing Gyr Parkinson's2 | 7.8 |
| Alzheimer's2 | | | |
| BA17 Parkinson's | 11.2 | Cing Gyr Huntington's | 41.2 |
| BA17 | 11.4 | Cing Gyr | 23.7 |
| Parkinson's2 | | Huntington's2 | |
| BA17 | 24.5 | Cing Gyr PSP | 12.8 |
| Huntington's | | | |
| BA17 | 3.0 | Cing GYr PSP2 | 19.6 |
| Huntington's2 | | | |
| BA17 Depression | 0.0 | Cing Gyr Depression | 10.7 |
| BA17 Depression2 | 6.9 | Cing Gyr Depression2 | 22.2 |

CNS_neurodegeneration_v1.0 Summary: Ag781/Ag4155 The CG55704-03 gene encodes a putative ephrin receptor, and shows a significant downregulation in the AD temporal cortex compared to nondemented controls when CT values are analyzed by ANCOVA. The temporal cortex (Brodmann area 21) shows severe neurodegeneration in Alzheimer's disease, though not as early as the hippocampus or entorhinal cortex. It is therefore likely that this gene is downregulated during the process of neurodegeneration, rather than the downregulation being a result of neuron loss. The ephrin receptors have been implicated in axonal and synapse guidance. Furthermore, individuals with Alzheimer's disease (especially late-onset AD with apoE4 genotype) show impaired compensatory synaptogenesis and dendritic arborization. Therefore, this gene is an excellent small molecule target for the treatment of Alzheimer's disease. Please note that one experiment with the probe and primer set Ag2879 is not included because the amp plot indicates that there were experimental difficulties with this run. References:Lai K O, Ip F C, Cheung J, Fu A K, Ip N Y. Expression of Eph receptors in skeletal muscle and their localization at the neuromuscular junction. Mol Cell Neurosci 2001 June; 17(6):1034-47. The participation of ephrins and Eph receptors in guiding motor axons during muscle innervation has been well documented, but little is known about their expression and functional significance in muscle at later developmental stages. Our present study investigates the expression and localization of Eph receptors and ephrins in skeletal muscle. Prominent expression of EphA4, EphA7, and ephrin-A ligands was detected in muscle during embryonic development. More importantly, both EphA4 and EphA7, as well as ephrin-A2, were localized at the neuromuscular junction (NMJ) of adult muscle. Despite their relative abundance, they were not localized at the synapses during embryonic stages. The concentration of EphA4, EphA7, and ephrin-A2 at the NMJ was observed at postnatal stages and the synaptic localization became prominent at later developmental stages. In addition, expression of Eph receptors was increased by neuregulin and after nerve injury. Furthermore, we demonstrated that overexpression of EphA4 led to tyrosine phosphorylation of the actin-binding protein cortactin and that EphA4 was coimmunoprecipitated with cortactin in muscle. Taken together, our findings indicate that EphA4 is associated with the actin cytoskeleton. Since actin cytoskeleton is critical to the formation and stability of NMJ, the present findings raise the intriguing possibility that Eph receptors may have a novel role in NMJ formation and/or maintenance. Arendt T, Schindler C, Bruckner M K, Eschrich K, Bigl V, Zedlick D, Marcova L. Plastic neuronal remodeling is impaired in patients with Alzheimer's disease carrying apolipoprotein epsilon 4 allele. J Neurosci Jan. 15, 1997;17(2):516-29. A relationship between the apolipoprotein E (apoE) genotype and the risk to develop Alzheimer's disease has been established recently. Apolipoprotein synthesis is implicated in developmental processes and in neuronal repair of the adult nervous system. In the present study, we investigated the influence of the apolipoprotein polymorphism on the severity of neuronal degeneration and the extent of plastic dendritic remodeling in Alzheimer's disease. Changes in length and arborization of dendrites of Golgi-impregnated neurons in the basal nucleus of Meynert, locus coeruleus, raphe magnus nucleus, medial amygdaloid nucleus, pedunculopontine tegmental nucleus, and substantia nigra were analyzed after three-dimensional reconstruction. Patients with either one or two apoE epsilon 4 alleles not only showed a more severe degeneration in all areas investigated than in patients lacking the apoE 4 allele but also revealed significantly less plastic dendritic changes. apoE epsilon 4 allele copy number, furthermore, had a significant effect on the pattern of dendritic arborization. Moreover, the relationship between the intensity of dendritic growth and both the extent of neuronal degeneration and the stage of the disease seen in patients lacking the apoE epsilon 4 allele was very weak in the presence of one epsilon 4 allele and completely lost in patients homozygous for the epsilon 4 allele. The results provide direct evidence that neuronal reorganization is affected severely in patients with Alzheimer's disease carrying the apoE epsilon 4 allele. This impairment of neuronal repair might lead to a more rapid functional decompensation, thereby contributing to an earlier onset and more rapid progression of the disease. Feldheim D A, Vanderhaeghen P, Hansen M J, Frisen J, Lu Q, Barbacid M, Flanagan J G. Topographic guidance labels in a sensory projection to the forebrain. Neuron 1998 December;21(6):1303-13. Visual connections to the mammalian forebrain are known to be patterned by neural activity, but it remains unknown whether the map topography of such higher sensory projections depends on axon guidance labels. Here, we show complementary expression and binding for the receptor EphA5 in mouse retina and its ligands ephrin-A2 and ephrin-A5 in multiple retinal targets, including the major forebrain target, the dorsal lateral geniculate nucleus (dLGN). These ligands can act in vitro as topographically specific repellents for mammalian retinal axons and are necessary for normal dLGN mapping in vivo. The results suggest a general and economic modular mechanism for brain mapping whereby a projecting field is mapped onto multiple targets by repeated use of the same labels. They also indicate the nature of a coordinate system for the mapping of sensory connections to the forebrain.

General_screening_panel_v1.4 Summary: Ag4155 The CG55704-03 gene shows a tissue expression profile that is highly brain-preferential, with highest expression in the fetal brain (CT=27.3). Please see panel CNS_Neurodegeneration for a discussion of utility of this gene in the central nervous system. Among metabolically relevant tissues, expression of this gene is highest in stomach, small intestine and pancreas, with lower levels in thyroid and very low levels in pituitary, fetal heart and adipose. Therefore, small molecule, peptide or antibody therapeutics designed using this gene product may be effective in modulating the development or activity of cellular processes in tissues that express this gene. Alternatively, these therapeutics may be used to alter the activity of these organs by modifying their innervation. In addition, this gene is expressed at higher levels in the adult lung (CT=30.9) when compared to expression in the fetal lung (CT=34.8). Thus, expression of this gene could be used to differentiate between adult and fetal sources of lung tissue. This gene is expressed at a low level in most of the cancer cell lines and normal tissues on this panel. Interestingly, pancreatic and brain cancer cell lines do not express this gene. Hence, the absence of expression of this gene could potentially be used as a diagnostic marker for pancreatic and brain cancer.

Panel 1.1 Summary: Ag568 Highest expression of the CG55704-03 gene is seen in the testis (CT=23.1). In addition, this gene is expressed at much higher levels in the testis than in any other samples on this panel. Thus, expression of this gene could be used as a marker of testis tissue. In addition, therapeutic modulation of the expression or function of this gene product may be beneficial in the treatment of male infertility. Expression of this gene among metabolically relevant tissues is highest in the small intestine, stomach and pancreas, with correlates well with expression in panel 1.4. Lower levels of expression are seen in heart, pituitary and adrenal. Therefore, small molecule, peptide or antibody therapeutics designed using this gene product may be effective in modulating the development or activity of cellular processes in tissues that express this gene. Alternatively, these therapeutics may be used to alter the activity of these organs by modifying their innervation. This panel also confirms a tissue expression profile that is highly brain-preferential for this gene. Please see panel CNS_Neurodegeneration for a discusion of utility of this gene in the central nervous system. Overall, this gene is expressed at a low level in most of the cancer cell lines and normal tissues on this panel. Interestingly, pancreatic and brain cancer cell lines do not express this gene. Hence the lack of expression of this gene can be used as a diagnostic marker for pancreatic and brain cancer.

Panel 1.2 Summary: Ag781 Highest expression of the CG55704-03 gene in this panel is seen in the cerebral cortex (CT=28.8). This panel confirms a tissue expression profile that is highly brain-preferential for this gene. Please see panel CNS_Neurodegeneration for a discusion of utility of this gene in the central nervous system. Among metabolically relevant tissues, expression of this gene is seen in pancreas, small intestine and stomach at moderate levels. This is consistent with expression in panel 1.4 and panel 1.1. Therefore, small molecule, peptide or antibody therapeutics designed using this gene product may be effective in modulating the development or activity of cellular processes in tissues that express this gene. Alternatively, these therapeutics may be used to alter the activity of these organs by modifying their innervation. Overall, this gene is expressed at a low level in most of the cancer cell lines and normal tissues on this panel. Interestingly, pancreatic and brain cancer cell lines do not express this gene. Hence the lack of expression of this gene can be used as a diagnostic marker for pancreatic and brain cancer.

Panel 1.3D Summary: Ag2879 Expression of the CG55704-03 gene is low/undetectable in all samples on this panel (CTs>35). (Data not shown.) A second experiment with probe and primer set Ag1486 is not included because the amp plot suggests that there were experimental difficulties with this run.

Panel 2.2 Summary: Ag1486 This gene is expressed at low but significant levels in this panel with highest expression seen in a normal colon tissue sample (CT=32.85). Single representatives of normal prostate, stomach, uterus and ovary samples also show higher expression compared to the adjacent cancer tissue. Hence, expression of this gene might be used as a marker to identify normal tissue from cancerous tissue in these organs.

Panel 2D Summary: Ag2879 Expression of the CG55704-03 gene is low/undetectable in all samples on this panel (CTs>35). (Data not shown.)

Panel 4.1D Summary: Ag4155 In two experiments with the same probe and primer set, the CG55704-03 transcript is expressed at low but significant levels in lung microvasculature treated with TNF-a and IL-4 and in colon. This transcript encodes an ephrin type receptor homolog, that belongs to a family of proteins that may play a role in integrin activity. Some members of this family have been described in vascular development. The regulation of the expression or activity of this protein product through the application of antibodies or small molecules may be important in controlling vascular morphogenesis, angiogenesis, leukocyte extravasation, and chemotaxis. Therefore, this gene product may be beneficial in the treatment of cancer. In addition, the protein encoded by this gene may also be useful in preventing the migration and accumulation of leukocytes to the lung to treat inflammatory lung diseases such asthma, emphysema or bronchitis. The presence of this transcript in the colon suggests that the protein encoded by this gene may also play a role in the development of the colon.Therapeutics that aim to regulate the function of this protein may function to regulate cellular processes within these tissues. Please note that a third run, Run 173333201, with the same probe and primer is not included, because the amp plot suggests that there were experimental difficulties with this run. References: Gu C, Park S. The EphA8 receptor regulates integrin activity through p110gamma phosphatidylinositol-3 kinase in a tyrosine kinase activity-independent manner. Mol Cell Biol 2001 July;21(14):4579-97. Recent genetic studies suggest that ephrins may function in a kinase-independent Eph receptor pathway. Here we report that expression of EphA8 in either NIH 3T3 or HEK293 cells enhanced cell adhesion to fibronectin via alpha(5)beta(1)- or beta(3) integrins. Interestingly, a kinase-inactive EphA8 mutant also markedly promoted cell attachment to fibronectin in these cell lines. Using a panel of EphA8 point mutants, we have demonstrated that EphA8 kinase activity does not correlate with its ability to promote cell attachment to fibronectin. Analysis using EphA8 extracellular and intracellular domain mutants has revealed that enhanced cell adhesion is dependent on ephrin A binding to the extracellular domain and the juxtamembrane segment of the cytoplasmic domain of the receptor. EphA8-promoted adhesion was efficiently inhibited by wortmannin, a phosphatidylinositol 3-kinase (PI 3-kinase) inhibitor. Additionally, we found that EphA8 had associated PI 3-kinase activity and that the p110gamma isoform of PI 3-kinase is associated with EphA8. In vitro binding experiments revealed that the EphA8 juxtamembrane segment was sufficient for the formation of a stable complex with p110gamma. Similar results were obtained in assay using cells stripped of endogenous ephrin A ligands by treatment with preclustered ephrin A5-Fc proteins. In addition, a membrane-targeted lipid kinase-inactive p110gamma mutant was demonstrated to stably associate with EphA8 and suppress EphA8-promoted cell adhesion to fibronectin. Taken together, these results suggest the presence of a novel mechanism by which the EphA8 receptor localizes p110gamma PI 3-kinase to the plasma membrane in a tyrosine kinase-independent fashion, thereby allowing access to lipid substrates to enable the signals required for integrin-mediated cell adhesion. Adams R H, Klein R. Eph receptors and ephrin ligands. essential mediators of vascular development. Trends Cardiovasc Med 2000 July; 10(5):183-8. The molecular and cellular mechanisms governing vascular development are still poorly understood. Prominent among the intercellular signals that control the initial establishment of the vascular network (termed vasculogenesis) and the subsequent remodeling process (called angiogenesis) are soluble ligands that signal through receptor tyrosine kinases (RTKs). Recent reports have added cell-bound ephrin ligands and their cognate Eph RTKs to the list of key players in vascular development.: J Biol Chem Apr. 27, 2001; 276(17):13771-7 Related Articles, Books, LinkOut. Adams R H, Wilkinson G A, Weiss C, Diella F, Gale N W, Deutsch U, Risau W, Klein R. Roles of ephrinB ligands and EphB receptors in cardiovascular development: demarcation of arterial/venous domains, Genes Dev Feb. 1, 1999; 13(3):295-306. Eph receptor tyrosine kinases and their cell-surface-bound ligands, the ephrins, regulate axon guidance and bundling in the developing brain, control cell migration and adhesion, and help patterning the embryo. Here we report that two ephrinB ligands and three EphB receptors are expressed in and regulate the formation of the vascular network. Mice lacking ephrinB2 and a proportion of double mutants deficient in EphB2 and EphB3 receptor signaling die in utero before embryonic day 11.5 (E11.5) because of defects in the remodeling of the embryonic vascular system. Our phenotypic analysis suggests complex interactions and multiple functions of Eph receptors and ephrins in the embryonic vasculature. Interaction between ephrinB2 on arteries and its EphB receptors on veins suggests a role in defining boundaries between arterial and venous domains. Expression of ephrinB1 by arterial and venous endothelial cells and EphB3 by veins and some arteries indicates that endothelial cell-to-cell interactions between ephrins and Eph receptors are not restricted to the border between arteries and veins. Furthermore, expression of ephrinB2 and EphB2 in mesenchyme adjacent to vessels and vascular defects in ephB2/ephB3 double mutants indicate a requirement for ephrin-Eph signaling between endothelial cells and surrounding mesenchymal cells. Finally, ephrinB ligands induce capillary sprouting in vitro with a similar efficiency as angiopoietin-1 (Ang1) and vascular endothelial growth factor (VEGF), demonstrating a stimulatory role of ephrins in the remodeling of the developing vascular system

Panel 4D Summary: Ag 1302/Ag1486 Two experiments with two different probe and primer sets show low but significant expression of the CG55704-03 gene in the colon and thymus. This expression is in agreement with the results from Panel 4.1D. The presence of this transcript in the thymus, and the colon suggests that the protein encoded by this gene may play a role in the development of these tissues. Thus, therapeutics that aim to regulate the function of the protein product may act to regulate the cellular processes within these tissues. Please note that a third experiment with the probe and primer set Ag2879 showed low/undetectable expression in all the samples on this panel (CTs>35). (Data not shown.)

Panel CNS_{—}1 Summary: Ag781 This panel confirms a tissue expression profile that is highly brain-preferential; see panel CNS_Neurodegeneration for a discussion of utility the CG55704-03 gene in the central nervous system.

H. CG95545-01/CG95545-01 and CG95545-02: Type IA Membrane Sushi-Containing Domain Protein

Expression of gene CG95545-01 and variant CG95545-02 was assessed using the primer-probe sets Ag4000, Ag1923 and Ag729, described in Tables 12HA, 12HB and 12HC. Results of the RTQ-PCR runs are shown in Tables 12HD, 12HE, 12HF, 12HG, 12HH and 12HI.

TABLE 12HA

Probe Name Ag4000

Start

| | | | |
|---|---|---|---|
| Primers | Sequences | Length | Position |

| | | | |
|---|---|---|---|
| Forward | 5′-atgcttgcagagaaggattctt-3′ | 22 | 919 |
| | (SEQ ID NO:161) | | |
| Probe | TET-5′-atacagtttcaagctgcacaggcctg-3′- | 26 | 955 |
| | TAMRA (SEQ ID NO:162) | | |
| Reverse | 5′-tctcttggcaatgtaattttgg-3′ | 22 | 996 |
| | (SEQ ID NO:163) | | |

TABLE 12HB

Probe Name Ag1923

Start

| | | | |
|---|---|---|---|
| Primers | Sequences | Length | Position |

| | | | |
|---|---|---|---|
| Forward | 5′-ccctacaaatccatagttgcaa-3′ | 22 | 782 |
| | (SEQ ID NO:164) | | |
| Probe | TET-5′-ttcttcccttctctttgctggcatgt-3′- | 26 | 447 |
| | TAMRA (SEQ ID NO:165) | | |
| Reverse | 5′-gtttagacgtctgtgccacttg-3′ | 22 | 412 |
| | (SEQ ID NO:166) | | |

TABLE 12HC

Probe Name Ag729

Start

| | | | |
|---|---|---|---|
| Primers | Sequences | Length | Position |

| | | | |
|---|---|---|---|
| Forward | 5′-ccctacaaatccatagttgcaa-3′ | 22 | 482 |
| | (SEQ ID NO:167) | | |
| Probe | TET-5′-ttcttcccttctctttgctggcatgt-3′- | 26 | 447 |
| | TAMRA (SEQ ID NO:168) | | |
| Reverse | 5′-gtttagacgtctgtgccacttg-3′ | 22 | 412 |
| | (SEQ ID NO:169) | | |

TABLE 12HD
CNS_neurodegeneration_v1.0

| | Rel. Exp. (%) Ag4000, | | Rel Exp. (%) Ag4000, |
|---|---|---|---|
| Tissue Name | Run 212391726 | Tissue Name | Run 212391726 |
| AD 1 Hippo | 5.3 | Control (Path) 3 | 5.8 |
| | | Temporal Ctx | |
| AD 2 Hippo | 19.2 | Control (Path) 4 | 44.4 |
| | | Temporal Ctx | |
| AD 3 Hippo | 4.0 | AD 1 Occipital Ctx | 11.7 |
| AD 4 Hippo | 6.3 | AD 2 Occipital Ctx | 0.0 |
| | | (Missing) | |
| AD 5 hippo | 92.7 | AD 3 Occipital Ctx | 3.4 |
| AD 6 Hippo | 21.9 | AD 4 Occipital Ctx | 16.8 |
| Control 2 Hippo | 28.9 | AD 5 Occipital Ctx | 12.9 |
| Control 4 Hippo | 6.0 | AD 6 Occipital Ctx | 47.0 |
| Control (Path) 3 | 5.2 | Control 1 Occipital | 3.3 |
| Hippo | | Ctx | |
| AD 1 Temporal Ctx | 8.7 | Control 2 Occipital | 57.0 |
| | | Ctx | |
| AD 2 Temporal Ctx | 29.1 | Control 3 Occipital | 13.9 |
| | | Ctx | |
| AD 3 Temporal Ctx | 4.6 | Control 4 Occipital | 3.5 |
| | | Ctx | |
| AD 4 Temporal Ctx | 21.5 | Control (Path) 1 | 77.9 |
| | | Occipital Ctx | |
| AD 5 Inf Temporal | 100.0 | Control (Path) 2 | 10.7 |
| Ctx | | Occipital Ctx | |
| AD 5 SupTemporal Ctx | 40.9 | Control (Path) 3 | 2.3 |
| | | Occipital Ctx | |
| AD 6 Inf Temporal | 27.0 | Control (Path) 4 | 13.8 |
| Ctx | | Occipital Ctx | |
| AD 6 Sup Temporal | 36.1 | Control 1 Parietal | 6.3 |
| Ctx | | Ctx | |
| Control 1 Temporal | 9.0 | Control 2 Parietal | 48.3 |
| Ctx | | Ctx | |
| Control 2 Temporal | 50.3 | Control 3 Parietal | 18.7 |
| Ctx | | Ctx | |
| Control 3 Temporal | 19.1 | Control (Path) 1 | 81.8 |
| Ctx | | Parietal Ctx | |
| Control 4 Temporal | 6.7 | Control (Path) 2 | 19.5 |
| Ctx | | Parietal Ctx | |
| Control (Path) 1 | 74.2 | Control (Path) 3 | 2.5 |
| Temporal Ctx | | Parietal Ctx | |
| Control (Path) 2 | 29.7 | Control (Path) 4 | 42.0 |
| Temporal Ctx | | Parietal Ctx | |

TABLE 12HE
General_screening_panel_v1.4

| | Rel. Exp. (%) Ag1923, | | Rel. Exp. (%) Ag1923, |
|---|---|---|---|
| Tissue Name | Run 216595201 | Tissue Name | Run 216595201 |
| Adipose | 12.9 | Renal ca. TK-10 | 56.6 |
| Melanoma* | 28.5 | Bladder | 14.8 |
| Hs688(A).T | | | |
| Melanoma* | 28.9 | Gastric ca. (liver | 45.4 |
| Hs688(B).T | | met.) NCI-N87 | |
| Melanoma* M14 | 1.6 | Gastric ca. KATO III | 55.9 |
| Melanoma* LOXIMVI | 0.6 | Colon ca. SW-948 | 7.6 |
| Melanoma* SK-MEL-5 | 0.3 | Colon ca. SW480 | 63.3 |
| Squamous cell | 3.0 | Colon ca.*(SW480 met) | 24.5 |
| carcinoma SCC-4 | | SW620 | |
| Testis Pool | 15.0 | Colon ca. HT29 | 19.5 |
| Prostate ca.* (bone | 8.5 | Colon ca. HCT-116 | 17.1 |
| met) PC-3 | | | |
| Prostate Pool | 8.8 | Colon ca. CaCo-2 | 100.0 |
| Placenta | 38.7 | Colon cancer tissue | 28.9 |
| Uterus Pool | 1.9 | Colon ca. SW1116 | 1.4 |
| Ovarian ca. OVCAR-3 | 18.4 | Colon ca. Colo-205 | 7.1 |
| Ovarian ca. SK-OV-3 | 24.8 | Colon ca. SW-48 | 15.3 |
| Ovarian ca. OVCAR-4 | 11.9 | Colon Pool | 16.6 |
| Ovarian ca. OVCAR-5 | 26.6 | Small Intestine Pool | 7.0 |
| Ovarian ca. IGROV-1 | 4.2 | Stomach Pool | 13.1 |
| Ovarian ca. OVCAR-8 | 10.7 | Bone Marrow Pool | 4.9 |
| Ovary | 6.8 | Fetal Heart | 14.7 |
| Breast ca. MCF-7 | 12.7 | Heart Pool | 6.3 |
| Breast ca. MDA-MB- | 68.3 | Lymph Node Pool | 13.3 |
| 231 | | | |
| Breast ca. BT 549 | 24.7 | Fetal Skeletal Muscle | 9.2 |
| Breast ca. T47D | 38.7 | Skeletal Muscle Pool | 3.1 |
| Breast ca. MDA-N | 0.0 | Spleen Pool | 13.6 |
| Breast Pool | 14.0 | Thymus Pool | 21.3 |
| Trachea | 16.7 | CNS cancer | 31.4 |
| | | (glio/astro) U87-MG | |
| Lung | 2.2 | CNS cancer | 4.1 |
| | | (glio/astro) U-118-MG | |
| Fetal Lung | 59.0 | CNS cancer (neuro; met) | 31.9 |
| | | SK-N-AS | |
| Lung ca. NCI-N417 | 2.9 | CNS cancer (astro) SF- | 12.9 |
| | | 539 | |
| Lung ca. LX-1 | 22.4 | CNS cancer (astro) | 14.5 |
| | | SNB-75 | |
| Lung ca. NCI-H146 | 8.1 | CNS cancer (glio) SNB- | 1.9 |
| | | 19 | |
| Lung ca. SHP-77 | 30.8 | CNS cancer (glio) SF- | 13.8 |
| | | 295 | |
| Lung ca. A549 | 38.7 | Brain (Amygdala) Pool | 15.1 |
| Lung ca. NCI-H526 | 1.8 | Brain (cerebellum) | 11.3 |
| Lung ca. NCI-H23 | 12.6 | Brain (fetal) | 59.0 |
| Lung ca. NCI-H460 | 13.3 | Brain (Hippocampus) | 19.3 |
| | | Pool | |
| Lung ca. HOP-62 | 15.6 | Cerebral Cortex Pool | 16.6 |
| Lung ca. NCI-H522 | 2.0 | Brain (Substantia | 14.2 |
| | | nigra) Pool | |
| Liver | 1.2 | Brain (Thalamus) Pool | 25.3 |
| Fetal Liver | 27.4 | Brain (whole) | 52.9 |
| Liver ca. HepG2 | 25.7 | Spinal Cord Pool | 7.2 |
| Kidney Pool | 15.2 | Adrenal Gland | 24.7 |
| Fetal Kidney | 51.8 | Pituitary gland Pool | 6.6 |
| Renal ca. 786-0 | 7.0 | Salivary Gland | 10.6 |
| Renal ca. A498 | 6.8 | Thyroid (female) | 3.7 |
| Renal ca. ACHN | 17.4 | Pancreatic ca. CAPAN2 | 45.4 |
| Renal ca. UO-31 | 67.4 | Pancreas Pool | 18.9 |

TABLE 12HF
Panel 1.2

| | Rel. Exp. (%) Ag729, | | Rel. Exp. (%) Ag729, |
|---|---|---|---|
| Tissue Name | Run 115216357 | Tissue Name | Run 115216357 |
| Endothelial cells | 19.2 | Renal ca. 786-0 | 4.7 |
| Heart (Fetal) | 3.0 | Renal ca. A498 | 8.2 |
| Pancreas | 24.1 | Renal ca. RXF 393 | 13.7 |
| Pancreatic ca. CAPAN 2 | 6.9 | Renal ca. ACHN | 20.0 |
| Adrenal Gland | 22.4 | Renal ca. UO-31 | 14.8 |
| Thyroid | 19.3 | Renal ca. TK-10 | 43.5 |
| Salivary gland | 19.9 | Liver | 9.2 |
| Pituitary gland | 48.3 | Liver (fetal) | 20.3 |
| Brain (fetal) | 49.0 | Liver ca. | 18.8 |
| | | (hepatoblast) HepG2 | |
| Brain (whole) | 27.7 | Lung | 11.2 |
| Brain (amygdala) | 15.9 | Lung (fetal) | 15.1 |
| Brain (cerebellum) | 6.0 | Lung ca. (small cell) | 24.8 |
| | | LX-1 | |
| Brain (hippocampus) | 16.0 | Lung ca. (small cell) | 5.3 |
| | | NCI-H69 | |
| Brain (thalamus) | 9.5 | Lung ca. (s.cell | 6.9 |
| | | var.) SHP-77 | |
| Cerebral Cortex | 41.2 | Lung ca. (large | 12.3 |
| | | cell)NCI-H460 | |
| Spinal cord | 5.5 | Lung ca. (non-sm. | 24.7 |
| | | cell) A549 | |
| glio/astro U87-MG | 23.5 | Lung ca. (non-s.cell) | 4.0 |
| | | NCI-H23 | |
| glio/astro U-118-MG | 1.0 | Lung ca. (non-s.cell) | 20.6 |
| | | HOP-62 | |
| astrocytoma SW1783 | 2.3 | Lung ca. (non-s.cl) | 2.2 |
| | | NCI-H522 | |
| neuro*; met SK-N-AS | 37.1 | Lung ca. (squam.) SW | 12.0 |
| | | 900 | |
| astrocytoma SF-539 | 16.6 | Lung ca. (squam.) | 18.9 |
| | | NCI-H596 | |
| astrocytoma SNB-75 | 2.0 | Mammary gland | 11.9 |
| glioma SNB-19 | 8.4 | Breast ca.* (pl.ef) | 17.1 |
| | | MCF-7 | |
| glioma U251 | 4.0 | Breast ca.* (pl.ef) | 43.8 |
| | | MDA-MB-231 | |
| glioma SF-295 | 3.6 | Breast ca.* (pl. ef) | 13.5 |
| | | T47D | |
| Heart | 19.2 | Breast ca. BT-549 | 4.5 |
| Skeletal Muscle | 11.8 | Breast ca. MDA-N | 0.2 |
| Bone marrow | 10.4 | Ovary | 5.3 |
| Thymus | 6.5 | Ovarian ca. OVCAR-3 | 19.6 |
| Spleen | 8.7 | Ovarian ca. OVCAR-4 | 3.2 |
| Lymph node | 17.2 | Ovarian ca. OVCAR-5 | 37.9 |
| Colorectal Tissue | 3.4 | Ovarian ca. OVCAR-8 | 33.0 |
| Stomach | 21.8 | Ovarian ca. IGROV-1 | 1.5 |
| Small intestine | 30.6 | Ovarian ca. (ascites) | 14.9 |
| | | SK-OV-3 | |
| Colon ca. SW480 | 17.9 | Uterus | 5.8 |
| Colon ca.* SW620 | 49.7 | Placenta | 72.7 |
| (SW480 met) | | | |
| Colon ca. HT29 | 17.9 | Prostate | 11.4 |
| Colon ca. HCT-116 | 13.2 | Prostate ca.* (bone | 12.0 |
| | | met) PC-3 | |
| Colon ca. CaCo-2 | 100.0 | Testis | 12.0 |
| Colon ca. Tissue | 11.3 | Melanoma Hs688(A).T | 6.8 |
| (ODO3866) | | | |
| Colon ca. HCC-2998 | 62.0 | Melanoma* (met) | 7.7 |
| | | Hs688(B).T | |
| Gastric ca.* (liver | 31.0 | Melanoma UACC-62 | 0.1 |
| met) NCI-N87 | | | |
| Bladder | 20.4 | Melanoma M14 | 0.2 |
| Trachea | 5.4 | Melanoma LOX IMVI | 0.0 |
| Kidney | 58.2 | Melanoma* (met) SK- | 0.0 |
| | | MEL-5 | |
| Kidney (fetal) | 46.3 | | |

TABLE 12HG
Panel 2.2

| | Rel. Exp. (%) | | Rel. Exp. (%) |
|---|---|---|---|
| | Ag1923, Run | | Ag1923, Run |
| Tissue Name | 174285446 | Tissue Name | 174285446 |
| Normal Colon | 17.3 | Kidney Margin | 100.0 |
| | | (OD04348) | |
| Colon cancer (OD06064) | 21.5 | Kidney malignant | 5.6 |
| | | cancer (OD06204B) | |
| Colon Margin (OD06064) | 18.3 | Kidney normal adjacent | 17.9 |
| | | tissue (OD06204E) | |
| Colon cancer (OD06159) | 5.0 | Kidney Cancer | 41.8 |
| | | (OD04450-01) | |
| Colon Margin (OD06159) | 19.1 | Kidney Margin | 25.2 |
| | | (OD04450-03) | |
| Colon cancer (OD06297- | 15.6 | Kidney Cancer 8120613 | 15.2 |
| 04) | | | |
| Colon Margin (OD06297- | 9.3 | Kidney Margin 8120614 | 12.8 |
| 015) | | | |
| CC Gr.2 ascend colon | 9.4 | Kidney Cancer 9010320 | 6.9 |
| (ODO3921) | | | |
| CC Margin (ODO3921) | 9.7 | Kidney Margin 9010321 | 8.2 |
| Colon cancer metastasis | 10.5 | Kidney Cancer 8120607 | 18.2 |
| (OD06104) | | | |
| Lung Margin (OD06104) | 17.2 | Kidney Margin 8120608 | 14.6 |
| Colon mets to lung | 33.9 | Normal Uterus | 29.1 |
| (OD04451-01) | | | |
| Lung Margin (OD04451- | 21.6 | Uterine Cancer 064011 | 11.5 |
| 02) | | | |
| Normal Prostate | 6.7 | Normal Thyroid | 2.3 |
| Prostate Cancer | 2.3 | Thyroid Cancer 064010 | 12.5 |
| (OD04410) | | | |
| Prostate Margin | 8.4 | Thyroid Cancer A302152 | 20.4 |
| (OD04410) | | | |
| Normal Ovary | 7.9 | Thyroid Margin A302153 | 1.0 |
| Ovarian cancer | 9.0 | Normal Breast | 26.6 |
| (OD06283-03) | | | |
| Ovarian Margin | 4.8 | Breast Cancer | 2.0 |
| (OD06283-07) | | (OD04566) | |
| Ovarian Cancer 064008 | 7.2 | Breast Cancer 1024 | 20.9 |
| Ovarian cancer | 6.0 | Breast Cancer | 18.2 |
| (OD06145) | | (OD04590-01) | |
| Ovarian Margin | 13.5 | Breast Cancer Mets | 12.5 |
| (OD06145) | | (OD04590-03) | |
| Ovarian cancer | 4.0 | Breast Cancer | 6.3 |
| (OD06455-03) | | Metastasis (OD04655- | |
| | | 05) | |
| Ovarian Margin | 7.9 | Breast Cancer 064006 | 8.7 |
| (OD06455-07) | | | |
| Normal Lung | 9.7 | Breast Cancer 9100266 | 11.0 |
| Invasive poor diff. | 19.6 | Breast Margin 9100265 | 23.5 |
| lung adeno (ODO4945-01 | | | |
| Lung Margin (ODO4945- | 26.2 | Breast Cancer A209073 | 6.1 |
| 03) | | | |
| Lung Malignant Cancer | 8.5 | Breast Margin A2090734 | 9.2 |
| (OD03126) | | | |
| Lung Margin (OD03126) | 7.9 | Breast cancer | 20.2 |
| | | (OD06083) | |
| Lung Cancer (OD05014A) | 12.3 | Breast cancer node | 11.7 |
| | | metastasis (OD06083) | |
| Lung Margin (OD05014B) | 45.1 | Normal Liver | 5.3 |
| Lung cancer (OD06081) | 13.6 | Liver Cancer 1026 | 5.5 |
| Lung Margin (OD06081) | 8.7 | Liver Cancer 1025 | 15.4 |
| Lung Cancer (OD04237- | 2.8 | Liver Cancer 6004-T | 11.0 |
| 01) | | | |
| Lung Margin (OD04237- | 18.7 | Liver Tissue 6004-N | 5.4 |
| 02) | | | |
| Ocular Melanoma | 11.4 | Liver Cancer 6005-T | 12.4 |
| Metastasis | | | |
| Ocular Melanoma Margin | 7.4 | Liver Tissue 6005-N | 19.8 |
| (Liver) | | | |
| Melanoma Metastasis | 6.5 | Liver Cancer 064003 | 4.7 |
| Melanoma Margin (Lung) | 26.1 | Normal Bladder | 4.3 |
| Normal Kidney | 17.9 | Bladder Cancer 1023 | 6.3 |
| Kidney Ca, Nuclear | 33.9 | Bladder Cancer A302173 | 6.7 |
| grade 2 (OD04338) | | | |
| Kidney Margin (OD04338) | 14.8 | Normal Stomach | 36.3 |
| Kidney Ca Nuclear grade | 72.2 | Gastric Cancer 9060397 | 5.2 |
| ½ (OD04339) | | | |
| Kidney Margin (OD04339) | 17.9 | Stomach Margin 9060396 | 20.4 |
| Kidney Ca, Clear cell | 2.5 | Gastric Cancer 9060395 | 9.2 |
| type (OD04340) | | | |
| Kidney Margin (OD04340) | 22.4 | Stomach Margin 9060394 | 14.9 |
| Kidney Ca, Nuclear | 6.3 | Gastric Cancer 064005 | 6.7 |
| grade 3 (OD04348) | | | |

TABLE 12HH
Panel 4.1D

| | Rel. Exp. (%) | | Rel. Exp. (%) |
|---|---|---|---|
| | Ag4000, Run | | Ag4000, Run |
| Tissue Name | 171492105 | Tissue Name | 171492105 |
| Secondary Th1 act | 27.0 | HUVEC IL-1beta | 25.3 |
| Secondary Th2 act | 44.1 | HUVEC IFN gamma | 36.3 |
| Secondary Tr1 act | 34.9 | HUVEC TNF alpha + IFN | 26.4 |
| | | gamma | |
| Secondary Th1 rest | 15.9 | HUVEC TNF alpha + IL4 | 22.2 |
| Secondary Th2 rest | 25.0 | HUVEC IL-11 | 19.3 |
| Secondary Tr1 rest | 21.3 | Lung Microvascular EC | 63.7 |
| | | none | |
| Primary Th1 act | 8.4 | Lung Microvascular EC | 34.6 |
| | | TNF alpha + IL-1beta | |
| Primary Th2 act | 13.6 | Microvascular Dermal EC | 18.0 |
| | | none | |
| Primary Tr1 act | 10.9 | Microsvasular Dermal EC | 14.0 |
| | | TNF alpha + IL-1beta | |
| Primary Th1 rest | 11.6 | Bronchial epithelium | 8.2 |
| | | TNF alpha + IL1beta | |
| Primary Th2 rest | 14.1 | Small airway epithelium | 11.5 |
| | | none | |
| Primary Tr1 rest | 13.7 | Small airway epithelium | 14.1 |
| | | TNF alpha + IL-1beta | |
| CD45RA CD4 lymphocyte | 19.2 | Coronery artery SMC rest | 15.7 |
| act | | | |
| CD45RO CD4 lymphocyte | 10.4 | Coronery artery SMC | 15.7 |
| act | | TNF alpha + IL-1beta | |
| CD8 lymphocyte act | 12.4 | Astrocytes rest | 17.3 |
| Secondary CD8 | 9.7 | Astrocytes TNF alpha + IL- | 17.2 |
| lymphocyte rest | | 1beta | |
| Secondary CD8 | 13.7 | KU-812 (Basophil) rest | 46.7 |
| lymphocyte act | | | |
| CD4 lymphocyte none | 8.0 | KU-812 (Basophil) | 100.0 |
| | | PMA/ionomycin | |
| 2ry Th1/Th2/Tr1_anti- | 31.9 | CCD1106 (Keratinocytes) | 9.1 |
| CD95 CH11 | | none | |
| LAK cells rest | 17.3 | CCD1106 (Keratinocytes) | 6.5 |
| | | TNF alpha + IL-1beta | |
| LAK cells IL-2 | 14.3 | Liver cirrhosis | 4.4 |
| LAK cells IL-2 + IL-12 | 15.2 | NCI-H292 none | 20.9 |
| LAK cells IL-2 + IFN | 13.7 | NCI-H292 IL-4 | 33.2 |
| gamma | | | |
| LAK cells IL-2 + IL-18 | 14.0 | NCI-H292 IL-9 | 31.9 |
| LAK cells | 14.1 | NCI-H292 IL-13 | 36.9 |
| PMA/ionomycin | | | |
| NK Cells IL-2 rest | 67.4 | NCI-H292 IFN gamma | 26.6 |
| Two Way MLR 3 day | 18.6 | HPAEC none | 27.5 |
| Two Way MLR 5 day | 15.2 | HPAEC TNF alpha + IL-1 | 34.4 |
| | | beta | |
| Two Way MLR 7 day | 17.6 | Lung fibroblast none | 33.4 |
| PBMC rest | 27.9 | Lung fibroblast TNF alpha + IL- | 26.6 |
| | | 1beta | |
| PBMC PWM | 21.5 | Lung fibroblast IL-4 | 23.3 |
| PBMC PHA-L | 12.7 | Lung fibroblast IL-9 | 30.1 |
| Ramos (B cell) none | 0.2 | Lung fibroblast IL-13 | 28.3 |
| Ramos (B cell) | 0.3 | Lung fibroblast IFN gamma | 33.9 |
| ionomycin | | | |
| B lymphocytes PWM | 7.9 | Dermal fibroblast CCD1070 | 27.2 |
| | | rest | |
| B lymphocytes CD40L | 7.9 | Dermal fibroblast CCD1070 | 66.0 |
| and IL-4 | | TNF alpha | |
| EOL-1 dbcAMP | 32.5 | Dermal fibroblast CCD1070 | 16.7 |
| | | IL-1beta | |
| EOL-1 dbcAMP | 17.6 | Dermal fibroblast IFN | 21.9 |
| PMA/ionomycin | | gamma | |
| Dendritic cells none | 34.4 | Dermal fibroblast IL-4 | 28.7 |
| Dendritic cells LPS | 40.9 | Dermal Fibroblasts rest | 26.6 |
| Dendritic cells anti- | 67.8 | Neutrophils TNFa + LPS | 8.7 |
| CD40 | | | |
| Monocytes rest | 62.4 | Neutrophils rest | 21.9 |
| Monocytes LPS | 78.5 | Colon | 7.5 |
| Macrophages rest | 45.4 | Lung | 39.8 |
| Macrophages LPS | 13.2 | Thymus | 26.8 |
| HUVEC none | 22.4 | Kidney | 40.3 |
| HUVEC starved | 33.4 | | |

TABLE 12HI
Panel 4D

| | Rel. Exp. (%) | | Rel. Exp. (%) |
|---|---|---|---|
| | Ag1923, Run | | Ag1923, Run |
| Tissue Name | 158535645 | Tissue Name | 158535645 |
| Secondary Th1 act | 14.3 | HUVEC IL-1beta | 20.2 |
| Secondary Th2 act | 24.7 | HUVEC IFN gamma | 35.6 |
| Secondary Tr1 act | 27.5 | HUVEC TNF alpha + IFN | 20.4 |
| | | gamma | |
| Secondary Th1 rest | 11.0 | HUVEC TNF alpha + IL4 | 22.1 |
| Secondary Th2 rest | 16.4 | HUVEC IL-11 | 18.7 |
| Secondary Tr1 rest | 12.3 | Lung Microvascular EC | 26.6 |
| | | none | |
| Primary Th1 act | 8.4 | Lung Microvascular EC | 20.9 |
| | | TNF alpha + IL-1beta | |
| Primary Th2 act | 11.6 | Microvascular Dermal EC | 22.4 |
| | | none | |
| Primary Tr1 act | 10.2 | Microsvasular Dermal EC | 18.9 |
| | | TNF alpha + IL-1beta | |
| Primary Th1 rest | 53.2 | Bronchial epithelium | 5.1 |
| | | TNF alpha + IL1beta | |
| Primary Th2 rest | 30.1 | Small airway epithelium | 6.7 |
| | | none | |
| Primary Tr1 rest | 13.4 | Small airway epithelium | 20.6 |
| | | TNF alpha + IL-1beta | |
| CD45RA CD4 lymphocyte | 10.5 | Coronery artery SMC rest | 18.7 |
| act | | | |
| CD45RO CD4 lymphocyte | 17.3 | Coronery artery SMC | 7.9 |
| act | | TNFalPha + IL-1beta | |
| CD8 lymphocyte act | 6.3 | Astrocytes rest | 26.8 |
| Secondary CD8 | 9.8 | Astrocytes TNF alpha + IL- | 20.4 |
| lymphocyte rest | | 1beta | |
| Secondary CD8 | 25.0 | KU-812 (Basophil) rest | 43.5 |
| lymphocyte act | | | |
| CD4 lymphocyte none | 7.9 | KU-812 (Basophil) | 100.0 |
| | | PMA/ionomycin | |
| 2ry Th1/Th2/Tr1_anti- | 25.2 | CCD1106 (Keratinocytes) | 4.5 |
| CD95 CH11 | | none | |
| LAK cells rest | 17.4 | CCD1106 (Keratinocytes) | 2.2 |
| | | TNF alpha + IL-1beta | |
| LAK cells IL-2 | 15.0 | Liver cirrhosis | 3.9 |
| LAK cells IL-2 + IL-12 | 14.4 | Lupus kidney | 3.1 |
| LAK cells IL-2 + IFN | 20.2 | NCI-H292 none | 33.9 |
| gamma | | | |
| LAK cells IL-2+ IL-18 | 25.7 | NCI-H292 IL-4 | 51.1 |
| LAK cells | 11.9 | NCI-H292 IL-9 | 36.3 |
| PMA/ionomycin | | | |
| NK Cells IL-2 rest | 38.4 | NCI-H292 IL-13 | 26.6 |
| Two Way MLR 3 day | 13.4 | NCI-H292 IFN gamma | 21.3 |
| Two Way MLR 5 day | 8.5 | HPAEC none | 18.7 |
| Two Way MLR 7 day | 8.7 | HPAEC TNF alpha + IL-1 | 16.7 |
| | | beta | |
| PBMC rest | 18.4 | Lung fibroblast none | 18.3 |
| PBMC PWM | 27.2 | Lung fibroblast TNF alpha + IL- | 13.4 |
| | | 1beta | |
| PBMC PHA-L | 11.9 | Lung fibroblast IL-4 | 35.4 |
| Ramos (B cell) none | 0.7 | Lung fibroblast IL-9 | 18.3 |
| Ramos (B cell) | 1.1 | Lung fibroblast IL-13 | 22.1 |
| ionomycin | | | |
| B lymphocytes PWM | 23.0 | Lung fibroblast IFN gamma | 29.3 |
| B lymphocytes CD40L | 6.8 | Dermal fibroblast CCD1070 | 20.3 |
| and IL-4 | | rest | |
| EOL-1 dbcAMP | 17.7 | Dermal fibroblast CCD1070 | 64.6 |
| | | TNF alpha | |
| EOL-1 dbcAMP | 20.6 | Dermal fibroblast CCD1070 | 18.0 |
| PMA/ionomycin | | IL-1beta | |
| Dendritic cells none | 25.7 | Dermal fibroblast IFN | 17.4 |
| | | gamma | |
| Dendritic cells LPS | 36.6 | Dermal fibroblast IL-4 | 19.2 |
| Dendritic cells anti- | 33.0 | IBD Colitis 2 | 0.5 |
| CD40 | | | |
| Monocytes rest | 63.3 | IBD Crohn's | 0.0 |
| Monocytes LPS | 21.3 | Colon | 10.8 |
| Macrophages rest | 41.5 | Lung | 21.5 |
| Macrophages LPS | 16.2 | Thymus | 34.4 |
| HUVEC none | 3.0 | Kidney | 27.5 |
| HUVEC starved | 57.8 | | |

CNS_neurodegeneration_v1.0 Summary: Ag4000 The CG95545-01 gene encodes a novel membrane receptor, and shows a significant downregulation in the AD temporal cortex compared to nondemented controls when CT values are analyzed by ANCOVA. The temporal cortex (Brodman area 21) shows severe neurodegeneration in Alzheimer's disease, though not as early as the hippocampus or entorhinal cortex.Thus, it is likely that this gene is downregulated during the process of neurodegeneration rather than the downregulation being a result of neuron loss. Levels in the brain are also moderate to high as determined by panels 1.2 and General Screening 1.4. Thus this gene is an excellent small molecule target for the treatment of Alzheimer's disease.

General_screening_panel_v1.4 Summary: Ag1923 The CG95545-01 gene is ubiquitously expressed in the cancer cell lines used on this panel as well as the normal tissues. The highest level of expression is in the colon cancer CaCo-2 cell line (CT=27.3). This widespread expression suggests that the protein encoded by this gene is potentially useful for cell growth and survival. This panel further confirms the expression of this gene in the CNS. See panel CNS_Neurodegeneration for a discussion of utility of this gene in the central nervous system. Among metabolic tissues, highest expression of this gene is in the placenta and pancreas. Lower levels of expression are seen in adrenal, adipose, pituitary, thyroid, small intestine, stomach, fetal skeletal muscle, fetal liver, fetal kidney, fetal heart, heart, skeletal muscle, liver and kidney. Thus, peptide and antibody therapeutics using this gene product may also be used to modulate the development and/or physiological activities in these tissues. Furthermore, higher levels of expression in the fetal liver and lung (CTs=28-29) when compared to expression in the adult liver and lung (CTs=32-33) suggest that expression of this gene could be used to differentiate between adult and fetal sources of these tissues. In addition, the higher levels of expression in the fetal tissues suggests that the protein encoded by this gene may be involved in the development of the liver and lung and thus may be useful in treatment of diseases of these organs in the adult.

Panel 1.2 Summary: Ag729 The CG95545-01 gene is ubiquitously expressed in the cancer cell lines used on this panel as well as the normal tissues. The highest level of expression is in the CaCo-2 cell line (CT=24). Both of these observations are in excellent agreeement with the results from General_screening_panel_v1.4. This expression profile suggests that expression of this gene is potentially useful for cell growth and survival. Among metabolically relevant tissues, highest expression is seen in the placenta, followed by the kidney, fetal kidney, pituitary, pancreas, small intestine, stomach and thyroid. Relatively high levels of expression according to the CT value are also seen in heart, skeletal muscle, liver and fetal liver. Thus, peptide and antibody therapeutics using this gene product may also be used to modulate the development and/or physiological activities in these tissues. This panel also confirms the expression of this gene in the CNS. See panel CNS_Neurodegeneration for a discussion of utility of this gene in the central nervous system.

Panel 2.2 Summary: Ag1923 The CG95545-01 gene is expressed at a low level in all normal and tumor samples on this panel. The highest level of expression is seen in a sample of normal adjacent kidney CT=29.6). A distinct difference is seen in gastric cancer where normal tissues express it at a slightly higher level than gastric tumors. Thus, expression of this gene could potentially be used as a marker for gastric tumors.

Panel 4.1D Summary: Ag 4000 The highest expression of the CG95545-01 transcript is found in Ku-812 after treatment with PMA and ionomycin(CT=27.4), a condition that stimulates the release of mediators such as histamine and proteases that are responsible for the symptomatology of diverse atopic diseases. This transcript is also expressed in a wide range of cells that participate in the immune response (monocytes, T, B and NK cells)and inflammatory processes (dermal and lung fibroblasts). Therefore, modulation of the expression or activity of the protein encoded by this transcript through the application of antibodies or peptides therapeutics may be beneficial for the treatment of lung inflammatory diseases such as asthma, and chronic obstructive pulmonary diseases, inflammatory skin diseases such as psoriasis, atopic dermatitis, ulcerative dermatitis, ulcerative colitis and autoimmune diseases such as Crohn's disease, lupus erythematosus, rheumatoid arthritis and osteoarthritis.

Panel 4D Summary: Ag4000 Expression of the CG95545-01 transcript is ubiquitous among the samples on this panel. Please see Panel 4.1D for discussion of utility of this gene in the immune/inflammatory response.

I. CG55746-01 and CG55746_{—}05: Butyrophilin-Like Protein

Expression of gene CG55746-01 and variant CG55746_{—}05 was assessed using the primer-probe set Ag2361, described in Table 12IA. Results of the RTQ-PCR runs are shown in Tables 12IB, 12IC and 12ID.

TABLE 12IA

Probe Name Ag2361

Start

| | | | |
|---|---|---|---|
| Primers | Sequences | Length | Position |

| | | | |
|---|---|---|---|
| Forward | 5′-acaccgtgaaagagccactt-3′ | 20 | 222 |
| | (SEQ ID NO:170) | | |
| Probe | TET-5′-cctagggaaggcctcgttccaca-3′- | 23 | 261 |
| | TAMRA (SEQ ID NO:171) | | |
| Reverse | 5′-ccctcacttggacttgaggta-3′ | 21 | 284 |
| | (SEQ ID NO:172) | | |

TABLE 12IB
Panel 1.3D

| | Rel. Exp. (%) Ag2361, | | Rel. Exp. (%) Ag2361, |
|---|---|---|---|
| Tissue Name | Run 156815394 | Tissue Name | Run 156815394 |
| Liver adenocarcinoma | 3.0 | Kidney (fetal) | 0.5 |
| Pancreas | 0.5 | Renal ca. 786-0 | 1.8 |
| Pancreatic ca. CAPAN 2 | 0.0 | Renal ca. A498 | 19.5 |
| Adrenal gland | 2.9 | Renal ca. RXF 393 | 1.3 |
| Thyroid | 3.1 | Renal ca. ACHN | 1.1 |
| Salivary gland | 2.8 | Renal ca. UO-31 | 26.1 |
| Pituitary gland | 0.8 | Renal ca. TK-10 | 0.1 |
| Brain (fetal) | 1.0 | Liver | 2.2 |
| Brain (whole) | 1.7 | Liver (fetal) | 3.6 |
| Brain (amygdala) | 2.8 | Liver ca. | 0.0 |
| | | (hepatoblast) HepG2 | |
| Brain (cerebellum) | 0.3 | Lung | 33.0 |
| Brain (hippocampus) | 5.6 | Lung (fetal) | 1.2 |
| Brain (substantia | 1.1 | Lung ca. (small | 0.0 |
| nigra) | | cell) LX-1 | |
| Brain (thalamus) | 1.1 | Lung ca. (small | 0.3 |
| | | cell) NCI-H69 | |
| Cerebral Cortex | 3.7 | Lung ca. (s.cell | 0.3 |
| | | var.) SHP-77 | |
| Spinal cord | 2.9 | Lung ca. (large | 3.7 |
| | | cell)NCI-H460 | |
| glio/astro U87-MG | 41.2 | Lung ca. (non-sm. | 0.2 |
| | | cell) A549 | |
| glio/astro U-118-MG | 100.0 | Lung ca. (non- | 0.0 |
| | | s.cell) NCI-H23 | |
| astrocytoma SW1783 | 22.5 | Lung ca. (non- | 11.7 |
| | | s.cell) HOP-62 | |
| neuro*; met SK-N-AS | 10.4 | Lung ca. (non-s.cl) | 0.0 |
| | | NCI-H522 | |
| astrocytoma SF-539 | 16.2 | Lung ca. (squam.) SW900 | 1.5 |
| astrocytoma SNB-75 | 25.9 | Lung ca. (squam.) | 0.0 |
| | | NCI-H596 | |
| glioma SNB-19 | 12.6 | Mammary gland | 10.2 |
| glioma U251 | 6.7 | Breast ca.* (pl.ef) | 1.0 |
| | | MCF-7 | |
| glioma SF-295 | 21.3 | Breast ca.* (pl.ef) | 67.4 |
| | | MDA-MB-231 | |
| Heart (fetal) | 1.1 | Breast ca.* (pl.ef) | 0.3 |
| | | T47D | |
| Heart | 2.5 | Breast ca. BT-549 | 70.7 |
| Skeletal muscle | 8.3 | Breast ca. MDA-N | 2.2 |
| (fetal) | | | |
| Skeletal muscle | 1.0 | Ovary | 4.1 |
| Bone marrow | 5.0 | Ovarian ca. OVCAR-3 | 0.0 |
| Thymus | 8.4 | Ovarian ca. OVCAR-4 | 0.0 |
| Spleen | 54.0 | Ovarian ca. OVCAR-5 | 0.5 |
| Lymph node | 14.4 | Ovarian ca. OVCAR-8 | 0.2 |
| Colorectal | 4.0 | Ovarian ca. IGROV-1 | 0.0 |
| Stomach | 4.7 | Ovarian ca.* | 2.5 |
| | | (ascites) SK-OV-3 | |
| Small intestine | 4.8 | Uterus | 4.7 |
| Colon ca. SW480 | 0.3 | Plancenta | 19.5 |
| Colon ca.* SW620(SW480 | 1.7 | Prostate | 1.4 |
| met) | | | |
| Colon ca. HT29 | 0.0 | Prostate ca.* (bone | 0.7 |
| | | met)PC-3 | |
| Colon ca. HCT-116 | 0.3 | Testis | 1.0 |
| Colon ca. CaCo-2 | 0.0 | Melanoma Hs688(A).T | 20.2 |
| Colon ca. | 8.5 | (Melanoma* (met) | 9.2 |
| tissue(ODO3866) | | Hs688(B).T | |
| Colon ca. HCC-2998 | 0.2 | Melanoma UACC-62 | 0.0 |
| Gastric ca.* (liver | 1.2 | Melanoma M14 | 0.2 |
| met) NCI-N87 | | | |
| Bladder | 4.0 | Melanoma* LOX IMVI | 12.1 |
| Trachea | 15.8 | Melanoma* (met) SK- | 0.0 |
| | | MEL-5 | |
| Kidney | 0.5 | Adipose | 7.0 |

TABLE 12IC
Panel 2D

| | Rel. Exp. (%) | | Rel. Exp. (%) |
|---|---|---|---|
| | Ag2361, Run | | Ag2361, Run |
| Tissue Name | 156823761 | Tissue Name | 156823761 |
| Normal Colon | 38.7 | Kidney Margin 8120608 | 2.4 |
| CC Well to Mod Diff | 10.5 | Kidney Cancer 8120613 | 1.6 |
| (ODO3866) | | | |
| CC Margin (ODO3866) | 6.1 | Kidney Margin 8120614 | 3.2 |
| CC Gr. 2 rectosigmoid | 6.1 | Kidney Cancer 9010320 | 26.2 |
| (ODO3868) | | | |
| CC Margin (ODO3868) | 3.8 | Kidney Margin 9010321 | 7.1 |
| CC Mod Diff (ODO3920) | 6.7 | Normal Uterus | 8.0 |
| CC Margin (ODO3920) | 6.7 | Uterus Cancer 064011 | 18.4 |
| CC Gr. 2 ascend colon | 12.2 | Normal Thyroid | 2.9 |
| (ODO3921) | | | |
| CC Margin (ODO3921) | 7.7 | Thyroid Cancer 064010 | 32.1 |
| CC from Partial | 27.2 | Thyroid Cancer | 6.5 |
| Hepatectomy (ODO4309) | | A302152 | |
| Mets | | | |
| Liver Margin (ODO4309) | 50.7 | Thyroid Margin | 14.2 |
| | | A302153 | |
| Colon mets to lung | 16.7 | Normal Breast | 39.5 |
| (OD04451-01) | | | |
| Lung Margin (ODO4451-02) | 33.0 | Breast Cancer | 18.4 |
| | | (OD04566) | |
| Normal Prostate 6546-1 | 3.9 | Breast Cancer | 26.2 |
| | | (OD04590-01) | |
| Prostate Cancer | 12.8 | Breast Cancer Mets | 45.1 |
| (OD04410) | | (OD04590-03) | |
| Prostate Margin | 19.1 | Breast Cancer | 24.0 |
| (OD04410) | | Metastasis (OD04655- | |
| | | 05) | |
| Prostate Cancer | 13.0 | Breast Cancer 064006 | 28.9 |
| (OD04720-1) | | | |
| Prostate Margin | 16.0 | Breast Cancer 1024 | 13.5 |
| (OD04720-02) | | | |
| Normal Lung 061010 | 94.0 | Breast Cancer 9100266 | 15.9 |
| Lung Met to Muscle | 62.9 | Breast Margin 9100265 | 9.5 |
| (ODO4286) | | | |
| Muscle Margin (ODO4286) | 14.1 | Breast Cancer A209073 | 9.3 |
| Lung Malignant Cancer | 26.1 | Breast Margin | 0.4 |
| (OD03126) | | A2090734 | |
| Lung Margin (OD03126) | 52.9 | Normal Liver | 12.2 |
| Lung Cancer (OD04404) | 73.7 | Liver Cancer 064003 | 6.3 |
| Lung Margin (OD04404) | 38.7 | Liver Cancer 1025 | 17.7 |
| Lung Cancer (OD04565) | 17.7 | Liver Cancer 1026 | 6.3 |
| Lung Margin (OD04565) | 34.6 | Liver Cancer 6004-T | 20.4 |
| Lung Cancer (OD04237-01) | 94.0 | Liver Tissue 6004-N | 17.8 |
| Lung Margin (OD04237-02) | 62.9 | Liver Cancer 6005-T | 8.2 |
| Ocular Mel Met to Liver | 4.7 | Liver Tissue 6005-N | 14.3 |
| (ODO4310) | | | |
| Liver Margin (ODO4310) | 25.5 | Normal Bladder | 23.5 |
| Melanoma Mets to Lung | 12.6 | Bladder Cancer 1023 | 4.2 |
| (OD04321) | | | |
| Lung Margin (OD04321) | 100.0 | Bladder Cancer | 44.8 |
| | | A302173 | |
| Normal Kidney | 27.4 | Bladder Cancer | 53.2 |
| | | (OD04718-01) | |
| Kidney Ca, Nuclear grade | 29.1 | Bladder Normal | 42.9 |
| 2 (OD04338) | | Adjacent (OD04718-03) | |
| Kidney Margin (OD04338) | 18.7 | Normal Ovary | 1.4 |
| Kidney Ca Nuclear grade | 14.7 | Ovarian Cancer 064008 | 43.2 |
| 1/2 (OD04339) | | | |
| Kidney Margin (OD04339) | 14.5 | Ovarian Cancer | 32.3 |
| | | (OD04768-07) | |
| Kidney Ca, Clear cell | 46.3 | Ovary Margin | 14.2 |
| type (OD04340) | | (OD04768-08) | |
| Kidney Margin (OD04340) | 23.3 | Normal Stomach | 16.7 |
| Kidney Ca, Nuclear grade | 27.0 | Gastric Cancer | 14.4 |
| 3 (OD04348) | | 9060358 | |
| Kidney Margin (OD04348) | 26.6 | Stomach Margin | 12.2 |
| | | 9060359 | |
| Kidney Cancer (OD04622- | 20.2 | Gastric Cancer | 18.6 |
| 01) | | 9060395 | |
| Kidney Margin (OD04622- | 1.5 | Stomach Margin | 12.9 |
| 03) | | 9060394 | |
| Kidney Cancer (OD04450- | 0.4 | Gastric Cancer | 15.6 |
| 01) | | 9060397 | |
| Kidney Margin (OD04450- | 13.5 | Stomach Margin | 9.7 |
| 03) | | 9060396 | |
| Kidney Cancer 8120607 | 1.8 | Gastric Cancer 064005 | 40.6 |

TABLE 12ID
Panel 4D

| | Rel. Exp. (%) | | Rel. Exp. (%) |
|---|---|---|---|
| | Ag2361, Run | | Ag2361, Run |
| Tissue Name | 156823763 | Tissue Name | 156823763 |
| Secondary Th1 act | 1.8 | HUVEC IL-1beta | 17.6 |
| Secondary Th2 act | 5.7 | HUVEC IFN gamma | 38.2 |
| Secondary Tr1 act | 6.8 | HUVEC TNF alpha + IFN | 72.7 |
| | | gamma | |
| Secondary Th1 rest | 4.4 | HUVEC TNF alpha + IL4 | 61.1 |
| Secondary Th2 rest | 4.3 | HUVEC IL-11 | 4.5 |
| Secondary Tr1 rest | 4.3 | Lung Microvascular EC | 15.2 |
| | | none | |
| Primary Th1 act | 0.3 | Lung Microvascular EC | 45.4 |
| | | TNF alpha + IL-1beta | |
| Primary Th2 act | 1.2 | Microvascular Dermal EC | 34.9 |
| | | none | |
| Primary Tr1 act | 2.2 | Microsvasular Dermal EC | 57.4 |
| | | TNF alpha + IL-1beta | |
| Primary Th1 rest | 12.0 | Bronchial epithelium | 0.4 |
| | | TNF alpha + IL-1beta | |
| Primary Th2 rest | 4.4 | Small airway epithelium | 8.1 |
| | | none | |
| Primary Tr1 rest | 0.4 | Small airway epithelium | 66.0 |
| | | TNF alpha + IL-1beta | |
| CD45RA CD4 lymphocyte | 33.2 | Coronery artery SMC rest | 41.2 |
| act | | | |
| CD45RO CD4 lymphocyte | 3.0 | Coronery artery SMC | 39.5 |
| act | | TNF alpha + IL-1beta | |
| CD8 lymphocyte act | 1.2 | Astrocytes rest | 17.8 |
| Secondary CD8 | 2.3 | Astrocytes TNF alpha + | 41.2 |
| lymphocyte rest | | IL-1beta | |
| Secondary CD8 | 3.2 | KU-812 (Basophil) rest | 0.0 |
| lymphocyte act | | | |
| CD4 lymphocyte none | 0.3 | KU-812 (Basophil) | 0.2 |
| | | PMA/ionomycin | |
| 2ry Th1/Th2/Tr1_anti- | 4.6 | CCD1106 (Keratinocytes) | 9.8 |
| CD95 CH11 | | none | |
| LAK cells rest | 27.5 | CCD1106 (Keratinocytes) | 4.2 |
| | | TNF alpha + IL-1beta | |
| LAK cells IL-2 | 4.5 | Liver cirrhosis | 1.6 |
| LAK cells IL-2 + IL-12 | 4.4 | Lupus kidney | 0.4 |
| LAK cells IL-2 + IFN | 13.2 | NCI-H292 none | 2.1 |
| gamma | | | |
| LAK cells IL-2 + IL-18 | 8.4 | NCI-H292 IL-4 | 17.2 |
| LAK cells | 16.8 | NCI-H292 IL-9 | 2.9 |
| PMA/ionomycin | | | |
| NK Cells IL-2 rest | 1.0 | NCI-H292 IL-13 | 9.2 |
| Two Way MLR 3 day | 13.2 | NCI-H292 IFN gamma | 18.4 |
| Two Way MLR 5 day | 4.9 | HPAEC none | 21.8 |
| Two Way MLR 7 day | 2.2 | HPAEC TNF alpha + | 79.6 |
| | | IL-1beta | |
| PBMC rest | 0.4 | Lung fibroblast none | 17.4 |
| PBMC PWM | 17.0 | Lung fibroblast TNF alpha + | 18.9 |
| | | IL-1beta | |
| PBMC PHA-L | 8.0 | Lung fibroblast IL-4 | 61.6 |
| Ramos (B cell) none | 3.6 | Lung fibroblast IL-9 | 42.0 |
| Ramos (B cell) | 15.0 | Lung fibroblast IL-13 | 29.5 |
| ionomycin | | | |
| B lymphocytes PWM | 9.0 | Lung fibroblast IFN gamma | 100.0 |
| B lymphocytes CD40L | 17.1 | Dermal fibroblast CCD1070 | 68.8 |
| and IL-4 | | rest | |
| EOL-1 dbcAMP | 0.1 | Dermal fibroblast CCD1070 | 96.6 |
| | | TNF alpha | |
| EOL-1 dbcAMP | 0.3 | Dermal fibroblast CCD1070 | 50.7 |
| PMA/ionomycin | | IL-1beta | |
| Dendritic cells none | 29.7 | Dermal fibroblast IFN | 26.2 |
| | | gamma | |
| Dendritic cells LPS | 43.2 | Dermal fibroblast IL-4 | 36.9 |
| Dendritic cells anti- | 10.5 | IBD Colitis 2 | 0.3 |
| CD40 | | | |
| Monocytes rest | 0.4 | IBD Crohn's | 0.6 |
| Monocytes LPS | 8.8 | Colon | 4.5 |
| Macrophages rest | 15.8 | Lung | 7.8 |
| Macrophages LPS | 13.6 | Thymus | 3.1 |
| HUVEC none | 11.0 | Kidney | 17.3 |
| HUVEC starved | 31.6 | | |

Panel 1.3D Summary: Ag2361 The CG55746-01 gene is expressed at a moderately high level in brain, breast and renal cancer cell lines compared to the normal tissue, with highest expression in a brain cancer cell line (CT=28.5). Hence, the expression of this gene could be of use as a marker for different grades/types of brain cancer, renal cancer and breast cancer that were used in the derivation of these cell lines. In addition, therapeutic inhibition of the activity of the product of this gene, through the use of small molecule drugs, may be useful in the therapy of brain, renal and breast cancer. Among metabolic tissues, expression of this butyrophilin-like gene is highest in the placenta, with lower levels in fetal skeletal muscle, adipose, stomach, small intestine, adrenal, thyroid, heart, liver and fetal liver. This molecule may be involved in the interaction of the immune system with these organs and modulation of this gene product by peptide and antibody therapeutics may alleviate disorders originating in these tissues. This gene, a butyrophilin homolog, shows moderate to low expression in the CNS. Butyrophilin has been shown to modulate the immune response in multiple sclerosis, suggesting that this protein may be useful in the treatment of this disease or other diseases associated with immune system-induced myelin damage. References: Steffer1 A, Schubart A, Storch2 M, Amini A, Mather I, Lassmann H, Linington C. Butyrophilin, a milk protein, modulates the encephalitogenic T cell response to myelin oligodendrocyte glycoprotein in experimental autoimmune encephalomyelitis. J Immunol Sep. 1, 2000;165(5):2859-65. Experimental autoimmune encephalomyelitis (EAE) induced by sensitization with myelin oligodendrocyte glycoprotein (MOG) is a T cell-dependent autoimmune disease that reproduces the inflammatory demyelinating pathology of multiple sclerosis. We report that an encephalitogenic T cell response to MOG can be either induced or alternatively suppressed as a consequence of immunological cross-reactivity, or “molecular mimicry” with the extracellular IgV-like domain of the milk protein butyrophilin (BTN). In the Dark Agouti rat, active immunization with native BTN triggers an inflammatory response in the CNS characterized by the formation of scattered meningeal and perivascular infiltrates of T cells and macrophages. We demonstrate that this pathology is mediated by a MHC class II-restricted T cell response that cross-reacts with the MOG peptide sequence 76-87, I GEG KVA LRIQ N (identities underlined). Conversely, molecular mimicry with BTN can be exploited to suppress disease activity in MOG-induced EAE. We demonstrate that not only is EAE mediated by the adoptive transfer of MOG74-90 T cell lines markedly ameliorated by i.v. treatment with the homologous BTN peptide, BTN74-90, but that this protective effect is also seen in actively induced disease following transmucosal (intranasal) administration of the peptide. These results identify a mechanism by which the consumption of milk products may modulate the pathogenic autoimmune response to MOG.

Panel 2D Summary: Ag2361 The CG55746-01 gene is ubiquitously expressed in all tissues in this panel, with highest expression in normal lung tissue adjacent to a tumor (CT=28.4). There is significantly higher expression in normal lung tissue compared to melanomas that have metastasized to lung. Thus, the expression can be used to differentiate between normal lung tissue and metastatic melanomas.

Panel 4D Summary: Ag 2361 The CG55746-01 transcript is ubiquitously expressed at moderate levels in all cell types of this panel, with highest expression of this transcript is found in lung fibroblasts upon IFN g treatment(CT=26.8). High levels of expression are also seen in dermal fibroblasts treated with TNF-a, HUVEC treated with TNF-a and IFNg and small airway epithelium treated with TNF-a and IL-1b. In all these cell types, the expression of this transcript, although constitutive, is dramatically up-regulated upon treatment with the potent inflammatory cytokines TNF-a and IFNg, suggesting a role for the protein encoded by this transcript in these cell types during inflammation. Therefore, modulation of this gene product by antibodies or small molecules therapeutics may be beneficial for the treatment of the symptoms associated with the inflammatory processes observed in asthma, chronic obstructive pulmonary diseases and psoriasis.

J. CG50329-01: Butyrophilin-Like Protein

Expression of gene CG50329-01 was assessed using the primer-probe sets Ag2563 and Ag2563b, described in Tables 12JA and 12JB. Results of the RTQ-PCR runs are shown in Tables 12JC, and 12D.

TABLE 12JA

Probe Name Ag2563

Start

| | | | |
|---|---|---|---|
| Primers | Sequences | Length | Position |

| | | | |
|---|---|---|---|
| Forward | 5′-atgcagtcattccctcactgt-3′ | 21 | 65 |
| | SEQ ID NO:173 | | |
| Probe | TET-5′-tccttgaactcctgacctcaggcaat-3′- | 26 | 110 |
| | TAMRA SEQ ID NO:174 | | |
| Reverse | 5′-gtgacatcaaagtcagctttcc-3′ | 22 | 137 |
| | SEQ ID NO:175 | | |

TABLE 12JB

Probe Name Ag2563b

Start

| | | | |
|---|---|---|---|
| Primers | Sequences | Length | Position |

| | | | |
|---|---|---|---|
| Forward | 5′-atgggaaagctgactttgatg-3′ | 21 | 134 |
| | SEQ ID NO:176 | | |
| Probe | TET-5′-ctcatgcccctattctggctatggct-3′- | 26 | 164 |
| | TAMRA SEQ ID NO:177 | | |
| Reverse | 5′-ggaacagctggcactgtaact-3′ | 21 | 203 |
| | SEQ ID NO:178 | | |

TABLE 12JC
General_screening_panel_vl.4

| | Rel. Exp. (%) Ag2563b, | | Rel. Exp. (%) Ag2563b, |
|---|---|---|---|
| Tissue Name | Run 216607737 | Tissue Name | Run 216607737 |
| Adipose | 0.0 | Renal ca. TK-10 | 1.9 |
| Melanoma* | 0.0 | Bladder | 0.4 |
| Hs688(A).T | | | |
| Melanoma* | 0.4 | Gastric ca. (liver | 7.9 |
| Hs688(B).T | | met.) NCI-N87 | |
| Melanoma* M14 | 0.0 | Gastric ca. KATO III | 0.0 |
| Melanoma* LOXIMVI | 0.4 | Colon ca. SW-948 | 0.0 |
| Melanoma* SK-MEL-5 | 0.4 | Colon ca. SW480 | 0.7 |
| Squamous cell | 0.0 | Colon ca.* (SW480 met) | 1.0 |
| carcinoma SCC-4 | | SW620 | |
| Testis Pool | 0.4 | Colon ca. HT29 | 0.5 |
| Prostate ca.* (bone | 0.5 | Colon ca. HCT-116 | 0.8 |
| met) PC-3 | | | |
| Prostate Pool | 0.6 | Colon ca. CaCo-2 | 0.6 |
| Placenta | 0.0 | Colon cancer tissue | 1.0 |
| Uterus Pool | 0.4 | Colon ca. SW1116 | 0.3 |
| Ovarian ca. OVCAR-3 | 2.5 | Colon ca. Colo-205 | 0.5 |
| Ovarian ca. SK-OV-3 | 1.4 | Colon ca. SW-48 | 0.0 |
| Ovarian ca. OVCAR-4 | 0.6 | Colon Pool | 0.4 |
| Ovarian ca. OVCAR-5 | 8.8 | Small Intestine Pool | 0.8 |
| Ovarian ca. IGROV-1 | 1.7 | Stomach Pool | 1.2 |
| Ovarian ca. OVCAR-8 | 0.6 | Bone Marrow Pool | 0.5 |
| Ovary | 0.0 | Fetal Heart | 0.3 |
| Breast ca. MCF-7 | 3.7 | Heart Pool | 0.0 |
| Breast ca. MDA-MB- | 0.3 | Lymph Node Pool | 0.8 |
| 231 | | | |
| Breast ca. BT 549 | 1.9 | Fetal Skeletal Muscle | 0.6 |
| Breast ca. T47D | 7.0 | Skeletal Muscle Pool | 0.0 |
| Breast ca. MDA-N | 2.5 | Spleen Pool | 2.3 |
| Breast Pool | 0.3 | Thymus Pool | 1.0 |
| Trachea | 0.1 | CNS cancer | 3.1 |
| | | (glio/astro) U87-MG | |
| Lung | 0.4 | CNS cancer | 2.2 |
| | | (glio/astro) U-118-MG | |
| Fetal Lung | 4.3 | CNS cancer (neuro;met) | 0.0 |
| | | SK-N-AS | |
| Lung ca. NCI-N417 | 0.0 | CNS cancer (astro) SF- | 0.4 |
| | | 539 | |
| Lung ca. LX-1 | 0.5 | CNS cancer (astro) | 2.3 |
| | | SNB-75 | |
| Lung ca. NCI-H146 | 0.0 | CNS cancer (glio) SNB- | 1.9 |
| | | 19 | |
| Lung ca. SHP-77 | 0.1 | CNS cancer (glio) SF- | 2.8 |
| | | 295 | |
| Lung ca. A549 | 0.3 | Brain (Amygdala) Pool | 0.0 |
| Lung ca. NCI-H526 | 0.0 | Brain (cerebellum) | 0.0 |
| Lung ca. NCI-H23 | 4.5 | Brain (fetal) | 0.5 |
| Lung ca. NCI-H460 | 1.0 | Brain (Hippocampus) | 0.0 |
| | | Pool | |
| Lung ca. HOP-62 | 0.0 | Cerebral Cortex Pool | 0.0 |
| Lung ca. NCI-H522 | 6.7 | Brain (Substantia | 0.0 |
| | | nigra) Pool | |
| Liver | 0.0 | Brain (Thalamus) Pool | 0.5 |
| Fetal Liver | 100.0 | Brain (whole) | 0.6 |
| Liver ca. HepG2 | 0.0 | Spinal Cord Pool | 0.9 |
| Kidney Pool | 1.1 | Adrenal Gland | 0.0 |
| Fetal Kidney | 0.2 | Pituitary gland Pool | 0.4 |
| Renal ca. 786-0 | 0.0 | Salivary Gland | 0.0 |
| Renal ca. A498 | 0.2 | Thyroid (female) | 0.0 |
| Renal ca. ACHN | 0.4 | Pancreatic ca. CAPAN2 | 0.0 |
| Renal ca. UO-31 | 0.0 | Pancreas Pool | 0.2 |

TABLE 12JD
Panel 4.1D

| | Rel. Exp. (%) | | Rel. Exp. (%) |
|---|---|---|---|
| | Ag2563b, Run | | Ag2563b, Run |
| Tissue Name | 172226101 | Tissue Name | 172226101 |
| Secondary Th1 act | 0.9 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 1.0 | HUVEC TNF alpha + IL4 | 0.0 |
| Secondary Th2 rest | 1.5 | HUVEC IL-11 | 0.0 |
| Secondary Tr1 rest | 0.0 | Lung Microvascular EC | 0.8 |
| | | none | |
| Primary Th1 act | 2.3 | Lung Microvascular EC | 0.0 |
| | | TNF alpha + IL-1beta | |
| Primary Th2 act | 0.4 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.7 |
| | | TNF alpha + IL1beta | |
| Primary Th2 rest | 0.0 | Small airway epithelium | 0.7 |
| | | none | |
| Primary Tr1 rest | 2.8 | Small airway epithelium | 2.0 |
| | | TNF alpha + IL-1beta | |
| CD45RA CD4 lymphocyte | 1.0 | Coronery artery SMC rest | 0.0 |
| act | | | |
| CD45RO CD4 lymphocyte | 0.4 | Coronery artery SMC | 1.0 |
| act | | TNF alpha + IL-1beta | |
| CD8 lymphocyte act | 1.7 | Astrocytes rest | 0.0 |
| Secondary CD8 | 2.5 | Astrocytes TNF alpha + IL- | 0.0 |
| lymphocyte rest | | 1beta | |
| Secondary CD8 | 0.0 | KU-812 (Basophil) rest | 29.7 |
| lymphocyte act | | | |
| CD4 lymphocyte none | 0.8 | KU-812 (Basophil) | 11.0 |
| | | PMA/ionomycin | |
| 2ry Th1/Th2/Tr1_anti- | 2.7 | CCD1106 (Keratinocytes) | 0.0 |
| CD95 CH11 | | none | |
| LAK cells rest | 0.4 | CCD1106 (Keratinocytes) | 0.0 |
| | | TNF alpha + IL-1beta | |
| LAK cells IL-2 | 0.9 | Liver cirrhosis | 0.0 |
| LAK cells IL-2 + IL-12 | 0.0 | NCI-H292 none | 2.3 |
| LAK cells IL-2 + IFN | 0.0 | NCI-H292 IL-4 | 2.4 |
| gamma | | | |
| LAK cells IL-2 + IL-18 | 0.8 | NCI-H292 IL-9 | 2.0 |
| LAK cells | 0.0 | NCI-H292 IL-13 | 1.5 |
| PMA/ionomycin | | | |
| NK Cells IL-2 rest | 0.7 | NCI-H292 IFN gamma | 2.6 |
| Two Way MLR 3 day | 2.0 | HPAEC none | 0.0 |
| Two Way MLR 5 day | 0.0 | HPAEC TNF alpha + IL-1 | 0.0 |
| | | beta | |
| Two Way MLR 7 day | 1.8 | Lung fibroblast none | 0.9 |
| PBMC rest | 1.9 | Lung fibroblast TNF alpha + IL- | 0.9 |
| | | 1beta | |
| PBMC PWM | 0.8 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 3.0 |
| Ramos (B cell) | 0.0 | Lung fibroblast IFN gamma | 5.3 |
| ionomycin | | | |
| B lymphocytes PWM | 0.0 | Dermal fibroblast CCD1070 | 2.5 |
| | | rest | |
| B lymphocytes CD40L | 0.0 | Dermal fibroblast CCD1070 | 2.8 |
| and IL-4 | | TNF alpha | |
| EOL-1 dbcAMP | 0.8 | Dermal fibroblast CCD1070 | 0.0 |
| | | IL-1beta | |
| EOL-1 dbcAMP | 0.0 | Dermal fibroblast IFN | 5.1 |
| PMA/ionomycin | | gamma | |
| Dendritic cells none | 1.7 | Dermal fibroblast IL-4 | 0.7 |
| Dendritic cells LPS | 0.0 | Dermal Fibroblasts rest | 2.1 |
| Dendritic cells anti- | 0.0 | Neutrophils TNFa + LPS | 0.6 |
| CD40 | | | |
| Monocytes rest | 0.0 | Neutrophils rest | 0.8 |
| Monocytes LPS | 0.0 | Colon | 2.0 |
| Macrophages rest | 0.0 | Lung | 9.0 |
| Macrophages LPS | 0.0 | Thymus | 20.7 |
| HUVEC none | 0.0 | Kidney | 100.0 |
| HUVEC starved | 0.0 | | |

CNS_neurodegeneration_v1.0 Summary: Ag2563 Expression of the CG50329-01 gene is insignificant/undetectable in all samples on this panel. (Data not shown.)

General_screening_panel_v1.4 Summary: Ag2563b Highest expression of the CG50329-01 gene is seen in fetal liver (CT=28.l1). Thus, this gene may be involved in development of the liver and may be used to differentiate fetal and adult liver. In addition, peptide or antibody therapeutics may be used to modulate the activity of its gene product to influence development or function of the liver. This gene is expressed at a low level in most of the cancer cell lines and normal tissues on this panel. Lung and ovarian cancer cell lines express this gene at a higher level than the normal lung and ovary tissues. Hence, expression of this gene can be used as a diagnostic marker for the lung and ovarian cancers used for the derivation of these cell lines. This gene encodes a novel butyrophilin-like protein with low expression in the spinal cord. Butyrophilin has been shown to modulate the immune response in multiple sclerosis, suggesting that this protein may be useful in the treatment of this or other diseases associated with immune system-induced myelin damage. References: Steffer1 A, Schubart A, Storch2 M, Amini A, Mather I, Lassmann H, Linington C. Butyrophilin, a milk protein, modulates the encephalitogenic T cell response to myelin oligodendrocyte glycoprotein in experimental autoimmune encephalomyelitis. J Immunol Sep. 1, 2000;165(5):2859-65. Experimental autoimmune encephalomyelitis (EAE) induced by sensitization with myelin oligodendrocyte glycoprotein (MOG) is a T cell-dependent autoimmune disease that reproduces the inflammatory demyelinating pathology of multiple sclerosis. We report that an encephalitogenic T cell response to MOG can be either induced or alternatively suppressed as a consequence of immunological cross-reactivity, or “molecular mimicry” with the extracellular IgV-like domain of the milk protein butyrophilin (BTN). In the Dark Agouti rat, active immunization with native BTN triggers an inflammatory response in the CNS characterized by the formation of scattered meningeal and perivascular infiltrates of T cells and macrophages. We demonstrate that this pathology is mediated by a MHC class 11-restricted T cell response that cross-reacts with the MOG peptide sequence 76-87, I GEG KVA LRIQ N (identities underlined). Conversely, molecular mimicry with BTN can be exploited to suppress disease activity in MOG-induced EAE. We demonstrate that not only is EAE mediated by the adoptive transfer of MOG74-90 T cell lines markedly ameliorated by i.v. treatment with the homologous BTN peptide, BTN74-90, but that this protective effect is also seen in actively induced disease following transmucosal (intranasal) administration of the peptide. These results identify a mechanism by which the consumption of milk products may modulate the pathogenic autoimmune response to MOG.

Panel 1.3D Summary: Ag2563 One experiment with this probe and primer set failed along with the genomic DNA control. (Data not shown.)

Panel 2D Summary: Ag2563 Ag2563 Expression of the CG50329-01 gene is low/undetectable in all samples on this panel. (CTs>35)(Data not shown.)

Panel 4.1D Summary: Ag 2563b: The highest expression of the CG50329-01 transcript is found in kidney, thymus and lung. Thus, the protein encoded by this transcript may play an important role in the normal homeostasis of these tissues. This gene is also expressed in KU-812, a basophil cell line. This cell type is involved in atopic diseases such as asthma, contact dermatitis and other inflammatory diseases such as inflammatory bowel disease. Therefore, antibodies or small molecule therapeutics designed with the protein encoded by this transcript may be important for maintaining or restoring normal function to thymus and lung during inflammation and in particular for the treatment of asthma, inflammatory bowel disease and allergies.

Panel 4D Summary: Ag2563 Two experiments with this probe and primer set failed along with the genomic DNA control. (Data not shown.)

### Example 3

### SNP Analysis of NOVX Clones

SeqCallingTM Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing (Pyrosequencing, Westborough, Mass.). Detailed protocols for Pyrosequencing can be found in: Alderborn et al. Determination of Single Nucleotide Polymorphisms by Real-time Pyrophosphate DNA Sequencing. (2000). Genome Research. 10, Issue 8, August. 1249-1265. In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels. The DNA and protein sequences for the novel single nucleotide polymorphic variants are reported. Variants are reported individually but any combination of all or a select subset of variants are also included. In addition, the positions of the variant bases and the variant amino acid residues are underlined.

### Results

Variants are reported individually but any combination of all or a select subset of variants are also included as contemplated NOVX embodiments of the invention.

NOV3

The DNA and protein sequences for the novel single nucleotide polymorphic variants of the Beta Adrenergic Receptor Kinase-like gene of CuraGen Acc. No. CG50345-01 are reported in Table 13. Variants are reported individually but any combination of all or a select subset of variants are also included. The positions of the variant bases and the variant amino acid residues are underlined. In summary, there are 5 variants reported, whose variant positions for its nucleotide and amino acid sequences is numbered according to SEQ ID NOs:11 and 12, respectively. Variant 13375845 is an A to C SNP at 203 bp of the nucleotide sequence that results in no charge in the protein sequence (silent), variant 13375846 is an A to G SNP at 292 bp of the nucleotide sequence that results in a Lys to Arg change at amino acid 62 of protein sequence, variant 13376064 is a G to A SNP at 1814 bp of the nucleotide sequence that results in a Trp to End change at amino acid 569 of protein sequence, variant 13376063 is a T to C SNP at 1885 bp of the nucleotide sequence that results in an Ile to Thr change at amino acid 593 of protein sequence, and variant 13376062 is a G to A SNP at 2001 bp of the nucleotide sequence that results in a Glu to Lys change at amino acid 632 of protein sequence.

TABLE 13
cSNP and Coding Variants for NOV3

| NT Position of | Wild Type | | Amino | Amino Acid |
|---|---|---|---|---|
| cSNP | NT | Variant NT | Acid position | Change |
| 203 | A | C | — | silent |
| 292 | A | G | 62 | Lys-Arg |
| 1814 | G | A | 569 | Trp-end |
| 1885 | T | C | 593 | Ile-Thr |
| 2001 | G | A | 632 | Glu-Lys |

NOV4

There are 3 variants reported in Table 14, whose variant positions for its nucleotide and amino acid sequences is numbered according to SEQ ID Nos:13 and 14, respectively. Variant 13374261 is an A to G SNP at 117 bp of the nucleotide sequence that results in an Asp to Gly change at amino acid 28 of protein sequence, variant 13374262 is a T to C SNP at 225 bp of the nucleotide sequence that results in a Val to Ala change at amino acid 64 of protein sequence, and variant 13374263 is a G to A SNP at 260 bp of the nucleotide sequence that results in an Ala to Thr change at amino acid 76 of protein sequence.

TABLE 14
cSNP and Coding Variants for NOV4

| NT Position | | | | |
|---|---|---|---|---|
| of | | | Amino Acid | Amino Acid |
| cSNP | Wild Type NT | Variant NT | position | Change |
| 117 | A | G | 28 | Asp-Gly |
| 225 | T | C | 64 | Val-Ala |
| 260 | G | A | 76 | Ala-Thr |

NOV5A

The DNA and protein sequences for the novel single nucleotide polymorphic variants of the Out-At-First-like gene of CuraGen Acc. No. CG55764-01 are reported in Table 15. Variants are reported individually but any combination of all or a select subset of variants are also included. There are 4 variants reported whose variant positions for its nucleotide and amino acid sequences which are numbered according to SEQ ID NOs:15 and 16, respectively. Variant 13374591 is an A to G SNP at 281 bp of the nucleotide sequence that results in a Gln to Arg change at amino acid 94 of protein sequence, variant 13374592 is an A to G SNP at 344 bp of the nucleotide sequence that results in a Glu to Gly change at amino acid 115 of protein sequence, variant 13374593 is a G to A SNP at 629 bp of the nucleotide sequence that results in an Arg to His change at amino acid 210 of protein sequence, and variant 13374594 is an A to G SNP at 650 bp of the nucleotide sequence that results in a His to Arg change at amino acid 217 of protein sequence.

TABLE 15
cSNP and Coding Variants for NOV5a

| NT Position | | | Amino Acid | Amino Acid |
|---|---|---|---|---|
| of cSNP | Wild Type NT | Variant NT | position | Change |
| 281 | A | G | 94 | Gln-Arg |
| 344 | A | G | 115 | Glu-Gly |
| 629 | G | A | 210 | Arg-His |
| 650 | A | G | 217 | His-Arg |

NOV6A

The DNA and protein sequences for the novel single nucleotide polymorphic variants of the EphA6 ehk 2-like gene of CuraGen Acc. No. CG55704-01 are reported in Table 16. Variants are reported individually but any combination of all or a select subset of variants are also included. There are 2 variants reported whose variant positions for its nucleotide and amino acid sequences are numbered according to SEQ ID NOs:19 and 20, respectively. Variant 13376314 is a C to T SNP at 1674 bp of the nucleotide sequence that results in no change in the protein sequence (silent), and variant 13376315 is a G to A SNP at 2889 bp of the nucleotide sequence that results in no change in the protein sequence (silent).

TABLE 16
cSNP and Coding Variants for NOV6a

| NT Position of | Wild | Variant | Amino | Amino |
|---|---|---|---|---|
| cSNP | Type NT | NT | Acid position | Acid Change |
| 1674 | C | T | — | silent |
| 2889 | G | A | — | silent |

NOV8 AND NOV9

The DNA and protein sequences for the novel single nucleotide polymorphic variants of the Type Ia Membrane Sushi-Containing Domain-like gene of CuraGen Acc. No. CG95545-01 are reported in Table 17. Variants are reported individually but any combination of all or a select subset of variants are also included. There is one variant reported whose variant position for its nucleotide and amino acid sequences are numbered according to SEQ ID NOs:25, 26, 27 and 28, respectively. Variant 13376324 is a T to G SNP at 2693 bp of the nucleotide sequence that results in no change in the protein sequence since the SNP is not in the amino acid coding region.

TABLE 17
cSNP and Coding Variants for NOV8 and NOV9

| NT Position | Wild Type | Variant | Amino Acid | Amino Acid |
|---|---|---|---|---|
| of cSNP | NT | NT | position | Change |
| 2693 | T | G | — | No change |

NOV10A

The DNA and protein sequences for the novel single nucleotide polymorphic variants of the BUTYROPHILIN-like gene of CuraGen Acc. No. CG55746-01 are reported in Table 18.

Variants are reported individually but any combination of all or a select subset of variants are also included. There are 6 variants reported whose variant positions for its nucleotide and amino acid sequences are numbered according to SEQ ID NOs:29 and 30, respectively. Variant 13376321 is an A to G SNP at 426 bp of the nucleotide sequence that results in no change in the protein sequence (silent), variant 13376320 is a C to T SNP at 506 bp of the nucleotide sequence that results in an Ala to Val change at amino acid 154 of protein sequence, variant 13376319 is a G to A SNP at 515 bp of the nucleotide sequence that results in a Ser to Asn change at amino acid 157 of protein sequence, variant 13376318 is an A to T SNP at 583 bp of the nucleotide sequence that results in an Arg to End change at amino acid 180 of protein sequence, variant 13376317 is a T to C SNP at 641 bp of the nucleotide sequence that results in an Ile to Thr change at amino acid 99 of protein sequence, and variant 13376316 is a T to C SNP at 743 bp of the nucleotide sequence that results in an Ile to Thr change at amino acid 233 of protein sequence.

TABLE 18
cSNP and Coding Variants for NOV10a

| NT Position | Wild Type | Variant | Amino Acid | Amino Acid |
|---|---|---|---|---|
| of cSNP | NT | NT | position | Change |
| 426 | A | G | — | Silent |
| 506 | C | T | 154 | Ala-Val |
| 515 | G | A | 157 | Ser-Asn |
| 583 | A | T | 180 | Arg-end |
| 641 | T | C | 199 | Ile-Thr |
| 743 | T | C | 233 | Ile-Thr |

### Example 4

### PCR CLONING

NOV4:CG50301-01: Human TENM4

The cDNA coding for a domain of the full length of CG50301-01 between residues 371 to 830 was targeted for “in-frame” cloning by PCR. The PCR template is based on human cDNA(s).

The following oligonucleotide primers identified as SEQ ID NOs:92 and 93 were used to clone the target cDNA sequence:

| | |
|---|---|
| F1 | 5′-GGATCC CACCTGCAGCCGATGGAGGGGCAGATGTATGAG-3′ |
| R1 | 5′-CTCGAG ACAGCCAGCTCCTCTCCAGCCCAGCTGGCAGACG-3′ |

For downstream cloning purposes, the forward primer (F1: SEQ ID NO:92) includes an in-frame BamHI restriction site and the reverse primer (R1: SEQ ID NO:93) contains an in-frame XhoI restriction site.

Two parallel PCR reactions were set up using a total of 0.5-1.0 ng human pooled cDNAs as template for each reaction. The pool is composed of 5 micrograms of each of the following human tissue cDNAs: adrenal gland, whole brain, amygdala, cerebellum, thalamus, bone marrow, fetal brain, fetal kidney, fetal liver, fetal lung, heart, kidney, liver, lymphoma, Burkitt's Raji cell line, mammary gland, pancreas, pituitary gland, placenta, prostate, salivary gland, skeletal muscle, small Intestine, spleen, stomach, thyroid, trachea, uterus.

When the tissue of expression is known and available, the second PCR was performed using the above primers and 0.5 ng-1.0 ng of one of the following human tissue cDNAs: skeleton muscle, testis, mammary gland, adrenal gland, ovary, colon, normal cerebellum, normal adipose, normal skin, bone marrow, brain amygdala, brain hippocampus, brain substantia nigra, brain thalamus, thyroid, fetal lung, fetal liver, fetal brain, kidney, heart, spleen, uterus, pituitary gland, lymph node, salivary gland, small intestine, prostate, placenta, spinal cord, peripheral blood, trachea, stomach, pancreas, hypothalamus.

The reaction mixtures contained 2 microliters of each of the primers (original concentration: 5 pmol/ul), 1 microliter of 10 mM DNTP (Clontech Laboratories, Palo Alto Calif.) and 1 microliter of 50xAdvantage-HF 2 polymerase (Clontech Laboratories) in 50 microliter-reaction volume. The following reaction conditions were used:
PCR condition 1:
a) 96° C. 3 minutes
b) 96° C. 30 seconds denaturation
c) 60° C. 30 seconds, primer annealing
d) 72° C. 6 minutes extension
Repeat steps b-d 15 times
e) 96° C. 15 seconds denaturation
f) 60° C. 30 seconds, primer annealing
g) 72° C. 6 minutes extension
Repeat steps e-g 29 times
e) 72° C. 10 minutes final extension
PCR condition 2:
a) 96° C. 3 minutes
b) 96° C. 15 seconds denaturation
c) 76° C. 30 seconds, primer annealing, reducing the temperature by 1° C. per cycle
d) 72° C. 4 minutes extension
Repeat steps b-d 34 times
e) 72° C. 10 minutes final extension

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers:

| | | |
|---|---|---|
| SEQ ID NO:94: | SF1: | TGGAGATCTCAAGTGTTCATAGACCATC |
| SEQ ID NO:95: | SF2: | ACAGGCTTCATCCAGTATTTGGATTC |
| SEQ ID NO:96: | SF3: | AAATGGCCAATACATGAAAGGGA |
| SEQ ID NO:97: | SF4: | ATTGCTTTGTGGGATGGGGAG |
| SEQ ID NO:98: | SF5: | AATGGCGAACACTGCACCATC |
| SEQ ID NO:99: | SR1: | AAGTGCCAGGAGGAATCTTCTGGGAGG |
| SEQ ID NO:100: | SR2: | GAAGCCTGTCTCATGGCTGGAG |
| SEQ ID NO:101: | SR3: | ATTTCCGCTACAGAGCACGGG |
| SEQ ID NO:102: | SR4: | ATTCGCCTCTCACGCAGACAC |
| SEQ ID NO:103: | SR5: | ACCACAGTCGGCAGCACAGAT |

The insert 172885447 was found to encode an open reading frame similar to that between residues 371 and 830 of the target sequence of CG50301-01. The cloned insert is 99% identical to the original sequence. It differs from the original sequence at 3 nucleotide positions and one amino acid position.

NOV 11: CG50329-01

The cDNA coding for a domian of CG50329-01 from residue 32 to 236 was targeted for “in-frame” cloning by PCR. The PCR template is based human cDNA(s).

The following oligonucleotide primers were used to clone the target cDNA sequence:

| | |
|---|---|
| F1 | 5′-GGATCC AAAGCTGACTTTGATGTCACTGGGCCTCATGC-3′ |
| R3 | 5′-CTCGAG CCTTTCAGGGAGGAGGGGGCTGGAGATGG-3′ |

For downstream cloning purposes, the forward primer (F1: SEQ ID NO:104) includes an in-frame BamHI restriction site and the reverse primer (R3: SEQ ID NO:105) contains an in-frame XhoI restriction site.

Two parallel PCR reactions were set up using a total of 0.5-1.0 ng human pooled cDNAs as template for each reaction. The pool is composed of 5 micrograms of each of the following human tissue cDNAs: adrenal gland, whole brain, amygdala, cerebellum, thalamus, bone marrow, fetal brain, fetal kidney, fetal liver, fetal lung, heart, kidney, liver, lymphoma, Burkitt's Raji cell line, mammary gland, pancreas, pituitary gland, placenta, prostate, salivary gland, skeletal muscle, small Intestine, spleen, stomach, thyroid, trachea, uterus.

When the tissue of expression is known and available, the second PCR was performed using the above primers and 0.5 ng-1.0 ng of one of the following human tissue cDNAs: skeleton muscle, testis, mammary gland, adrenal gland, ovary, colon, normal cerebellum, normal adipose, normal skin, bone marrow, brain amygdala, brain hippocampus, brain substantia nigra, brain thalamus, thyroid, fetal lung, fetal liver, fetal brain, kidney, heart, spleen, uterus, pituitary gland, lymph node, salivary gland, small intestine, prostate, placenta, spinal cord, peripheral blood, trachea, stomach, pancreas, hypothalamus.

The reaction mixtures contained 2 microliters of each of the primers (original concentration: 5 pmol/ul), 1 microliter of 10 mM DNTP (Clontech Laboratories, Palo Alto Calif.) and 1 microliter of 50xAdvantage-HF 2 polymerase (Clontech Laboratories) in 50 microliter-reaction volume. The following reaction conditions were used:
PCR condition 1:
a) 96° C. 3 minutes
b) 96° C. 30 seconds denaturation
c) 60° C. 30 seconds, primer annealing
d) 72° C. 6 minutes extension
Repeat steps b-d 15 times
e) 96° C. 15 seconds denaturation
f) 60° C. 30 seconds, primer annealing
g) 72° C. 6 minutes extension
Repeat steps e-g 29 times
e) 72° C. 10 minutes final extension
PCR condition 2:
a) 96° C. 3 minutes
b) 96° C. 15 seconds denaturation
c) 76° C. 30 seconds, primer annealing, reducing the temperature by 1° C. per cycle
d) 72° C. 4 minutes extension
Repeat steps b-d 34 times
e) 72° C. 10 minutes final extension

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers:

| | | |
|---|---|---|
| SEQ ID NO:106: | SF1: | CCACCTTCATGAGTGACCACG |
| SEQ ID NO:107: | SF2: | ACTGTGCAGGTGCAGGTGGCAGGTAAG |
| SEQ ID NO:108: | SR1: | GAAGGTGGTCCTTCCTCTGTACT |
| SEQ ID NO:109: | SR2: | CGCCGAACTTTACACCATCCT |

The insert assemblies 174124888, 174124900, and 174124912 were all found to encode an open reading frame between residues 32 to 236 of the target sequence of CG50329-01. All of the assemblies have an 3 amino acid deletion as compared to the original sequence. 174124888 and 174124912 also differ from the original sequence at 3 nucleotide positions and 2 amino acid positions. 174124900 also differs from the original sequence at 2 nucleotide positions and 1 amino acid position.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, 32, and 34;
(b) a variant of a mature form of an amino acid sequence selected from the group consisting of SEQ ID NOS:2, 4, 6, 8, 10, 12, 14, 16, 18, 20, 22, 24, 26, 28, 30, 32, and 34, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS:2, 4, 6, 8, 10, 12, 14, 16, 18, 20, 22, 24, 26, 28, 30, 32, and 34; and
(d) a variant of an amino acid sequence selected from the group consisting of SEQ ID NOS:2, 4, 6, 8, 10, 12, 14, 16, 18, 20, 22, 24, 26, 28, 30, 32, and 34, wherein one or more amino acid residues in said variant differs from the amino acid sequence of said mature form, provided that said variant differs in no more than 15% of amino acid residues from said amino acid sequence.

2-4. (canceled)

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, 32, and 34;
(b) a variant of a mature form of an amino acid sequence selected from the group consisting of SEQ ID NOS:2, 4, 6, 8, 10, 12, 14, 16, 18, 20, 22, 24, 26, 28, 30, 32, and 34, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 26, 28, 30, 32, and 34;
(d) a variant of an amino acid sequence selected from the group consisting SEQ ID NOS:2, 4, 6, 8, 10, 12, 14, 16, 18, 20, 22, 24, 26, 28, 30, 32, 34, and 34, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 20, 22, 24, 26, 28, 30, 32, and 34, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6-8. (canceled)

9. The nucleic acid molecule of claim 5, wherein said nucleic acid molecule comprises a nucleotide sequence selected from the group consisting of:
(a) a nucleotide sequence selected from the group consisting of SEQ ID NOS:1, 3, 5, 7, 9, 11, 13, 15, 17, 19, 21, 23, 25, 27, 29, 31, 33, and 35;
(b) a nucleotide sequence differing by one or more nucleotides from a nucleotide sequence selected from the group consisting of SEQ ID NOS:1, 3, 5, 7, 9, 11, 13, 15, 17, 19, 21, 23, 25, 27, 29, 31, 33, and 35, provided that no more than 20% of the nucleotides differ from said nucleotide sequence;
(c) a nucleic acid fragment of (a); and
(d) a nucleic acid fragment of (b).

10. A complement of the nucleic acid molecule of claim 5, wherein said nucleic acid molecule hybridizes under stringent conditions to a nucleotide sequence chosen from the group consisting SEQ ID NOS:1, 3, 5, 7, 9, 11, 13, 15, 17, 19, 21, 23, 25, 27, 29, 31, 33, and 35, or a complement of said nucleotide sequence.

11. (canceled)

12. A vector comprising the nucleic acid molecule of claim 5.

13. The vector of claim 12, further comprising a promoter operably-linked to said nucleic acid molecule.

14. An isolated cell comprising the vector of claim 12.

15. An isolated antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20-33. (canceled)

34. A method of treating or preventing a NOVX-associated disorder, said method comprising administering to a subject in which such treatment or prevention is desired the antibody of claim 15 in an amount sufficient to treat or prevent said NOVX-associated disorder in said subject.

35-37. (canceled)

38. A composition comprising the polypeptide of claim 1 and a pharmaceutically-acceptable carrier.

39. A composition comprising the nucleic acid molecule of claim 5 and a pharmaceutically-acceptable carrier.

40. A composition comprising the antibody of claim 15 and a pharmaceutically-acceptable carrier.

41. A kit comprising in one or more containers, the composition of claim 38.

42. A kit comprising in one or more containers, the composition of claim 39.

43. A kit comprising in one or more containers, the composition of claim 40.

44-45. (canceled)

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 20, 22, 24, 26, 28, 30, 32, and 34, or a biologically active fragment thereof.

49. (canceled)
